SEQUENCE LISTING (632 sequences)

SEQ ID NO: 1 - RNA, 108 nt, synthetic construct
  misc_feature 1..108: note = source = /note="Description of Artificial Sequence: Synthetic polynucleotide"
  source 1..108: mol_type = other RNA
gctggtgctgcagcttattatgtgggtgtgtatctcacgaagcgaccctttggaaaatataatgaaaatggaaccattacagatgctgtagactgtgcacttgaccct

SEQ ID NO: 2 - RNA, 108 nt, synthetic construct
  misc_feature 1..108: note = source = /note="Description of Artificial Sequence: Synthetic polynucleotide"
  source 1..108: mol_type = other RNA
gctggtgctgcagcttattatgtgggtcctcgctaggacgtcgctatgacgccaaaatataatgaaaatggaaccattacagatgctgtagactgtgcacttgaccct

SEQ ID NO: 3 - RNA, 108 nt, synthetic construct
  misc_feature 1..108: note = source = /note="Description of Artificial Sequence: Synthetic polynucleotide"
  source 1..108: mol_type = other RNA
gctggtgctgcagcttattatgtgggtagcacgacttgatctaactgacactaaaaatataatgaaaatggaaccattacagatgctgtagactgtgcacttgaccct

SEQ ID NO: 4 - RNA, 108 nt, synthetic construct
  misc_feature 1..108: note = source = /note="Description of Artificial Sequence: Synthetic polynucleotide"
  source 1..108: mol_type = other RNA
gctggtgctgcagcttattatgtgggttaagtaggacttcgattggatggaataaaatataatgaaaatggaaccattacagatgctgtagactgtgcacttgaccct

SEQ ID NO: 5 - RNA, 82 nt, synthetic construct
  misc_feature 1..82: note = source = /note="Description of Artificial Sequence: Synthetic oligonucleotide"
  source 1..82: mol_type = other RNA
tctggttactgccagttgaatctgagggtccggacggatatcgcactaagtgtacctggtgcatttcgctgattttggggtc

SEQ ID NO: 6 - RNA, 82 nt, synthetic construct
  misc_feature 1..82: note = source = /note="Description of Artificial Sequence: Synthetic oligonucleotide"
  source 1..82: mol_type = other RNA
tctggttactgccagttgaatctgagggtcccatgaccatgtcactggctacactgaggtgcatttcgctgattttggggtc

SEQ ID NO: 7 - RNA, 82 nt, synthetic construct
  misc_feature 1..82: note = source = /note="Description of Artificial Sequence: Synthetic oligonucleotide"
  source 1..82: mol_type = other RNA
tctggttactgccagttgaatctgagggtccttgacatggcatgtgactccactgtcggtgcatttcgctgattttggggtc

SEQ ID NO: 8 - RNA, 82 nt, synthetic construct
  misc_feature 1..82: note = source = /note="Description of Artificial Sequence: Synthetic oligonucleotide"
  source 1..82: mol_type = other RNA
tctggttactgccagttgaatctgagggtccacctttgccagatgactgagtggaagggtgcatttcgctgattttggggtc

SEQ ID NO: 9 - RNA, 82 nt, synthetic construct
  misc_feature 1..82: note = source = /note="Description of Artificial Sequence: Synthetic oligonucleotide"
  source 1..82: mol_type = other RNA
tctggttactgccagttgaatctgagggtccagcttgaagcgttcgcgacaagtgtcggtgcatttcgctgattttggggtc

SEQ ID NO: 10 - RNA, 82 nt, synthetic construct
  misc_feature 1..82: note = source = /note="Description of Artificial Sequence: Synthetic oligonucleotide"
  source 1..82: mol_type = other RNA
tctggttactgccagttgaatctgagggtcctcacgtcctgagatcaactgctacatggtgcatttcgctgattttggggtc

SEQ ID NO: 11 - RNA, 82 nt, synthetic construct
  misc_feature 1..82: note = source = /note="Description of Artificial Sequence: Synthetic oligonucleotide"
  source 1..82: mol_type = other RNA
tctggttactgccagttgaatctgagggtccgttgactgatcacatgctgctccacgggtgcatttcgctgattttggggtc

SEQ ID NO: 12 - RNA, 82 nt, synthetic construct
  misc_feature 1..82: note = source = /note="Description of Artificial Sequence: Synthetic oligonucleotide"
  source 1..82: mol_type = other RNA
tctggttactgccagttgaatctgagggtcccagacagtcatcggattgatgagtgaggtgcatttcgctgattttggggtc

SEQ ID NO: 13 - DNA, 74 bp, synthetic construct
  misc_feature 1..74: note = source = /note="Description of Artificial Sequence: Synthetic oligonucleotide"
  source 1..74: mol_type = other DNA
taatacgactcactatagggctggtgctgcagcttattatgtgggtatagaacaacctaggacttttctattaa

SEQ ID NO: 14 - DNA, 26 bp, synthetic construct
  misc_feature 1..26: note = source = /note="Description of Artificial Sequence: Synthetic oligonucleotide"
  source 1..26: mol_type = other DNA
aacgtacactttgtttctgagagagg

SEQ ID NO: 15 - DNA, 37 bp, synthetic construct
  misc_feature 1..37: note = source = /note="Description of Artificial Sequence: Synthetic oligonucleotide"
  source 1..37: mol_type = other DNA
ctgacctgaaggctgacgccggacttgtggagacagc

SEQ ID NO: 16 - DNA, 59 bp, synthetic construct
  misc_feature 1..59: note = source = /note="Description of Artificial Sequence: Synthetic oligonucleotide"
  source 1..59: mol_type = other DNA
taatacgactcactatagggagatttggacctgcgagcgggttctgacctgaaggctga

SEQ ID NO: 17 - DNA, 39 bp, synthetic construct
  misc_feature 1..39: note = source = /note="Description of Artificial Sequence: Synthetic oligonucleotide"
  source 1..39: mol_type = other DNA
ggtttttcaatttcctgtttcttttccttaaagtcaacg

SEQ ID NO: 18 - DNA, 27 bp, synthetic construct
  misc_feature 1..27: note = source = /note="Description of Artificial Sequence: Synthetic primer"
  source 1..27: mol_type = other DNA
gctggtgctgcagcttattatgtgggt

SEQ ID NO: 19 - DNA, 20 bp, synthetic construct
  misc_feature 1..20: note = source = /note="Description of Artificial Sequence: Synthetic primer"
  source 1..20: mol_type = other DNA
agggtcaagtgcacagtcta

SEQ ID NO: 20 - DNA, 20 bp, synthetic construct
  misc_feature 1..20: note = source = /note="Description of Artificial Sequence: Synthetic primer"
  source 1..20: mol_type = other DNA
gaccccaaaatcagcgaaat

SEQ ID NO: 21 - DNA, 24 bp, synthetic construct
  misc_feature 1..24: note = source = /note="Description of Artificial Sequence: Synthetic primer"
  source 1..24: mol_type = other DNA
tctggttactgccagttgaatctg

SEQ ID NO: 22 - DNA, 53 bp, synthetic construct
  variation 25..34: note = a, c, t, g, unknown or other
  source 1..53: mol_type = other DNA; note = Description of Artificial Sequence: Synthetic oligonucleotide
caagcagaagacggcatacgagatnnnnnnnnnnaccccaaaatcagcgaaat

SEQ ID NO: 23 - DNA, 63 bp, synthetic construct
  source 1..63: mol_type = other DNA; note = Description of Artificial Sequence: Synthetic oligonucleotide
  variation 30..39: note = a, c, t, g, unknown or other
aatgatacggcgaccaccgagatctacacnnnnnnnnnntctggttactgccagttgaatctg

SEQ ID NO: 24 - DNA, 22 bp, synthetic construct
  misc_feature 1..22: note = source = /note="Description of Artificial Sequence: Synthetic primer"
  source 1..22: mol_type = other DNA
gaccaatcytgtcacctctgac

SEQ ID NO: 25 - DNA, 22 bp, synthetic construct
  misc_feature 1..22: note = source = /note="Description of Artificial Sequence: Synthetic primer"
  source 1..22: mol_type = other DNA
gaccaatyctgtcacctytgac

SEQ ID NO: 26 - DNA, 24 bp, synthetic construct
  misc_feature 1..24: note = source = /note="Description of Artificial Sequence: Synthetic primer"
  source 1..24: mol_type = other DNA
agggcattttggayaaagcgtcta

SEQ ID NO: 27 - DNA, 18 bp, synthetic construct
  misc_feature 1..18: note = source = /note="Description of Artificial Sequence: Synthetic primer"
  source 1..18: mol_type = other DNA
ttggggtcctcatcggag

SEQ ID NO: 28 - DNA, 21 bp, synthetic construct
  misc_feature 1..21: note = source = /note="Description of Artificial Sequence: Synthetic primer"
  source 1..21: mol_type = other DNA
ttctccaagcgaatctctgta

SEQ ID NO: 29 - DNA, 18 bp, synthetic construct
  misc_feature 1..18: note = source = /note="Description of Artificial Sequence: Synthetic primer"
  source 1..18: mol_type = other DNA
aagatggccatcggatcc

SEQ ID NO: 30 - DNA, 23 bp, synthetic construct
  misc_feature 1..23: note = source = /note="Description of Artificial Sequence: Synthetic primer"
  source 1..23: mol_type = other DNA
gtctccctcttctggtgataatc

SEQ ID NO: 31 - DNA, 106 bp, synthetic construct
  misc_feature 1..106: note = source = /note="Description of Artificial Sequence: Synthetic polynucleotide"
  source 1..106: mol_type = other DNA
gaccgatcctgtcacctctgactaagggtggcacaacgcagtgtgttgagctcccttagtagggtgactagcaccggcagcgtagacgctttgtccaaaatgccct

SEQ ID NO: 32 - DNA, 81 bp, synthetic construct
  misc_feature 1..81: note = source = /note="Description of Artificial Sequence: Synthetic oligonucleotide"
  source 1..81: mol_type = other DNA
ttggggtcctcatcggaggacgcttaaataatagtaggaacgttcgagtctctaaaaatatacagagattcgcttggagaa

SEQ ID NO: 33 - DNA, 140 bp, synthetic construct
  misc_feature 1..140: note = source = /note="Description of Artificial Sequence: Synthetic polynucleotide"
  source 1..140: mol_type = other DNA
aagatggccatcggatcctcaactcactcctgtaatgagtcgtagacaaggataagaggcccgatcggcagtatttcgccctttctaaaaataatgtgacctgggacgcactgcaccgattatcaccagaagagggagac

SEQ ID NO: 34-99 - skipped (66 entries)

SEQ ID NO: 100 - DNA, 25 bp, synthetic construct
  misc_feature 1..25: note = source = /note="Description of Artificial Sequence: Synthetic oligonucleotide"
  source 1..25: mol_type = other DNA
tggggtcttacacggcgatcttgcc

SEQ ID NO: 101 - DNA, 25 bp, synthetic construct
  misc_feature 1..25: note = source = /note="Description of Artificial Sequence: Synthetic oligonucleotide"
  source 1..25: mol_type = other DNA
gggtcaacgtgtcggcatggattct

SEQ ID NO: 102 - DNA, 28 bp, synthetic construct
  misc_feature 1..28: note = source = /note="Description of Artificial Sequence: Synthetic oligonucleotide"
  source 1..28: mol_type = other DNA
agttacattcacgccagttgtgtctggt

SEQ ID NO: 103 - DNA, 25 bp, synthetic construct
  misc_feature 1..25: note = source = /note="Description of Artificial Sequence: Synthetic oligonucleotide"
  source 1..25: mol_type = other DNA
agttacattcacgccagttgtggcg

SEQ ID NO: 104 - DNA, 26 bp, synthetic construct
  misc_feature 1..26: note = source = /note="Description of Artificial Sequence: Synthetic oligonucleotide"
  source 1..26: mol_type = other DNA
gtttgtaattacacggcgatcttgcc

SEQ ID NO: 105 - DNA, 28 bp, synthetic construct
  misc_feature 1..28: note = source = /note="Description of Artificial Sequence: Synthetic oligonucleotide"
  source 1..28: mol_type = other DNA
gtccaaatctttacacggcgatcttgcc

SEQ ID NO: 106 - DNA, 26 bp, synthetic construct
  misc_feature 1..26: note = source = /note="Description of Artificial Sequence: Synthetic oligonucleotide"
  source 1..26: mol_type = other DNA
agttacattcacgccagttgtggagc

SEQ ID NO: 107 - DNA, 43 bp, synthetic construct
  misc_feature 1..43: note = source = /note="Description of Artificial Sequence: Synthetic oligonucleotide"
  source 1..43: mol_type = other DNA
taatacgactcactatagggtctgataatggaccccaaaatca

SEQ ID NO: 108 - DNA, 21 bp, synthetic construct
  misc_feature 1..21: note = source = /note="Description of Artificial Sequence: Synthetic oligonucleotide"
  source 1..21: mol_type = other DNA
ttaggcctgagttgagtcagc

SEQ ID NO: 109 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcgaccccaaaatcagcgaaat

SEQ ID NO: 110 - DNA, 61 bp, synthetic construct
  misc_feature 1..61: note = source = /note="Description of Artificial Sequence: Synthetic oligonucleotide"
  source 1..61: mol_type = other DNA
aatgatacggcgaccaccgagatctacacaagatctgtctggttactgccagttgaatctg

SEQ ID NO: 111 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcttacaaacattggccgcaaa

SEQ ID NO: 112 - DNA, 55 bp, synthetic construct
  misc_feature 1..55: note = source = /note="Description of Artificial Sequence: Synthetic oligonucleotide"
  source 1..55: mol_type = other DNA
aatgatacggcgaccaccgagatctacacaagatctggcgcgacattccgaagaa

SEQ ID NO: 113 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgttctatcagatttggacctgcgagcg

SEQ ID NO: 114 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctggagcggctgtctccacaagt

SEQ ID NO: 115 - DNA, 54 bp, synthetic construct
  misc_feature 1..54: note = source = /note="Description of Artificial Sequence: Synthetic oligonucleotide"
  source 1..54: mol_type = other DNA
caagcagaagacggcatacgagatgttctatctaatcagacaaggaactgatta

SEQ ID NO: 116 - DNA, 56 bp, synthetic construct
  misc_feature 1..56: note = source = /note="Description of Artificial Sequence: Synthetic oligonucleotide"
  source 1..56: mol_type = other DNA
aatgatacggcgaccaccgagatctacacaagatctgcgaaggtgtgacttccatg

SEQ ID NO: 117 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctggatcaaaacaacgtcggccc

SEQ ID NO: 118 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcccatgttgagtgagagcggt

SEQ ID NO: 119 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctgtggaccccaaaatcagcgaa

SEQ ID NO: 120 - DNA, 53 bp, synthetic construct
  misc_feature 1..53: note = source = /note="Description of Artificial Sequence: Synthetic oligonucleotide"
  source 1..53: mol_type = other DNA
caagcagaagacggcatacgagatgttctatcactgcgttctccattctggtt

SEQ ID NO: 121 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctgcagcgttcttcggaatgtcg

SEQ ID NO: 122 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcgcacctgtgtaggtcaacca

SEQ ID NO: 123 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctggaaatgcaccccgcattacg

SEQ ID NO: 124 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatccccactgcgttctccattct

SEQ ID NO: 125 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctggtcttggttcaccgctctca

SEQ ID NO: 126 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgttctatcttggaacgccttgtcctcg

SEQ ID NO: 127 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctggcagtcaagcctcttctcgt

SEQ ID NO: 128 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcgaagttcccctactgctgcc

SEQ ID NO: 129 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctgcgtttggtggaccctcagat

SEQ ID NO: 130 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcgacgttgttttgatcgcgcc

SEQ ID NO: 131 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctgaaggccaacaacaacaaggc

SEQ ID NO: 132 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcggcagtacgtttttgccgag

SEQ ID NO: 133 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctgaccagaatggagaacgcagt

SEQ ID NO: 134 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatccggtgaaccaagacgcagta

SEQ ID NO: 135 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctgccgcattacgtttggtggac

SEQ ID NO: 136 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcggccgacgttgttttgatcg

SEQ ID NO: 137 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctggcctcggcaaaaacgtactg

SEQ ID NO: 138 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcttgttctggaccacgtctgc

SEQ ID NO: 139 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctgaattccctcgaggacaaggc

SEQ ID NO: 140 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatctcgtctggtagctcttcggt

SEQ ID NO: 141 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctggcttcagcgttcttcggaat

SEQ ID NO: 142 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatctggcacctgtgtaggtcaac

SEQ ID NO: 143 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctgagaatagagctcgcaccgta

SEQ ID NO: 144 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcctcctctagtggcggctatt

SEQ ID NO: 145 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctggctggtgctgcagcttatta

SEQ ID NO: 146 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcagggtcaagtgcacagtcta

SEQ ID NO: 147 - DNA, 59 bp, synthetic construct
  misc_feature 1..59: note = source = /note="Description of Artificial Sequence: Synthetic oligonucleotide"
  source 1..59: mol_type = other DNA
aatgatacggcgaccaccgagatctacacaagatctgttcggaagagacaggtacgtta

SEQ ID NO: 148 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgttctatcagcagtacgcacacaatcg

SEQ ID NO: 149 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacaagatctgcaatgctgcaatcgtgctac

SEQ ID NO: 150 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttctatcgttgcgactacgtgatgagg

SEQ ID NO: 151 - DNA, 19 bp, synthetic construct
  misc_feature 1..19: note = source = /note="Description of Artificial Sequence: Synthetic oligonucleotide"
  source 1..19: mol_type = other DNA
agatttggacctgcgagcg

SEQ ID NO: 152 - DNA, 20 bp, synthetic construct
  misc_feature 1..20: note = source = /note="Description of Artificial Sequence: Synthetic oligonucleotide"
  source 1..20: mol_type = other DNA
gagcggctgtctccacaagt

SEQ ID NO: 153 - DNA, 20 bp, synthetic construct
  misc_feature 1..20: note = source = /note="Description of Artificial Sequence: Synthetic oligonucleotide"
  source 1..20: mol_type = other DNA
agaatagagctcgcaccgta

SEQ ID NO: 154 - DNA, 20 bp, synthetic construct
  misc_feature 1..20: note = source = /note="Description of Artificial Sequence: Synthetic oligonucleotide"
  source 1..20: mol_type = other DNA
ctcctctagtggcggctatt

SEQ ID NO: 155 - DNA, 20 bp, synthetic construct
  misc_feature 1..20: note = source = /note="Description of Artificial Sequence: Synthetic oligonucleotide"
  source 1..20: mol_type = other DNA
gctggtgctgcagcttatta

SEQ ID NO: 156 - DNA, 20 bp, synthetic construct
  misc_feature 1..20: note = source = /note="Description of Artificial Sequence: Synthetic oligonucleotide"
  source 1..20: mol_type = other DNA
agggtcaagtgcacagtcta

SEQ ID NO: 157 - DNA, 22 bp, synthetic construct
  misc_feature 1..22: note = source = /note="Description of Artificial Sequence: Synthetic oligonucleotide"
  source 1..22: mol_type = other DNA
ttcggaagagacaggtacgtta

SEQ ID NO: 158 - DNA, 19 bp, synthetic construct
  misc_feature 1..19: note = source = /note="Description of Artificial Sequence: Synthetic oligonucleotide"
  source 1..19: mol_type = other DNA
agcagtacgcacacaatcg

SEQ ID NO: 159 - DNA, 20 bp, synthetic construct
  misc_feature 1..20: note = source = /note="Description of Artificial Sequence: Synthetic oligonucleotide"
  source 1..20: mol_type = other DNA
caatgctgcaatcgtgctac

SEQ ID NO: 160 - DNA, 20 bp, synthetic construct
  misc_feature 1..20: note = source = /note="Description of Artificial Sequence: Synthetic oligonucleotide"
  source 1..20: mol_type = other DNA
gttgcgactacgtgatgagg

SEQ ID NO: 161 - DNA, 20 bp, synthetic construct
  misc_feature 1..20: note = source = /note="Description of Artificial Sequence: Synthetic oligonucleotide"
  source 1..20: mol_type = other DNA
gaccccaaaatcagcgaaat

SEQ ID NO: 162 - DNA, 24 bp, synthetic construct
  misc_feature 1..24: note = source = /note="Description of Artificial Sequence: Synthetic oligonucleotide"
  source 1..24: mol_type = other DNA
tctggttactgccagttgaatctg

SEQ ID NO: 163 - DNA, 20 bp, synthetic construct
  misc_feature 1..20: note = source = /note="Description of Artificial Sequence: Synthetic oligonucleotide"
  source 1..20: mol_type = other DNA
ttacaaacattggccgcaaa

SEQ ID NO: 164 - DNA, 18 bp, synthetic construct
  misc_feature 1..18: note = source = /note="Description of Artificial Sequence: Synthetic oligonucleotide"
  source 1..18: mol_type = other DNA
gcgcgacattccgaagaa

SEQ ID NO: 165 - DNA, 22 bp, synthetic construct
  misc_feature 1..22: note = source = /note="Description of Artificial Sequence: Synthetic oligonucleotide"
  source 1..22: mol_type = other DNA
taatcagacaaggaactgatta

SEQ ID NO: 166 - DNA, 19 bp, synthetic construct
  misc_feature 1..19: note = source = /note="Description of Artificial Sequence: Synthetic oligonucleotide"
  source 1..19: mol_type = other DNA
cgaaggtgtgacttccatg

SEQ ID NO: 167 - DNA, 20 bp, synthetic construct
  misc_feature 1..20: note = source = /note="Description of Artificial Sequence: Synthetic oligonucleotide"
  source 1..20: mol_type = other DNA
gatcaaaacaacgtcggccc

SEQ ID NO: 168 - DNA, 20 bp, synthetic construct
  misc_feature 1..20: note = source = /note="Description of Artificial Sequence: Synthetic oligonucleotide"
  source 1..20: mol_type = other DNA
ccatgttgagtgagagcggt

SEQ ID NO: 169 - DNA, 20 bp, synthetic construct
  misc_feature 1..20: note = source = /note="Description of Artificial Sequence: Synthetic oligonucleotide"
  source 1..20: mol_type = other DNA
tggaccccaaaatcagcgaa

SEQ ID NO: 170 - DNA, 21 bp, synthetic construct
  misc_feature 1..21: note = source = /note="Description of Artificial Sequence: Synthetic oligonucleotide"
  source 1..21: mol_type = other DNA
actgcgttctccattctggtt

SEQ ID NO: 171 - DNA, 20 bp, synthetic construct
  misc_feature 1..20: note = source = /note="Description of Artificial Sequence: Synthetic oligonucleotide"
  source 1..20: mol_type = other DNA
cagcgttcttcggaatgtcg

SEQ ID NO: 172 - DNA, 20 bp, synthetic construct
  misc_feature 1..20: note = source = /note="Description of Artificial Sequence: Synthetic oligonucleotide"
  source 1..20: mol_type = other DNA
gcacctgtgtaggtcaacca

SEQ ID NO: 173 - DNA, 20 bp, synthetic construct
  misc_feature 1..20: note = source = /note="Description of Artificial Sequence: Synthetic oligonucleotide"
  source 1..20: mol_type = other DNA
gaaatgcaccccgcattacg

SEQ ID NO: 174 - DNA, 20 bp, synthetic construct
  misc_feature 1..20: note = source = /note="Description of Artificial Sequence: Synthetic oligonucleotide"
  source 1..20: mol_type = other DNA
cccactgcgttctccattct

SEQ ID NO: 175 - DNA, 20 bp, synthetic construct
  misc_feature 1..20: note = source = /note="Description of Artificial Sequence: Synthetic oligonucleotide"
  source 1..20: mol_type = other DNA
gtcttggttcaccgctctca

SEQ ID NO: 176 - DNA, 19 bp, synthetic construct
  misc_feature 1..19: note = source = /note="Description of Artificial Sequence: Synthetic oligonucleotide"
  source 1..19: mol_type = other DNA
ttggaacgccttgtcctcg

SEQ ID NO: 177 - DNA, 20 bp, synthetic construct
  misc_feature 1..20: note = source = /note="Description of Artificial Sequence: Synthetic oligonucleotide"
  source 1..20: mol_type = other DNA
gcagtcaagcctcttctcgt

SEQ ID NO: 178 - DNA, 20 bp, synthetic construct
  misc_feature 1..20: note = source = /note="Description of Artificial Sequence: Synthetic oligonucleotide"
  source 1..20: mol_type = other DNA
gaagttcccctactgctgcc

SEQ ID NO: 179 - DNA, 20 bp, synthetic construct
  misc_feature 1..20: note = source = /note="Description of Artificial Sequence: Synthetic oligonucleotide"
  source 1..20: mol_type = other DNA
cgtttggtggaccctcagat

SEQ ID NO: 180 - DNA, 20 bp, synthetic construct
  misc_feature 1..20: note = source = /note="Description of Artificial Sequence: Synthetic oligonucleotide"
  source 1..20: mol_type = other DNA
gacgttgttttgatcgcgcc

SEQ ID NO: 181 - DNA, 20 bp, synthetic construct
  misc_feature 1..20: note = source = /note="Description of Artificial Sequence: Synthetic oligonucleotide"
  source 1..20: mol_type = other DNA
aaggccaacaacaacaaggc

SEQ ID NO: 182 - DNA, 20 bp, synthetic construct
  misc_feature 1..20: note = source = /note="Description of Artificial Sequence: Synthetic oligonucleotide"
  source 1..20: mol_type = other DNA
ggcagtacgtttttgccgag

SEQ ID NO: 183 - DNA, 20 bp, synthetic construct
  misc_feature 1..20: note = source = /note="Description of Artificial Sequence: Synthetic oligonucleotide"
  source 1..20: mol_type = other DNA
accagaatggagaacgcagt

SEQ ID NO: 184 - DNA, 20 bp, synthetic construct
  misc_feature 1..20: note = source = /note="Description of Artificial Sequence: Synthetic oligonucleotide"
  source 1..20: mol_type = other DNA
cggtgaaccaagacgcagta

SEQ ID NO: 185 - DNA, 20 bp, synthetic construct
  misc_feature 1..20: note = source = /note="Description of Artificial Sequence: Synthetic oligonucleotide"
  source 1..20: mol_type = other DNA
ccgcattacgtttggtggac

SEQ ID NO: 186 - DNA, 20 bp, synthetic construct
  misc_feature 1..20: note = source = /note="Description of Artificial Sequence: Synthetic oligonucleotide"
  source 1..20: mol_type = other DNA
ggccgacgttgttttgatcg

SEQ ID NO: 187 - DNA, 20 bp, synthetic construct
  misc_feature 1..20: note = source = /note="Description of Artificial Sequence: Synthetic oligonucleotide"
  source 1..20: mol_type = other DNA
gcctcggcaaaaacgtactg

SEQ ID NO: 188 - DNA, 20 bp, synthetic construct
  misc_feature 1..20: note = source = /note="Description of Artificial Sequence: Synthetic oligonucleotide"
  source 1..20: mol_type = other DNA
ttgttctggaccacgtctgc

SEQ ID NO: 189 - DNA, 20 bp, synthetic construct
  misc_feature 1..20: note = source = /note="Description of Artificial Sequence: Synthetic oligonucleotide"
  source 1..20: mol_type = other DNA
aattccctcgaggacaaggc

SEQ ID NO: 190 - DNA, 20 bp, synthetic construct
  misc_feature 1..20: note = source = /note="Description of Artificial Sequence: Synthetic oligonucleotide"
  source 1..20: mol_type = other DNA
tcgtctggtagctcttcggt

SEQ ID NO: 191 - DNA, 20 bp, synthetic construct
  misc_feature 1..20: note = source = /note="Description of Artificial Sequence: Synthetic oligonucleotide"
  source 1..20: mol_type = other DNA
gcttcagcgttcttcggaat

SEQ ID NO: 192 - DNA, 20 bp, synthetic construct
  misc_feature 1..20: note = source = /note="Description of Artificial Sequence: Synthetic oligonucleotide"
  source 1..20: mol_type = other DNA
tggcacctgtgtaggtcaac

SEQ ID NO: 193 - DNA, 42 bp, synthetic construct
  misc_feature 1..42: note = source = /note="Description of Artificial Sequence: Synthetic oligonucleotide"
  source 1..42: mol_type = other DNA
gggtcacgcgtaggagttctatcagatttggacctgcgagcg

SEQ ID NO: 194 - DNA, 43 bp, synthetic construct
  misc_feature 1..43: note = source = /note="Description of Artificial Sequence: Synthetic oligonucleotide"
  source 1..43: mol_type = other DNA
gttccgcagccacacaagatctggagcggctgtctccacaagt

SEQ ID NO: 195 - DNA, 42 bp, synthetic construct
  misc_feature 1..42: note = source = /note="Description of Artificial Sequence: Synthetic oligonucleotide"
  source 1..42: mol_type = other DNA
cgcgtcgagtagggtgttctatcagatttggacctgcgagcg

SEQ ID NO: 196 - DNA, 43 bp, synthetic construct
  misc_feature 1..43: note = source = /note="Description of Artificial Sequence: Synthetic oligonucleotide"
  source 1..43: mol_type = other DNA
gccgtgtgaagctggaagatctggagcggctgtctccacaagt

SEQ ID NO: 197 - DNA, 42 bp, synthetic construct
  misc_feature 1..42: note = source = /note="Description of Artificial Sequence: Synthetic oligonucleotide"
  source 1..42: mol_type = other DNA
cgatcgcccttggtggttctatcagatttggacctgcgagcg

SEQ ID NO: 198 - DNA, 43 bp, synthetic construct
  misc_feature 1..43: note = source = /note="Description of Artificial Sequence: Synthetic oligonucleotide"
  source 1..43: mol_type = other DNA
ggtttagccggcgtgaagatctggagcggctgtctccacaagt

SEQ ID NO: 199 - DNA, 42 bp, synthetic construct
  misc_feature 1..42: note = source = /note="Description of Artificial Sequence: Synthetic oligonucleotide"
  source 1..42: mol_type = other DNA
ggtcgagccggaactgttctatcagatttggacctgcgagcg

SEQ ID NO: 200 - DNA, 43 bp, synthetic construct
  misc_feature 1..43: note = source = /note="Description of Artificial Sequence: Synthetic oligonucleotide"
  source 1..43: mol_type = other DNA
ggatgcgcacccagaaagatctggagcggctgtctccacaagt

SEQ ID NO: 201 - DNA, 42 bp, synthetic construct
  misc_feature 1..42: note = source = /note="Description of Artificial Sequence: Synthetic oligonucleotide"
  source 1..42: mol_type = other DNA
tcccggcgttgtcctgttctatcagatttggacctgcgagcg

SEQ ID NO: 202 - DNA, 43 bp, synthetic construct
  misc_feature 1..43: note = source = /note="Description of Artificial Sequence: Synthetic oligonucleotide"
  source 1..43: mol_type = other DNA
gctccgtcactgcccaagatctggagcggctgtctccacaagt

SEQ ID NO: 203 - DNA, 42 bp, synthetic construct
  misc_feature 1..42: note = source = /note="Description of Artificial Sequence: Synthetic oligonucleotide"
  source 1..42: mol_type = other DNA
cgcagggtccagagtgttctatcagatttggacctgcgagcg

SEQ ID NO: 204 - DNA, 43 bp, synthetic construct
  misc_feature 1..43: note = source = /note="Description of Artificial Sequence: Synthetic oligonucleotide"
  source 1..43: mol_type = other DNA
gttcgcgcgaaggaaaagatctggagcggctgtctccacaagt

SEQ ID NO: 205 - DNA, 47 bp, synthetic construct
  misc_feature 1..47: note = source = /note="Description of Artificial Sequence: Synthetic oligonucleotide"
  source 1..47: mol_type = other DNA
atatagatgccgtcctagcggttctatcagatttggacctgcgagcg

SEQ ID NO: 206 - DNA, 48 bp, synthetic construct
  misc_feature 1..48: note = source = /note="Description of Artificial Sequence: Synthetic oligonucleotide"
  source 1..48: mol_type = other DNA
aagtatctttcctgtgcccaaagatctggagcggctgtctccacaagt

SEQ ID NO: 207 - DNA, 47 bp, synthetic construct
  misc_feature 1..47: note = source = /note="Description of Artificial Sequence: Synthetic oligonucleotide"
  source 1..47: mol_type = other DNA
ccctttaatcagatgcgtcggttctatcagatttggacctgcgagcg

SEQ ID NO: 208 - DNA, 48 bp, synthetic construct
  misc_feature 1..48: note = source = /note="Description of Artificial Sequence: Synthetic oligonucleotide"
  source 1..48: mol_type = other DNA
tggtagtaataagggcgaccaagatctggagcggctgtctccacaagt

SEQ ID NO: 209 - DNA, 47 bp, synthetic construct
  misc_feature 1..47: note = source = /note="Description of Artificial Sequence: Synthetic oligonucleotide"
  source 1..47: mol_type = other DNA
ttggtcatgtgcttttcgttgttctatcagatttggacctgcgagcg

SEQ ID NO: 210 - DNA, 48 bp, synthetic construct
  misc_feature 1..48: note = source = /note="Description of Artificial Sequence: Synthetic oligonucleotide"
  source 1..48: mol_type = other DNA
aggggtatcggatactcagaaagatctggagcggctgtctccacaagt

SEQ ID NO: 211 - DNA, 47 bp, synthetic construct
  misc_feature 1..47: note = source = /note="Description of Artificial Sequence: Synthetic oligonucleotide"
  source 1..47: mol_type = other DNA
gggtgggtaaatggtaatgcgttctatcagatttggacctgcgagcg

SEQ ID NO: 212 - DNA, 48 bp, synthetic construct
  misc_feature 1..48: note = source = /note="Description of Artificial Sequence: Synthetic oligonucleotide"
  source 1..48: mol_type = other DNA
atcgattccccggatatagcaagatctggagcggctgtctccacaagt

SEQ ID NO: 213 - DNA, 47 bp, synthetic construct
  misc_feature 1..47: note = source = /note="Description of Artificial Sequence: Synthetic oligonucleotide"
  source 1..47: mol_type = other DNA
tccgacggggagtatatactgttctatcagatttggacctgcgagcg

SEQ ID NO: 214 - DNA, 48 bp, synthetic construct
  misc_feature 1..48: note = source = /note="Description of Artificial Sequence: Synthetic oligonucleotide"
  source 1..48: mol_type = other DNA
tactaactgcttcaggccaaaagatctggagcggctgtctccacaagt

SEQ ID NO: 215 - DNA, 47 bp, synthetic construct
  misc_feature 1..47: note = source = /note="Description of Artificial Sequence: Synthetic oligonucleotide"
  source 1..47: mol_type = other DNA
catgtttaggaacgctaccggttctatcagatttggacctgcgagcg

SEQ ID NO: 216 - DNA, 48 bp, synthetic construct
  misc_feature 1..48: note = source = /note="Description of Artificial Sequence: Synthetic oligonucleotide"
  source 1..48: mol_type = other DNA
aataatctccgttccctcccaagatctggagcggctgtctccacaagt

SEQ ID NO: 217 - DNA, 79 bp, synthetic construct
  misc_feature 1..79: note = source = /note="Description of Artificial Sequence: Synthetic oligonucleotide"
  source 1..79: mol_type = other DNA
taatacgactcactatagggaccccaaaatcagcgaaatgcaccccgcattacgaaaccaggaccctcagattcaactg

SEQ ID NO: 218 - DNA, 22 bp, synthetic construct
  misc_feature 1..22: note = source = /note="Description of Artificial Sequence: Synthetic oligonucleotide"
  source 1..22: mol_type = other DNA
cgcagtattattgggtaaacct

SEQ ID NO: 219 - DNA, 74 bp, synthetic construct; identical to SEQ ID NO: 13
  misc_feature 1..74: note = source = /note="Description of Artificial Sequence: Synthetic oligonucleotide"
  source 1..74: mol_type = other DNA

SEQ ID NO: 220 - DNA, 26 bp, synthetic construct
  misc_feature 1..26: note = source = /note="Description of Artificial Sequence: Synthetic oligonucleotide"
  source 1..26: mol_type = other DNA
aacgtacactttgtttctgagagagg

SEQ ID NO: 221 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgagtcttcgaccccaaaatcagcgaaat

SEQ ID NO: 222 - DNA, 52 bp, synthetic construct; identical to SEQ ID NO: 109
  misc_feature 1..52: note = source = /note="Description of Artificial Sequence: Synthetic oligonucleotide"
  source 1..52: mol_type = other DNA

SEQ ID NO: 223 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattgggccaagaccccaaaatcagcgaaat

SEQ ID NO: 224 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatattgttgggaccccaaaatcagcgaaat

SEQ ID NO: 225 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcccgttggaccccaaaatcagcgaaat

SEQ ID NO: 226 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatacacacttgaccccaaaatcagcgaaat

SEQ ID NO: 227 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatccattccagaccccaaaatcagcgaaat

SEQ ID NO: 228 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctaacggggaccccaaaatcagcgaaat

SEQ ID NO: 229 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatccataggagaccccaaaatcagcgaaat

SEQ ID NO: 230 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcagtgtaggaccccaaaatcagcgaaat

SEQ ID NO: 231 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgattctcagaccccaaaatcagcgaaat

SEQ ID NO: 232 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatccttcttagaccccaaaatcagcgaaat

SEQ ID NO: 233 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattctaagacgaccccaaaatcagcgaaat

SEQ ID NO: 234 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgcggcatagaccccaaaatcagcgaaat

SEQ ID NO: 235 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatattgacgagaccccaaaatcagcgaaat

SEQ ID NO: 236 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgctcctgagaccccaaaatcagcgaaat

SEQ ID NO: 237 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgcaatcctgaccccaaaatcagcgaaat

SEQ ID NO: 238 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatatgtcgttgaccccaaaatcagcgaaat

SEQ ID NO: 239 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatttctcggcgaccccaaaatcagcgaaat

SEQ ID NO: 240 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcagggctagaccccaaaatcagcgaaat

SEQ ID NO: 241 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagccaagcgaccccaaaatcagcgaaat

SEQ ID NO: 242 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataagcctgagaccccaaaatcagcgaaat

SEQ ID NO: 243 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctacagaggaccccaaaatcagcgaaat

SEQ ID NO: 244 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgtagtcggaccccaaaatcagcgaaat

SEQ ID NO: 245 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatttctgctcgaccccaaaatcagcgaaat

SEQ ID NO: 246 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtgcacacgaccccaaaatcagcgaaat

SEQ ID NO: 247 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataaagctcagaccccaaaatcagcgaaat

SEQ ID NO: 248 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgacctcaggaccccaaaatcagcgaaat

SEQ ID NO: 249 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctttccaagaccccaaaatcagcgaaat

SEQ ID NO: 250 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcttggctgaccccaaaatcagcgaaat

SEQ ID NO: 251 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgcgtctagaccccaaaatcagcgaaat

SEQ ID NO: 252 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcgcgctagaccccaaaatcagcgaaat

SEQ ID NO: 253 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatatccattcgaccccaaaatcagcgaaat

SEQ ID NO: 254 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgcccagtagaccccaaaatcagcgaaat

SEQ ID NO: 255 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattaccgacggaccccaaaatcagcgaaat

SEQ ID NO: 256 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattccatacggaccccaaaatcagcgaaat

SEQ ID NO: 257 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataacatgtcgaccccaaaatcagcgaaat

SEQ ID NO: 258 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgactatagaccccaaaatcagcgaaat

SEQ ID NO: 259 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatacccaaaggaccccaaaatcagcgaaat

SEQ ID NO: 260 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatatcgatcggaccccaaaatcagcgaaat

SEQ ID NO: 261 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttggatggaccccaaaatcagcgaaat

SEQ ID NO: 262 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctatgtgagaccccaaaatcagcgaaat

SEQ ID NO: 263 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattatttcgcgaccccaaaatcagcgaaat

SEQ ID NO: 264 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatccatgtatgaccccaaaatcagcgaaat

SEQ ID NO: 265 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgccacgttgaccccaaaatcagcgaaat

SEQ ID NO: 266 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtcgtgtagaccccaaaatcagcgaaat

SEQ ID NO: 267 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattaaagtcggaccccaaaatcagcgaaat

SEQ ID NO: 268 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcttcggacgaccccaaaatcagcgaaat

SEQ ID NO: 269 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgcactctcgaccccaaaatcagcgaaat

SEQ ID NO: 270 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcagatacgaccccaaaatcagcgaaat

SEQ ID NO: 271 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcagtccctgaccccaaaatcagcgaaat

SEQ ID NO: 272 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgccctaacgaccccaaaatcagcgaaat

SEQ ID NO: 273 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctgcatcagaccccaaaatcagcgaaat

SEQ ID NO: 274 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcggtatcggaccccaaaatcagcgaaat

SEQ ID NO: 275 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataagtatgggaccccaaaatcagcgaaat

SEQ ID NO: 276 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatattcgcgcgaccccaaaatcagcgaaat

SEQ ID NO: 277 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatatcaaggtgaccccaaaatcagcgaaat

SEQ ID NO: 278 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatttgtgcatgaccccaaaatcagcgaaat

SEQ ID NO: 279 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctgtgctggaccccaaaatcagcgaaat

SEQ ID NO: 280 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtccgtaggaccccaaaatcagcgaaat

SEQ ID NO: 281 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttcaagagaccccaaaatcagcgaaat

SEQ ID NO: 282 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcaccgttcgaccccaaaatcagcgaaat

SEQ ID NO: 283 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgagttgagaccccaaaatcagcgaaat

SEQ ID NO: 284 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgagcacgagaccccaaaatcagcgaaat

SEQ ID NO: 285 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagttcgtggaccccaaaatcagcgaaat

SEQ ID NO: 286 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcatcaactgaccccaaaatcagcgaaat

SEQ ID NO: 287 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgagatctgaccccaaaatcagcgaaat

SEQ ID NO: 288 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattggccagagaccccaaaatcagcgaaat

SEQ ID NO: 289 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatttcaccatgaccccaaaatcagcgaaat

SEQ ID NO: 290 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgaatgcatgaccccaaaatcagcgaaat

SEQ ID NO: 291 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattggaccctgaccccaaaatcagcgaaat

SEQ ID NO: 292 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgatagcacgaccccaaaatcagcgaaat

SEQ ID NO: 293 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatacgacgacgaccccaaaatcagcgaaat

SEQ ID NO: 294 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctcagtatgaccccaaaatcagcgaaat

SEQ ID NO: 295 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcttagctagaccccaaaatcagcgaaat

SEQ ID NO: 296 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctgtttacgaccccaaaatcagcgaaat

SEQ ID NO: 297 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattgtcccacgaccccaaaatcagcgaaat

SEQ ID NO: 298 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcctgagggaccccaaaatcagcgaaat

SEQ ID NO: 299 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattagttccagaccccaaaatcagcgaaat

SEQ ID NO: 300 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcatgactcgaccccaaaatcagcgaaat

SEQ ID NO: 301 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtaagcgcgaccccaaaatcagcgaaat

SEQ ID NO: 302 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataacccagtgaccccaaaatcagcgaaat

SEQ ID NO: 303 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattttgaggggaccccaaaatcagcgaaat

SEQ ID NO: 304 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagccgacagaccccaaaatcagcgaaat

SEQ ID NO: 305 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataaacccgcgaccccaaaatcagcgaaat

SEQ ID NO: 306 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtagggctgaccccaaaatcagcgaaat

SEQ ID NO: 307 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagacgattgaccccaaaatcagcgaaat

SEQ ID NO: 308 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataggatgatgaccccaaaatcagcgaaat

SEQ ID NO: 309 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatataatggcgaccccaaaatcagcgaaat

SEQ ID NO: 310 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcttggcgtgaccccaaaatcagcgaaat

SEQ ID NO: 311 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagctgtgcgaccccaaaatcagcgaaat

SEQ ID NO: 312 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgagtccaagaccccaaaatcagcgaaat

SEQ ID NO: 313 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgaataccagaccccaaaatcagcgaaat

SEQ ID NO: 314 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataggagcttgaccccaaaatcagcgaaat

SEQ ID NO: 315 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtgacttagaccccaaaatcagcgaaat

SEQ ID NO: 316 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattttggaacgaccccaaaatcagcgaaat

SEQ ID NO: 317 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgagtcttcagggtcaagtgcacagtcta

SEQ ID NO: 318 - DNA, 52 bp, synthetic construct; identical to SEQ ID NO: 146
  misc_feature 1..52: note = source = /note="Description of Artificial Sequence: Synthetic oligonucleotide"
  source 1..52: mol_type = other DNA

SEQ ID NO: 319 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattgggccaaagggtcaagtgcacagtcta

SEQ ID NO: 320 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatattgttggagggtcaagtgcacagtcta

SEQ ID NO: 321 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcccgttgagggtcaagtgcacagtcta

SEQ ID NO: 322 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatacacacttagggtcaagtgcacagtcta

SEQ ID NO: 323 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatccattccaagggtcaagtgcacagtcta

SEQ ID NO: 324 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctaacgggagggtcaagtgcacagtcta

SEQ ID NO: 325 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatccataggaagggtcaagtgcacagtcta

SEQ ID NO: 326 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcagtgtagagggtcaagtgcacagtcta

SEQ ID NO: 327 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgattctcaagggtcaagtgcacagtcta

SEQ ID NO: 328 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatccttcttaagggtcaagtgcacagtcta

SEQ ID NO: 329 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattctaagacagggtcaagtgcacagtcta

SEQ ID NO: 330 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgcggcataagggtcaagtgcacagtcta

SEQ ID NO: 331 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatattgacgaagggtcaagtgcacagtcta

SEQ ID NO: 332 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgctcctgaagggtcaagtgcacagtcta

SEQ ID NO: 333 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgcaatcctagggtcaagtgcacagtcta

SEQ ID NO: 334 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatatgtcgttagggtcaagtgcacagtcta

SEQ ID NO: 335 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatttctcggcagggtcaagtgcacagtcta

SEQ ID NO: 336 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcagggctaagggtcaagtgcacagtcta

SEQ ID NO: 337 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagccaagcagggtcaagtgcacagtcta

SEQ ID NO: 338 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataagcctgaagggtcaagtgcacagtcta

SEQ ID NO: 339 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctacagagagggtcaagtgcacagtcta

SEQ ID NO: 340 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgtagtcgagggtcaagtgcacagtcta

SEQ ID NO: 341 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatttctgctcagggtcaagtgcacagtcta

SEQ ID NO: 342 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtgcacacagggtcaagtgcacagtcta

SEQ ID NO: 343 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataaagctcaagggtcaagtgcacagtcta

SEQ ID NO: 344 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgacctcagagggtcaagtgcacagtcta

SEQ ID NO: 345 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctttccaaagggtcaagtgcacagtcta

SEQ ID NO: 346 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcttggctagggtcaagtgcacagtcta

SEQ ID NO: 347 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgcgtctaagggtcaagtgcacagtcta

SEQ ID NO: 348 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcgcgctaagggtcaagtgcacagtcta

SEQ ID NO: 349 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatatccattcagggtcaagtgcacagtcta

SEQ ID NO: 350 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgcccagtaagggtcaagtgcacagtcta

SEQ ID NO: 351 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattaccgacgagggtcaagtgcacagtcta

SEQ ID NO: 352 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattccatacgagggtcaagtgcacagtcta

SEQ ID NO: 353 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataacatgtcagggtcaagtgcacagtcta

SEQ ID NO: 354 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgactataagggtcaagtgcacagtcta

SEQ ID NO: 355 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatacccaaagagggtcaagtgcacagtcta

SEQ ID NO: 356 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatatcgatcgagggtcaagtgcacagtcta

SEQ ID NO: 357 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttggatgagggtcaagtgcacagtcta

SEQ ID NO: 358 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctatgtgaagggtcaagtgcacagtcta

SEQ ID NO: 359 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattatttcgcagggtcaagtgcacagtcta

SEQ ID NO: 360 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatccatgtatagggtcaagtgcacagtcta

SEQ ID NO: 361 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgccacgttagggtcaagtgcacagtcta

SEQ ID NO: 362 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtcgtgtaagggtcaagtgcacagtcta

SEQ ID NO: 363 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattaaagtcgagggtcaagtgcacagtcta

SEQ ID NO: 364 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcttcggacagggtcaagtgcacagtcta

SEQ ID NO: 365 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgcactctcagggtcaagtgcacagtcta

SEQ ID NO: 366 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcagatacagggtcaagtgcacagtcta

SEQ ID NO: 367 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcagtccctagggtcaagtgcacagtcta

SEQ ID NO: 368 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgccctaacagggtcaagtgcacagtcta

SEQ ID NO: 369 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctgcatcaagggtcaagtgcacagtcta

SEQ ID NO: 370 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcggtatcgagggtcaagtgcacagtcta

SEQ ID NO: 371 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataagtatggagggtcaagtgcacagtcta

SEQ ID NO: 372 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatattcgcgcagggtcaagtgcacagtcta

SEQ ID NO: 373 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatatcaaggtagggtcaagtgcacagtcta

SEQ ID NO: 374 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatttgtgcatagggtcaagtgcacagtcta

SEQ ID NO: 375 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctgtgctgagggtcaagtgcacagtcta

SEQ ID NO: 376 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtccgtagagggtcaagtgcacagtcta

SEQ ID NO: 377 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgttcaagaagggtcaagtgcacagtcta

SEQ ID NO: 378 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcaccgttcagggtcaagtgcacagtcta

SEQ ID NO: 379 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgagttgaagggtcaagtgcacagtcta

SEQ ID NO: 380 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgagcacgaagggtcaagtgcacagtcta

SEQ ID NO: 381 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagttcgtgagggtcaagtgcacagtcta

SEQ ID NO: 382 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcatcaactagggtcaagtgcacagtcta

SEQ ID NO: 383 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcgagatctagggtcaagtgcacagtcta

SEQ ID NO: 384 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattggccagaagggtcaagtgcacagtcta

SEQ ID NO: 385 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatttcaccatagggtcaagtgcacagtcta

SEQ ID NO: 386 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgaatgcatagggtcaagtgcacagtcta

SEQ ID NO: 387 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattggaccctagggtcaagtgcacagtcta

SEQ ID NO: 388 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgatagcacagggtcaagtgcacagtcta

SEQ ID NO: 389 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatacgacgacagggtcaagtgcacagtcta

SEQ ID NO: 390 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctcagtatagggtcaagtgcacagtcta

SEQ ID NO: 391 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcttagctaagggtcaagtgcacagtcta

SEQ ID NO: 392 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatctgtttacagggtcaagtgcacagtcta

SEQ ID NO: 393 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattgtcccacagggtcaagtgcacagtcta

SEQ ID NO: 394 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattcctgaggagggtcaagtgcacagtcta

SEQ ID NO: 395 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattagttccaagggtcaagtgcacagtcta

SEQ ID NO: 396 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcatgactcagggtcaagtgcacagtcta

SEQ ID NO: 397 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtaagcgcagggtcaagtgcacagtcta

SEQ ID NO: 398 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataacccagtagggtcaagtgcacagtcta

SEQ ID NO: 399 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattttgagggagggtcaagtgcacagtcta

SEQ ID NO: 400 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagccgacaagggtcaagtgcacagtcta

SEQ ID NO: 401 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataaacccgcagggtcaagtgcacagtcta

SEQ ID NO: 402 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtagggctagggtcaagtgcacagtcta

SEQ ID NO: 403 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagacgattagggtcaagtgcacagtcta

SEQ ID NO: 404 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataggatgatagggtcaagtgcacagtcta

SEQ ID NO: 405 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatataatggcagggtcaagtgcacagtcta

SEQ ID NO: 406 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatcttggcgtagggtcaagtgcacagtcta

SEQ ID NO: 407 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatagctgtgcagggtcaagtgcacagtcta

SEQ ID NO: 408 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgagtccaaagggtcaagtgcacagtcta

SEQ ID NO: 409 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgaataccaagggtcaagtgcacagtcta

SEQ ID NO: 410 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagataggagcttagggtcaagtgcacagtcta

SEQ ID NO: 411 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagatgtgacttaagggtcaagtgcacagtcta

SEQ ID NO: 412 - DNA, 52 bp, synthetic construct
  misc_feature 1..52: note = source = /note="Description of Artificial Sequence: Synthetic oligonucleotide"
  source 1..52: mol_type = other DNA
caagcagaagacggcatacgagattttggaacagggtcaagtgcacagtcta

SEQ ID NO: 413 - DNA, 61 bp, synthetic construct; identical to SEQ ID NO: 110
  misc_feature 1..61: note = source = /note="Description of Artificial Sequence: Synthetic oligonucleotide"
  source 1..61: mol_type = other DNA

SEQ ID NO: 414 - DNA, 61 bp, synthetic construct
  misc_feature 1..61: note = source = /note="Description of Artificial Sequence: Synthetic oligonucleotide"
  source 1..61: mol_type = other DNA
aatgatacggcgaccaccgagatctacactgtcatgatctggttactgccagttgaatctg

SEQ ID NO: 415 - DNA, 61 bp, synthetic construct
  misc_feature 1..61: note = source = /note="Description of Artificial Sequence: Synthetic oligonucleotide"
  source 1..61: mol_type = other DNA
aatgatacggcgaccaccgagatctacactgtaacagtctggttactgccagttgaatctg

SEQ ID NO: 416 - DNA, 61 bp, synthetic construct
  misc_feature 1..61: note = source = /note="Description of Artificial Sequence: Synthetic oligonucleotide"
  source 1..61: mol_type = other DNA
aatgatacggcgaccaccgagatctacacgcgcaacttctggttactgccagttgaatctg

SEQ ID NO: 417 - DNA, 52 bp, synthetic construct
  misc_feature 1..52: note = source = /note="Description of Artificial Sequence: Synthetic oligonucleotide"
  source 1..52: mol_type = other DNA
aatgatacggcgaccaccgatcgatggctctggttactgccagttgaatctg

SEQ ID NO: 418 - DNA, 52 bp, synthetic construct
  misc_feature 1..52: note = source = /note="Description of Artificial Sequence: Synthetic oligonucleotide"
  source 1..52: mol_type = other DNA
aatgatacggcgaccaccgacatggttttctggttactgccagttgaatctg

SEQ ID NO: 419 - DNA, 52 bp, synthetic construct
  misc_feature 1..52: note = source = /note="Description of Artificial Sequence: Synthetic oligonucleotide"
  source 1..52: mol_type = other DNA
aatgatacggcgaccaccgaacgaaagctctggttactgccagttgaatctg

SEQ ID NO: 420 - DNA, 52 bp, synthetic construct
  misc_feature 1..52: note = source = /note="Description of Artificial Sequence: Synthetic oligonucleotide"
  source 1..52: mol_type = other DNA
aatgatacggcgaccaccgagctctgattctggttactgccagttgaatctg

SEQ ID NO: 421 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacatgccctcgctggtgctgcagcttatta

SEQ ID NO: 422 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacctcagatggctggtgctgcagcttatta

SEQ ID NO: 423 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacgtaatctggctggtgctgcagcttatta

SEQ ID NO: 424 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacgcaagattgctggtgctgcagcttatta

SEQ ID NO: 425 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgaatacgccagctggtgctgcagcttatta

SEQ ID NO: 426 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgaaccagtcggctggtgctgcagcttatta

SEQ ID NO: 427 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgaaacacggagctggtgctgcagcttatta

SEQ ID NO: 428 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgactgcctaggctggtgctgcagcttatta

SEQ ID NO: 429 - DNA, 31 bp, synthetic construct
  misc_feature 1..31: note = source = /note="Description of Artificial Sequence: Synthetic oligonucleotide"
  source 1..31: mol_type = other DNA
tctggttactgccagttgaatctgagggtcc

SEQ ID NO: 430 - DNA, 25 bp, synthetic construct
  misc_feature 1..25: note = source = /note="Description of Artificial Sequence: Synthetic oligonucleotide"
  source 1..25: mol_type = other DNA
ggtgcatttcgctgattttggggtc

SEQ ID NO: 431 - DNA, 31 bp, synthetic construct
  misc_feature 1..31: note = source = /note="Description of Artificial Sequence: Synthetic oligonucleotide"
  source 1..31: mol_type = other DNA
ggaccctcagattcaactggcagtaaccaga

SEQ ID NO: 432 - DNA, 27 bp, synthetic construct
  misc_feature 1..27: note = source = /note="Description of Artificial Sequence: Synthetic oligonucleotide"
  source 1..27: mol_type = other DNA
gctggtgctgcagcttattatgtgggt

SEQ ID NO: 433 - DNA, 28 bp, synthetic construct
  misc_feature 1..28: note = source = /note="Description of Artificial Sequence: Synthetic oligonucleotide"
  source 1..28: mol_type = other DNA
agatgctgtagactgtgcacttgaccct

SEQ ID NO: 434 - DNA, 27 bp, synthetic construct
  misc_feature 1..27: note = source = /note="Description of Artificial Sequence: Synthetic oligonucleotide"
  source 1..27: mol_type = other DNA
acccacataataagctgcagcaccagc

SEQ ID NO: 435 - DNA, 23 bp, synthetic construct
  misc_feature 1..23: note = source = /note="Description of Artificial Sequence: Synthetic oligonucleotide"
  source 1..23: mol_type = other DNA
gagcggctgtctccacaagtccg

SEQ ID NO: 436 - DNA, 23 bp, synthetic construct
  misc_feature 1..23: note = source = /note="Description of Artificial Sequence: Synthetic oligonucleotide"
  source 1..23: mol_type = other DNA
acccgctcgcaggtccaaatctg

SEQ ID NO: 437 - DNA, 23 bp, synthetic construct
  misc_feature 1..23: note = source = /note="Description of Artificial Sequence: Synthetic oligonucleotide"
  source 1..23: mol_type = other DNA
cggacttgtggagacagccgctc

SEQ ID NO: 438 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgagtcttcagatttggacctgcgagcg

SEQ ID NO: 439 - DNA, 51 bp, synthetic construct; identical to SEQ ID NO: 113
  misc_feature 1..51: note = source = /note="Description of Artificial Sequence: Synthetic oligonucleotide"
  source 1..51: mol_type = other DNA

SEQ ID NO: 440 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattgggccaaagatttggacctgcgagcg

SEQ ID NO: 441 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatattgttggagatttggacctgcgagcg

SEQ ID NO: 442 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattcccgttgagatttggacctgcgagcg

SEQ ID NO: 443 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatacacacttagatttggacctgcgagcg

SEQ ID NO: 444 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatccattccaagatttggacctgcgagcg

SEQ ID NO: 445 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatctaacgggagatttggacctgcgagcg

SEQ ID NO: 446 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatccataggaagatttggacctgcgagcg

SEQ ID NO: 447 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcagtgtagagatttggacctgcgagcg

SEQ ID NO: 448 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgattctcaagatttggacctgcgagcg

SEQ ID NO: 449 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatccttcttaagatttggacctgcgagcg

SEQ ID NO: 450 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattctaagacagatttggacctgcgagcg

SEQ ID NO: 451 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgcggcataagatttggacctgcgagcg

SEQ ID NO: 452 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatattgacgaagatttggacctgcgagcg

SEQ ID NO: 453 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgctcctgaagatttggacctgcgagcg

SEQ ID NO: 454 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgcaatcctagatttggacctgcgagcg

SEQ ID NO: 455 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatatgtcgttagatttggacctgcgagcg

SEQ ID NO: 456 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatttctcggcagatttggacctgcgagcg

SEQ ID NO: 457 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcagggctaagatttggacctgcgagcg

SEQ ID NO: 458 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatagccaagcagatttggacctgcgagcg

SEQ ID NO: 459 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagataagcctgaagatttggacctgcgagcg

SEQ ID NO: 460 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatctacagagagatttggacctgcgagcg

SEQ ID NO: 461 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcgtagtcgagatttggacctgcgagcg

SEQ ID NO: 462 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatttctgctcagatttggacctgcgagcg

SEQ ID NO: 463 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgtgcacacagatttggacctgcgagcg

SEQ ID NO: 464 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagataaagctcaagatttggacctgcgagcg

SEQ ID NO: 465 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgacctcagagatttggacctgcgagcg

SEQ ID NO: 466 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatctttccaaagatttggacctgcgagcg

SEQ ID NO: 467 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattcttggctagatttggacctgcgagcg

SEQ ID NO: 468 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcgcgtctaagatttggacctgcgagcg

SEQ ID NO: 469 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattcgcgctaagatttggacctgcgagcg

SEQ ID NO: 470 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatatccattcagatttggacctgcgagcg

SEQ ID NO: 471 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgcccagtaagatttggacctgcgagcg

SEQ ID NO: 472 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattaccgacgagatttggacctgcgagcg

SEQ ID NO: 473 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattccatacgagatttggacctgcgagcg

SEQ ID NO: 474 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagataacatgtcagatttggacctgcgagcg

SEQ ID NO: 475 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcgactataagatttggacctgcgagcg

SEQ ID NO: 476 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatacccaaagagatttggacctgcgagcg

SEQ ID NO: 477 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatatcgatcgagatttggacctgcgagcg

SEQ ID NO: 478 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgttggatgagatttggacctgcgagcg

SEQ ID NO: 479 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatctatgtgaagatttggacctgcgagcg

SEQ ID NO: 480 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattatttcgcagatttggacctgcgagcg

SEQ ID NO: 481 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatccatgtatagatttggacctgcgagcg

SEQ ID NO: 482 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgccacgttagatttggacctgcgagcg

SEQ ID NO: 483 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgtcgtgtaagatttggacctgcgagcg

SEQ ID NO: 484 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattaaagtcgagatttggacctgcgagcg

SEQ ID NO: 485 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcttcggacagatttggacctgcgagcg

SEQ ID NO: 486 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgcactctcagatttggacctgcgagcg

SEQ ID NO: 487 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattcagatacagatttggacctgcgagcg

SEQ ID NO: 488 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcagtccctagatttggacctgcgagcg

SEQ ID NO: 489 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgccctaacagatttggacctgcgagcg

SEQ ID NO: 490 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatctgcatcaagatttggacctgcgagcg

SEQ ID NO: 491 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcggtatcgagatttggacctgcgagcg

SEQ ID NO: 492 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagataagtatggagatttggacctgcgagcg

SEQ ID NO: 493 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatattcgcgcagatttggacctgcgagcg

SEQ ID NO: 494 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatatcaaggtagatttggacctgcgagcg

SEQ ID NO: 495 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatttgtgcatagatttggacctgcgagcg

SEQ ID NO: 496 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatctgtgctgagatttggacctgcgagcg

SEQ ID NO: 497 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgtccgtagagatttggacctgcgagcg

SEQ ID NO: 498 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgttcaagaagatttggacctgcgagcg

SEQ ID NO: 499 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcaccgttcagatttggacctgcgagcg

SEQ ID NO: 500 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcgagttgaagatttggacctgcgagcg

SEQ ID NO: 501 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgagcacgaagatttggacctgcgagcg

SEQ ID NO: 502 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatagttcgtgagatttggacctgcgagcg

SEQ ID NO: 503 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcatcaactagatttggacctgcgagcg

SEQ ID NO: 504 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcgagatctagatttggacctgcgagcg

SEQ ID NO: 505 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattggccagaagatttggacctgcgagcg

SEQ ID NO: 506 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatttcaccatagatttggacctgcgagcg

SEQ ID NO: 507 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgaatgcatagatttggacctgcgagcg

SEQ ID NO: 508 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattggaccctagatttggacctgcgagcg

SEQ ID NO: 509 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgatagcacagatttggacctgcgagcg

SEQ ID NO: 510 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatacgacgacagatttggacctgcgagcg

SEQ ID NO: 511 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatctcagtatagatttggacctgcgagcg

SEQ ID NO: 512 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcttagctaagatttggacctgcgagcg

SEQ ID NO: 513 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatctgtttacagatttggacctgcgagcg

SEQ ID NO: 514 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattgtcccacagatttggacctgcgagcg

SEQ ID NO: 515 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattcctgaggagatttggacctgcgagcg

SEQ ID NO: 516 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattagttccaagatttggacctgcgagcg

SEQ ID NO: 517 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcatgactcagatttggacctgcgagcg

SEQ ID NO: 518 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgtaagcgcagatttggacctgcgagcg

SEQ ID NO: 519 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagataacccagtagatttggacctgcgagcg

SEQ ID NO: 520 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattttgagggagatttggacctgcgagcg

SEQ ID NO: 521 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatagccgacaagatttggacctgcgagcg

SEQ ID NO: 522 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagataaacccgcagatttggacctgcgagcg

SEQ ID NO: 523 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgtagggctagatttggacctgcgagcg

SEQ ID NO: 524 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatagacgattagatttggacctgcgagcg

SEQ ID NO: 525 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagataggatgatagatttggacctgcgagcg

SEQ ID NO: 526 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatataatggcagatttggacctgcgagcg

SEQ ID NO: 527 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatcttggcgtagatttggacctgcgagcg

SEQ ID NO: 528 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatagctgtgcagatttggacctgcgagcg

SEQ ID NO: 529 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgagtccaaagatttggacctgcgagcg

SEQ ID NO: 530 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgaataccaagatttggacctgcgagcg

SEQ ID NO: 531 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagataggagcttagatttggacctgcgagcg

SEQ ID NO: 532 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagatgtgacttaagatttggacctgcgagcg

SEQ ID NO: 533 - DNA, 51 bp, synthetic construct
  misc_feature 1..51: note = source = /note="Description of Artificial Sequence: Synthetic oligonucleotide"
  source 1..51: mol_type = other DNA
caagcagaagacggcatacgagattttggaacagatttggacctgcgagcg

SEQ ID NO: 534 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacctctctatgagcggctgtctccacaagt

SEQ ID NO: 535 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacactatcctctgagcggctgtctccacaagt

SEQ ID NO: 536 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacgtaaggaggagcggctgtctccacaagt

SEQ ID NO: 537 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacactgcatagagcggctgtctccacaagt

SEQ ID NO: 538 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgaaaggagtagagcggctgtctccacaagt

SEQ ID NO: 539 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgactaagcctgagcggctgtctccacaagt

SEQ ID NO: 540 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgacgtctaatgagcggctgtctccacaagt

SEQ ID NO: 541 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgatctctccggagcggctgtctccacaagt

SEQ ID NO: 542 - DNA, 57 bp, synthetic construct; identical to SEQ ID NO: 114
  misc_feature 1..57: note = source = /note="Description of Artificial Sequence: Synthetic oligonucleotide"
  source 1..57: mol_type = other DNA

SEQ ID NO: 543 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacactgtcatgagagcggctgtctccacaagt

SEQ ID NO: 544 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacactgtaacaggagcggctgtctccacaagt

SEQ ID NO: 545 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacgcgcaactgagcggctgtctccacaagt

SEQ ID NO: 546 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgatcgatggcgagcggctgtctccacaagt

SEQ ID NO: 547 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgacatggtttgagcggctgtctccacaagt

SEQ ID NO: 548 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgaacgaaagcgagcggctgtctccacaagt

SEQ ID NO: 549 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgagctctgatgagcggctgtctccacaagt

SEQ ID NO: 550 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacatgccctcgagcggctgtctccacaagt

SEQ ID NO: 551 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacctcagatggagcggctgtctccacaagt

SEQ ID NO: 552 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacgtaatctggagcggctgtctccacaagt

SEQ ID NO: 553 - DNA, 57 bp, synthetic construct
  misc_feature 1..57: note = source = /note="Description of Artificial Sequence: Synthetic oligonucleotide"
  source 1..57: mol_type = other DNA
aatgatacggcgaccaccgagatctacacgcaagattgagcggctgtctccacaagt

SEQ ID NO: 554 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgaatacgccagagcggctgtctccacaagt

SEQ ID NO: 555 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgaaccagtcggagcggctgtctccacaagt

SEQ ID NO: 556 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgaaacacggagagcggctgtctccacaagt

SEQ ID NO: 557 - DNA, 48 bp, synthetic construct
  misc_feature 1..48: note = source = /note="Description of Artificial Sequence: Synthetic oligonucleotide"
  source 1..48: mol_type = other DNA
aatgatacggcgaccaccgactgcctaggagcggctgtctccacaagt

SEQ ID NO: 558 - DNA, 43 bp, synthetic construct
  misc_feature 1..43: note = source = /note="Description of Artificial Sequence: Synthetic oligonucleotide"
  source 1..43: mol_type = other DNA
gggtcacgcgtaggagttctatcgaccccaaaatcagcgaaat

SEQ ID NO: 559 - DNA, 47 bp, synthetic construct
  misc_feature 1..47: note = source = /note="Description of Artificial Sequence: Synthetic oligonucleotide"
  source 1..47: mol_type = other DNA
gttccgcagccacacaagatctgtctggttactgccagttgaatctg

SEQ ID NO: 560 - DNA, 43 bp, synthetic construct
  misc_feature 1..43: note = source = /note="Description of Artificial Sequence: Synthetic oligonucleotide"
  source 1..43: mol_type = other DNA
cgcgtcgagtagggtgttctatcgaccccaaaatcagcgaaat

SEQ ID NO: 561 - DNA, 47 bp, synthetic construct
  misc_feature 1..47: note = source = /note="Description of Artificial Sequence: Synthetic oligonucleotide"
  source 1..47: mol_type = other DNA
gccgtgtgaagctggaagatctgtctggttactgccagttgaatctg

SEQ ID NO: 562 - DNA, 43 bp, synthetic construct
  misc_feature 1..43: note = source = /note="Description of Artificial Sequence: Synthetic oligonucleotide"
  source 1..43: mol_type = other DNA
cgatcgcccttggtggttctatcgaccccaaaatcagcgaaat

SEQ ID NO: 563 - DNA, 47 bp, synthetic construct
  misc_feature 1..47: note = source = /note="Description of Artificial Sequence: Synthetic oligonucleotide"
  source 1..47: mol_type = other DNA
ggtttagccggcgtgaagatctgtctggttactgccagttgaatctg

SEQ ID NO: 564 - DNA, 43 bp, synthetic construct
  misc_feature 1..43: note = source = /note="Description of Artificial Sequence: Synthetic oligonucleotide"
  source 1..43: mol_type = other DNA
ggtcgagccggaactgttctatcgaccccaaaatcagcgaaat

SEQ ID NO: 565 - DNA, 47 bp, synthetic construct
  misc_feature 1..47: note = source = /note="Description of Artificial Sequence: Synthetic oligonucleotide"
  source 1..47: mol_type = other DNA
ggatgcgcacccagaaagatctgtctggttactgccagttgaatctg

SEQ ID NO: 566 - DNA, 43 bp, synthetic construct
  misc_feature 1..43: note = source = /note="Description of Artificial Sequence: Synthetic oligonucleotide"
  source 1..43: mol_type = other DNA
tcccggcgttgtcctgttctatcgaccccaaaatcagcgaaat

SEQ ID NO: 567 - DNA, 47 bp, synthetic construct
  misc_feature 1..47: note = source = /note="Description of Artificial Sequence: Synthetic oligonucleotide"
  source 1..47: mol_type = other DNA
gctccgtcactgcccaagatctgtctggttactgccagttgaatctg

SEQ ID NO: 568 - DNA, 43 bp, synthetic construct
  misc_feature 1..43: note = source = /note="Description of Artificial Sequence: Synthetic oligonucleotide"
  source 1..43: mol_type = other DNA
cgcagggtccagagtgttctatcgaccccaaaatcagcgaaat

SEQ ID NO: 569 - DNA, 47 bp, synthetic construct
  misc_feature 1..47: note = source = /note="Description of Artificial Sequence: Synthetic oligonucleotide"
  source 1..47: mol_type = other DNA
gttcgcgcgaaggaaaagatctgtctggttactgccagttgaatctg

SEQ ID NO: 570 - DNA, 48 bp, synthetic construct
  misc_feature 1..48: note = source = /note="Description of Artificial Sequence: Synthetic oligonucleotide"
  source 1..48: mol_type = other DNA
atatagatgccgtcctagcggttctatcgaccccaaaatcagcgaaat

SEQ ID NO: 571 - DNA, 52 bp, synthetic construct
  misc_feature 1..52: note = source = /note="Description of Artificial Sequence: Synthetic oligonucleotide"
  source 1..52: mol_type = other DNA
aagtatctttcctgtgcccaaagatctgtctggttactgccagttgaatctg

SEQ ID NO: 572 - DNA, 48 bp, synthetic construct
  misc_feature 1..48: note = source = /note="Description of Artificial Sequence: Synthetic oligonucleotide"
  source 1..48: mol_type = other DNA
ccctttaatcagatgcgtcggttctatcgaccccaaaatcagcgaaat

SEQ ID NO: 573 - DNA, 52 bp, synthetic construct
  misc_feature 1..52: note = source = /note="Description of Artificial Sequence: Synthetic oligonucleotide"
  source 1..52: mol_type = other DNA
tggtagtaataagggcgaccaagatctgtctggttactgccagttgaatctg

SEQ ID NO: 574 - DNA, 48 bp, synthetic construct
  misc_feature 1..48: note = source = /note="Description of Artificial Sequence: Synthetic oligonucleotide"
  source 1..48: mol_type = other DNA
ttggtcatgtgcttttcgttgttctatcgaccccaaaatcagcgaaat

SEQ ID NO: 575 - DNA, 52 bp, synthetic construct
  misc_feature 1..52: note = source = /note="Description of Artificial Sequence: Synthetic oligonucleotide"
  source 1..52: mol_type = other DNA
aggggtatcggatactcagaaagatctgtctggttactgccagttgaatctg

SEQ ID NO: 576 - DNA, 48 bp, synthetic construct
  misc_feature 1..48: note = source = /note="Description of Artificial Sequence: Synthetic oligonucleotide"
  source 1..48: mol_type = other DNA
gggtgggtaaatggtaatgcgttctatcgaccccaaaatcagcgaaat

SEQ ID NO: 577 - DNA, 52 bp, synthetic construct
  misc_feature 1..52: note = source = /note="Description of Artificial Sequence: Synthetic oligonucleotide"
  source 1..52: mol_type = other DNA
atcgattccccggatatagcaagatctgtctggttactgccagttgaatctg

SEQ ID NO: 578 - DNA, 48 bp, synthetic construct
  misc_feature 1..48: note = source = /note="Description of Artificial Sequence: Synthetic oligonucleotide"
  source 1..48: mol_type = other DNA
tccgacggggagtatatactgttctatcgaccccaaaatcagcgaaat

SEQ ID NO: 579 - DNA, 52 bp, synthetic construct
  misc_feature 1..52: note = source = /note="Description of Artificial Sequence: Synthetic oligonucleotide"
  source 1..52: mol_type = other DNA
tactaactgcttcaggccaaaagatctgtctggttactgccagttgaatctg

SEQ ID NO: 580 - DNA, 48 bp, synthetic construct
  misc_feature 1..48: note = source = /note="Description of Artificial Sequence: Synthetic oligonucleotide"
  source 1..48: mol_type = other DNA
catgtttaggaacgctaccggttctatcgaccccaaaatcagcgaaat

SEQ ID NO: 581 - DNA, 52 bp, synthetic construct
  misc_feature 1..52: note = source = /note="Description of Artificial Sequence: Synthetic oligonucleotide"
  source 1..52: mol_type = other DNA
aataatctccgttccctcccaagatctgtctggttactgccagttgaatctg

SEQ ID NO: 582 - DNA, 43 bp, synthetic construct
  misc_feature 1..43: note = source = /note="Description of Artificial Sequence: Synthetic oligonucleotide"
  source 1..43: mol_type = other DNA
gggtcacgcgtaggagttctatcgctggtgctgcagcttatta

SEQ ID NO: 583 - DNA, 43 bp, synthetic construct
  misc_feature 1..43: note = source = /note="Description of Artificial Sequence: Synthetic oligonucleotide"
  source 1..43: mol_type = other DNA
gttccgcagccacacaagatctgagggtcaagtgcacagtcta

SEQ ID NO: 584 - DNA, 43 bp, synthetic construct
  misc_feature 1..43: note = source = /note="Description of Artificial Sequence: Synthetic oligonucleotide"
  source 1..43: mol_type = other DNA
cgcgtcgagtagggtgttctatcgctggtgctgcagcttatta

SEQ ID NO: 585 - DNA, 43 bp, synthetic construct
  misc_feature 1..43: note = source = /note="Description of Artificial Sequence: Synthetic oligonucleotide"
  source 1..43: mol_type = other DNA
gccgtgtgaagctggaagatctgagggtcaagtgcacagtcta

SEQ ID NO: 586 - DNA, 43 bp, synthetic construct
  misc_feature 1..43: note = source = /note="Description of Artificial Sequence: Synthetic oligonucleotide"
  source 1..43: mol_type = other DNA
cgatcgcccttggtggttctatcgctggtgctgcagcttatta

SEQ ID NO: 587 - DNA, 43 bp, synthetic construct
  misc_feature 1..43: note = source = /note="Description of Artificial Sequence: Synthetic oligonucleotide"
  source 1..43: mol_type = other DNA
ggtttagccggcgtgaagatctgagggtcaagtgcacagtcta

SEQ ID NO: 588 - DNA, 43 bp, synthetic construct
  misc_feature 1..43: note = source = /note="Description of Artificial Sequence: Synthetic oligonucleotide"
  source 1..43: mol_type = other DNA
ggtcgagccggaactgttctatcgctggtgctgcagcttatta

SEQ ID NO: 589 - DNA, 43 bp, synthetic construct
  misc_feature 1..43: note = source = /note="Description of Artificial Sequence: Synthetic oligonucleotide"
  source 1..43: mol_type = other DNA
ggatgcgcacccagaaagatctgagggtcaagtgcacagtcta

SEQ ID NO: 590 - DNA, 43 bp, synthetic construct
  misc_feature 1..43: note = source = /note="Description of Artificial Sequence: Synthetic oligonucleotide"
  source 1..43: mol_type = other DNA
tcccggcgttgtcctgttctatcgctggtgctgcagcttatta

SEQ ID NO: 591 - DNA, 43 bp, synthetic construct
  misc_feature 1..43: note = source = /note="Description of Artificial Sequence: Synthetic oligonucleotide"
  source 1..43: mol_type = other DNA
gctccgtcactgcccaagatctgagggtcaagtgcacagtcta

SEQ ID NO: 592 - DNA, 43 bp, synthetic construct
  misc_feature 1..43: note = source = /note="Description of Artificial Sequence: Synthetic oligonucleotide"
  source 1..43: mol_type = other DNA
cgcagggtccagagtgttctatcgctggtgctgcagcttatta

SEQ ID NO: 593 - DNA, 43 bp, synthetic construct
  misc_feature 1..43: note = source = /note="Description of Artificial Sequence: Synthetic oligonucleotide"
  source 1..43: mol_type = other DNA
gttcgcgcgaaggaaaagatctgagggtcaagtgcacagtcta

SEQ ID NO: 594 - DNA, 48 bp, synthetic construct
  misc_feature 1..48: note = source = /note="Description of Artificial Sequence: Synthetic oligonucleotide"
  source 1..48: mol_type = other DNA
atatagatgccgtcctagcggttctatcgctggtgctgcagcttatta

SEQ ID NO: 595 - DNA, 48 bp, synthetic construct
  misc_feature 1..48: note = source = /note="Description of Artificial Sequence: Synthetic oligonucleotide"
  source 1..48: mol_type = other DNA
aagtatctttcctgtgcccaaagatctgagggtcaagtgcacagtcta

SEQ ID NO: 596 - DNA, 48 bp, synthetic construct
  misc_feature 1..48: note = source = /note="Description of Artificial Sequence: Synthetic oligonucleotide"
  source 1..48: mol_type = other DNA
ccctttaatcagatgcgtcggttctatcgctggtgctgcagcttatta

SEQ ID NO: 597 - DNA, 48 bp, synthetic construct
  misc_feature 1..48: note = source = /note="Description of Artificial Sequence: Synthetic oligonucleotide"
  source 1..48: mol_type = other DNA
tggtagtaataagggcgaccaagatctgagggtcaagtgcacagtcta

SEQ ID NO: 598 - DNA, 48 bp, synthetic construct
  misc_feature 1..48: note = source = /note="Description of Artificial Sequence: Synthetic oligonucleotide"
  source 1..48: mol_type = other DNA
ttggtcatgtgcttttcgttgttctatcgctggtgctgcagcttatta

SEQ ID NO: 599 - DNA, 48 bp, synthetic construct
  misc_feature 1..48: note = source = /note="Description of Artificial Sequence: Synthetic oligonucleotide"
  source 1..48: mol_type = other DNA
aggggtatcggatactcagaaagatctgagggtcaagtgcacagtcta

SEQ ID NO: 600 - DNA, 48 bp, synthetic construct
  misc_feature 1..48: note = source = /note="Description of Artificial Sequence: Synthetic oligonucleotide"
  source 1..48: mol_type = other DNA
gggtgggtaaatggtaatgcgttctatcgctggtgctgcagcttatta

SEQ ID NO: 601 - DNA, 48 bp, synthetic construct
  misc_feature 1..48: note = source = /note="Description of Artificial Sequence: Synthetic oligonucleotide"
  source 1..48: mol_type = other DNA
atcgattccccggatatagcaagatctgagggtcaagtgcacagtcta

SEQ ID NO: 602 - DNA, 48 bp, synthetic construct
  misc_feature 1..48: note = source = /note="Description of Artificial Sequence: Synthetic oligonucleotide"
  source 1..48: mol_type = other DNA
tccgacggggagtatatactgttctatcgctggtgctgcagcttatta

SEQ ID NO: 603 - DNA, 48 bp, synthetic construct
  misc_feature 1..48: note = source = /note="Description of Artificial Sequence: Synthetic oligonucleotide"
  source 1..48: mol_type = other DNA
tactaactgcttcaggccaaaagatctgagggtcaagtgcacagtcta

SEQ ID NO: 604 - DNA, 48 bp, synthetic construct
  misc_feature 1..48: note = source = /note="Description of Artificial Sequence: Synthetic oligonucleotide"
  source 1..48: mol_type = other DNA
catgtttaggaacgctaccggttctatcgctggtgctgcagcttatta

SEQ ID NO: 605 - DNA, 48 bp, synthetic construct
  misc_feature 1..48: note = source = /note="Description of Artificial Sequence: Synthetic oligonucleotide"
  source 1..48: mol_type = other DNA
aataatctccgttccctcccaagatctgagggtcaagtgcacagtcta

SEQ ID NO: 606 - DNA, 108 bp, synthetic construct
  misc_feature 1..108: note = source = /note="Description of Artificial Sequence: Synthetic polynucleotide"
  source 1..108: mol_type = other DNA
gctggtgctgcagcttattatgtgggtgtgtatctcacgaagcgaccctttggaaaatataatgaaaatggaaccattacagatgctgtagactgtgcacttgaccct

SEQ ID NO: 607 - DNA, 108 bp, synthetic construct
  misc_feature 1..108: note = source = /note="Description of Artificial Sequence: Synthetic polynucleotide"
  source 1..108: mol_type = other DNA
gctggtgctgcagcttattatgtgggtcctcgctaggacgtcgctatgacgccaaaatataatgaaaatggaaccattacagatgctgtagactgtgcacttgaccct

SEQ ID NO: 608 - DNA, 108 bp, synthetic construct
  misc_feature 1..108: note = source = /note="Description of Artificial Sequence: Synthetic polynucleotide"
  source 1..108: mol_type = other DNA
gctggtgctgcagcttattatgtgggtagcacgacttgatctaactgacactaaaaatataatgaaaatggaaccattacagatgctgtagactgtgcacttgaccct

SEQ ID NO: 609 - DNA, 108 bp, synthetic construct
  misc_feature 1..108: note = source = /note="Description of Artificial Sequence: Synthetic polynucleotide"
  source 1..108: mol_type = other DNA
gctggtgctgcagcttattatgtgggttaagtaggacttcgattggatggaataaaatataatgaaaatggaaccattacagatgctgtagactgtgcacttgaccct

SEQ ID NO: 610 - DNA, 82 bp, synthetic construct
  misc_feature 1..82: note = source = /note="Description of Artificial Sequence: Synthetic oligonucleotide"
  source 1..82: mol_type = other DNA
tctggttactgccagttgaatctgagggtccggacggatatcgcactaagtgtacctggtgcatttcgctgattttggggtc

SEQ ID NO: 611 - DNA, 82 bp, synthetic construct
  misc_feature 1..82: note = source = /note="Description of Artificial Sequence: Synthetic oligonucleotide"
  source 1..82: mol_type = other DNA
tctggttactgccagttgaatctgagggtcccatgaccatgtcactggctacactgaggtgcatttcgctgattttggggtc

SEQ ID NO: 612 - DNA, 82 bp, synthetic construct
  misc_feature 1..82: note = source = /note="Description of Artificial Sequence: Synthetic oligonucleotide"
  source 1..82: mol_type = other DNA
tctggttactgccagttgaatctgagggtccttgacatggcatgtgactccactgtcggtgcatttcgctgattttggggtc

SEQ ID NO: 613 - DNA, 82 bp, synthetic construct
  misc_feature 1..82: note = source = /note="Description of Artificial Sequence: Synthetic oligonucleotide"
  source 1..82: mol_type = other DNA
tctggttactgccagttgaatctgagggtccacctttgccagatgactgagtggaagggtgcatttcgctgattttggggtc

SEQ ID NO: 614 - DNA, 82 bp, synthetic construct
  misc_feature 1..82: note = source = /note="Description of Artificial Sequence: Synthetic oligonucleotide"
  source 1..82: mol_type = other DNA
tctggttactgccagttgaatctgagggtccagcttgaagcgttcgcgacaagtgtcggtgcatttcgctgattttggggtc

SEQ ID NO: 615 - DNA, 82 bp, synthetic construct
  misc_feature 1..82: note = source = /note="Description of Artificial Sequence: Synthetic oligonucleotide"
  source 1..82: mol_type = other DNA
tctggttactgccagttgaatctgagggtcctcacgtcctgagatcaactgctacatggtgcatttcgctgattttggggtc

SEQ ID NO: 616 - DNA, 82 bp, synthetic construct
  misc_feature 1..82: note = source = /note="Description of Artificial Sequence: Synthetic oligonucleotide"
  source 1..82: mol_type = other DNA
tctggttactgccagttgaatctgagggtccgttgactgatcacatgctgctccacgggtgcatttcgctgattttggggtc

SEQ ID NO: 617 - DNA, 82 bp, synthetic construct
  misc_feature 1..82: note = source = /note="Description of Artificial Sequence: Synthetic oligonucleotide"
  source 1..82: mol_type = other DNA
tctggttactgccagttgaatctgagggtcccagacagtcatcggattgatgagtgaggtgcatttcgctgattttggggtc

SEQ ID NO: 618 - DNA, 26 bp, synthetic construct
  misc_feature 1..26: note = source = /note="Description of Artificial Sequence: Synthetic oligonucleotide"
  source 1..26: mol_type = other DNA
tatcttcaacctaggacttttctatt

SEQ ID NO: 619 - DNA, 26 bp, synthetic construct
  misc_feature 1..26: note = source = /note="Description of Artificial Sequence: Synthetic oligonucleotide"
  source 1..26: mol_type = other DNA
atagaacaacctaggacttttctatt

SEQ ID NO: 620 - DNA, 92 bp, synthetic construct
  variation 30..39: note = a, c, t, g, unknown or other
  variation 67..72: note = /replace="atagaa"
  source 1..92: mol_type = other DNA; note = Variant positions given in the sequence have no preference with respect to those in the annotations for variant positions; note = Description of Artificial Sequence: Synthetic oligonucleotide
aatgatacggcgaccaccgagatctacacnnnnnnnnnngctggtgctgcagcttattatgtgggttatcttcaacctaggacttttctatt

SEQ ID NO: 621 - DNA, 62 bp, synthetic construct
  variation 29..38: note = a, c, t, g, unknown or other
  source 1..62: mol_type = other DNA; note = Description of Artificial Sequence: Synthetic oligonucleotide
agatgctgtagactgtgcacttgaccctnnnnnnnnnnatctcgtatgccgtcttctgcttg

SEQ ID NO: 622 - DNA, 28 bp, synthetic construct
  misc_feature 1..28: note = source = /note="Description of Artificial Sequence: Synthetic primer"
  source 1..28: mol_type = other DNA
agatgctgtagactgtgcacttgaccct

SEQ ID NO: 623 - DNA, 27 bp, synthetic construct
  misc_feature 1..27: note = source = /note="Description of Artificial Sequence: Synthetic primer"
  source 1..27: mol_type = other DNA
acccacataataagctgcagcaccagc

SEQ ID NO: 624 - DNA, 138 bp, synthetic construct
  variation 30..39: note = a, c, t, g, unknown or other
  variation 63..67: note = /replace="gcgtc"
  variation 105..114: note = a, c, t, g, unknown or other
  source 1..138: mol_type = other DNA; note = Variant positions given in the sequence have no preference with respect to those in the annotations for variant positions; note = Description of Artificial Sequence: Synthetic polynucleotide
aatgatacggcgaccaccgagatctacacnnnnnnnnnngagcggctgtctccacaagtccgcgcagagccttcaggtcagaacccgctcgcaggtccaaatctnnnnnnnnnnatctcgtatgccgtcttctgcttg

SEQ ID NO: 625 - DNA, 23 bp, synthetic construct
  misc_feature 1..23: note = source = /note="Description of Artificial Sequence: Synthetic primer"
  source 1..23: mol_type = other DNA
gagcggctgtctccacaagtccg

SEQ ID NO: 626 - DNA, 22 bp, synthetic construct
  misc_feature 1..22: note = source = /note="Description of Artificial Sequence: Synthetic primer"
  source 1..22: mol_type = other DNA
acccgctcgcaggtccaaatct

SEQ ID NO: 627 - DNA, 23 bp, synthetic construct
  misc_feature 1..23: note = source = /note="Description of Artificial Sequence: Synthetic primer"
  source 1..23: mol_type = other DNA
cggacttgtggagacagccgctc

SEQ ID NO: 628 - DNA, 108 bp, Severe acute respiratory syndrome coronavirus 2
  source 1..108: mol_type = genomic DNA
gctggtgctgcagcttattatgtgggttatcttcaacctaggacttttctattaaaatataatgaaaatggaaccattacagatgctgtagactgtgcacttgaccct

SEQ ID NO: 629 - DNA, 108 bp, synthetic construct
  misc_feature 1..108: note = source = /note="Description of Artificial Sequence: Synthetic polynucleotide"
  source 1..108: mol_type = other DNA
gctggtgctgcagcttattatgtgggtatagaacaacctaggacttttctattaaaatataatgaaaatggaaccattacagatgctgtagactgtgcacttgaccct

SEQ ID NO: 630 - DNA, 108 bp, synthetic construct
  misc_feature 1..108: note = source = /note="Description of Artificial Sequence: Synthetic polynucleotide"
  source 1..108: mol_type = other DNA
gctggtgctgcagcttattatgtgggttggactacctaggactgcgcgactaaaaaatataatgaaaatggaaccattacagatgctgtagactgtgcacttgaccct

SEQ ID NO: 631 - DNA, 108 bp, synthetic construct
  misc_feature 1..108: note = source = /note="Description of Artificial Sequence: Synthetic polynucleotide"
  source 1..108: mol_type = other DNA
gctggtgctgcagcttattatgtgggtcacctcgtaggacttctgaaatagccaaaatataatgaaaatggaaccattacagatgctgtagactgtgcacttgaccct

SEQ ID NO: 632 - DNA, 108 bp, synthetic construct
  misc_feature 1..108: note = source = /note="Description of Artificial Sequence: Synthetic polynucleotide"
  source 1..108: mol_type = other DNA
gctggtgctgcagcttattatgtgggtgcttggtggatctcagaacgcggcggaaaatataatgaaaatggaaccattacagatgctgtagactgtgcacttgaccct

(19) United States
(12) Patent Application Publication
(10) Pub. No.: US 2025/0084492 A1
(43) Pub. Date: Mar. 13, 2025

(54) PATHOGEN DIAGNOSTIC TEST

(71) Applicant: Octant, Inc., Emeryville, CA (US)

(72) Inventors: Sriram KOSURI, Berkeley, CA (US); Eric Matthew JONES, Oakland, CA (US); Aaron COOPER, Berkeley, CA (US); Molly Jeanette GASPERINI, Oakland, CA (US)

(21) Appl. No.: 18/885,280

(22) Filed: Sep. 13, 2024

Related U.S. Application Data
(63) Continuation of application No. 17/222,807, filed on Apr. 5, 2021, now Pat. No. 12,129,523.
(60) Provisional application No. 63/005,996, filed on Apr. 6, 2020.
(60) Provisional application No. 63/062,406, filed on Aug. 6, 2020.
(60) Provisional application No. 63/136,449, filed on Jan. 12, 2021.
(60) Provisional application No. 63/154,571, filed on Feb. 26, 2021.

(51) Int. Cl. C12Q 1/70

(52) U.S. Cl. CPC C12Q 1/701; C12Q 2600/158

(57) ABSTRACT

Described herein are methods useful for detecting and diagnosing pathogen infection using PCR and sequencing.

### CROSS REFERENCE TO RELATED APPLICATIONS

This application is a continuation of U.S. patent application Ser. No. 17/222,807, filed Apr. 5, 2021, which claims the benefit of U.S. Provisional App. Ser. No. 63/005,996 filed Apr. 6, 2020; U.S. Provisional App. Ser. No. 63/062,406 filed Aug. 6, 2020; U.S. Provisional App. Ser. No. 63/136,449 filed Jan. 12, 2021; and U.S. Provisional App. Ser. No. 63/154,571 filed Feb. 26, 2021; each of which is incorporated by reference herein in its entirety.

### SEQUENCE LISTING

The instant application contains a Sequence Listing which has been submitted in XML format via EFS-Web and is hereby incorporated by reference in its entirety. Said XML copy, created on Nov. 28, 2024, is named 52652-707301 SL_v2 18sep.2024.xml and is 772 Kilobytes in size.

### BACKGROUND

The inventors and the Applicant intend to freely license certain subject matter described herein to entities advancing the shared cause of ending the COVID-19 Pandemic consistent with the Open Covid Pledge dated Mar. 31, 2020.

Viral diseases and outbreaks of viral diseases have plagued mankind for millennia. An important part of identifying, monitoring, and guiding the response to viral disease is the ability to efficiently and accurately test individuals for viral infection. The novel coronavirus SARS-Cov-2, which causes COVID-19, emerged in late 2019 in Wuhan province of China, from there, and throughout the first part of 2020 the virus quickly spread around the world overwhelming medical systems and paralyzing commerce. There exists a need for tests that are quick, sensitive, and cost-effective.

### SUMMARY

Described herein is a method of diagnosing an individual with a pathogen infection. The method uses PCR and sequencing to achieve highly specific and sensitive detection of viral genomes from biological samples. Features of the methods described herein that allow for such detection include: 1) reverse transcription and/or amplification directly in a lysis agent or after lysis conditions without purification or isolation; 2) the presence of a synthetic nucleic acid that is able to amplified by oligonucleotide primers that target a viral sequence of interest, but that comprises a different distinguishable intervening sequence, spiked into the reverse transpiration amplification mixture; and allowing for more accurate quantification and lower thresholds of detection; 3) indexes to allow multiplexing by next generation sequencing. In certain embodiments, the pathogen is a viral infection (e.g., SARS-CoV-2). The methods herein can also be multiplexed to allow more than one viral pathogen to be detected (e.g., SARS-CoV-2 and influenza A or B or both).

Described herein in one aspect is a method of diagnosing an individual with a pathogen infection, the method comprising: a) providing a biological sample from said individual; a) contacting said biological sample from said individual with a lysis agent, to obtain a lysed biological sample; b) performing a polymerase chain reaction (PCR) on said lysed biological sample to obtain a PCR amplified lysed biological sample, wherein said PCR reaction on said lysed biological sample is performed with a first set of PCR primers, wherein said first set of PCR primers amplifies a pathogen nucleic acid sequence; c) sequencing said PCR amplified lysed biological sample using next generation sequencing; and d) providing a positive diagnosis for said pathogen infection if a pathogen sequence is detected by said PCR or by said sequencing or providing a negative diagnosis for said individual if a pathogen sequence is not detected by said PCR or by said sequencing. In certain embodiments, said individual is a human individual. In certain embodiments, said pathogen infection comprises a bacterial infection, a viral infection, a fungal infection, and combinations thereof. In certain embodiments, said bacterial infection is an infection by the genera Streptococcus, Pseudomonas, Shigella, Campylobacter, Salmonella, Clostridium, or Escherichia, and combinations thereof. In certain embodiments, said fungal infection is an infection by Candida, Blastomyces, Cryptococcus, Coccidoides, Histoplasma, Paracoccidioides, Sporothrix, or Pneumocystis, and combinations thereof. In certain embodiments, the viral infection is an infection by a DNA virus. In certain embodiments, said DNA virus comprises Hepatitis A, Hepatitis B, Hepatitis C, papillomavirus, Epstein-Barr virus, varicella, or variola, and combinations thereof. In certain embodiments, said viral infection is an infection by an RNA virus. In certain embodiments, said RNA virus comprises an influenza virus, a coronavirus, a polio virus, a measles virus, an Ebola virus, a retrovirus, or an Orthomyxovirus. In certain embodiments, said viral infection is a coronavirus infection. In certain embodiments, said coronavirus infection is SARS-COV-2 infection. In certain embodiments, said biological sample from said individual is from a blood sample, a plasma sample, a serum sample, a cheek swab, a urine sample, a semen sample, a vaginal swab, a stool sample, a nasopharyngeal swab, mid-turbinate swab, or any combination thereof. In certain embodiments, said biological sample from said individual is from a nasopharyngeal swab, a mid-turbinate swab, or any combination thereof. In certain embodiments, the method comprises adding a synthetic nucleic acid to said lysis agent or said lysed biological sample. In certain embodiments, said synthetic nucleic acid is an RNA. In certain embodiments, said synthetic nucleic acid is a DNA. In certain embodiments, said synthetic nucleic acid comprises a set of sequences configured to be bound by said first set of PCR primers. In certain embodiments, said first set of primers amplifies both said pathogen nucleic acid sequence and said synthetic nucleic acid. In certain embodiments, said synthetic nucleic acid sequence comprises a nucleotide sequence that is not identical to said pathogen nucleic acid sequence. In certain embodiments, the method comprises performing a reverse transcription reaction on said lysed biological sample. In certain embodiments, said reverse transcription reaction is performed before said performing said polymerase chain reaction. In certain embodiments, said reverse transcription reaction is performed without further purification of said lysed biological sample. In certain embodiments, said reverse transcription reaction on said lysed biological sample produces viral cDNA. In certain embodiments, said viral cDNA is coronavirus cDNA. In certain embodiments, said coronavirus cDNA is SARS-COV-2 cDNA. In certain embodiments, said reverse transcription reaction and said PCR is a single-step reaction. In certain embodiments, said PCR is an end-point analysis. In certain embodiments, said PCR is not a real-time PCR reaction. In certain embodiments, said first set of PCR primers amplifies a coronavirus nucleic acid sequence. In certain embodiments, said coronavirus nucleic acid sequence is a SARS-COV-2 nucleic acid sequence. In certain embodiments, said SARS-COV-2 nucleic acid sequence comprises the N1 or S2 gene. In certain embodiments, the method comprises a second set of primers. In certain embodiments, said second set of PCR primers amplifies a human nucleic acid sequence. In certain embodiments, said second set of PCR primers amplifies a human nucleic acid sequence selected from GAPDH, ACTB, RPP30, and combinations thereof. In certain embodiments, said second set of PCR primers amplifies human RPP30. In certain embodiments, the second set of PCR primer comprises a mixture of primers with sequencing adaptor sequences and primers without sequencing adaptor sequences. In certain embodiments, a ratio of primers with sequencing adaptor sequences to primers without sequencing adaptor sequences is about 1:1, about 1:2, about 1:3 or about 1:4. In certain embodiments, said PCR comprises from 30 to 45 amplification cycles. In certain embodiments, said PCR comprises from 35 to 45 amplification cycles. In certain embodiments, said PCR comprises from 39 to 42 amplification cycles. In certain embodiments, said first set of PCR primers, said second set of PCR primers, or both said first set of PCR primers and said second set of PCR primers comprises a nucleic acid sequence comprising a variable nucleotide sequence. In certain embodiments, said variable nucleotide sequence is a sample ID unique for said individual. In certain embodiments, said first set of PCR primers, said second set of PCR primers, or both said first set of PCR primers and said second set of PCR primers comprises an adapter sequence for a next-generation sequencing reaction. In certain embodiments, said method can detect less than 10 copies of pathogen genome. In certain embodiments, said method can detect less than 5 copies of pathogen genome. In certain embodiments, said pathogen genome is a coronavirus genome. In certain embodiments, said coronavirus genome is a SARS-COV-2 genome. In certain embodiments, said positive diagnosis for coronavirus if a coronavirus sequence is detected by said PCR is a SARS-COV-2 diagnosis. In certain embodiments, the method determines a strain of coronavirus. In certain embodiments, the method determines a strain of COVD-19.

Also described herein is a method of diagnosing an individual with a pathogen infection, the method comprising: (a) providing a biological sample from said individual; (c) performing a polymerase chain reaction (PCR) on said biological sample to obtain a PCR amplified biological sample, wherein said PCR reaction on said biological sample is performed with a first set of PCR primers, wherein said first set of PCR primers amplifies a pathogen nucleic acid sequence; (d) sequencing said PCR amplified biological sample using next generation sequencing; and (e) providing a positive diagnosis for said pathogen infection if a pathogen sequence is detected by said PCR or by said sequencing or providing a negative diagnosis for said individual if a pathogen sequence is not detected by said PCR or by said sequencing. In certain embodiments, said individual is a human individual. In certain embodiments, said pathogen infection comprises a bacterial infection, a viral infection, a fungal infection, and combinations thereof. In certain embodiments, said bacterial infection is an infection by the genera Streptococcus, Pseudomonas, Shigella, Campylobacter, Salmonella, Clostridium, or Escherichia, and combinations thereof. In certain embodiments, said fungal infection is an infection by Candida, Blastomyces, Cryptococcus, Coccidoides, Histoplasma, Paracoccidioides, Sporothrix, or Pneumocystis, and combinations thereof. In certain embodiments, the viral infection is an infection by a DNA virus. In certain embodiments, said DNA virus comprises Hepatitis A, Hepatitis B, Hepatitis C, papillomavirus, Epstein-Barr virus, varicella, or variola, and combinations thereof. In certain embodiments, said viral infection is an infection by an RNA virus. In certain embodiments, said RNA virus comprises an influenza virus, a coronavirus, a polio virus, a measles virus, an Ebola virus, a retrovirus, or an Orthomyxovirus. In certain embodiments, said viral infection is a coronavirus infection. In certain embodiments, said coronavirus infection is SARS-COV-2 infection. In certain embodiments, said biological sample from said individual is from a blood sample, a plasma sample, a serum sample, a cheek swab, a urine sample, a semen sample, a vaginal swab, a stool sample, a nasopharyngeal swab, mid-turbinate swab, or any combination thereof. In certain embodiments, said biological sample from said individual is from a nasopharyngeal swab, a mid-turbinate swab, or any combination thereof. In certain embodiments, the method comprises adding a synthetic nucleic acid to a lysis agent or said biological sample. In certain embodiments, said synthetic nucleic acid is an RNA. In certain embodiments, said synthetic nucleic acid is a DNA. In certain embodiments, said synthetic nucleic acid comprises a set of sequences configured to be bound by said first set of PCR primers. In certain embodiments, said first set of primers amplifies both said pathogen nucleic acid sequence and said synthetic nucleic acid. In certain embodiments, said synthetic nucleic acid sequence comprises a nucleotide sequence that is not identical to said pathogen nucleic acid sequence. In certain embodiments, the method comprises performing a reverse transcription reaction on said biological sample. In certain embodiments, said reverse transcription reaction is performed before said performing said polymerase chain reaction. In certain embodiments, said reverse transcription reaction is performed without further purification of said biological sample. In certain embodiments, said reverse transcription reaction on said biological sample produces viral cDNA. In certain embodiments, said viral cDNA is coronavirus cDNA. In certain embodiments, said coronavirus cDNA is SARS-COV-2 cDNA. In certain embodiments, said reverse transcription reaction and said PCR is a single-step reaction. In certain embodiments, said PCR is an end-point analysis. In certain embodiments, said PCR is not a real-time PCR reaction. In certain embodiments, said first set of PCR primers amplifies a coronavirus nucleic acid sequence. In certain embodiments, said coronavirus nucleic acid sequence is a SARS-COV-2 nucleic acid sequence. In certain embodiments, said SARS-COV-2 nucleic acid sequence comprises the N1 or S2 gene. In certain embodiments, the method comprises a second set of primers. In certain embodiments, said second set of PCR primers amplifies a human nucleic acid sequence. In certain embodiments, said second set of PCR primers amplifies a human nucleic acid sequence selected from GAPDH, ACTB, RPP30, and combinations thereof. In certain embodiments, said second set of PCR primers amplifies human RPP30. In certain embodiments, the second set of PCR primer comprises a mixture of primers with sequencing adaptor sequences and primers without sequencing adaptor sequences. In certain embodiments, a ratio of primers with sequencing adaptor sequences to primers without sequencing adaptor sequences is about 1:1, about 1:2, about 1:3 or about 1:4. In certain embodiments, said PCR comprises from 30 to 45 amplification cycles. In certain embodiments, said PCR comprises from 35 to 45 amplification cycles. In certain embodiments, said PCR comprises from 39 to 42 amplification cycles. In certain embodiments, said first set of PCR primers, said second set of PCR primers, or both said first set of PCR primers and said second set of PCR primers comprises a nucleic acid sequence comprising a variable nucleotide sequence. In certain embodiments, said variable nucleotide sequence is a sample ID unique for said individual. In certain embodiments, said first set of PCR primers, said second set of PCR primers, or both said first set of PCR primers and said second set of PCR primers comprises an adapter sequence for a next-generation sequencing reaction. In certain embodiments, said method can detect less than 10 copies of pathogen genome. In certain embodiments, said method can detect less than 5 copies of pathogen genome. In certain embodiments, said pathogen genome is a coronavirus genome. In certain embodiments, said coronavirus genome is a SARS-COV-2 genome. In certain embodiments, said positive diagnosis for coronavirus if a coronavirus sequence is detected by said PCR is a SARS-COV-2 diagnosis. In certain embodiments, the method determines a strain of coronavirus. In certain embodiments, the method determines a strain of COVD-19.

Described herein in another aspect is a method of diagnosing an individual with a pathogen infection, the method comprising amplifying nucleic acids from a biological sample from said individual using a first set of PCR primers thereby obtaining amplified nucleic acids, wherein said first set of PCR primers amplifies a pathogen nucleic acid sequence and a synthetic nucleic acid sequence from said biological sample, wherein said synthetic nucleic acid sequence differs from said pathogen nucleic acid sequence by at least one nucleotide. In certain embodiments, said individual is a human individual. In certain embodiments, said pathogen infection comprises a bacterial infection, a viral infection, or a fungal infection. In certain embodiments, said bacterial infection is an infection by the genera Streptococcus, Pseudomonas, Shigella, Campylobacter, Salmonella, Clostridium, or Escherichia, and combinations thereof. In certain embodiments, said fungal infection is an infection by Candida, Blastomyces, Cryptococcus, Coccidoides, Histoplasma, Paracoccidioides, Sporothrix, or Pneumocystis, and combinations thereof. In certain embodiments, the viral infection is an infection by a DNA virus. In certain embodiments, said DNA virus comprises hepatitis B, Hepatitis C, papillomavirus, Epstein-Barr virus, varicella, variola, or any combination thereof. In certain embodiments, said viral infection is an infection by an RNA virus. In certain embodiments, said RNA virus comprises an influenza virus, a coronavirus, a polio virus, a measles virus, an Ebola virus, a retrovirus, or an Orthomyxovirus. In certain embodiments, said viral infection is a coronavirus infection. In certain embodiments, said coronavirus infection is SARS-COV-2 infection. In certain embodiments, said biological sample from said individual is from a blood sample, a plasma sample, a serum sample, a cheek swab, a urine sample, a semen sample, a vaginal swab, a stool sample, a nasopharyngeal swab, mid-turbinate swab, or any combination thereof. In certain embodiments, said biological sample from said individual is from a nasopharyngeal swab, a mid-turbinate swab, or any combination thereof. In certain embodiments, said synthetic nucleic acid is an RNA. In certain embodiments, said synthetic nucleic acid is a DNA. In certain embodiments, said synthetic nucleic acid comprises a set of sequences configured to be bound by said first set of PCR primers. In certain embodiments, said synthetic nucleic acid sequence differs from said pathogen nucleic acid sequence by at least 5 nucleotides. In certain embodiments, said pathogen infection is diagnosed based upon the ratio of synthetic nucleic acid sequence to pathogen nucleic acid sequence. In certain embodiments, the method comprises performing a reverse transcription reaction on said nucleic acids from said biological sample. In certain embodiments, said reverse transcription reaction on said nucleic acids from said biological sample produces coronavirus cDNA. In certain embodiments, said coronavirus cDNA is SARS-COV-2 cDNA. In certain embodiments, said amplifying nucleic acids comprises a PCR reaction. In certain embodiments, said PCR reaction is an end-point analysis. In certain embodiments, said PCR reaction is not a real-time PCR reaction. In certain embodiments, said first set of PCR primers amplifies a coronavirus nucleic acid sequence. In certain embodiments, said coronavirus nucleic acid sequence is a SARS-COV-2 nucleic acid sequence. In certain embodiments, said SARS-COV-2 nucleic acid sequence comprises the N1 or S2 gene. In certain embodiments, said first set of PCR primers comprises a nucleic acid sequence comprising a variable nucleotide sequence. In certain embodiments, said variable nucleotide sequence is a sample ID unique for said individual. In certain embodiments, said first set of PCR primers comprises an adapter sequence for a next-generation sequencing reaction. In certain embodiments, the method comprises amplifying nucleic acids from said biological sample using a second set of PCR primers, wherein said second set of PCR primers amplifies a human nucleic acid sequence. In certain embodiments, said second set of PCR primers amplifies a nucleic acid sequence selected from GAPDH, ACTB, RPP30, and combinations thereof. In certain embodiments, said second set of PCR primers amplifies human RPP30. In certain embodiments, the second set of PCR primer comprises a mixture of primers with sequencing adaptor sequences and primers without sequencing adaptor sequences. In certain embodiments, a ratio of primers with sequencing adaptor sequences to primers without sequencing adaptor sequences is about 1:1, about 1:2, about 1:3 or about 1:4. In certain embodiments, said PCR comprises from 30 to 45 amplification cycles. In certain embodiments, said PCR comprises from 35 to 45 amplification cycles. In certain embodiments, said PCR comprises from 39 to 42 amplification cycles. In certain embodiments, said second set of PCR primers comprises a nucleic acid sequence comprising a variable nucleotide sequence. In certain embodiments, said variable nucleotide sequence is a sample ID unique for said individual. In certain embodiments, said second set of PCR primers comprises an adapter sequence a next-generation sequencing reaction. In certain embodiments, sequencing said amplified nucleic acids from said biological sample using a next-generation sequencing technology. In certain embodiments, said method can detect less than 10 copies of pathogen genome. In certain embodiments, said method can detect less than 5 copies of pathogen genome. In certain embodiments, said pathogen genome is coronavirus genome. In certain embodiments, said pathogen genome is SARS-COV-2 genome. In certain embodiments, the method determines a strain of coronavirus. In certain embodiments, the method determines a strain of SARS-COV-2.

Also described herein in another aspect is a synthetic nucleic acid comprising a 5′ proximal region, a 3′ proximal region and an intervening nucleic acid sequence. In certain embodiments, said synthetic nucleic acid comprises RNA. In certain embodiments, said synthetic nucleic acid comprises DNA. In certain embodiments, said 5′ proximal region comprises a viral nucleic acid sequence. In certain embodiments, said viral nucleic acid sequence comprises a coronavirus sequence. In certain embodiments, said viral nucleic acid sequence comprises a SARS-COV-2 sequence. In certain embodiments, said 3′ proximal region comprises a viral nucleic acid sequence. In certain embodiments, said viral nucleic acid sequence comprises a coronavirus sequence. In certain embodiments, said viral nucleic acid sequence comprises a SARS-COV-2 sequence. In certain embodiments, said 5′ proximal region, said 3′ proximal region, or both said 5′ proximal region and said 3′ proximal region are less than about 30 nucleotides in length. In certain embodiments, said 5′ proximal region, said 3′ proximal region, or both said 5′ proximal region and said 3′ proximal region are less than about 25 nucleotides in length. In certain embodiments, said 5′ proximal region, said 3′ proximal region, or both said 5′ proximal region and said 3′ proximal region are less than about 20 nucleotides in length. In certain embodiments, said 5′ proximal region is at the 5′ terminus of said synthetic nucleic acid. In certain embodiments, said 3′ proximal region is at the 3′ terminus of said synthetic nucleic acid. In certain embodiments, said intervening nucleic acid sequence is less than about 99%, 98%, 97%, 95%, 90%, 85%, 80%, or 75%, identical to a viral nucleic acid sequence. In certain embodiments, said synthetic nucleic acid sequence is a coronavirus sequence. In certain embodiments, said synthetic nucleic acid sequence is a SARS-COV-2 sequence. In certain embodiments the synthetic nucleic acid is for use in a method to detect pathogen infection in said individual. In certain embodiments, said pathogen infection is a coronavirus infection. In certain embodiments, said viral infection is a SARS-COV-2 infection.

In another aspect described herein is a composition, comprising a synthetic nucleic acid molecule comprising a first nucleic acid sequence and a second nucleic sequence, wherein (1) the first nucleic acid sequence is identical to a sequence from a pathogen nucleic acid molecule, and (2) the second nucleic acid sequence is not identical to a sequence the pathogen nucleic acid molecule. In certain embodiments, the first nucleic acid sequence is located to the 3′ the second nucleic acid sequence. In certain embodiments, the synthetic nucleic acid molecule further comprises a third nucleic acid sequence, wherein the third nucleic acid sequence is identical to a second sequence from the pathogen nucleic acid molecule. In certain embodiments, the third nucleic acid sequence is 5′ to the second nucleic acid sequence. In certain embodiments, the first nucleic acid sequence or the third nucleic acid sequence is less than 5, 10, 15, 20, 25, or 30 nucleotides. In certain embodiments, the second nucleic acid sequence comprises a total number of nucleotides less than 25, 50, 100, 150, 200, or 500 nucleotides. In certain embodiments, the second nucleic acid sequence comprises a total number of nucleotides greater than 25, 50, 100, 150, 200, or 500 nucleotides. In certain embodiments, the synthetic nucleic acid molecule is a ribonucleic acid (RNA) molecule, a deoxyribonucleic acid (DNA) molecule, or an RNA-DNA hybrid molecule. In certain embodiments, the first nucleic acid sequence, the third nucleic acid sequence, or both the first nucleic acid sequence and the third nucleic acid sequence comprise a primer binding site. In certain embodiments, the composition further comprises the pathogen nucleic acid molecule. In certain embodiments, the pathogen nucleic molecule is from a pathogen, wherein the pathogen comprises a bacterium, a virus, a fungus, or combinations thereof. In certain embodiments, the bacterium is from the genera Streptococcus, Pseudomonas, Shigella, Campylobacter, Salmonella, Clostridium, or Escherichia, and combinations thereof. In certain embodiments, the fungus is Candida, Blastomyces, Cryptococcus, Coccidoides, Histoplasma, Paracoccidioides, Sporothrix, or Pneumocystis, and combinations thereof. In certain embodiments, the virus is a DNA virus. In certain embodiments, the DNA virus comprises hepatitis B, Hepatitis C, papillomavirus, Epstein-Barr virus, varicella, or variola, and combinations thereof. In certain embodiments, the virus is an RNA virus. In certain embodiments, the RNA virus comprises an influenza virus, a coronavirus, a polio virus, a measles virus, an Ebola virus, a retrovirus, or an Orthomyxovirus. In certain embodiments, the virus is a coronavirus. In certain embodiments, the coronavirus is a SARS-COV-2 virus. In certain embodiments, the composition further comprises a plurality of primers, wherein a primer of the plurality of primers is configured to hybridize to a sequence of the synthetic nucleic acid molecule or a sequence of the pathogen nucleic acid molecule. In certain embodiments, a sequence of the synthetic nucleic acid molecule and the sequence the pathogen nucleic acid molecule are identical. In certain embodiments, the synthetic nucleic acid molecule is amplified with the same efficiency as the pathogen nucleic acid molecule. In certain embodiments, the synthetic nucleic acid molecule is configured to create an amplification produce the same size or within 10 base pairs in size as an amplification product of the pathogen nucleic acid molecule.

In another aspect described herein is a method of diagnosing an individual with a pathogen infection, the method comprising: (a) providing a biological sample from said individual; (b) contacting said biological sample from said individual with a lysis agent, to obtain a lysed biological sample; (c) performing a polymerase chain reaction (PCR) on said lysed biological sample to obtain a PCR amplified lysed biological sample, wherein said PCR reaction on said lysed biological sample is performed with a first set of PCR primers, wherein said first set of PCR primers amplifies a pathogen nucleic acid sequence; (d) sequencing said PCR amplified lysed biological sample using next generation sequencing; and (e) providing a positive diagnosis for said pathogen infection if a pathogen sequence is detected by said PCR or by said sequencing or providing a negative diagnosis for said individual if a pathogen sequence is not detected by said PCR or by said sequencing.

In another aspect described herein is a composition comprising a plurality of synthetic nucleic acids with a distinct nucleic acid sequence, said plurality of synthetic nucleic acids with a distinct nucleic acid sequence composing a common 5′ sequence identical to a pathogen nucleic acid sequence, a common 3′ sequence identical to a pathogen nucleic acid sequence, and an intervening sequence that differs among the plurality of sequences. In certain embodiments, said plurality of synthetic nucleic acids with a distinct nucleic acid sequence are single-stranded. In certain embodiments, said plurality of synthetic nucleic acids with a distinct nucleic acid sequence are double-stranded. In certain embodiments, said plurality of synthetic nucleic acids with a distinct nucleic acid sequences consist of or comprise RNA. In certain embodiments, said plurality of synthetic nucleic acids with a distinct nucleic acid sequences consist of or comprise DNA. In certain embodiments, said common 5′ sequence identical to a pathogen nucleic acid sequence is 30 nucleotides or less. In certain embodiments, said common 3′ sequence identical to a pathogen nucleic acid sequence is 30 nucleotides or less. In certain embodiments, said intervening sequence is 50 nucleotides or less. In certain embodiments, said intervening sequence is 30 nucleotides or less. In certain embodiments, the pathogen nucleic acid sequence is from a bacterial pathogen, a fungal pathogen, or a viral pathogen. In certain embodiments, said bacterial pathogen is of the genera Streptococcus, Pseudomonas, Shigella, Campylobacter, Salmonella, Clostridium, or Escherichia, and combinations thereof. In certain embodiments, said fungal pathogen is Candida, Blastomyces, Cryptococcus, Coccidoides, Histoplasma, Paracoccidioides, Sporothrix, or Pneumocystis, and combinations thereof. In certain embodiments, said viral pathogen is a DNA virus. In certain embodiments, said DNA virus comprises hepatitis B, Hepatitis C, papillomavirus, Epstein-Barr virus, varicella, or variola, and combinations thereof. In certain embodiments, said viral pathogen is an RNA virus. In certain embodiments, said RNA virus comprises an influenza virus, a coronavirus, a polio virus, a measles virus, an Ebola virus, a retrovirus, an Orthomyxovirus, or combinations thereof. In certain embodiments, said viral pathogen is a coronavirus. In certain embodiments, said coronavirus is SARS-COV-2. In certain embodiments, said pathogen nucleic acid sequence is a nucleic acid sequence that encodes a coronavirus spike protein. In certain embodiments, said plurality of synthetic nucleic acids with a distinct nucleic acid sequence comprise or consist of a sequence selected from any one or more of S2_001, S2_002, S2_003, and S2_004. In certain embodiments, the composition is for use in a method of diagnosing or detecting infection with a pathogen. In certain embodiments, the composition is for use in a method of normalizing pathogen next-generation sequence reads.

Described herein is a method of detecting a Coronavirus infection in an individual, the method comprising: (a) providing a biological sample from said individual, wherein said biological sample comprises a Coronavirus synthetic RNA, wherein a sequence of said Coronavirus synthetic RNA differs from a naturally occurring Coronavirus nucleic acid sequence; (b) lysing said biological sample thereby producing a lysed biological sample; (c) performing a reverse transcription reaction on said lysed biological sample to obtain a lysed, reverse transcribed biological sample; (d) performing an amplification reaction on said lysed, reverse transcribed biological sample to obtain an amplified biological sample, wherein said amplification reaction on said lysed, reverse transcribed biological sample is performed with a set of Coronavirus primers specific for a Coronavirus nucleic acid sequence, wherein said set of Coronavirus primers amplifies said Coronavirus nucleic acid sequence and said Coronavirus synthetic RNA; and (e) sequencing said amplified biological sample using next generation sequencing. In certain embodiments, the method further comprises providing a positive diagnosis for Coronavirus infection if sequence reads from said Coronavirus nucleic acid sequence are detected.

In certain embodiments, said Coronavirus infection is a SARS-Cov-2 infection. In certain embodiments, providing said positive diagnosis for said Coronavirus infection or SARS-Cov-2 infection is provided if a ratio or a mathematical equivalent thereof of said sequence reads from said Coronavirus nucleic acid sequence to sequence reads of said Coronavirus synthetic RNA exceed a ratio of about 0.1. In certain embodiments, providing said positive diagnosis for Coronavirus infection is provided if said sequence reads from said Coronavirus nucleic acid and said sequence reads of Coronavirus synthetic RNA exceed about 100. In certain embodiments, said lysed biological sample is not isolated or purified before performing said reverse transcription reaction. In certain embodiments, lysing said biological sample and performing said reverse transcription reaction on said lysed biological sample occur in the same well, tube, or reaction vessel. In certain embodiments, lysing said biological sample comprises thermal lysis. In certain embodiments, said thermal lysis comprises heating said biological sample to a temperature of at least about 50° C. In certain embodiments, said biological sample from said individual, comprises a plurality of Coronavirus synthetic RNA sequences, wherein said plurality of Coronavirus synthetic RNA sequences comprise at least two distinct synthetic Coronavirus RNA sequences. In certain embodiments, said plurality of synthetic Coronavirus RNA sequences comprise at least four distinct synthetic Coronavirus RNA nucleic acid sequences. In certain embodiments, the synthetic RNA nucleic acid or the plurality of synthetic nucleic acids comprises an amount of guanine nucleotide that is from about 20% to about 30%, an amount of adenine nucleotide that is from about 20% to about 30%, an amount of cytosine nucleotide that is from about 20% to about 30%, an amount of uracil nucleotide that is from about 20% to about 30%. In certain embodiments, said synthetic Coronavirus RNA nucleic acid or said plurality of synthetic Coronavirus RNA nucleic acids comprise a ratio of guanine to cytosine to adenine to uracil that is about equal. In certain embodiments, said synthetic Coronavirus RNA nucleic acid or said plurality of synthetic Coronavirus RNA nucleic acids comprise a synthetic SARS-Cov-2 RNA nucleic acid or a plurality of synthetic SARS-Cov-2 RNA nucleic acids. In certain embodiments, the method further comprises detecting an Influenza A infection, an Influenza B infection, or a combination thereof. In certain embodiments, said amplification reaction on said lysed biological sample is performed with a set of influenza A primers specific for an influenza A nucleic acid sequence or a set of influenza B primers specific for an influenza B nucleic acid sequence. In certain embodiments, said set of influenza A primers specific for said influenza A nucleic acid sequence comprises the sequences set forth in SEQ ID NO: 24 or 25 and SEQ ID NO: 26, or SEQ ID NO: 27 and SEQ ID NO: 28. In certain embodiments, said set of influenza B primers specific for said influenza B nucleic acid sequence comprises the sequences set forth in SEQ ID NO: 29 or 30. In certain embodiments, said amplification reaction on said lysed biological sample is performed with a set of Influenza A primers specific for an influenza A nucleic acid sequence and a set of influenza B primers specific for an influenza B nucleic acid sequence. In certain embodiments, said biological sample from said individual further comprises an influenza A synthetic RNA, an influenza B synthetic RNA, or a combination thereof wherein said influenza A synthetic RNA, said influenza B synthetic RNA, or said combination thereof differs from a naturally occurring influenza A or influenza B nucleic acid sequence. In certain embodiments, the method further comprises providing a positive diagnosis for influenza A infection if a ratio or a mathematical equivalent thereof of sequence reads from influenza A to sequence reads of said influenza A synthetic RNA exceed a ratio of about 0.1. In certain embodiments, the method further comprises providing a positive diagnosis for influenza B infection if a ratio or a mathematical equivalent thereof of sequence reads from influenza A to sequence reads of said influenza B synthetic RNA exceed a ratio of about 0.1. In certain embodiments, said Coronavirus nucleic acid sequence is an N1 sequence, an S2 sequence, or a combination thereof. In certain embodiments, said Coronavirus nucleic acid sequence is a SARS-Cov-2 N1 sequence, a SARS-Cov-2 S2 sequence, or a combination thereof. In certain embodiments, said set of Coronavirus primers comprise the sequences set forth in SEQ ID NO: 13 and SEQ ID NO: 14, or SEQ ID NO: 18 and SEQ ID NO: 19. In certain embodiments, said set of Coronavirus primers comprise the sequences set forth in SEQ ID NO: 20 and SEQ ID NO: 21, or SEQ ID NO: 22 and SEQ ID NO: 23. In certain embodiments, said set of Coronavirus primers are present at a concentration from about 50 nanomolar to about 250 nanomolar. In certain embodiments, said set of Coronavirus primers are present at a concentration of about 100 nanomolar. In certain embodiments, said influenza A nucleic acid sequence is an influenza A matrix sequence, an influenza A non-structural protein 1 sequence, an influenza A hemagglutinin sequence, an influenza A neuraminidase sequence, an influenza A nucleoprotein sequence, and a combination thereof. In certain embodiments, said influenza B nucleic acid sequence is an influenza B matrix sequence, an influenza B non-structural protein 1 sequence, an influenza B hemagglutinin sequence, an influenza B neuraminidase sequence, an influenza B nucleoprotein sequence, and a combination thereof. In certain embodiments, any one or more of said set of Coronavirus primers, said set of influenza A primers, or said set of influenza B primers comprises one or more index sequences allowing sample multiplexing. In certain embodiments, the Coronavirus synthetic RNA comprises a nucleic acid sequence at least about 90% homologous to any one or more of the sequences set forth in any one of SEQ ID NOs: 1 to 12. In certain embodiments, said Coronavirus synthetic RNA comprises a plurality of distinct nucleic acid sequences at least about 90% homologous to any one or more of the sequences set forth in any one of SEQ ID NOs: 1 to 12. In certain embodiments, said Influenza A synthetic RNA comprises an RNA sequence at least about 90% homologous to any one or more of the sequences set forth in any one of SEQ ID NOs: 31 or 32. In certain embodiments, said Influenza B synthetic RNA comprises an RNA sequence at least about 90% homologous to the sequence set forth in SEQ ID NO: 33. In certain embodiments, said Coronavirus synthetic RNA comprises a plurality of four distinct nucleic acid sequences at least about 90% homologous to the four the sequences set forth in SEQ ID NOs: 1 to 4. In certain embodiments, said Coronavirus synthetic RNA, said influenza A synthetic RNA, or said influenza B synthetic RNA is present at a concentration from about 10 copies per/reaction to about 500 copies per reaction. In certain embodiments, said Coronavirus synthetic RNA is present at a concentration from about 10 copies per/reaction to about 500 copies per reaction. In certain embodiments, said Coronavirus synthetic RNA, said Influenza A synthetic RNA, or said Influenza B synthetic RNA is present at a concentration of about 200 copies per reaction. In certain embodiments, said Coronavirus synthetic RNA is present at a concentration of about 200 copies per/reaction. In certain embodiments, said biological sample comprises a nasal swab or a saliva sample. In certain embodiments, said biological sample comprises less than about 10 microliters of saliva of the individual or less than about 10 microliters of a buffer that has been inoculated with a nasal swab of the individual. In certain embodiments, said biological sample comprises less than about 10 microliters of a buffer that has been inoculated with a nasal swab. In certain embodiments, said amplification reaction on said lysed biological sample is performed with a primer pair specific for a sample control. In certain embodiments, said sample control is a housekeeping gene. In certain embodiments, said primer pair specific for said sample control is specific for RPP30. In certain embodiments, said primer pair specific for said sample control comprises a sequence set forth in SEQ ID NO: 15 or 16, and SEQ ID NO: 17.

Also described herein is a synthetic nucleic acid comprising: a 5′ proximal region comprising a first nucleotide sequence from a virus; a 3′ proximal region comprising a second nucleotide sequence from said virus; and an intervening nucleotide sequence, wherein said intervening nucleotide sequence comprises a percentage of guanine nucleotide that is from about 20% to about 30%, an amount of adenine nucleotide that is from about 20% to about 30%, an amount of cytosine nucleotide that is from about 20% to about 30%, an amount of uracil or thymidine nucleotide that is from about 20% to about 30%, and said intervening sequence differs from a naturally occurring sequence of the virus. In certain embodiments, said synthetic nucleic acid comprises DNA. In certain embodiments, said synthetic nucleic acid consists of DNA. In certain embodiments, said synthetic nucleic acid comprises RNA. In certain embodiments, said synthetic nucleic acid consists of RNA. In certain embodiments, said virus is an influenza A virus, an influenza B virus, or a coronavirus. In certain embodiments, said virus is a coronavirus. In certain embodiments, said coronavirus is SARS-Cov-2. In certain embodiments, said intervening nucleotide sequence nucleic acids comprise an about equal ratio of guanine to cytosine to adenine to uracil or thymidine nucleotides. In certain embodiments, said 3′ proximal region and said 5′ proximal region comprise a nucleotide sequence at least 90% homologous to a coronavirus S2 gene sequence. In certain embodiments, said 5′ proximal region and said 3′ proximal region comprise a nucleotide sequence at least 95% homologous to a coronavirus S2 gene sequence. In certain embodiments, said 5′ proximal region and said 3′ proximal region comprise a nucleotide sequence identical to a coronavirus S2 gene sequence. In certain embodiments, said 5′ proximal region and said 3′ proximal region comprise a nucleotide sequence at least 90% homologous to a coronavirus N1 gene sequence. In certain embodiments, said 5′ proximal region and said 3′ proximal region comprise a nucleotide sequence at least 95% homologous to a coronavirus N1 gene sequence. In certain embodiments, said 5′ proximal region and said 3′ proximal region comprise a nucleotide sequence identical to a coronavirus N1 gene sequence. In certain embodiments, said coronavirus N1 gene sequence or said coronavirus S2 gene sequence is a SARS-CoV-2 gene sequence. In certain embodiments, said sequence nucleic acid comprises a sequence that is at least 90% homologous to any one or more sequences set forth in any one of SEQ ID NOs: 1 to 12. In certain embodiments, said sequence nucleic acid comprises a sequence that is at least 95% homologous to any one or more sequences set forth in any one of SEQ ID NOs: 1 to 12. In certain embodiments, said sequence nucleic acid comprises a sequence that is identical to any one or more sequences set forth in any one of SEQ ID NOs: 1 to 12. In certain embodiments, described herein is a plurality of synthetic nucleic acids, wherein said plurality comprises synthetic nucleic acids comprising at least two distinct nucleotide sequences. In certain embodiments, said plurality comprises synthetic nucleic acids comprising at least two distinct nucleotide sequences. In certain embodiments, said plurality comprises at least four distinct nucleotide sequences. In certain embodiments, said four distinct nucleotide sequences are those set forth in SEQ ID NOs: 1 to 4. In certain embodiments, said four distinct nucleotide sequences are selected from those set forth in SEQ ID NOs: 5 to 12.

Also described herein is a reaction mixture for determining the presence or absence of a viral nucleic acid in a biological sample comprising a synthetic nucleic acid described herein or a plurality of synthetic nucleic acids described herein, at least a portion of said biological sample, and one or more enzyme or reagents sufficient to amplify said viral nucleic acid in said biological sample, if present. In certain embodiments, said biological sample is a human biological sample. In certain embodiments, said biological sample comprises saliva, a cheek swab, a nasopharyngeal swab, or a mid-turbinate swab. In certain embodiments, said biological sample comprises saliva or a nasopharyngeal swab. In certain embodiments, said viral nucleic acid is an influenza A, influenza B, or a coronavirus nucleic acid. In certain embodiments, said coronavirus nucleic acid is a is a Sars-Cov-2 nucleic acid. In certain embodiments, In certain embodiments, said one or more reagents are selected from the list consisting of a reverse transcriptase enzyme, dNTPs, a primer pair specific for said viral nucleotide sequence, a primer pair specific for a sample control nucleotide sequence, a magnesium salt, and combinations thereof. In certain embodiments, said primer pair specific for said sample control nucleotide sequence is specific for a human nucleotide sequence. In certain embodiments, said primer pair specific for said sample control nucleotide sequence is specific for a housekeeping gene. In certain embodiments, said primer pair specific for said sample control is specific for RPP30. In certain embodiments, said primer pair specific for said sample control comprises a sequence set forth in SEQ ID NO: 15 or 16, and SEQ ID NO: 17. In certain embodiments, said primer pair specific for said viral nucleotide sequence is specific for an influenza A nucleotide sequence, an influenza B nucleotide sequence and a coronavirus nucleotide sequence. In certain embodiments, said primer pair specific for said viral nucleotide sequence is specific for a coronavirus S1 or N2 sequence. In certain embodiments, said coronavirus S1 or N2 sequence is a CORONAVIRUS S1 or N2 nucleic acid sequence. In certain embodiments, said primer pair specific for said viral nucleotide sequence or said primer pair specific for said sample control nucleotide sequence comprises the sequence set forth in any one of SEQ ID NOs: 13 to 30 or 100 to 605. In certain embodiments, said primer pair specific for said viral nucleotide sequence or said primer pair specific for said sample control nucleotide sequence comprise the sequences set forth in SEQ ID NO: 13 and SEQ ID NO: 14; SEQ ID NO: 18 and SEQ ID NO: 19; SEQ ID NO: 20 and SEQ ID NO: 21; SEQ ID NO: 24 or 25 and SEQ ID NO: 26; SEQ ID NO: 29 and SEQ ID NO: 30. In certain embodiments, said primer pair specific for said viral nucleotide sequence or said primer pair specific for said sample control nucleotide sequence is present at a concentration from about 50 micromolar to about 250 micromolar. In certain embodiments, said primer pair specific for said viral nucleotide sequence or said primer pair specific for said sample control nucleotide sequence is present at a concentration of about 100 micromolar. In certain embodiments, said primer pair specific for said viral nucleotide sequence or said primer pair specific for said sample control nucleotide sequence is present at a concentration of about 200 micromolar. In certain embodiments, said Coronavirus synthetic RNA is present at a concentration from about 10 copies per/reaction to about 500 copies per reaction mixture. In certain embodiments, said Coronavirus synthetic RNA is present at a concentration of about 200 copies per rection mixture. In certain embodiments, the volume of said reaction mixture is from about 10 microliters to about 100 microliters. In certain embodiments, the volume of said reaction mixture is about 20 microliters.

Also described herein is a kit for determining the presence or absence of a viral nucleic acid in a biological sample comprising a synthetic nucleic acid described herein or a plurality of synthetic nucleic acids described herein and one or more enzyme or reagents sufficient to amplify said viral nucleic acid from said biological sample. In certain embodiments, the viral nucleic acid is an influenza A, influenza B, or a coronavirus nucleic acid. In certain embodiments, said coronavirus nucleic acid is a Coronavirus nucleic acid. In certain embodiments, said one or more reagents are selected from the list consisting of a reverse transcriptase enzyme, dNTPs, a primer pair specific for said viral nucleotide sequence, a primer pair specific for a sample control nucleotide sequence, a magnesium salt, and combinations thereof. In certain embodiments, said primer pair specific for said sample control nucleotide sequence is specific for a human nucleotide sequence. In certain embodiments, said primer pair specific for said sample control nucleotide sequence is specific for a housekeeping gene. In certain embodiments, said primer pair specific for said sample control is specific for RPP30. In certain embodiments, said primer pair specific for said sample control comprises a sequence set forth in SEQ ID NO: 15 or 16, and SEQ ID NO: 17. In certain embodiments, said primer pair specific for said viral nucleotide sequence is specific for an influenza A nucleotide sequence, an influenza B nucleotide sequence and a coronavirus nucleotide sequence. In certain embodiments, said primer pair specific for said viral nucleotide sequence is specific for a coronavirus S1 or N2 sequence. In certain embodiments, said coronavirus S1 or N2 sequence is a CORONAVIRUS S1 or N2 nucleic acid sequence. In certain embodiments, said primer pair specific for said viral nucleotide sequence or said primer pair specific for said sample control nucleotide sequence comprises the sequence set forth in any one of SEQ ID NOs: 13 to 30 or 100 to 605. In certain embodiments, said primer pair specific for said viral nucleotide sequence or said primer pair specific for said sample control nucleotide sequence comprise the sequences set forth in SEQ ID NO: 13 and SEQ ID NO: 14; SEQ ID NO: 18 and SEQ ID NO: 19; SEQ ID NO: 20 and SEQ ID NO: 21; SEQ ID NO: 24 or 25 and SEQ ID NO: 26; SEQ ID NO: 29 and SEQ ID NO: 30.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 illustrates an exemplary schematic of the diagnosis of viral infections.

FIGS. 2A and 2B illustrates data demonstrating the detection of SARS-CoV-2 nucleic acids in a sample.

FIG. 3 illustrates an exemplary priming scheme according to the current disclosure.

FIG. 4 illustrates the SwabSeq Diagnostic Testing Platform for COVID19.

FIG. 5 shows validation in clinical specimens and demonstrates a limit of detection equivalent to sensitive RT-qPCR reactions.

FIG. 6 shows a sequencing library design.

FIG. 7 shows that S2 primers show equivalent PCR efficiency when amplifying the COVID-19 amplicon and the synthetic S2 spike.

FIG. 8 shows that at very high viral concentrations SwabSeq maintains linearity.

FIG. 9 shows sequencing performed on MiSeq or NextSeq Machine exhibits similar sensitivity.

FIG. 10 show preliminary and Confirmatory Limit of Detection Data for RNA purified Samples using the NextSeq550.

FIG. 11 shows extraction-Free protocols into traditional collection medias and buffers require dilution to overcome effects of RT and PCR inhibition.

FIG. 12 shows exemplary development of a lightweight sample accessioning, collection and processing system to allow for scalable testing into the thousands of samples per day.

FIG. 13 show that preheating Saliva to 95 C for 30 minutes improves RT-PCR.

FIG. 14 shows that PCR inhibition has significant effect on amplification products.

FIG. 15 shows that tapestation increasing the number of PCR cycles and working with unpurified or inhibitory samples types (e.g. Saliva) was seen to increase the size of a nonspecific peak in the library preparation.

FIG. 16 shows that TaqPath decreases the number of S2 reads in SARS-CoV2-negative samples relative to NEB Luna.

FIG. 17 shows carryover contamination from template line in a MiSeq contributes to cross contamination.

FIG. 18 shows sequencing errors in amplicon read and potential amplicon mis-assignment.

FIG. 19 shows a visualization of different indexing strategies.

FIG. 20 shows computational correction for index mis-assignment using a mixed-model.

FIG. 21 shows quantifying the role of index mis-assignment as a source of noise in the S2 reads.

FIG. 22 shows the effects of lowered primer concentration on primer dimers and non-specific amplification products.

FIG. 23 shows diversified synthetic nucleic acid spike-in sequences.

FIG. 24 shows data obtained with N1 spike in (top) or S2 spike in (bottom) and detection of different amplicons N1 (top) or (S2) bottom.

FIG. 25 shows that combining N1 and S2 can increase sensitivity and of SARS-CoV2-detection compared to either individually.

FIG. 26 shows results for detection using different volumes of saliva sample.

FIG. 27 shows results for detection using different volumes of nasal swab sample.

FIG. 28 shows that SwabSeq can detect Influenza A or Influenza B.

FIG. 29 shows an exemplary algorithm for calling positive and negative samples.

### DETAILED DESCRIPTION

The frequency of outbreaks of highly contagious or highly pathogenic diseases is increasing. One third of worldwide deaths are attributable to infectious diseases, which are the second cause of mortality and disability worldwide. Obtaining a rapid, accurate readout for the identifying and diagnosing the infectious diseases that cause human illness is a critical component of diagnostic medicine, particularly in the case of viral infections, in order to implement effective public health responses and improve the delivery of human healthcare. Numerous methods for detecting viral infections have been developed for clinical diagnostic purposes, however most of these tests do not provide sufficiently rapid or high throughput readouts and/or are not feasible due to the resource burden associated with rapidly testing a rapidly increasing number of subjects.

Additionally, provided herein are methods and systems of diagnosing an individual with a viral infection. Such methods and systems, as describe herein, leverage polymerase chain reaction (PCR), library preparation strategies, and next generation sequencing to yield readouts or information that accurately identifies a viral infection and can be effectively scaled to meet the challenges associated with the need to efficiently test an increasing number of subjects. To facilitate the identification and diagnosis of a viral infection within a sample, the methods provide advances in or solutions for (1) the effective and efficient isolation of a nucleic acid sequence from a virus, (2) the effective and efficient processing of nucleic acid molecules corresponding to or derived from the virus, and (3) the effective and efficient multiplexing of samples that allow for the testing of multiple samples in parallel and reduces the overall resource burden of testing.

Described herein, is a method of detecting pathogen genome in a sample, the method comprising amplifying nucleic acids from a sample using a first set of PCR primers obtaining amplified nucleic acids, wherein said first set of PCR primers amplifies a viral nucleic acid sequence and a synthetic nucleic acid sequence. The synthetic nucleic acid sequence provides a sample and amplification control in the assay allowing for a lower limit of detection compared to amplification without the synthetic nucleic acid. The synthetic nucleic acid can be added (“spiked”) into a lysate that has been contacted to the biological sample or it can be present in the lysis buffer before the lysis buffer is contacted to the biological sample. The sample can be a biological sample. The biological sample can be from an individual. The synthetic nucleic acid can be added at any step along the way. More than one synthetic nucleic acid can be used in the method described herein. For example, one synthetic nucleic acid can serve as a control for one or more viral nucleic acids, and one synthetic nucleic acid can serve as a control for one or more human nucleic acids (e.g. a house keeping control), or for example a plurality of synthetic nucleic acids can be added to serve as a control for a plurality of pathogen sequences.

Also described herein is a synthetic nucleic acid comprising a 5′ proximal region, a 3′ proximal region, and an intervening nucleic acid sequence. The synthetic nucleic acid can comprise any sequence at the 5′ and 3′ ends that allows amplification of a pathogen sequence, provided that the sequence between the 5′ and 3′ ends is distinguishable by sequencing. In certain embodiments the sequence between the 5′ and 3′ ends is identical to the pathogen sequence except for a sequence of 1, 2, 3, 4, 5, 6, 7, 8, 9, or 10 nucleotides that differs from the pathogen sequence.

Also described herein is a synthetic nucleic acid comprising a 5′ proximal region, a 3′ proximal region, and an intervening nucleic acid sequence. The synthetic nucleic acid can comprise any sequence at the 5′ and 3′ ends that allows amplification of a pathogen sequence, provided that the sequence between the 5′ and 3′ ends is distinguishable by sequencing. In certain embodiments the sequence between the 5′ and 3′ ends is identical to the pathogen sequence except for a sequence of 1, 2, 3, 4, 5, 6, 7, 8, 9, or 10 nucleotides that differs from the pathogen sequence.

Also described herein is a method of detecting a Coronavirus infection in an individual, the method comprising: (a) providing a biological sample from said individual, wherein said biological sample comprises a Coronavirus synthetic RNA, wherein a sequence of said Coronavirus synthetic RNA differs from a naturally occurring Coronavirus nucleic acid sequence; (b) lysing said biological sample thereby producing a lysed biological sample; (c) performing a reverse transcription reaction on said lysed biological sample to obtain a lysed, reverse transcribed biological sample; (d) performing an amplification reaction on said lysed, reverse transcribed biological sample to obtain an amplified biological sample, wherein said amplification reaction on said lysed, reverse transcribed biological sample is performed with a set of Coronavirus primers specific for a Coronavirus nucleic acid sequence, wherein said set of Coronavirus primers amplifies said Coronavirus nucleic acid sequence and said Coronavirus synthetic RNA; and (e) sequencing said amplified biological sample using next generation sequencing. In certain embodiments, the method further comprising providing a positive diagnosis for Coronavirus infection if sequence reads from said Coronavirus nucleic acid sequence are detected. In certain embodiments, said Coronavirus infection is a SARS-Cov-2 infection. In certain embodiments, providing said positive diagnosis for said Coronavirus infection or SARS-Cov-2 infection is provided if a ratio or a mathematical equivalent thereof of said sequence reads from said Coronavirus nucleic acid sequence to sequence reads of said Coronavirus synthetic RNA exceed a ratio of about 0.1. In certain embodiments, providing said positive diagnosis for Coronavirus infection is provided if said sequence reads from said Coronavirus nucleic acid and said sequence reads of Coronavirus synthetic RNA exceed about 100.

Also described herein is a method of detecting a Coronavirus infection in an individual, the method comprising: (a) providing a biological sample from said individual, wherein said biological sample comprises a Coronavirus synthetic RNA, wherein a sequence of said Coronavirus synthetic RNA differs from a naturally occurring Coronavirus nucleic acid sequence; (b) lysing said biological sample thereby producing a lysed biological sample; (c) performing a reverse transcription reaction on said lysed biological sample to obtain a lysed, reverse transcribed biological sample; (d) performing an amplification reaction on said lysed, reverse transcribed biological sample to obtain an amplified biological sample, wherein said amplification reaction on said lysed, reverse transcribed biological sample is performed with a set of Coronavirus primers specific for a Coronavirus nucleic acid sequence, wherein said set of Coronavirus primers amplifies said Coronavirus nucleic acid sequence and said Coronavirus synthetic RNA. In certain embodiments, said Coronavirus infection is a SARS-Cov-2 infection.

Also described herein is a method of detecting a Coronavirus infection in an individual, the method comprising: (a) providing a biological sample from said individual, wherein said biological sample comprises a Coronavirus synthetic RNA, wherein a sequence of said Coronavirus synthetic RNA differs from a naturally occurring Coronavirus nucleic acid sequence; (b) lysing said biological sample thereby producing a lysed biological sample; (c) performing a reverse transcription reaction on said lysed biological sample to obtain a lysed, reverse transcribed biological sample. In certain embodiments, said Coronavirus infection is a SARS-Cov-2 infection.

Compositions comprising a synthetic nucleic acid molecule are also provided and useful in the method disclosed herein. The synthetic nucleotide is, generally, an in vitro transcribed or synthetic control RNA that is identical to the viral sequence targeted for amplification, except for a short, altered stretch that allows for distinguishing sequencing reads corresponding to the synthetic control from those corresponding to the pathogen sequence. For example, compositions are disclosed comprising a synthetic nucleic acid molecule comprising a first nucleic acid sequence and a second nucleic sequence, wherein the first nucleic acid sequence is identical to a sequence from a pathogen nucleic acid molecule, and the second nucleic acid sequence is not identical to a sequence the pathogen nucleic acid sequence. In some embodiments, the first nucleic acid sequence is located at the 3′ region of the synthetic nucleic acid molecule. In some embodiments, the synthetic nucleic acid molecule further comprises a third nucleic acid sequence, wherein the first nucleic acid sequence is identical to a first sequence from a pathogen nucleic acid molecule and wherein the third nucleic acid sequence is identical to a second sequence from a pathogen nucleic acid molecule. In some embodiments, the first nucleic acid sequence is located at a region 3′ of the second nucleic acid molecule, and the third nucleic acid sequence is located at a region 5′ of the second nucleic acid molecule. In some embodiments, the second nucleic acid sequence is less than 5, 10, 15, 20, 25, or 30 nucleotides. In some embodiments, the second nucleic synthetic nucleic acid molecule comprises a total number of nucleotides less than 25, 50, 100, 150, 200, or 500 nucleotides. In some embodiments, the second nucleic synthetic nucleic acid molecule comprises a total number of nucleotides greater than 25, 50, 100, 150, 200, or 500 nucleotides. In some embodiments, the synthetic nucleic acid mole is a ribonucleic acid (RNA) molecule, a deoxyribonucleic acid (DNA) molecule, or an RNA-DNA hybrid molecule. In certain embodiments, the synthetic nucleic acid molecule is PCR amplified with an efficiency within about 10% of the corresponding pathogen sequence. In certain embodiments, the synthetic nucleic acid molecule is PCR amplified with an efficiency within about 5% of the corresponding pathogen sequence. In certain embodiments, the synthetic nucleic acid molecule is PCR amplified with the same efficiency of the corresponding pathogen sequence.

A plurality of synthetic nucleic acids can be used according to the methods described herein to further increase sensitivity, reduce false positives, or improve the accuracy and or precision of quantification of nucleic acid sequences. The plurality may comprise 2, 3, 4, 5, 6, 7, 8, 9 or more distinct sequences capable of co-amplification with a set of primers specific for the pathogen sequence to be detected. The plurality may have certain characteristics that are desirable for the plurality. In certain embodiments, the melting temperature of the distinct sequences of the plurality may be substantially the same or within about 0.5° 10, 2°, 3°, 4°, or 5° Celsius of an average melting temperature of the plurality. In certain embodiments, the nucleotide make-up of the plurality targeted to be between about 30% and about 20% A, about 30% and about 20% G, about 30% and about 20% C, about 30% and 20% about T. In certain embodiments, the nucleotide make-up of the plurality targeted to be about 25% A, about 25% G, about 25% C, about 25% about T. In certain embodiments, the nucleotide make-up of the plurality targeted to be one or more of between about 30% and about 20% A, about 30% and about 20% G, about 30% and about 20% C, about 30% and 20% about T. In certain embodiments, the nucleotide make-up of the plurality targeted to be one or more of about 25% A, about 25% G, about 25% C, about 25% about T. In certain embodiments, the plurality of synthetic nucleic acids is selected or designed to minimize secondary structure or dimerization amongst the distinct sequences of the plurality.

Also described herein is a method of diagnosing an individual with a viral infection, the method comprising: (a) providing a biological sample from said individual; (b) contacting said biological sample from said individual with a lysis agent, to obtain a lysed biological sample; (c) performing a reverse transcription reaction on said lysed biological sample; (d) performing a polymerase chain reaction (PCR) on said lysed biological sample to obtain a PCR amplified lysed biological sample, wherein said PCR reaction on said lysed biological sample is performed with a first set of PCR primers and a second set of PCR primers, wherein said first set of PCR primers amplifies a viral nucleic acid sequence, wherein said second set of primers amplifies a genomic sequence of a species to which said individual belongs; (e) sequencing said PCR amplified lysed biological sample using next generation sequencing; and (f) optionally, providing a positive diagnosis for said viral infection if a viral sequence is detected by said PCR or by said sequencing or providing a negative diagnosis for said human individual if a viral sequence is not detected by said PCR or by said sequencing. In certain embodiments, said first set of PCR primers can amplify a synthetic nucleic acid sequence present in the lysed biological sample. The synthetic nucleic acid sequence comprises the same primer binding sites as the viral nucleic acid sequence, except the intervening nucleic acid sequence is different, such that it can be discriminated by sequencing. In certain embodiments, the synthetic nucleic acid sequence is an RNA.

Also described herein is a method of diagnosing an individual with a pathogen infection, the method comprising: (a) providing a biological sample from said individual; (b) contacting said biological sample from said individual with a lysis agent, to obtain a lysed biological sample; (c) performing a polymerase chain reaction (PCR) on said lysed biological sample to obtain a PCR amplified lysed biological sample, wherein said PCR reaction on said lysed biological sample is performed with a first set of PCR primers and a second set of PCR primers, wherein said first set of PCR primers amplifies a pathogen nucleic acid sequence, wherein said second set of primers amplifies a nucleic acid sequence of said individual; (d) sequencing said PCR amplified lysed biological sample using next generation sequencing; and (e) optionally, providing a positive diagnosis for said pathogen infection if a pathogen sequence is detected by said PCR or by said sequencing or providing a negative diagnosis for said human individual if a pathogen sequence is not detected by said PCR or by said sequencing. In certain embodiments, said first set of PCR primers can amplify a synthetic nucleic acid sequence present in the lysed biological sample. The synthetic nucleic acid sequence comprises the same primer binding sites as the viral nucleic acid sequence, except the intervening nucleic acid sequence is different, such that it can be discriminated by sequencing. In certain embodiments, the synthetic nucleic acid sequence is an RNA.

The methods described herein can be used to surveil pathogen presence in any number of samples, including non-human samples. Surveillance can comprise monitoring a herd of domesticated or wild animals, a wild-animal population, or enclosed live animals (e.g., zoos, wild-animal parks, or live animal markets where the animals are sold for food or as pets).

Also described herein is a method of surveilling for the presence of pathogen in a sample, the method comprising: (a) providing a sample; (b) contacting said sample with an extraction agent, to obtain an extracted sample; (c) performing a polymerase chain reaction (PCR) on said extracted sample to obtain a PCR amplified extracted sample, wherein said PCR reaction on said extracted sample is performed with a first set of PCR primers; (d) sequencing said PCR amplified lysed biological sample using next generation sequencing; and (e) optionally, providing a positive readout for said pathogen if a pathogen sequence is detected by said PCR or by said sequencing or providing a negative readout if a pathogen sequence is not detected by said PCR or by said sequencing. In certain embodiments, said first set of PCR primers can amplify a synthetic nucleic acid sequence present in the extracted biological sample. The synthetic nucleic acid sequence comprises the same primer binding sites as the pathogen nucleic acid sequence, except the intervening nucleic acid sequence is different, such that it can be discriminated by sequencing. In certain embodiments, the synthetic nucleic acid sequence is an RNA. In certain embodiments, the synthetic nucleic acid sequence is a DNA.

To achieve the identification, detection, and/or diagnosis of a viral infection, the methods and systems disclosed herein comprise (a) providing a biological sample from said individual; (b) contacting said biological sample from said individual with a lysis agent to obtain a lysed biological sample; (c) performing an initial nucleic acid extension reaction on said lysed biological sample; (d) performing a polymerase chain reaction (PCR) on said lysed biological sample to obtain a PCR amplified lysed biological sample, wherein said PCR reaction on said lysed biological sample is performed with a first set of PCR primers and a second set of PCR primers, wherein said first set of PCR primers amplifies a viral nucleic acid sequence, wherein said second set of primers amplifies a genomic sequence of a species to which said individual belongs; (e) sequencing said PCR amplified lysed biological sample using next generation sequencing; and (f) providing a positive diagnosis for viral infection if a viral sequence, or derivative thereof, is detected by said PCR or by said sequencing, or providing a negative diagnosis for said human individual if a coronavirus sequence is not detected by said PCR or by said sequencing.

Disclosed herein are also method of nucleic acid processing for the detection of a viral infection. Such methods comprise (a) providing a sample comprising a viral nucleic acid molecule and a host nucleic acid molecule; (b) generating a barcoded viral nucleic acid molecule by performing a nucleic acid extension reaction on said viral nucleic acid molecule using a first primer comprising a barcode sequence; (c) generating a barcoded host nucleic acid molecule by performing a nucleic acid extension reaction on said host nucleic acid molecule using a second primer comprising said barcode sequence; (d) sequencing said barcoded viral nucleic acid molecule and said barcoded host nucleic acid molecule to identify (i) said barcode sequence and (ii) a sequence corresponding to said viral nucleic acid molecule, or a derivative thereof, and said host nucleic acid molecule; and (e) providing a positive diagnosis for a viral infection if said sequence corresponding to said viral nucleic acid molecule is identified in (d).

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real-time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection, or identification.

The term “genome,” as used herein, refers to genomic information from a plant, animal, bacteria, fungi, or virus, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). “Next-generation sequencing” refers to method of high throughput sequencing that is not Sanger sequencing. Sequencing can be performed by various systems currently available, such as, without limitation, a sequencing system by Illumina® (e.g., iSeq 100, MiniSeq, MiSeq or NextSeq series of machines) Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “sample,” as used herein, is used broadly and can refer to environmental samples (e.g., water samples, sewage samples), samples of raw or prepared food, samples generated from a population of non-human individuals (e.g., wild- or domesticated animals), or biological samples from human or non-human animals. The sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a nasopharyngeal swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, host cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

As used herein the term “pathogen” includes any organism or virus capable of causing disease in a population of individuals, such population could include animals or plants. This also encompasses pathogens that reside in a carrier individual or species, where the pathogen does not cause disease in the carrier individual or species, but can be transmitted to another induvial or species to cause disease. As used herein, pathogens include, but are not limited to bacteria, protozoa, fungi, nematodes, viroids and viruses, or any combination thereof, wherein each pathogen is capable, either by itself or in concert with another pathogen, of eliciting disease in vertebrates including but not limited to mammals, and including but not limited to humans. As used herein the term “host” refers to an organism that can be infected by a pathogen and includes plants, animals, vertebrates, mammals, rodents, cows, horses, pigs, fowl, chickens, geese, ducks, fish, shellfish and the like.

As used herein, the term “Bacteria,” or “Eubacteria”, refers to a domain of prokaryotic organisms. Bacteria include at least 11 distinct groups as follows: (1) Gram-positive (gram+) bacteria, of which there are two major subdivisions: (i) high G+C group (Actinomycetes, Mycobacteria, Micrococcus, others) (ii) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles. “Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium. “Gram-positive bacteria” include cocci, nonsporulating rods, and sporulating rods. The genera of Grampositive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces. “Pathogenic bacteria” or “pathogenic bacterium” are bacterial species that cause disease(s) in another host organism (e.g., animals and plants) by directly infecting the other organism, or by producing agents that causes disease(s) in another organism (e.g., bacteria that produce pathogenic toxins and the like).

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “housekeeping gene,” “housekeeping control,” or similar such term as used herein, generally, refers to a gene expressed in an organism under both normal and patho-physiological conditions or that is expressed by different tissue and cell types. In some cases, the housekeeping gene is a constitutive gene that is required for the maintenance of basic cellular function. The housekeeping gene is generally expressed at a relatively constant rate in most normal and patho-physiological conditions. Specific examples of housekeeping genes include, without limitation, RPP30, Beta actin, and/or GAPDH.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The terms “a,” “an,” and “the,” as used herein, generally refers to singular and plural references unless the context clearly dictates otherwise.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

As used herein the term “individual,” “patient,” or “subject” refers to individuals diagnosed with, suspected of being afflicted with, or at-risk of developing at least one disease for which the described compositions and method are useful for detecting. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

### Pathogenic Infections

The methods described herein can allow the detection of many different pathogens including bacteria, viruses, fungi, protists, nematodes, and viroids.

For example, pathogens that can be detected or diagnosed include, but are not limited to, Bacillus anthracis (anthrax), Clostridium botulinum toxin (botulism), Yersinia pestis (plague). Variola major (smallpox) and other related pox viruses, Francisella tularensis (tularemia), Viral hemorrhagic fevers, Arenaviruses, (e.g., Junin, Machupo, Guanarito, Chapare, Lassa, and/or Lujo), Bunyaviruses (e.g. Hantaviruses causing Hanta Pulmonary syndrome, Rift Valley Fever, and/or Crimean Congo Hemorrhagic Fever), Flaviviruses, Dengue, Filoviruses (e.g. Ebola and Marburg viruses), Burkholderia pseudomallei (melioidosis), Coxiella burnetii (Q fever), Brucella species (brucellosis), Burkholderia mallei (glanders), Chlamydia psittaci (Psittacosis), Ricin toxin (Ricinus communis), Epsilon toxin (Clostridium perfringens), Staphylococcus enterotoxin B (SEB), Typhus fever (Rickettsia prowazekii), Food- and waterborne pathogens, Diarrheagenic E. coli, Pathogenic Vibrios, Shigella species, Salmonella, Listeria monocytogenes, Campylobacter jejuni, Yersinia enterocolitica, Caliciviruses, Hepatitis A, Cryptosporidium parvum, Cyclospora cayatanensis, Giardia lamblia, Entamoeba histolytica, Toxoplasma gondii, Naegleria fowleri, Balamuthia mandrillaris, Fungi, Microsporidia, Mosquito-borne viruses (e.g. West Nile virus (WNV), LaCrosse encephalitis (LACV), California encephalitis, Venezuelan equine encephalitis (VEE), Eastern equine encephalitis (EEE), Western equine encephalitis (WEE), Japanese encephalitis virus (JE), St. Louis encephalitis virus (SLEV), Yellow fever virus (YFV), Chikungunya virus, Zika virus, Nipah and Hendra viruses, Additional hantaviruses, Tickborne hemorrhagic fever viruses, Bunyaviruses, Severe Fever with Thrombocytopenia Syndrome virus (SFTSV), Heartland virus, Flaviviruses (e.g. Omsk Hemorrhagic Fever virus, Alkhurma virus, Kyasanur Forest virus), Tickborne encephalitis complex flaviviruses, Tickborne encephalitis viruses, Powassan/Deer Tick virus, Tuberculosis, including drug-resistant TB, Influenza virus, Rabies virus, Prions, Streptococcus, Pseudomonas, Shigella, Campylobacter, Salmonella, Clostridium, Escherichia, Hepatitis B, Hepatitis C, papillomavirus, Epstein-Barr virus, varicella, variola, Orthomyxovirus, Severe acute respiratory syndrome associated coronavirus (SARS-CoV), SARS-CoV-2 (COVID-19), MERS-CoV, other highly pathogenic human coronaviruses, or any combination thereof.

The methods described herein can also be used to detect diagnoses viruses. In certain embodiments, the virus comprises a DNA virus. In certain embodiments, the virus comprises an RNA virus.

Detection of viral nucleic acids is the basis of the method described herein to detect and diagnose viral infection. For the detection and diagnosis of a viral infection, the viral nucleic acid molecule is part of a genome of a virus being tested. In some embodiments, said virus is a coronavirus. In some embodiments, said coronavirus is selected from the group consisting of severe acute respiratory syndrome coronavirus 2 (COVID-19), severe acute respiratory syndrome coronavirus (SARS-CoV), and Middle East respiratory syndrome coronavirus (MERS-CoV). In some embodiments, said coronavirus is COVID-19. The methods disclosed are useful in the classification of other RNA and DNA genome viruses. In some embodiments, said virus is an RNA virus. In some embodiments, said RNA virus comprises a double-stranded RNA genome. In some embodiments, said RNA virus comprises a single-stranded RNA genome. In some embodiments, said RNA virus is selected from the group consisting of coronavirus, influenza, human immunodeficiency virus, and Ebola virus. In some embodiments, said virus is a DNA virus. In some embodiments, the viral infection is COVID-19

For example, the methods herein are useful for the detection of coronaviruses. The methods for diagnosing a coronavirus infection are applicable to other viruses. Coronaviruses are members of the subfamily Coronavirinae in the family Coronaviridae and the order Nidovirales (International Committee on Taxonomy of Viruses). The coronavirus subfamily consists of four genera: Alphacoronavirus, Betacoronavirus, Gammacoronavirus and Deltacoronavirus. These genera are distinguished on the basis of phylogenetic relationships and genomic structures. Generally, alphacoronaviruses and betacoronaviruses infect mammals. Also, generally, the gammacoronaviruses and deltacoronaviruses infect birds, can also infect mammals. Alphacoronaviruses and betacoronaviruses can be associated with and the cause of respiratory illness and gastroenteritis in humans. Highly pathogenic viruses (e.g. severe acute respiratory syndrome coronavirus (SARS-CoV) and Middle East respiratory syndrome coronavirus (MERS-CoV), can cause severe respiratory syndrome in humans. Other four coronaviruses (e.g. HCoV-NL63, HCoV-229E, HCoV-OC43 and HKU1) generally induce mild upper respiratory diseases in immunocompetent hosts, infants, young children, and elderly individuals. Alphacoronaviruses and betacoronaviruses can pose a heavy disease burden not only to humans but also to livestock; these viruses include porcine transmissible gastroenteritis virus, porcine enteric diarrhea virus (PEDV), and swine acute diarrhea syndrome coronavirus (SADS-CoV). On the basis of current sequence databases, all human coronaviruses have zoonotic origins. For example, SARS-CoV, MERS-CoV, HCoV-NL63 and HCoV-229E are considered to have originated in bats. Domestic animals may have important roles as intermediate hosts that enable virus transmission from natural hosts to humans. In addition, domestic animals themselves can suffer disease caused by coronaviruses.

### Biological Samples

Provided herein are methods that use and process biological samples from individuals to diagnose viral infection. Such biological samples can be from individuals previously exposed to the virus, previously diagnosed as positive for the virus, or individuals deemed to be at risk of viral exposure. The methods described herein may also be useful for population surveillance, that is using samples provided by a plurality of individuals with the goal of providing information on the amount of viral infection in a given population. The biological samples can be oral or nasal mucosa. The samples can be blood, serum, or plasma. The biological samples, in certain embodiments, comprise a nasal swab, nasopharyngeal swab, buccal swab, oral fluid swab, mid-turbinate swab, or any combination thereof, wherein the swab has been used to collect a sample from an individual.

In practice, biological samples are collected using a myriad of collection devices, all of which can be used with the apparatus of the invention. The collection devices will generally be commercially available but can also be specifically designed and manufactured for a given application. For clinical samples, a variety of commercial swab types are available including nasal, nasopharyngeal, buccal, oral fluid, stool, tonsil, vaginal, cervical, and wound swabs. The dimensions and materials of the sample collection devices vary, and the devices may contain specialized handles, caps, scores to facilitate and direct breakage, and collection matrices.

Blood samples are collected in a wide variety of commercially available tubes of varying volumes, some of which contain additives (including anticoagulants such as heparin, citrate, and EDTA), a vacuum to facilitate sample entry, a stopper to facilitate needle insertion, and coverings to protect the operator from exposure to the sample. Tissue and bodily fluids (e.g. sputum, purulent material, aspirates) are also collected in tubes, generally distinct from blood tubes. These clinical sample collection devices are generally sent to sophisticated hospital or commercial clinical laboratories for testing (although certain testing such as the evaluation of throat/tonsillar swabs for rapid streptococcal tests can be performed at the point of care). Environmental samples may be present as filters or filter cartridges (e.g. from air breathers, aerosols or water filtration devices), swabs, powders, or fluids.

### Processing of Samples

After collection biological samples are contacted with a lysis agent in order to release viral nucleic acids that are present in cells that are obtained from the sample. Such lysis also releases genomic DNA of the individual being tested. Such genomic DNA can serve as a sample/amplification control. Sample collection and lysing can be performed before the method described herein for the amplification of viral sequences, or at the site of amplification and sequencing of viral sequences.

In accordance with methods and systems disclosed, a sample may be collected or partitioned along with lysis reagents in order to release the contents of the sample within the partition. Partitions include vials, tubes, and/or wells in a plate. In such embodiments, the lysis agents can be contacted with the sample suspension concurrently with, or prior to, the addition of reagents used in the extension and amplification of nucleic acid molecules. In some embodiments, the processing (e.g. amplification, primer extension, reverse transcriptase, etc.) of nucleic acids within the sample occurs in the same conditions used for cellular lysis. A discrete partition may include an individual sample and/or one or more reagents. In some embodiments, a discrete partition generated may include a primer and enzymes for the amplification of nucleic acids (e.g. reverse transcriptase and/or a polymerase). In some embodiments, a discrete partition generated can include a barcoded oligonucleotide (e.g. primer comprising a barcode sequence). In some embodiments, a discrete partition generated can include a barcode carrying bead. In some embodiments, a discrete partition may be unoccupied (e.g., no reagents, no samples).

Beneficially, when lysis reagents and samples are co-partitioned, the lysis reagents can facilitate the release of the contents of the samples within the partition. The contents released in a partition may remain discrete from the contents of other partitions.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lytiembodiment, and a variety of other lysis enzymes available from, e.g., Sigma-Aldrich, Inc. (St Louis, Mo.), as well as other commercially available lysis enzymes. Other lysis agents may additionally or alternatively be co-partitioned with the samples to cause the release of the sample's contents into the partitions. For example, in some embodiments, surfactant-based lysis solutions may be used to lyse cells, although these may be less desirable for emulsion-based systems where the surfactants can interfere with stable emulsions. In some embodiments, lysis solutions may include non-ionic surfactants such as, for example, TritonX-100 and/or Tween 20. In some embodiments, lysis solutions may include ionic surfactants such as, for example, sarcosyl and sodium dodecyl sulfate (SDS). Lysis agents described herein may comprise one or more proteinases (e.g. proteinase K). Electroporation, thermal, acoustic or mechanical cellular disruption may also be used in certain embodiments, e.g., non-emulsion based partitioning such as encapsulation of samples that may be in addition to or in place of partition partitioning, where any pore size of the encapsulate is sufficiently small to retain nucleic acid fragments of a given size, following cellular disruption.

Lysis agents may also comprise high temperature water or reaction buffers that are heated before or after addition of the sample. For example, a lysis agent can be a heated PCR or reverse transcriptase reaction mix that is heated to at least about 50, 55, 60, 65, 70, 75, 80, 85, 90, or 95° C. For an amount of time that is about 1, 2, 3, 4, 5, 6, 7, 8, 9, 10 minutes or more.

Alternatively or in addition to the lysis agents co-partitioned with the samples described above, other reagents can also be co-partitioned with the samples, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the embodiment of encapsulated samples, the samples may be exposed to an appropriate stimulus to release the samples or their contents from a co-partitioned microcapsule. For example, in some embodiments, a chemical stimulus may be co-partitioned along with an encapsulated sample to allow for the degradation of the microcapsule and release of the cell or its contents into the larger partition. In some embodiments, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated sample to be released into a partition at a different time from the release of nucleic acid molecules into the same partition.

A partition may comprise species (e.g., reagents) for conducting one or more reactions. Species may include, for example, reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for reverse transcription (e.g., reverse transcriptase enzymes), reagents for nucleic acid modification reactions such as polymerization, ligation, or digestion, and/or reagents for template preparation. In some cases, primers may be attached to the precursors. Primers may be used for reverse transcription. Primers may comprise a poly-T sequence or be specific for a target nucleic acid molecule (i.e. complementary to a sequence of the target nucleic acid). In some embodiments, the primers hybridize to specific target nucleic acid sequences (e.g. a viral nucleic acid sequence).

Additional reagents may also be co-partitioned with the samples, such as endonucleases to fragment a sample's DNA, DNA polymerase enzymes and dNTPs used to amplify the sample's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes if the lysed sample comprises an RNA based virus.

One advantage of the methods described herein is the ability to use saliva samples for detection of one or more of influenza A/B, coronavirus, or other pathogen sequences. The saliva sample for use in the methods described herein may comprise less than about 100, 50, 25, 10, 9, 8, 7, 6, 5, 4, 3, or 2 microliters of saliva. In certain embodiments, the saliva sample is less than about 20 microliters. In certain embodiments, the saliva sample is less than about 10 microliters. In certain embodiments, the saliva sample is less than about 8 microliters. In certain embodiments, the saliva sample is less than about 7 microliters. In certain embodiments, the saliva sample is less than about 6 microliters. In certain embodiments, the saliva sample is less than about 5 microliters.

One advantage of the methods described herein is the ability to use small amounts of nasal swab samples for detection of one or more of influenza A/B, coronavirus, or other pathogen sequences. Nasal swabs may be inoculated into about 1 milliliter of buffer such as PBS or saline, and then a small amount of that sample may be used to detect viral infection. The nasal swab sample for use in the methods described herein may comprise less than about 100, 50, 25, 10, 9, 8, 7, 6, 5, 4, 3, or 2 microliters of inoculated nasal swab. In certain embodiments, the inoculated nasal swab sample is less than about 20 microliters. In certain embodiments, the inoculated nasal swab sample is less than about 10 microliters. In certain embodiments, the inoculated nasal swab sample is less than about 8 microliters. In certain embodiments, the inoculated nasal swab sample is less than about 7 microliters. In certain embodiments, the inoculated nasal swab sample is less than about 6 microliters. In certain embodiments, the inoculated nasal swab sample is less than about 5 microliters.

### Detection of Pathogen Sequences

Viruses generally comprise a genomic structure comprising deoxyribonucleic acid (DNA) or ribonucleic acid (RNA). Such genomes can consist single-stranded or double-stranded nucleic acids. The genomes of viruses also generally comprise nucleic acid sequence that are different than the host they infect. Therefore, the differential nucleic acid sequences within a viral genome provide a target for detection using molecular and genetic amplification and sequencing technologies. Furthermore, differences in the genetic sequences and structures among viruses allows for the distinguishing classes, subclasses, and individual strains of viruses apart from one another. Accordingly, primers (e.g. probes) that recognize nucleic acid sequences, elements, templates, or loci specific to a virus can be used in the identification and detection of a viral infection.

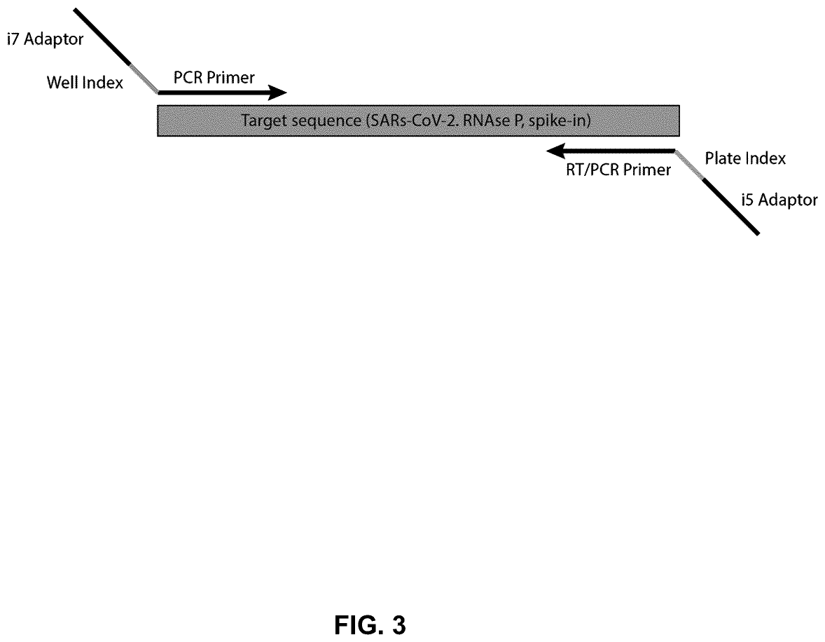

Described herein the detection of viral sequences comprises amplifying viral nucleic acid using a PCR reaction and sequencing the results of the PCR amplification. In instances where the virus being detected is an RNA based virus amplification also includes a reverse transcription reaction. The reverse transcription reaction can be a distinct step before PCT amplification or a one-step reaction that occurs in the presence of PCR enzymes and primers. PCR amplification is carried out via oligonucleotide primer pairs. Such primers in addition to comprising a target specific portion, may also comprise index sequences and/or sequencing adaptor sequences as shown in FIG. 3.

Amplification of viral and host nucleic acid molecules can be achieved through the use of enzymes that extend or amplify a primer hybridized to the viral and host nucleic acid molecules. Particularly, reverse transcriptase enzymes are used to generate cDNA corresponding to the host and viral nucleic acids. For example, for viruses having a genome comprising RNA, said nucleic acid extension reaction is a reverse transcription reaction. RNA nucleic acid products of DNA transcription in a host cell can also be processed using a reverse transcriptase enzyme. Known reverse transcriptase enzymes are readily available for use. Avian Myeloblastosis Virus (AMV) Reverse Transcriptase and Moloney Murine Leukemia Virus (M-MuLV, MMLV) Reverse Transcriptase are RTs that are commonly used in molecular biology workflows. M-MuLV Reverse Transcriptase lacks 3′→5′ exonuclease activity. ProtoScript II Reverse Transcriptase is a recombinant M-MuLV reverse transcriptase with reduced RNase H activity and increased thermostability. It can be used to synthesize first strand cDNA at higher temperatures than the wild-type M-MuLV. The enzyme is active up to 50° C., providing higher specificity, higher yield of cDNA and more full-length cDNA product, up to 12 kb in length. The use of engineered RTs improves the efficiency of full-length product formation, ensuring the copying of the 5′ end of the mRNA transcript is complete, and enabling the propagation and characterization of a faithful DNA copy of an RNA sequence. The use of the more thermostable RTs, where reactions are performed at higher temperatures, can be helpful when dealing with RNA that contains high amounts of secondary structure. In some embodiments, said nucleic acid extension reaction comprises a reverse transcriptase reaction, a polymerase chain reaction, or a combination thereof. In certain embodiments, said nucleic acid extension reaction comprises (i) hybridizing a primer to a viral nucleic acid molecule; and (ii) using a reverse transcriptase enzyme; extending said primer.

After or during reverse transcription (if applicable) the samples are subjected to an amplification reaction. In certain embodiments, the amplification reaction is a PCR reaction. In certain embodiments, the PCR reaction is not a real-time PCR reaction. In certain embodiments the PCR is carried out for a set number of cycles sufficient to amplify viral nucleic acid sequences. The lysed and reverse transcribed sample can be amplified for N cycles. In certain embodiments, N is greater than 30, 35, 40, or 45 cycles. In certain embodiments, N is between 30 and 50 cycles, between 40 and 50 cycles, between 35 and 45 cycles, between 36 and 44 cycles, between 37 and 43 cycles, between 38 and 42 cycles, between 39 and 41 cycles, or between 40 and 45 cycles. In certain embodiments, the lysed and reverse transcribed sample can be amplified for 40 cycles.

Primer pairs can be added to the amplification PCR reaction at an optimized concentration. In certain embodiments, the concentration of the primer sets that amplify the viral nucleic acid/synthetic nucleic acid and/or the host control is Primer pairs can be added to the PCR reaction at a concentration below 1 micromolar. In certain embodiments, the concentration is about 800 nM, 400 nM, 200 nM, 100 nM, 150 nM, or 50 nM.

Polymerase chain reaction amplification can be used to further incorporate functional sequences such as a variable nucleotide sequence (barcode). In some embodiments, said polymerase chains reaction incorporates one or more additional sequences into one or both of said barcoded viral nucleic acid molecule and barcoded host nucleic acid molecule, selected from the group consisting of a sample index sequence, an adapter sequence, primer sequence, a primer binding sequence, a sequence configured to couple to the flow cell of a sequencer, and an additional barcode sequence.

The method described herein includes a synthetic nucleic acid that is co-reverse transcribed and/or amplified with viral nucleic acid. In a certain embodiment a set of oligonucleotide primers that amplify a viral sequence also amplifies the synthetic nucleic acid. A sample can be “spiked” with a synthetic nucleic acid molecule that provides information regarding the processing of nucleic acids in a sample. In certain embodiments, the synthetic nucleic acid molecule is added to the biological sample prior to processing or amplification. In certain embodiments, the synthetic nucleic acid is spiked into the biological sample at a known concentration or amount. In certain embodiments, the known amount is 1×10⁴, 1×10³, 1×10², 10, 5, 4, 3, 2, or 1 copies of synthetic nucleic acid. In certain embodiments, the synthetic control nucleic acid is an RNA. Ideally, the length of the amplified portion of the synthetic nucleic acid matches the length of viral nucleic acid target to be amplified, and produces an amplicon that is the same length as the viral nucleic acid target or within about 1, 2, 3, 4, 5, 6, 7, 8, 9, 10, 15, 20, 30, 40, or 50 nucleotides. Also, the sequence of the synthetic nucleic acid should be highly homologous to the viral sequence but vary by at least one nucleotide such that the synthetic nucleic acid can be discriminated by sequencing. In certain embodiments, the synthetic nucleic acid varies by at least about 1, 2, 3, 4, 5, 6, 7, 8, 9, 10, 11, 12, 13 14 or 15 nucleotides compared to the sequence of the viral nucleic acid to be amplified.

The synthetic nucleic acid sequence may be used to normalize sequence reads and account for losses during sample preparation or inefficiencies/bias introduced during amplification or sequencing.

The synthetic nucleic acid may comprise a plurality of nucleic acids with distinct sequences to further improve the performance of the “spiked” synthetic nucleic acid for downstream processing and analysis. The plurality may comprise 2, 3, 4, 5, 6, 7, 8, 9, 10, or more distinct sequences. The plurality may comprise 2, 3, 4, 5 distinct sequences. The plurality may comprise 4 distinct sequences. The plurality may be for use with primers that amplify the S2 spike sequence, the N1 nucleoprotein sequence, or a combination thereof. Exemplary sequences are shown in the table below (as the synthetic nucleic acids are RNA all Ts are Us in the RNA version of the sequence). Any one or more of the synthetic nucleic acids or the plurality of synthetic nucleic acids can possess at least about 90%, 95%, 97%, 98%, 99% or identical homology to any of SEQ ID NOs. 1 to 12 shown below.

Exemplary sequences for COVID 19 S2 and N1 synthetic spike sequences

| | |
|---|---|
| S2_001 | GCUGGUGCUGCAGCUUAUUAUGUGGGUGUGUAUCUCACGAAGCG |
| SEQ ID NO: 1 | ACCCUUUGGAAAAUAUAAUGAAAAUGGAACCAUUACAGAUGCUG |
| | UAGACUGUGCACUUGACCCU |
| S2_002 | GCUGGUGCUGCAGCUUAUUAUGUGGGUCCUCGCUAGGACGUCGC |
| SEQ ID NO: 2 | UAUgacgccAAAAUAUAAUGAAAAUGGAACCAUUACAGAUGCUGUA |
| | GACUGUGCACUUGACCCU |
| S2_003 | GCUGGUGCUGCAGCUUAUUAUGUGGGUAGCACGACUUGAUCUAA |
| SEQ ID NO: 3 | CUgacacuaAAAAUAUAAUGAAAAUGGAACCAUUACAGAUGCUGUA |
| | GACUGUGCACUUGACCCU |
| S2_004 | GCUGGUGCUGCAGCUUAUUAUGUGGGUUAAGUAGGACUUCGAUU |
| SEQ ID NO: 4 | ggaUggaauAAAAUAUAAUGAAAAUGGAACCAUUACAGAUGCUGUA |
| | GACUGUGCACUUGACCCU |
| N1_001 | UCUGGUUACUGCCAGUUGAAUCUGAGGGUCCGGACGGAUAUCGC |
| SEQ ID NO: 5 | ACUAAGUGUACCUGGUGCAUUUCGCUGAUUUUGGGGUC |
| N1_002 | UCUGGUUACUGCCAGUUGAAUCUGAGGGUCCCAUGACCAUGUCA |
| SEQ ID NO: 6 | CUGGCUACACUGAGGUGCAUUUCGCUGAUUUUGGGGUC |
| N1_003 | UCUGGUUACUGCCAGUUGAAUCUGAGGGUCCUUGACAUGGCAUG |
| SEQ ID NO: 7 | UGACUCCACUGUCGGUGCAUUUCGCUGAUUUUGGGGUC |
| N1_004 | UCUGGUUACUGCCAGUUGAAUCUGAGGGUCCACCUUUGCCAGAU |
| SEQ ID NO: 8 | GACUGAGUGGAAGGGUGCAUUUCGCUGAUUUUGGGGUC |
| N1_005 | UCUGGUUACUGCCAGUUGAAUCUGAGGGUCCAGCUUGAAGCGUU |
| SEQ ID NO: 9 | CGCGACAAGUGUCGGUGCAUUUCGCUGAUUUUGGGGUC |
| N1_006 | UCUGGUUACUGCCAGUUGAAUCUGAGGGUCCUCACGUCCUGAGA |
| SEQ ID NO: 10 | UCAACUGCUACAUGGUGCAUUUCGCUGAUUUUGGGGUC |
| N1_007 | UCUGGUUACUGCCAGUUGAAUCUGAGGGUCCGUUGACUGAUCAC |
| SEQ ID NO: 11 | AUGCUGCUCCACGGGUGCAUUUCGCUGAUUUUGGGGUC |
| N1_008 | UCUGGUUACUGCCAGUUGAAUCUGAGGGUCCCAGACAGUCAUCG |
| SEQ ID NO: 12 | GAUUGAUGAGUGAGGUGCAUUUCGCUGAUUUUGGGGUC |

The synthetic nucleic acid comprises 5′ and 3′ proximal sequences that can be bound by the same primers that amplify the viral nucleic acid sequence being tested for, but that comprise a distinct intervening sequence distinguishable from the viral nucleic acid sequence being tested for by sequencing. In some embodiments, the method described herein further comprises providing a synthetic nucleic acid molecule. In some embodiments, the method described herein further comprises providing a synthetic nucleic acid molecule compromising 5′ and 3′ proximal sequences that can be bound by the same primers that amplify the viral nucleic acid. In certain embodiments, the 5′ proximal region, said 3′ proximal region, or both said 5′ proximal region and said 3′ proximal region are less than about 30 nucleotides in length. In certain embodiments, the 5′ proximal region, the 3′ proximal region, or both the 5′ proximal region and the 3′ proximal region are less than about 25 nucleotides in length. In certain embodiments, the 5′ proximal region, the 3′ proximal region, or both the 5′ proximal region and said 3′ proximal region are less than about 20 nucleotides in length. In certain embodiments, the 5′ proximal region is at the 5′ terminus of the synthetic nucleic acid. In certain embodiments, the 3′ proximal region is at the 3′ terminus of the synthetic nucleic acid. In certain embodiments, the intervening nucleic acid sequence is less than about 99%, 98%, 97%, 95%, 90%, 85%, 80%, or 75%, identical to a viral nucleic acid sequence. In some embodiments, the synthetic nucleic acid molecule comprises a synthetic sequence that is different from said viral nucleic acid molecule and said human nucleic acid molecule. In some embodiments, the synthetic nucleic acid molecule comprises a nucleotide sequence identity to a host and/or viral sequence no greater than 10%, 25%, 50%, 75%, 90%, 95%, or 98%.

In certain embodiments, the synthetic nucleic acid is added to a lysate to achieve a concentration in the method of about 1 copy/well to about 1,000,000 copies/well. In certain embodiments, the synthetic nucleic acid is added at about 1 copy/well to about 50 copies/well, about 1 copy/well to about 100 copies/well, about 1 copy/well to about 500 copies/well, about 1 copy/well to about 1,000 copies/well, about 1 copy/well to about 5,000 copies/well, about 1 copy/well to about 10,000 copies/well, about 1 copy/well to about 100,000 copies/well, about 1 copy/well to about 1,000,000 copies/well, about 50 copies/well to about 100 copies/well, about 50 copies/well to about 500 copies/well, about 50 copies/well to about 1,000 copies/well, about 50 copies/well to about 5,000 copies/well, about 50 copies/well to about 10,000 copies/well, about 50 copies/well to about 100,000 copies/well, about 50 copies/well to about 1,000,000 copies/well, about 100 copies/well to about 500 copies/well, about 100 copies/well to about 1,000 copies/well, about 100 copies/well to about 5,000 copies/well, about 100 copies/well to about 10,000 copies/well, about 100 copies/well to about 100,000 copies/well, about 100 copies/well to about 1,000,000 copies/well, about 500 copies/well to about 1,000 copies/well, about 500 copies/well to about 5,000 copies/well, about 500 copies/well to about 10,000 copies/well, about 500 copies/well to about 100,000 copies/well, about 500 copies/well to about 1,000,000 copies/well, about 1,000 copies/well to about 5,000 copies/well, about 1,000 copies/well to about 10,000 copies/well, about 1,000 copies/well to about 100,000 copies/well, about 1,000 copies/well to about 1,000,000 copies/well, about 5,000 copies/well to about 10,000 copies/well, about 5,000 copies/well to about 100,000 copies/well, about 5,000 copies/well to about 1,000,000 copies/well, about 10,000 copies/well to about 100,000 copies/well, about 10,000 copies/well to about 1,000,000 copies/well, or about 100,000 copies/well to about 1,000,000 copies/well. In certain embodiments, the synthetic nucleic acid is added at about 1 copy/well, about 50 copies/well, about 100 copies/well, about 500 copies/well, about 1,000 copies/well, about 5,000 copies/well, about 10,000 copies/well, about 100,000 copies/well, or about 1,000,000 copies/well. In certain embodiments, the synthetic nucleic acid is added at least about 1 copy/well, about 50 copies/well, about 100 copies/well, about 500 copies/well, about 1,000 copies/well, about 5,000 copies/well, about 10,000 copies/well, or about 100,000 copies/well. In certain embodiments, the synthetic nucleic acid is added at most about 50 copies/well, about 100 copies/well, about 500 copies/well, about 1,000 copies/well, about 5,000 copies/well, about 10,000 copies/well, about 100,000 copies/well, or about 1,000,000 copies/well.

After amplification and/or sequencing the resulting ratio of detected pathogen nucleic acid molecule and synthetic oligonucleotide is useful in detecting and diagnosing a pathogenic infection. The components of the ratio used in the detection and diagnosis of an infectious pathogen can be based on the number of reads for a specific sequence or set of sequences, the copy number of a specific sequence or set of sequences (e.g. the number of different unique molecular identifier sequences associated with a specific sequence), an number or amount derived from the sequencing information, or any combination thereof. In some embodiments, the ratio of pathogen nucleic acids to synthetic nucleic acids is greater than 1. In some embodiments, the ratio of pathogen nucleic acids to synthetic nucleic acids is equal to or about 1. In some embodiments, the ratio of pathogen nucleic acids to synthetic nucleic acids is less than 1.

In certain embodiments, the ratio of pathogen nucleic acids to synthetic nucleic acids is about 0.00001:1 to about 0.5:1. In certain embodiments, the ratio of pathogen nucleic acids to synthetic nucleic acids is about 0.00001:1 to about 0.00005:1, about 0.00001:1 to about 0.0001:1, about 0.00001:1 to about 0.0002:1, about 0.00001:1 to about 0.0003:1, about 0.00001:1 to about 0.0004:1, about 0.00001:1 to about 0.0005:1, about 0.00001:1 to about 0.001:1, about 0.00001:1 to about 0.005:1, about 0.00001:1 to about 0.01:1, about 0.00001:1 to about 0.1:1, about 0.00001:1 to about 0.5:1, about 0.00005:1 to about 0.0001:1, about 0.00005:1 to about 0.0002:1, about 0.00005:1 to about 0.0003:1, about 0.00005:1 to about 0.0004:1, about 0.00005:1 to about 0.0005:1, about 0.00005:1 to about 0.001:1, about 0.00005:1 to about 0.005:1, about 0.00005:1 to about 0.01:1, about 0.00005:1 to about 0.1:1, about 0.00005:1 to about 0.5:1, about 0.0001:1 to about 0.0002:1, about 0.0001:1 to about 0.0003:1, about 0.0001:1 to about 0.0004:1, about 0.0001:1 to about 0.0005:1, about 0.0001:1 to about 0.001:1, about 0.0001:1 to about 0.005:1, about 0.0001:1 to about 0.01:1, about 0.0001:1 to about 0.1:1, about 0.0001:1 to about 0.5:1, about 0.0002:1 to about 0.0003:1, about 0.0002:1 to about 0.0004:1, about 0.0002:1 to about 0.0005:1, about 0.0002:1 to about 0.001:1, about 0.0002:1 to about 0.005:1, about 0.0002:1 to about 0.01:1, about 0.0002:1 to about 0.1:1, about 0.0002:1 to about 0.5:1, about 0.0003:1 to about 0.0004:1, about 0.0003:1 to about 0.0005:1, about 0.0003:1 to about 0.001:1, about 0.0003:1 to about 0.005:1, about 0.0003:1 to about 0.01:1, about 0.0003:1 to about 0.1:1, about 0.0003:1 to about 0.5:1, about 0.0004:1 to about 0.0005:1, about 0.0004:1 to about 0.001:1, about 0.0004:1 to about 0.005:1, about 0.0004:1 to about 0.01:1, about 0.0004:1 to about 0.1:1, about 0.0004:1 to about 0.5:1, about 0.0005:1 to about 0.001:1, about 0.0005:1 to about 0.005:1, about 0.0005:1 to about 0.01:1, about 0.0005:1 to about 0.1:1, about 0.0005:1 to about 0.5:1, about 0.001:1 to about 0.005:1, about 0.001:1 to about 0.01:1, about 0.001:1 to about 0.1:1, about 0.001:1 to about 0.5:1, about 0.005:1 to about 0.01:1, about 0.005:1 to about 0.1:1, about 0.005:1 to about 0.5:1, about 0.01:1 to about 0.1:1, about 0.01:1 to about 0.5:1, or about 0.1:1 to about 0.5:1. In certain embodiments, the ratio of pathogen nucleic acids to synthetic nucleic acids is about 0.00001:1, about 0.00005:1, about 0.0001:1, about 0.0002:1, about 0.0003:1, about 0.0004:1, about 0.0005:1, about 0.001:1, about 0.005:1, about 0.01:1, about 0.1:1, or about 0.5:1. In certain embodiments, the ratio of pathogen nucleic acids to synthetic nucleic acids is at least about 0.00001:1, about 0.00005:1, about 0.0001:1, about 0.0002:1, about 0.0003:1, about 0.0004:1, about 0.0005:1, about 0.001:1, about 0.005:1, about 0.01:1, or about 0.1:1.

In certain embodiments, the ratio of pathogen nucleic acids to synthetic nucleic acids is about 1:1 to about 1:100. In certain embodiments, the ratio of pathogen nucleic acids to synthetic nucleic acids is about 1:100 to about 1:50, about 1:100 to about 1:25, about 1:100 to about 1:10, about 1:100 to about 1:5, about 1:100 to about 1:4, about 1:100 to about 1:3, about 1:100 to about 1:2, about 1:100 to about 1:1, about 1:100 to about 1:1, about 1:50 to about 1:25, about 1:50 to about 1:10, about 1:50 to about 1:5, about 1:50 to about 1:4, about 1:50 to about 1:3, about 1:50 to about 1:2, about 1:50 to about 1:1, about 1:50 to about 1:1, about 1:25 to about 1:10, about 1:25 to about 1:5, about 1:25 to about 1:4, about 1:25 to about 1:3, about 1:25 to about 1:2, about 1:25 to about 1:1, about 1:25 to about 1:1, about 1:10 to about 1:5, about 1:10 to about 1:4, about 1:10 to about 1:3, about 1:10 to about 1:2, about 1:10 to about 1:1, about 1:10 to about 1:1, about 1:5 to about 1:4, about 1:5 to about 1:3, about 1:5 to about 1:2, about 1:5 to about 1:1, about 1:5 to about 1:1, about 1:4 to about 1:3, about 1:4 to about 1:2, about 1:4 to about 1:1, about 1:4 to about 1:1, about 1:3 to about 1:2, about 1:3 to about 1:1, about 1:3 to about 1:1, about 1:2 to about 1:1, about 1:2 to about 1:1, or about 1:1 to about 1:1. In certain embodiments, the ratio of pathogen nucleic acids to synthetic nucleic acids is about 1:100, about 1:50, about 1:25, about 1:10, about 1:5, about 1:4, about 1:3, about 1:2, about 1:1, or about 1:1. In certain embodiments, the ratio of pathogen nucleic acids to synthetic nucleic acids is at least about 1:100, about 1:50, about 1:25, about 1:10, about 1:5, about 1:4, about 1:3, about 1:2, or about 1:1. In certain embodiments, the ratio of pathogen nucleic acids to synthetic nucleic acids is at most about 1:50, about 1:25, about 1:10, about 1:5, about 1:4, about 1:3, about 1:2, about 1:1, or about 1:1.

In certain embodiments, the ratio of synthetic nucleic acids to pathogen nucleic acids is about 1:100 to about 1:50, about 1:100 to about 1:25, about 1:100 to about 1:10, about 1:100 to about 1:5, about 1:100 to about 1:4, about 1:100 to about 1:3, about 1:100 to about 1:2, about 1:100 to about 1:1, about 1:100 to about 1:1, about 1:50 to about 1:25, about 1:50 to about 1:10, about 1:50 to about 1:5, about 1:50 to about 1:4, about 1:50 to about 1:3, about 1:50 to about 1:2, about 1:50 to about 1:1, about 1:50 to about 1:1, about 1:25 to about 1:10, about 1:25 to about 1:5, about 1:25 to about 1:4, about 1:25 to about 1:3, about 1:25 to about 1:2, about 1:25 to about 1:1, about 1:25 to about 1:1, about 1:10 to about 1:5, about 1:10 to about 1:4, about 1:10 to about 1:3, about 1:10 to about 1:2, about 1:10 to about 1:1, about 1:10 to about 1:1, about 1:5 to about 1:4, about 1:5 to about 1:3, about 1:5 to about 1:2, about 1:5 to about 1:1, about 1:5 to about 1:1, about 1:4 to about 1:3, about 1:4 to about 1:2, about 1:4 to about 1:1, about 1:4 to about 1:1, about 1:3 to about 1:2, about 1:3 to about 1:1, about 1:3 to about 1:1, about 1:2 to about 1:1, about 1:2 to about 1:1, or about 1:1 to about 1:1. In certain embodiments, the ratio of pathogen nucleic acids to synthetic nucleic acids is about 1:100, about 1:50, about 1:25, about 1:10, about 1:5, about 1:4, about 1:3, about 1:2, about 1:1, or about 1:1. In certain embodiments, the ratio of pathogen nucleic acids to synthetic nucleic acids is at least about 1:100, about 1:50, about 1:25, about 1:10, about 1:5, about 1:4, about 1:3, about 1:2, or about 1:1. In certain embodiments, the ratio of pathogen nucleic acids to synthetic nucleic acids is at most about 1:50, about 1:25, about 1:10, about 1:5, about 1:4, about 1:3, about 1:2, about 1:1, or about 1:1.

The ratio of pathogen reads to pathogen synthetic nucleic acid (with sequence distinguishable from pathogen read) can be used to indicate positive diagnosis with a particular pathogen (e.g., coronavirus, Influenza A, Influenza B). Alternatively, a negative diagnosis is made if the ratio does not exceed a threshold for positivity. In some embodiments, a positive diagnosis for the pathogen is made if the sequence reads from pathogen nucleic acids to synthetic nucleic acids exceeds a ratio of from about 0.01 to about 0.5. In some embodiments, a positive diagnosis for the pathogen is made if the sequence reads from pathogen nucleic acids to synthetic nucleic acids exceeds a ratio of from about 0.01 to about 0.4, from about 0.01 to about 0.3, from about 0.01 to about 0.2, from about 0.02 to about 0.5, from about 0.02 to about 0.2, from about 0.03 to about 0.5, from about 0.03 to about 0.2, from about 0.04 to about 0.5, from about 0.03 to about 0.2, from about 0.05 to about 0.5, from about 0.05 to about 0.2, from about 0.06 to about 0.5, from about 0.06 to about 0.2, from about 0.07 to about 0.5, from about 0.07 to about 0.2, from about 0.08 to about 0.5, from about 0.08 to about 0.2. In some embodiments, a positive diagnosis for the pathogen is made if the sequence reads from pathogen nucleic acids to synthetic nucleic acids exceeds a ratio of about 0.01, about 0.02, about 0.03, about 0.04, about 0.05, about 0.06, about 0.07, about 0.08, about 0.09, or about 0.1.

The total amount of pathogen reads plus pathogen synthetic nucleic acid (with sequence distinguishable from pathogen read) can be used to indicate if enough sequence data is present to attempt a positive or negative diagnosis for the presence of the pathogen (e.g., coronavirus, Influenza A, Influenza B). In some embodiments, the positive or negative diagnosis can only be made if the combined number of sequence reads of the pathogen nucleic acids together with the synthetic nucleic acids exceeds a minimum threshold, or else the result is inconclusive. In some embodiments, the minimum threshold is at least about 10 reads, at least about 20 reads, at least about 30 reads, at least about 40 reads, at least about 50 reads, at least about 60 reads, at least about 70 reads, at least about 80 reads, at least about 90 reads, at least about 100, at least about 150 reads, at least about 200 reads, or at least about 250 reads.

In some cases, a sample control can be used to verify that enough genetic material was present in the sample to reliably call a positive or negative diagnosis. The amount of reads can be counted for the sample control. This sample control is usually a housekeeping gene of the species of individual that the test is being performed on. In certain embodiments, when the individual being tested is human the sample control is beta actin, GAPDH, or RPP30. In certain embodiments, when the individual being tested is human the sample control is RPP30. In certain embodiments, the amount of reads from a sample control present in order to deliver a positive or negative diagnosis exceeds 5, 6, 7, 8, 9, 10, 11, 12, 13, 14, 15, 16, 17, 128, 19, 20, 25, or 30 reads. In certain embodiments, the amount of reads from a sample control present in order to deliver a positive or negative diagnosis equals or exceeds 10 reads.

The methods disclosed can also be combined with multiplexing strategies to effectively and efficiently allow for the testing of multiple samples in parallel. As used herein, “multiplex” refers to simultaneously conducting a plurality of assays on one or more samples. Multiplexing can further include simultaneously conducting a plurality of assays in each of a plurality of separate samples. For example, the number of reaction mixtures analyzed can be based on the number of partitions and the number of assays conducted in each partition can be based on the number of probes that contact the contents of each well. Multiplexing design and strategies can be effectively employed in the application of the method disclosed. For example, one or more unique barcode or adapter nucleic acid molecules can be coupled to a target nucleic acid molecule, wherein the unique barcode or adapter nucleic acid molecule identifies or barcodes the target nucleic acid molecule. Once barcoded, samples can be combined or pooled into a single sequencing library, wherein a barcoded target nucleic acid (e.g. a pathogen nucleic acid molecule and/or a synthetic nucleic acid molecule) can be distinguished from other barcoded samples. Accordingly, sample multiplexing and the use of nucleic acid barcodes can be used to identify sequencing reads from a first individual from a group or plurality of individuals. In some embodiments, the samples are pooled prior to sequencing. In certain embodiments, greater than 10, 25, 50, 75, 100, or 500 samples are pooled and analyzed in in a single sequencing library.

Furthermore, one or more pathogens may be tested in a single assay. For example, multiple respiratory viruses (or viral subtypes) can be analyzed in a single assay. Accordingly, the method disclosed for a single pathogen can be combined for the detection and analysis of multiple pathogens in a single reaction. (e.g. using multiple primers specific for multiple pathogen nucleic acid molecules and multiple synthetic nucleic acid molecule can be added to a sample). By way of further example, in some embodiments, a first pathogen is a coronavirus and a second pathogen is an influenza virus. Examples of multiple pathogens also include different subtypes or clades of viruses (e.g. influenza A H1N1 and influenza A H3N2).

The method and reaction mixtures described herein may make use of amplification and sequencing of a plurality of coronavirus sequences. In certain embodiments, the plurality of coronavirus sequences that are amplified and sequenced comprises an S2 sequence. In certain embodiments, the plurality of coronavirus sequences that are amplified and sequenced comprises an N1 sequence. In certain embodiments, the plurality of coronavirus sequences that are amplified and sequenced comprises an S2 and NIsequence. Such tests may make use of a plurality of synthetic nucleic acid spike-ion controls, for each viral target.

The methods, reaction mixtures, and kits described herein can be used to test for a coronavirus (e.g., SARS-CoV2) and Influenza A and/or Influenza B. Such tests would be useful for healthcare providers and health agencies in monitoring outbreaks of different common respiratory pathogens simultaneously. The methods described herein can be used in a triplex test to simultaneously detect coronavirus and Influenzas A and B. Such tests may make use of three different synthetic nucleic acid spike-ion controls, one for each viral target.

The methods described herein may also comprise a second set of oligonucleotide primers that targets a nucleic acid sequence of the viral host (e.g., the individual being tested) such a primer set provides a positive control for the sample and for amplification. In certain embodiments, said second set of PCR primers amplifies a human nucleic acid sequence selected from GAPDH, ACTB, RPP30, and combinations thereof. In certain embodiments, said second set of PCR primers amplifies a human RPP30 sequence. In certain embodiments, the second set of PCR primer comprises a mixture of primers with sequencing adaptor sequences and primers without sequencing adaptor sequences. In certain embodiments, a ratio of primers with sequencing adaptor sequences to primers without sequencing adaptor sequences is about 1:1, about 1:2, about 1:3 or about 1:4. A mixture of primers with and without adaptors allows for detection of the viral host nucleic acid, but allows more sequencing reads to be devoted to viral sequences.

In some embodiments, a result of a diagnosis of infection of a pathogen is inconclusive if a minimum predetermined number of reads of the human nucleic acid sequence is not detected. In some embodiments, the minimum predetermined number of reads of the human nucleic acid sequence must exceed at least about 2, at least about 3, at least about 4, at least about 5, at least about 6, at least about 7, at least about 8, at least about 9, at least about 10 reads, at least about 15 reads, at least about 20 reads, or at least about 25 reads of the human nucleic acid sequence. In some embodiments, the minimum predetermined number of reads of the human nucleic acid sequence must exceed at least about 10 reads.

Coronaviruses form enveloped viral particles of 100-160 nm in diameter. Coronaviruses are single-stranded ribonucleic acid (ssRNA) viruses, comprising a positive-sense RNA genome of 27-32 kb in size. The 5′ region of the coronavirus the genome encodes a polyprotein, pplab, which is further cleaved into 16 non-structural proteins that are involved in genome transcription and replication. The 3′ region encodes structural proteins, including envelope glycoproteins spike (e.g. viral spike), envelope, membrane, and nucleocapsid. The genes encoding structural proteins can also function as accessory genes that are species-specific and can be dispensable for virus replication.

COVID-19 (also referred to as HCoV-19 or SARS-CoV-2) is a betacoronoavirus and the seventh coronavirus known to infect humans. The COVID-19 coronavirus can cause severe disease (as also observed with SARS-CoV, MERS-CoV). COVID-19 demonstrates efficient targeting of the human receptor ACE2. The receptor-binding domain (RBD) in the spike protein is the most variable part of the coronavirus genome. Six receptor-binding domain (RBD) amino acids have been shown to be critical for binding to ACE2 receptors and for determining the host range of SARS-CoV-like viruses. Based on the SARS-CoV protein sequence, the residues that participate in ACE2 binding, and contribute to receptor targeting, are Y442, L472, N479, D480, T487 and Y4911 (these residues also correspond to L455, F486, Q493, S494, N501 and Y505 in SARS-CoV-2). In COVID-19, five of these six residues differ between COVID-19 and SARS-CoV, corresponding to L455, F486, Q493, S494, and N501 of the COVID-19 receptor-binding domain (RBD) protein. Such sequence properties of COVID-19 can be used to identify, detect, and/or diagnose COVID-19. For example, the unique sequence of the sequence of the COVID-19 receptor-binding domain (RBD) can be used to identify, detect, or diagnose COVID-19.

COVID-19 also comprises a functional polybasic (furin) cleavage site within the S1 subunit and S2 subunit of the viral spike protein boundary through the insertion of 12 nucleotides. The inserted sequence also potentiates the acquisition of three O-linked glycans. Both the polybasic cleavage site and the three adjacent predicted O-linked glycans are unique to COVID-19 and have not previously identified in alpha- or betacoronaviruses. Accordingly, the viral spike sequence properties of COVID-19 can be used to identify, detect, and/or diagnose COVID-19. For example, the unique sequence of the sequence of the COVID-19 viral spike insertion can be used to identify, detect, or diagnose COVID-19.

The methods described herein are useful for the detection and diagnosis of viral infections in individuals. In certain embodiments, the viral infection is influenza. In certain embodiments, the viral infection is a coronavirus. In certain embodiments, the viral infection is COVID-19. In certain embodiments, the viral infection is MERS-CoV. In certain embodiments, the viral infection is SARS-CoV-2.

In principle any suitable viral nucleic acid sequence can be targeted by an oligo nucleotide primer pair. Such targets include nucleic acid sequences that encode the viral nucleocapsid protein, viral spike protein, viral envelop protein, or viral membrane protein. Such targets include non-structural proteins.

If the viral infection to be detected is SARS-CoV-2 the viral nucleic acid detected can be one that encodes one or more of the viral nucleocapsid protein (N1), viral spike protein (S2), viral envelop protein, or viral membrane protein. In certain embodiments, the nucleic acid detected is one that encodes any one or more of the 16 SARS-CoV-2 non-structural proteins; NSP1, NSP2, NSP3, NSP4, NSP5, NSP6, NSP7, NSP8, NSP9, NSP10, NSP111, NSP12, NSP13, NSP14, NSP15, or NSP16.

Exemplary primer pairs that can be used in the methods described herein can bind to sequences described in the sequence Appendix following this disclosure. In certain embodiments, any of the primers listed in the appendix can be used in the methods described herein.

Primers comprising barcoded oligos are useful in sample analysis (e.g. sample identification, tracing, quantification, etc.). For examples, primers within a partition comprising barcoded oligonucleotides that further comprise a common barcode sequence and a unique molecular identifier sequence can be used to (1) identify sequences belong to a partition through the use of the common barcode and (2) identify transcript copy number through the use of unique molecular identifiers. In some embodiments, said first primer set further comprises one or more additional functional sequences selected from the group consisting of primer sequences, adapter sequences, primer annealing sequences, a unique molecular identifier sequence, and capture sequences. In some embodiments, said second primer further comprises one or more additional functional sequences selected from the group consisting of primer sequences, adapter sequences, primer annealing sequences, a unique molecular identifier sequence, and capture sequences. Generally, the primers are specific for (i.e. complementary to or comprising a sequence complementary to) a target sequence of a viral, host or synthetic nucleic acid molecule.

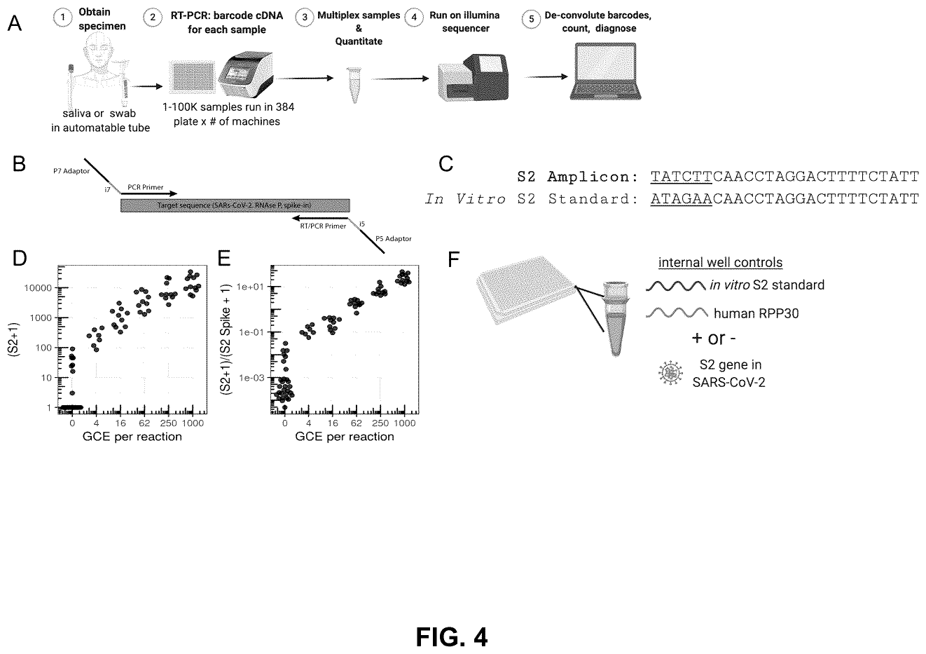

FIG. 4-FIG. 21 further demonstrate and/or exemplify the methods and compositions disclosed herein, and the advantages of their use and/or application. FIG. 4 exemplifies the SwabSeq Diagnostic Testing Platform for COVID19. In (A) the workflow for Swab Seq is a five-step process that takes approximately 12 hours from start to finish. In (B) each well, perform RT-PCR on clinical samples is performed. Each well has two sets of indexed primers that generate cDNA and amplicons for SARS-CoV-2 S2 gene and the human RPP30 gene. Each primer is synthesized with the P5 and P7 adaptors for Illumina sequencing, a unique i7 and i5 molecular barcodes, and the unique primer pair. Importantly, every well has a synthetic in vitro S2 standard that is key to allowing the method to work at scale. In (C) the in vitro S2 standard (abbreviated as S2-Spike) differs from the virus S2 gene by 6 base pairs that are complemented (underlined). In (D) read count at various viral concentrations. In (E) ratiometric normalization allow for in-well normalization for each amplicon. In (F) every well has two internal well controls for amplification, the in vitro S2 standard and the human RPP30. The RPP30 amplicon serves as a control for specimen collection. The in vitro S2 standard is critical to SwabSeq's ability to distinguish true negatives.

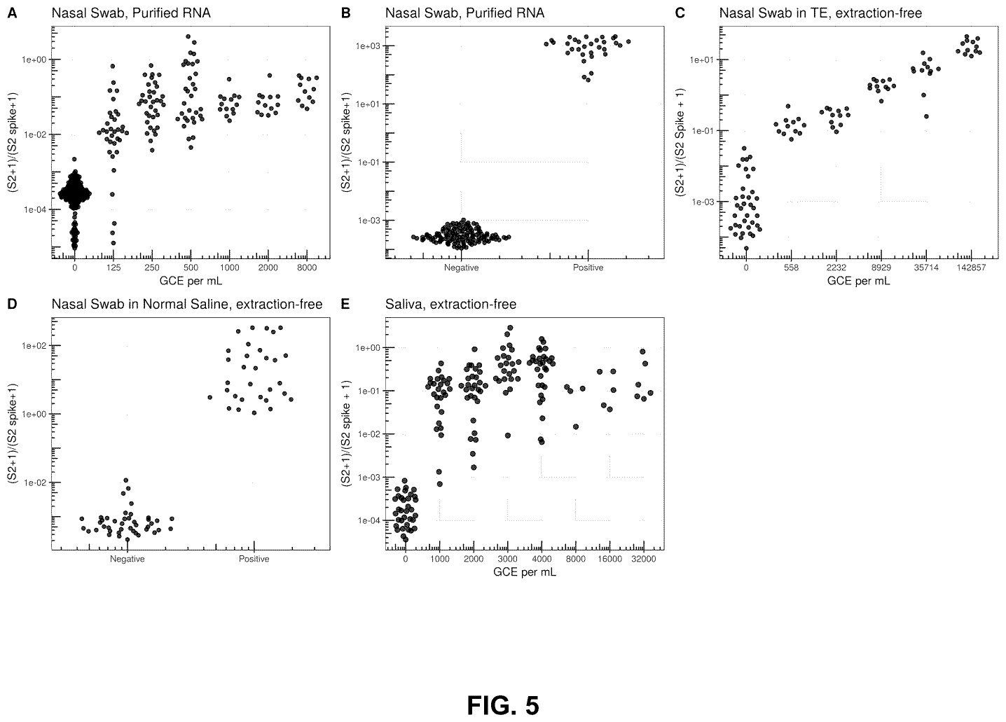

FIG. 5 shows that validation in clinical specimens demonstrate a limit of detection equivalent to sensitive RT-qPCR reactions. In (A) Limit of Detection (LOD) in nasal swab samples with no SARS-CoV2 were pooled and ATCC inactivated virus was added at different concentrations. Nasal Swab sample was RNA purified and using SwabSeq showed a limit of detection of 250 genome copy equivalents (GCE) per mL. In (B) RNA-purified clinical nasal swab specimens obtained through the UCLA Health Clinical Microbiology Laboratory were tested based on clinical protocols using FDA authorized platforms and then also tested using SwabSeq. 100% agreement with samples that tested positive for SARS-CoV-2 (n=31) and negative for SARS-CoV-2 (n=35) is shown. In (C) tested RNA purified samples from extraction-free nasopharyngeal swab were tested and showed a limit of detection of 558 GCE/mL and in (D) clinical samples, 100% agreement between tests run in the UCLA Health Clinical Microbiology Laboratory is shown, negative (n=20) and positive (n=20). In (E) extraction free processing of saliva specimens show a limit or detection down to 1000 GCE per mL.

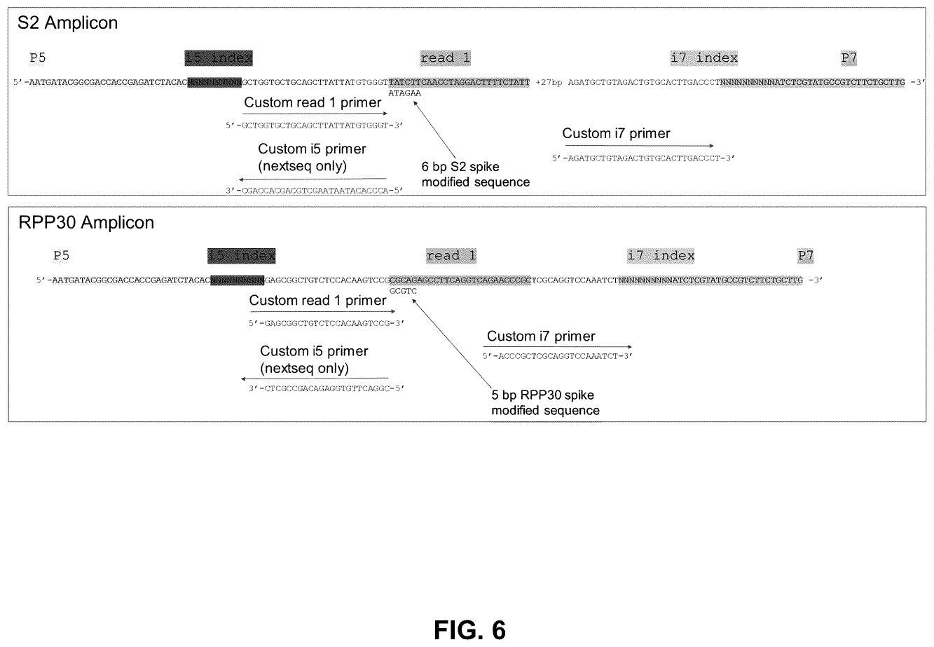

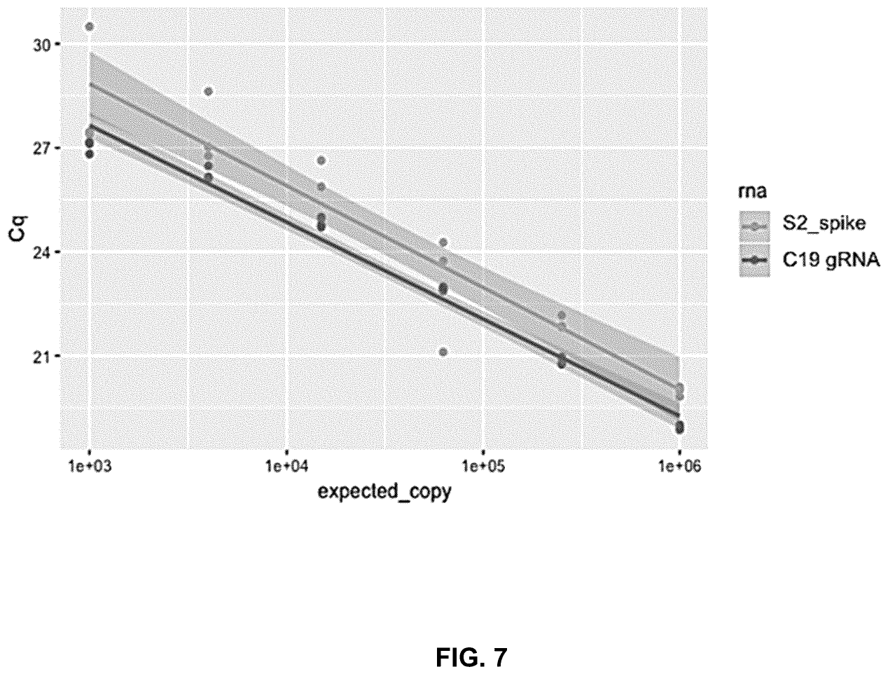

FIG. 6 shows sequencing library design. The amplicon designs are shown for the S2 (top) and RPP30 (bottom) amplicons. Amplicons were designed such that the i5 and i7 molecular indexes uniquely identify each sample. SwabSeq was designed to be compatible with all Illumina platforms. FIG. 7 shows that S2 primers show equivalent PCR efficiency when amplifying the COVID-19 amplicon and the synthetic S2 spike. Slope of PCR efficiency of the primers with either the S2_spike or the SARS-CoV-2 viral (labeled in green as C19gRNA) input are as follows: S2_spike slope=−6.68e-6 and C19gRNA(Twist Control) slope=−6.74e-6. The slopes are expected to equivalent (parallel) if the primers do not show preferential amplification of the S2 spike RNA versus the C19gRNA. This shows that the S2 spike and C19gRNA have equivalent amplification efficiencies using the S2 primer pair. The bands represent 95% confidence intervals for predicted values, are non-overlapping due to different intercepts, and are not relevant for this analysis of slopes.

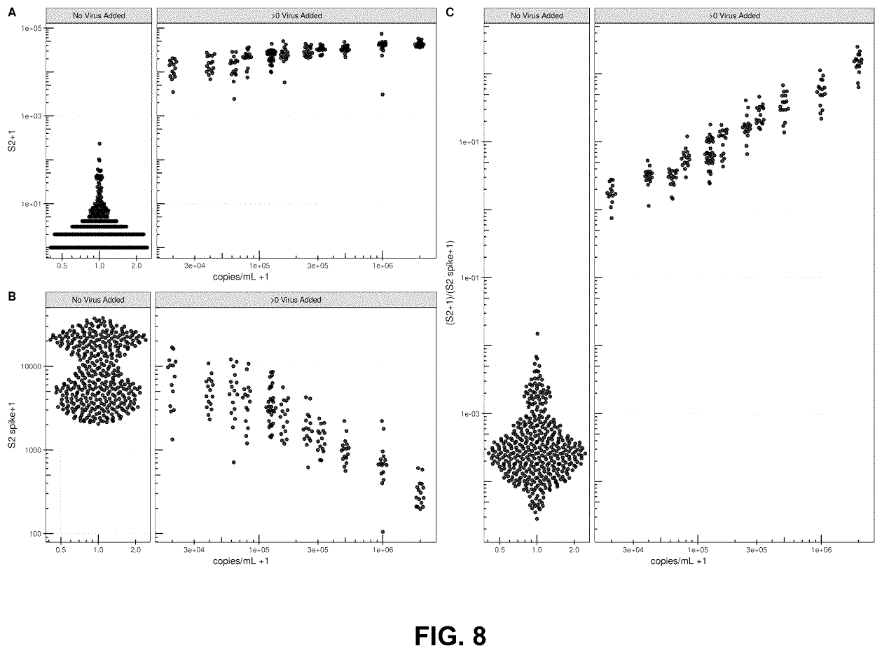

FIG. 8 shows that at very high viral concentrations SwabSeq maintains linearity. An internal well control is included, the S2 Spike, to enable calling negative samples, even in the presence of heterogeneous sample types and PCR inhibition. In (A) as virus concentration increases, increased reads attributed to S2 are observed and, in (B), decreased reads attributed to the S2 Spike. In (C), The ratio between the S2 and S2 Spike provides an additional level of ratiometric normalization and exhibits linearity up to at least 2 million copies/mL of lysate. Note that ticks on both axes are spaced on a log 10 scale.

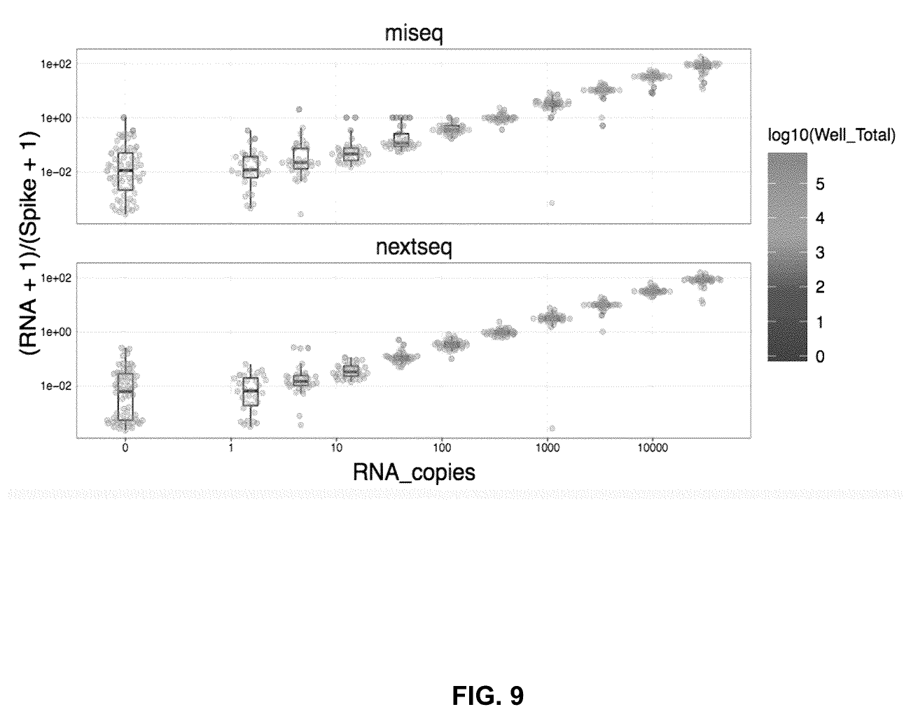

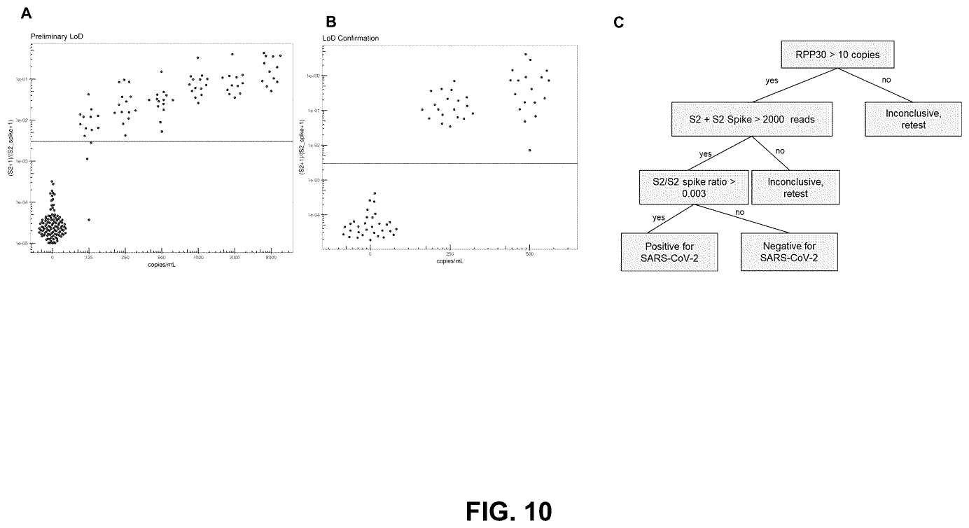

FIG. 9 shows sequencing performed on MiSeq or NextSeq Machine exhibits similar sensitivity. Multiplexed libraries run on both MiSeq and NextSeq showed linearity across a wide range of SARS-CoV2 virus copies in a purified RNA background. FIG. 10 shows preliminary and Confirmatory Limit of Detection Data for RNA purified Samples using the NextSeq550. In (A) the preliminary LOD data identified a LOD of 250 copies/mL, and in (B), confirmatory studies showed an LOD of 250 copies/mL. In (C) exemplary result interpretation guidelines are shown for purified RNA.

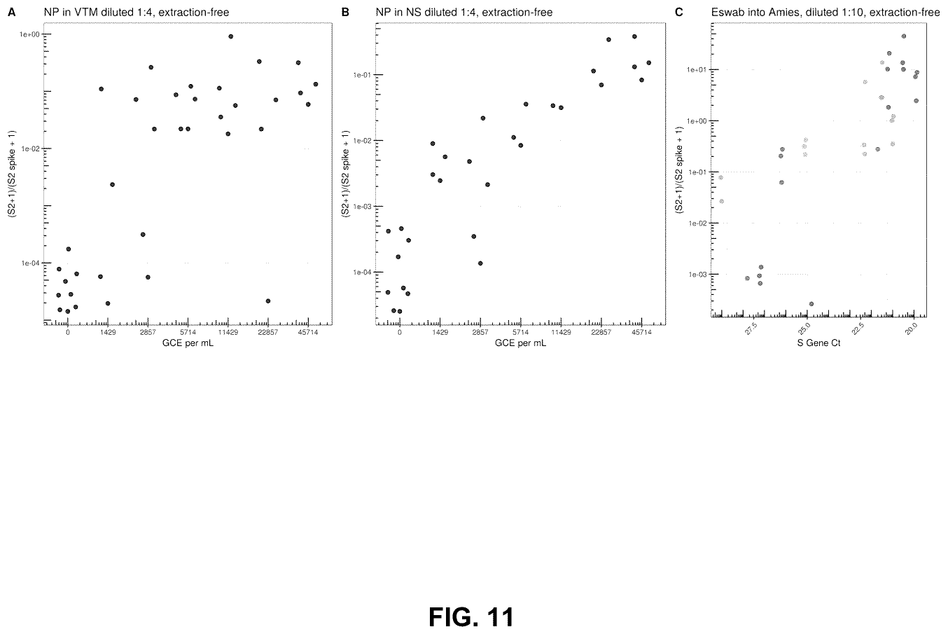

FIG. 11 shows extraction-Free protocols into traditional collection medias and buffers require dilution to overcome effects of RT and PCR inhibition. In (A) tested extraction free protocols for nasopharyngeal (NP) swabs that were placed into viral transport media (VTM). ATCC live inactivated virus were spiked at varying concentrations into pooled VTM and then diluted samples 1:4 with water before adding to the RT-PCR reaction. Limit of detection of 5714 copies per mL was observed. In (B) tested nasopharyngeal (NP) swabs that were collected in normal saline (NS), pooled and then spiked with ATCC live inactivated virus at varying concentrations. Contrived samples were diluted 1:4 in water. Here, the early studies show a similar limit of detection between 2857 and 5714 copies per mL. In (C) tested natural clinical samples that were collected into Amies Buffer (ESwab). S gene Ct count (x-axis) from positive samples were compared to the SwabSeq S2 to S2 spike ratio (y-axis). Samples were run in triplicate (colors). High concordance for Ct counts of 27 and lower but more variability for Ct counts greater than 27 was observed suggesting that RT and PCR inhibition were affecting the limit of detection.

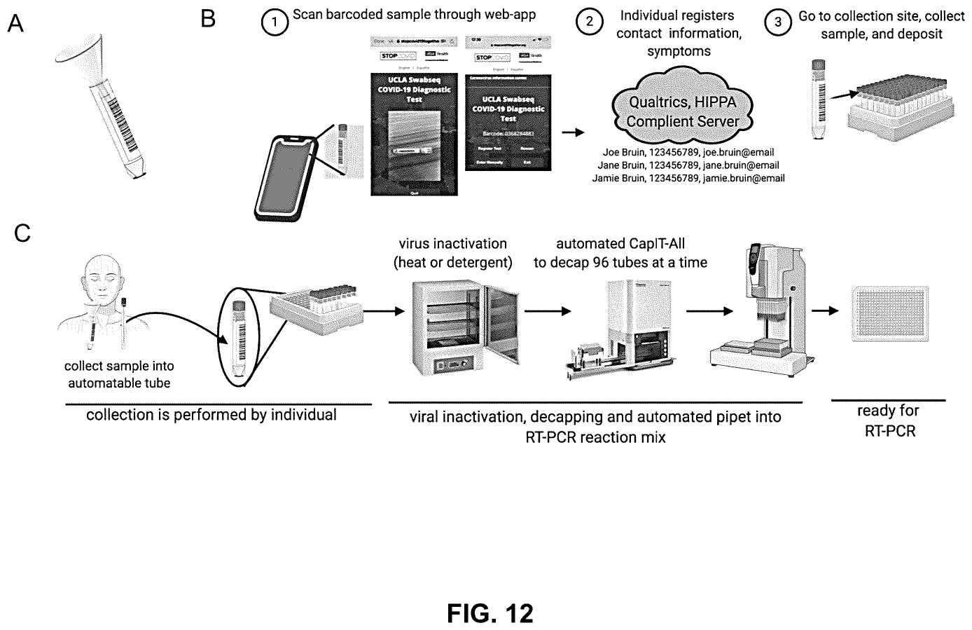

FIG. 12 shows developing a lightweight sample accessioning, collection and processing to allow for scalable testing into the thousands of samples per day. In (A) to address the challenge of sample collection, lightweight collection methods were developed that collect sample directly into an automatable tube. Here a funnel is used for an individual to deposit a small sample of saliva (0.25 mL into the funnel and tube). This setup can accommodate multiple sample types. In (B) to facilitate the sample accessioning and collection, a web-based app was developed for individuals to register their sample tube using a barcode reader and send their identifying information into a secure instance of Qualtrics. Individuals then collect their sample and then place the tube in the rack. This low-touch pre-analytic process allows for thousands of samples a day without heavy administrative burden. In (C) The overall workflow streamlines processing in the lab. First, individuals collect samples into an automatable tube and place them into a 96-tube rack. Samples arrive in the lab in a 96-rack format allowing for efficient inactivation and processing of the samples, drastically increasing the flow of samples through the platform.

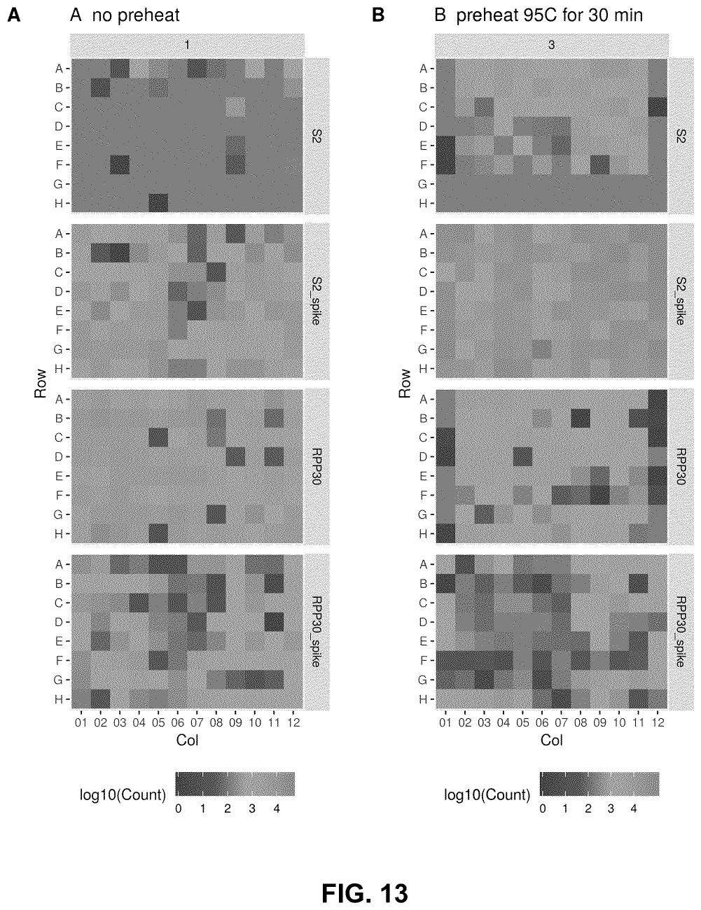

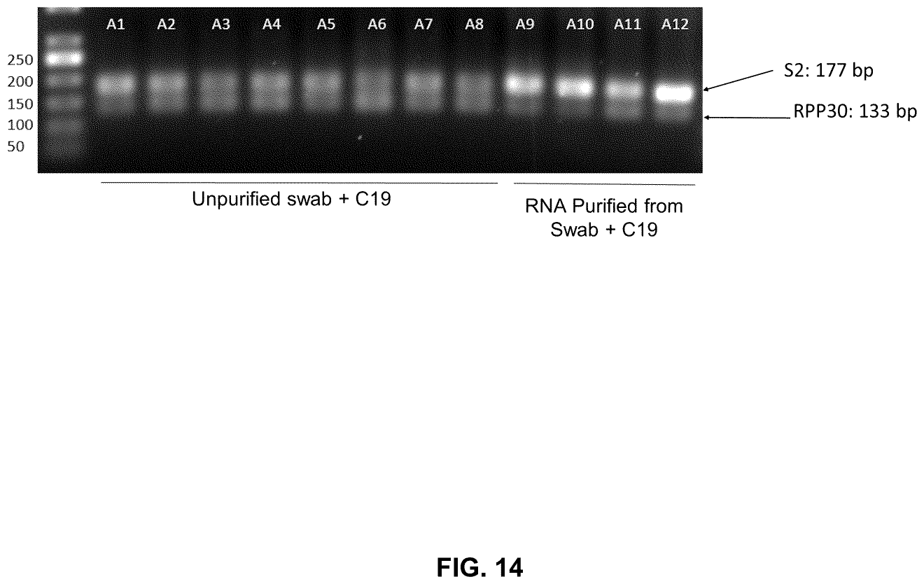

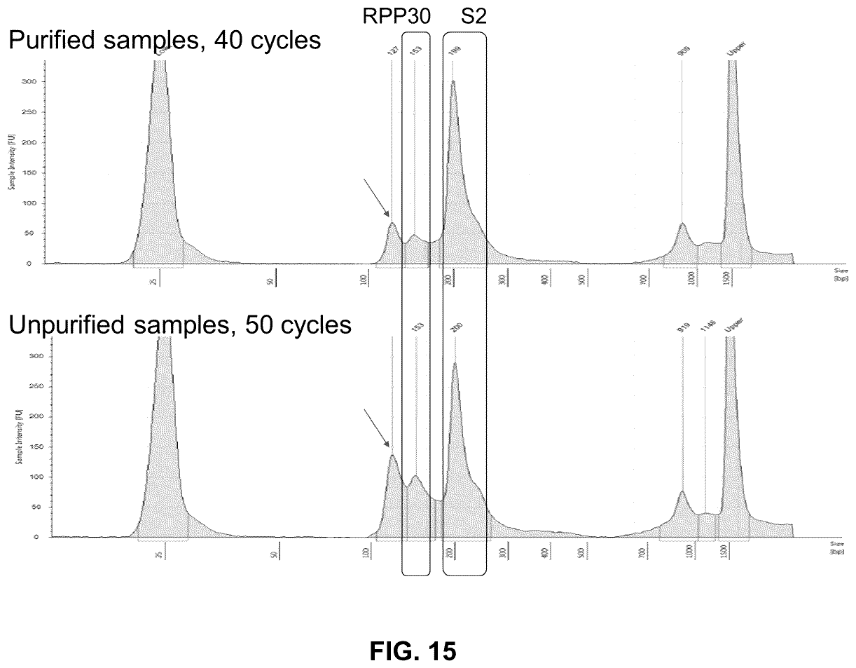

FIG. 13 show that preheating Saliva to 95° C. for 30 minutes drastically improves RT-PCR. Detection of viral genome and shows improved robustness in detection of the controls. In (A) Without preheating, detection of S2 spike is minimal and there are lower counts for the control amplicons. In (B) with a 95° C. preheating step for 30 minutes, robust detection of the S2 amplicon and synthetic S2 Spike was observed. FIG. 14 shows that PCR inhibition has a significant effect on amplification products 2% Agarose gene was run for a subset of wells from the Rt-PCR reactions. RT-PCR inhibition from swabs in unpurified lysate (A1-A8) as compared to purified RNA (A9-A12) was observed. Two bands were observed in this subset of wells representing 2 amplicons for the S2 or S2 spike (177 bp) and RPP30 (133 bp) primer pairs. FIG. 15 shows that tapestation increasing the number of PCR cycles and working with unpurified or inhibitory samples types (e.g. Saliva) was seen to increase the size of a nonspecific peak in the library preparation. Representative result from Agilent TapeStation for the purified amplicon libraries. It was observed that a nonspecific peak slightly above 100 bp (arrow) in both library traces, but this peak increases in size with unpurified samples and an increased number of PCR cycles. Importantly, it was observed that that an increase in the size of this nonspecific peak leads to inaccurate library quantification. Therefore, in order to optimize cluster density on Illumina sequencers, it is suggested that quantifying the loading concentration of the final library based on the proportion of the desired peaks (RPP30 and S2).

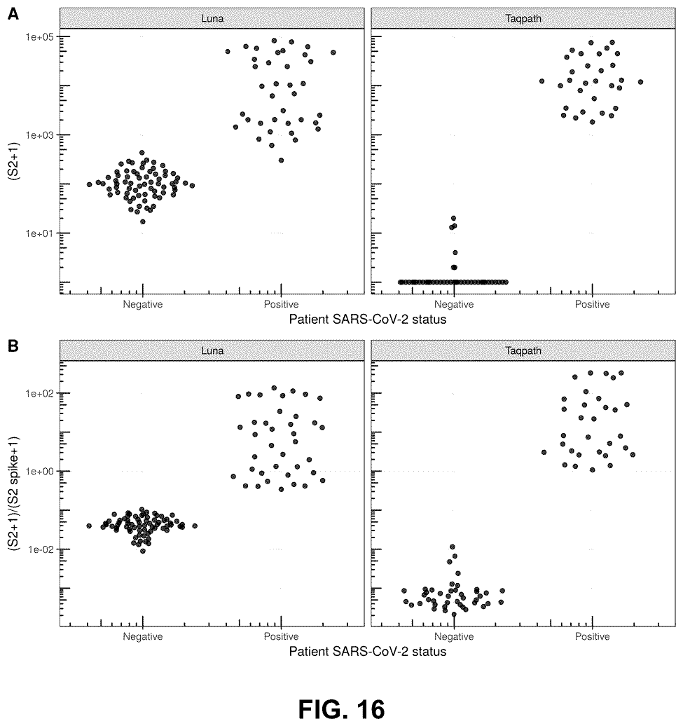

FIG. 16 shows that TaqPath decreases the number of S2 reads in SARS-CoV2-negative samples relative to NEB Luna. In (A) and (B) Luna One Step RT-PCR Mix (New England Biosciences) to TaqPath™ 1-Step RT-qPCR Master Mix (Thermofisher Scientific) was compared. It is likely that the presence of UNG in the TaqPath Mastermix significantly reduced the number of S2 reads in the SARS-CoV-2-negative samples allowing a more accurate diagnosis of SARS-CoV-2-positive and SARS-CoV-2-negative samples.

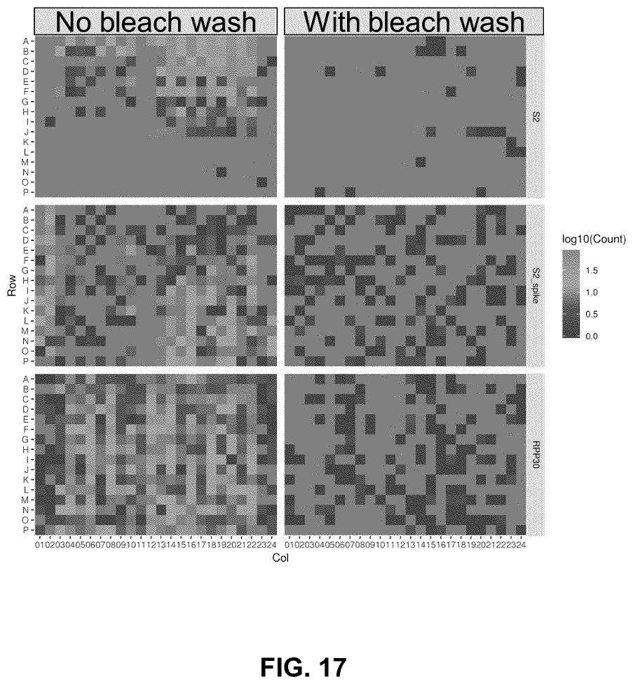

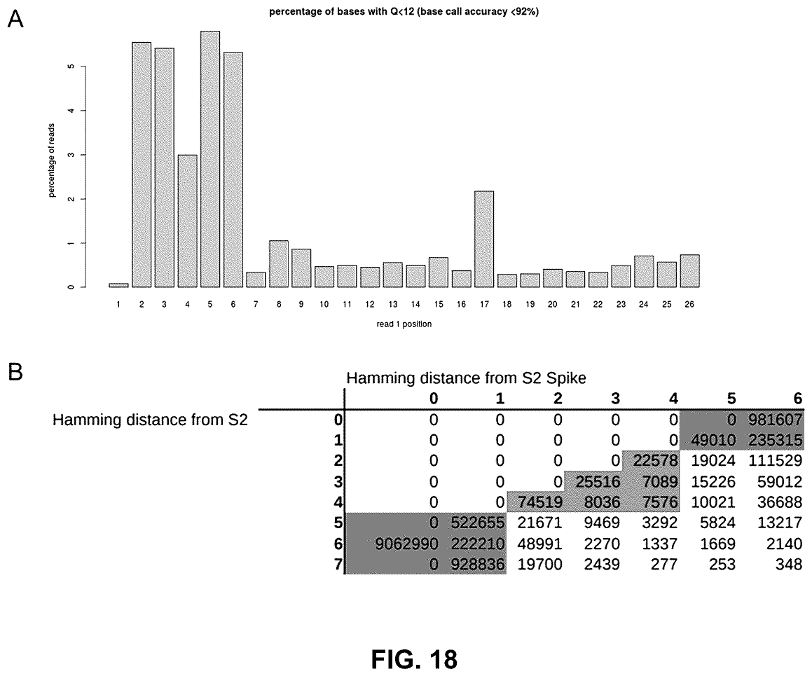

FIG. 17 shows carryover contamination from template line in a MiSeq contributes to cross contamination. In this experiment RT-PCR was performed on four 384-well plates but only pooled three plates. On the left are observed counts of each of the amplicons for each sample for the 384-well plate not included in the run (but for which the indices were used in the previous run). Amplicon reads for indices used in the previous run are present at a low level (0-150 reads). A bleach wash in addition to regular wash was performed prior to the subsequent run. In this subsequent run, three different plates were pooled and left out the fourth 384 well plate. On the right are observed counts of each of the amplicons for sample indices corresponding to the left-out plate (again, for which the indices were used in the previous run). A remarkable decrease in the amount of carryover contamination was observed, where carryover reads are <10 per sample. Alternatively, in some embodiments, in order to reduce contamination and confounding of results therefrom, different combinations of reverse and forward primers are used on subsequent runs on the same instrument. Rotating the primers on the sequences allows for a determination to be made if the reads are true reads from samples or crossover contamination from a previous run. FIG. 18 shows sequencing errors in amplicon read and potential amplicon mis-assignment. In experiment v18 less PhiX was loaded than usual (11%) and the overall quality of reads was lower. Trends noticed here persist in other runs but this run more clearly highlights issues that can occur due to sequencing errors and overly tolerant error-correction. In (A) The percentage of reads with base quality scores less than 12 for each position in read 1. Note that the first 6 bases of read1 distinguish S2 from S2 spike and have the highest percentage of low-quality base calls. In (B) The hamming distance between each read1 sequence and either the expected S2 sequence (rows) or S2 spike sequence (columns), In yellow are perfect match and edit distance 1 sequences that can be clearly identified as S2 or S2 spike. In red are sequences with errors that may be mis-assigned (S2 spike assigned as S2 is most problematic for this assay.)

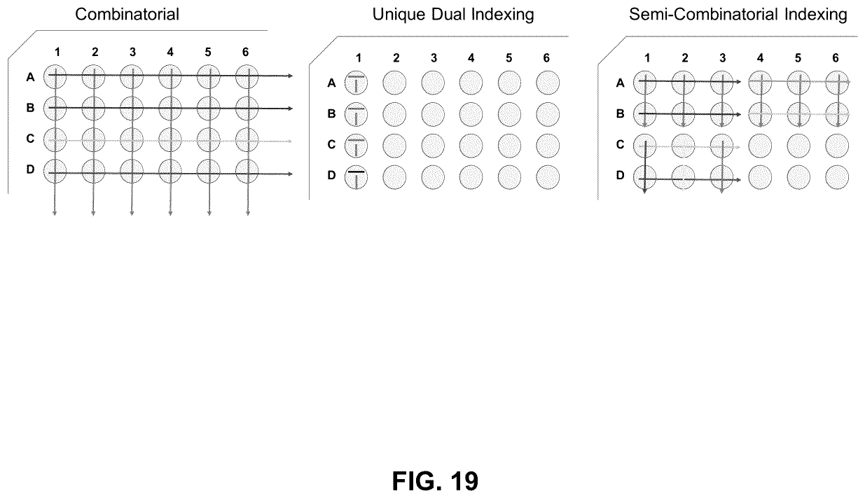

FIG. 19 shows visualization of different indexing strategies. Here i5 indices are depicted as horizontal lines, i7 indices are depicted as vertical lines, and colors represent unique indices. In combinatorial (or fully-combinatorial) indexing, the i5 and i7 indices are combined to make unique combinations, but each i5 and i7 index may be used multiple times within a plate, and all possible i5 and i7. For unique dual indexing, each i5 and i7 index are only used 1 time per plate. This requires many more oligos to be synthesized. For Semi-combinatorial indexing, the combinations used are more limited, such that indices are only repeated for a subset of wells and many possible combinations are not used. In practice (not depicted here), a design where the i7 index is unique but the i5 index can be repeated up to four times across a 384-well plate was used. For the majority of the Swabseq development, either semi-combinatorial indexing (384×96) that allowed for 1536 combinations or samples to be run or unique dual indexing (384 UDI) was used.

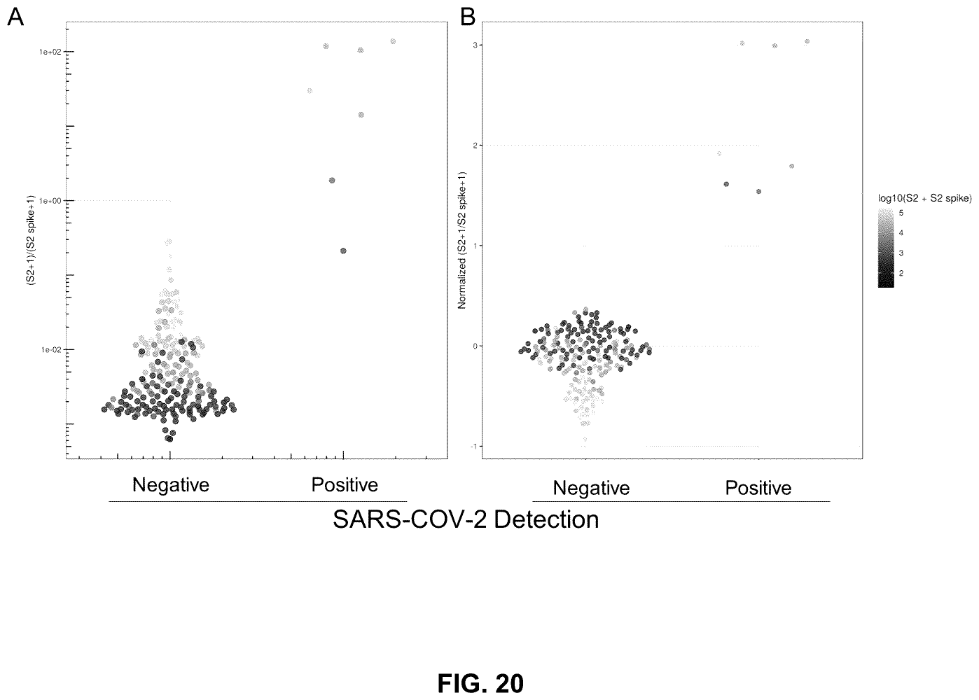

FIG. 20 shows computational correction for index mis-assignment using a mixed-model. To expand the number of samples capable of testing, a combinatorial indexing strategy can be used. In this experiment a single index on i5 was used to uniquely identify a plate and 96 i7 indices to identify wells. In (A) the ratio of S2 to S2 spike (y-axis) is plotted for clinical samples based on whether Covid was detected by RT-qPCR (x-axis). SARS-CoV-2 positive samples were filtered to have Ct<32. The effects of index mis-assignment across plates can be observed as i7 indices that have high a sum of S2 and S2 spike across all samples that share the same i7 barcode across plates (colors). In (B) best linear unbiased predictor residuals are plotted (y-axis) for data in A, after computational correction of the log 10(S2+1/S2_spike+1) ratio by treating the identity of the i7 barcode as a random effect.

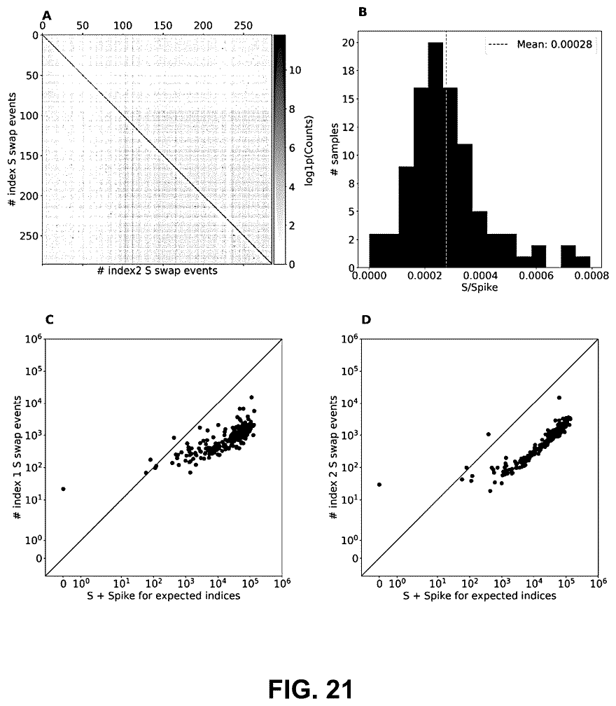

FIG. 21 shows quantifying the role of index mis-assignment as a source of noise in the S2 reads. In (A) a matching matrix for the viral S2+S2 spike count for each pair of i5 and i7 index pairs from run v19 that used a unique dual index design. The index pairs along the diagonal correspond to expected index pairs for samples present in the experiment (expected matching indices) and the index pairs off of the diagonal correspond to index mis-assignment events. In (B) the distribution of ratios of viral S counts to Spike counts for samples with known zero amount of viral RNA. The mean ratio is 0.00028. In (C) the number of i7 mis-assignment events vs the number of viral S2+S2 Spike counts for each sample. In (D) the number of i5 mis-assignment events vs the number of viral S2+S2 Spike counts for each sample.

### Barcodes

Variable nucleotide sequences (barcodes) that serve as an index can be included on any of the first or second set of PCR primers described herein. Additionally, barcodes may be added in a separate library preparation reaction. The variable nucleotide sequences described herein can be used as a sample index in order to deconvolve results obtained from a sequencing reaction used herein.

Once the contents of the cells are released into their respective partitions by a lysis agent, the macromolecular components (e.g., macromolecular constituents of samples, such as RNA, DNA, or proteins) contained therein may be further processed within the partitions. In accordance with the methods and systems described herein, the macromolecular component contents of individual samples can be provided with unique identifiers such that, upon characterization of those macromolecular components they may be attributed as having been derived from the same sample or particles. The ability to attribute characteristics to individual samples or groups of samples is provided by the assignment of unique identifiers specifically to an individual sample or groups of samples. Unique identifiers, e.g., in the form of nucleic acid barcodes can be assigned or associated with individual samples or populations of samples, in order to tag or label the sample's macromolecular components (and as a result, its characteristics) with the unique identifiers. These unique identifiers can then be used to attribute the sample's components and characteristics to an individual sample or group of samples.

In some aspects, this is performed by co-partitioning the individual sample or groups of samples with the unique identifiers or barcodes comprising an unique molecular identifier sequence (UMI). In some aspects, the unique identifiers are provided in the form of nucleic acid molecules (e.g., oligonucleotides) that comprise nucleic acid barcode sequences that may be attached to or otherwise associated with the nucleic acid contents of individual sample, or to other components of the sample, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some embodiments, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some embodiments, the length of a barcode sequence may be about 6, 7, 8, 9, 10, 11, 12, 13, 14, 15, 16, 17, 18, 19, 20 nucleotides or longer. In some embodiments, the length of a barcode sequence may be at least about 6, 7, 8, 9, 10, 11, 12, 13, 14, 15, 16, 17, 18, 19, 20 nucleotides or longer. In some embodiments, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some embodiments, separated barcode subsequences can be from about 4 to about 16 nucleotides in length. In some embodiments, the barcode subsequence may be about 4, 5, 6, 7, 8, 9, 10, 11, 12, 13, 14, 15, 16 nucleotides or longer. In some embodiments, the barcode subsequence may be at least about 4, 5, 6, 7, 8, 9, 10, 11, 12, 13, 14, 15, 16 nucleotides or longer. In some embodiments, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned samples. These sequences include, e.g., targeted or random/universal amplification primer sequences for amplifying the genomic DNA from the individual samples within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more partitions, where one partition contains oligonucleotides, or microdispensing of oligonucleotides into partitions, e.g., partitions within microfluidic systems. In some embodiments, a primer comprises a barcode oligonucleotide. In some embodiments the primer sequence is a targeted primer sequence complementary to a sequence in the template nucleic acid molecule. In some embodiments, the first nucleic acid molecule further comprises one or more functional sequences and wherein the second nucleic acid molecule comprises the one or more functional sequences. In some embodiments, the one or more functional sequences are selected from the group consisting of an adapter sequence, an additional primer sequence, a primer annealing sequence, a sequencing primer sequence, a sequence configured to attach to a flow cell of a sequencer, and a unique molecular identifier sequence.

For example, the above described barcoded nucleic acid molecules (e.g., barcoded oligonucleotides) are added to a sample. In some embodiments, a partition comprises barcoded oligonucleotides having the same barcode sequence. In some embodiments, a partition among a plurality of partitions comprises barcoded oligonucleotides having an identical barcode sequence, wherein each partition among within the plurality of partitions comprises a unique barcode sequence. In some embodiments, the population of barcoded oligonucleotides provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each barcoded oligonucleotide can be provided with large numbers of nucleic acid (e.g., oligonucleotide) molecules attached. In particular, the number of molecules of nucleic acid molecules including the barcode sequence on an individual barcoded oligonucleotide can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some embodiments at least about 1 billion nucleic acid molecules, or more. Nucleic acid molecules of a given barcoded oligonucleotide can include identical (or common) barcode sequences, different barcode sequences, or a combination of both. Nucleic acid molecules of a given barcoded oligonucleotide can include multiple sets of nucleic acid molecules. Nucleic acid molecules of a given set can include identical barcode sequences. The identical barcode sequences can be different from barcode sequences of nucleic acid molecules of another set

Moreover, when the population of barcoded oligonucleotides is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some embodiments at least about 1 billion nucleic acid molecules.

In some embodiments, it may be desirable to incorporate multiple different barcodes within a given partition. For example, in some embodiments, a barcoded oligonucleotide within a partition can comprise (1) a common barcode sequence shared by all barcoded oligonucleotides within the partition and (2) a unique molecular identifier or additional barcode sequence that is different among each barcoded oligonucleotide. The common barcode sequences may provide greater assurance of identification in the subsequent processing, e.g., by providing a stronger address or attribution of the barcodes to a given partition, as a duplicate or independent confirmation of the output from a given partition.

In some embodiments, the barcoded oligonucleotides are attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein.

The nucleic acid molecules (e.g., oligonucleotides) can be releasable from the beads upon the application of a particular stimulus to the beads. In some embodiments, the stimulus may be a photo-stimulus, e.g., through cleavage of a photo-labile linkage that releases the nucleic acid molecules. In other embodiments, a thermal stimulus may be used, where elevation of the temperature of the beads environment will result in cleavage of a linkage or other release of the nucleic acid molecules form the beads. In still other embodiments, a chemical stimulus can be used that cleaves a linkage of the nucleic acid molecules to the beads, or otherwise results in release of the nucleic acid molecules from the beads. In one embodiment, such compositions include the polyacrylamide matrices described above for encapsulation of samples, and may be degraded for release of the attached nucleic acid molecules through exposure to a reducing agent, such as DTT.

A support can be contemplated for use in a method of the present disclosure may be, for example, a well, matrix, rod, container, or bead(s). A support may have any useful features and characteristics, such as any useful size, surface chemistry, fluidity, solidity, density, porosity, and composition. In some embodiments, a support is a surface of a well on a plate. In some embodiments, a support may be a bead such as a gel bead. A bead may be solid or semi-solid. Additional details of beads are provided elsewhere herein.

A support (e.g., a bead) may comprise an anchor sequence functionalized thereto (e.g., as described herein). An anchor sequence may be attached to the support via, for example, a disulfide linkage. An anchor sequence may comprise a partial read sequence and/or flow cell functional sequence. Such a sequence may permit sequencing of nucleic acid molecules attached to the sequence by a sequencer (e.g., an Illumina sequencer). Different anchor sequences may be useful for different sequencing applications. An anchor sequence may comprise, for example, a TruSeq or Nextera sequence. An anchor sequence may have any useful characteristics such as any useful length and nucleotide composition. For example, an anchor sequence may comprise 2, 3, 4, 5, 6, 7, 8, 9, 10, 11, 12, 13, 14, 15, 16, 17, 18, 19, 20, or more nucleotides. In some embodiments, an anchor sequence may comprise 15 nucleotides. Nucleotides of an anchor sequence may be naturally occurring or non-naturally occurring (e.g., as described herein). A bead may comprise a plurality of anchor sequences attached thereto. For example, a bead may comprise a plurality of first anchor sequences attached thereto. In some embodiments, a bead may comprise two or more different anchor sequences attached thereto. For example, a bead may comprise both a plurality of first anchor sequences (e.g., Nextera sequences) and a plurality of second anchor sequences (e.g., TruSeq sequences) attached thereto. For a bead comprising two or more different anchor sequences attached thereto, the sequence of each different anchor sequence may be distinguishable from the sequence of each other anchor sequence at an end distal to the bead. For example, the different anchor sequences may comprise one or more nucleotide differences in the 2, 3, 4, 5, 6, 7, 8, 9, 10, or more nucleotides furthest from the bead.

In some embodiments, multiple different barcode molecules (e.g., nucleic acid barcode molecules) may be generated on the same support (e.g., bead). For example, two different barcode molecules may be generated on the same support. Alternatively, three or more different barcode molecules may be generated on the same support. Different barcode molecules attached to the same support may comprise one or more different sequences. For example, different barcode molecules may comprise one or more different barcode sequences, and/or other sequences (e.g., starter sequences). In some embodiments, different barcode molecules attached to the same support may comprise the same barcode sequences. Different barcode molecules attached to the same support may comprise barcode sequences that are the same or different. Similarly, different barcode molecules may comprise unique molecular identifiers (UMIs) that are the same or different.

### Next Generation Sequencing

As described in the methods disclosed herein, the sequencing of nucleic acid molecules is used and is useful for the detection and diagnosis of a pathogenic infection. Generally, sequencing refers to methods and technologies for determining the sequence of nucleotide bases in one or more polynucleotides. The polynucleotides can be, for example, deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina, Pacific Biosciences, Oxford Nanopore, or Life Technologies (Ion Torrent). Such devices may provide a plurality of raw genetic data corresponding to the genetic information of a subject (e.g., human), as generated by the device from a sample provided by the subject. In some situations, systems and methods provided herein may be used with proteomic information. Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

Next generation sequencing includes many technologies capable of generating large amounts of sequence information and excluding Sanger sequencing or Maxam-Gilbert sequencing. Generally, next generation sequencing encompasses single molecule real-time sequencing, sequencing-by-synthesis, ion semiconductor sequencing and the like. Exemplary next-gneration sequencing machines may comprise the MiniSeq, the iSeq100, the NextSeq 1000, the NextSeq 2000, the NovaSeq 6000, the NextSeq 550 series and the like from Illumina, Inc; Ion Torrent machines from Thermo Fisher Scientific; or the Sequel systems from Pacific Biosciences.

Next generation sequencing machines used with the method herein can generate at least 1, 5, 10, 15, 25, 50, 75, 100, 200, 300 gigabases of data or more in a 24 hour period from a single machine.

Next generation sequencing machines used with the method herein can generate at least 1, 1, 4, 10, 15, 25, 50, 75, 100, 200, 300, 500, or 1,000 million sequence reads of data or more in a 24 hour period from a single machine.

Also included is a computer program, computing device, or analysis platform/system to receive and analyze sequencing data, and output one or more reports that can be transmitted or accessed electronically via a server, an analysis portal, or by e-mail. The computing device or analysis platform can operate according to the algorithms and methods described herein.

### Reaction Mixtures

Also provided herein are reaction mixtures for determining the presence or absence of a viral nucleic acid in a biological sample. In some embodiments, the reaction mixture comprises a synthetic nucleic acid provided herein, at least a portion of said biological sample, and one or more enzyme or reagents sufficient to amplify said viral nucleic acid in said biological sample, if present.

The biological sample may be any of the biological samples provided herein. In some embodiments, the biological sample is a human biological sample. In some embodiments, said biological sample comprises saliva, a cheek swab, a nasopharyngeal swab, or a mid-turbinate swab. In some embodiments, said biological sample comprises saliva or a nasopharyngeal swab. In some embodiments, said biological sample comprises saliva. In some embodiments, said biological sample comprises a nasopharyngeal swab.

The synthetic nucleic acid may be any of the synthetic nucleic acids provided herein. In some embodiments, the synthetic nucleic acid is a SARS-CoV-2 synthetic RNA nucleic acid provided herein. In some embodiments, said SARS-CoV-2 synthetic RNA nucleic acid is present at a concentration from about 10 copies per reaction mixture to about 500 copies per reaction mixture. In some embodiments, said SARS-CoV-2 synthetic RNA nucleic acid is present at a concentration of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 320, 340, 360, 380, 400, 420, 440, 460, 480, or 500 copies per reaction mixture.

The viral nucleic acid may be any of the viral nucleic acids provided herein. In some embodiments, said viral nucleic acid is an influenza A, influenza B, or a coronavirus nucleic acid. In some embodiments, said coronavirus nucleic acid is a is SARS-COV-2 nucleic acid.

In some embodiments, the enzymes or reagents comprise a reverse transcriptase enzyme, dNTPs, a primer pair specific for said viral nucleotide sequence, a primer pair specific for a sample control nucleotide sequence, a magnesium salt, or combinations thereof.

In some embodiments, the reaction mixture comprises one or more enzymes which can be used to amplify the viral nucleic acid. In some embodiments, the enzyme is a reverse transcriptase. Non-limiting examples of reverse-transcriptase enzymes include Avian Myeloblastosis Virus (AMV) Reverse Transcriptase and Moloney Murine Leukemia Virus (M-MuLV, MMLV), and variants thereof.

In some embodiments, the reaction comprises deoxynucleotide triphosphates (dNTPs). In some embodiments, the kit comprises a mixture of each of the dNTPs necessary for amplification of the viral nucleic acid, as well as any other desired nucleic acids (e.g., dATG, dCTP, dTTP, dGTP).

In some embodiments, the reaction mixture comprises a primer pair specific for the viral nucleotide sequence. The primer pair specific for the viral nucleotide sequence can be any of the primer pairs provided herein. said primer pair specific for said viral nucleotide sequence is specific for an influenza A nucleotide sequence, an influenza B nucleotide sequence and a coronavirus nucleotide sequence. In some embodiments, said primer pair specific for said viral nucleotide sequence is specific for a coronavirus S1 or N2 sequence.

In some embodiments, the reaction mixture comprises a primer pair specific for a sample control nucleotide sequence. In some embodiments, the primer pair specific for the sample control nucleotide sequence is specific for an endogenous nucleotide sequence expressed by the organism from which the biological sample is derived. In some embodiments, the primer pair specific for the sample control nucleotide is specific for a housekeeping gene. In some embodiments, the primer pair specific for the sample control nucleotide sequence is specific for GAPDH, RPP30, or ACTB. In some embodiments, the primer pair specific for the sample control nucleotide is specific for RPP30.

In some embodiments, said primer pair specific for said viral nucleotide sequence or said primer pair specific for said sample control nucleotide sequence is present at a concentration from about 50 micromolar to about 250 micromolar. In some embodiments, said primer pair specific for said viral nucleotide sequence or said primer pair specific for said sample control nucleotide sequence is present at a concentration of about 50, 60, 70, 80, 90, 100, 110, 120, 130, 140, 150, 160, 170, 180, 190, 200, 210, 220, 230, 240, or 250 micromolar. In some embodiments, said primer pair specific for said viral nucleotide sequence or said primer pair specific for said sample control nucleotide sequence is present at a concentration of about 100 micromolar. In some embodiments, said primer pair specific for said viral nucleotide sequence or said primer pair specific for said sample control nucleotide sequence is present at a concentration of about 200 micromolar.

In some embodiments, the reaction mixture comprises a magnesium salt. In some embodiments, the magnesium salt is included in a sufficient quantity to allow the enzymes of the reaction (e.g., the reverse transcriptase enzyme) to function and to amplify targeted nucleic acids. In some embodiments, the magnesium salt is magnesium chloride. In some embodiments, the reaction mixture comprises a concentration of magnesium ions of about 0.1 mM to about 50 mM. In some embodiments, the concentration of magnesium ion is from about 1 mM to about 10 mM.

In some embodiments, the volume of said reaction mixture is from about 10 microliters to about 100 microliters. In some embodiments, the volume of said reaction mixture is from about 20 microliters to about 90 microliters, from about 30 microliters to about 80 microliters, or from about 40 microliters to about 60 microliters. In some embodiments, the volume of said reaction mixture is about 10, 20, 30, 40, 50, 60, 70, 80, 90, or 100 microliters.

### Kits

Also described herein is a kit comprising an oligonucleotide primer pair described herein. In certain embodiments, is a kit comprising an oligonucleotide primer pair described herein and a synthetic nucleic acid according to this disclosure. The synthetic nucleic acid may be for an S1, N2 amplification. The kit may also contain reagents sufficient for amplification: such as buffers (provided at 10×, 5×, 2×, or 1× concentration) for reverse transcription, PCR amplification, and/or sequencing reactions, dNTPs, reverse transcriptase enzymes, PCR amplification enzymes (e.g., Taq polymerase and/or variants of the same). Theses components may be packaged in a vial or container singly or in combination as appropriate.

Also described herein is a kit for determining the presence or absence of a viral nucleic acid in a biological sample. In some embodiments, the kit comprises a synthetic nucleic acid provided herein and one or more enzymes or reagents sufficient to amplify the viral nucleic acid form the biological sample.

In some embodiments, the enzymes or reagents comprise a reverse transcriptase enzyme, dNTPs, a primer pair specific for said viral nucleotide sequence, a primer pair specific for a sample control nucleotide sequence, a magnesium salt, or combinations thereof.

In some embodiments, the kit comprises one or more enzymes which can be used to amplify the viral nucleic acid. In some embodiments, the enzyme is a reverse transcriptase. Non-limiting examples of reverse-transcriptase enzymes include Avian Myeloblastosis Virus (AMV) Reverse Transcriptase and Moloney Murine Leukemia Virus (M-MuLV, MMLV), and variants thereof.

In some embodiments, the kit comprises deoxynucleotide triphosphates (dNTPs). In some embodiments, the kit comprises a mixture of each of the dNTPs necessary for amplification of the viral nucleic acid, as well as any other desired nucleic acids (e.g., dATG, dCTP, dTTP, dGTP).

In some embodiments, the kit comprises a primer pair specific for the viral nucleotide sequence. The primer pair specific for the viral nucleotide sequence can be any of the primer pairs provided herein. said primer pair specific for said viral nucleotide sequence is specific for an influenza A nucleotide sequence, an influenza B nucleotide sequence and a coronavirus nucleotide sequence. In some embodiments, said primer pair specific for said viral nucleotide sequence is specific for a coronavirus S1 or N2 sequence.

In some embodiments, the kit comprises a primer pair specific for a sample control nucleotide sequence. In some embodiments, the primer pair specific for the sample control nucleotide sequence is specific for an endogenous nucleotide sequence expressed by the organism from which the biological sample is derived. In some embodiments, the primer pair specific for the sample control nucleotide is specific for a housekeeping gene. In some embodiments, the primer pair specific for the sample control nucleotide sequence is specific for GAPDH, RPP30, or ACTB. In some embodiments, the primer pair specific for the sample control nucleotide is specific for RPP30.

In some embodiments, the kit comprises a magnesium salt. In some embodiments, the magnesium salt is included in a sufficient quantity to allow the enzymes of the kit (e.g., the reverse transcriptase enzyme) to function. In some embodiments, the magnesium salt is magnesium chloride.

The following embodiments recite nonlimiting permutations of combinations of features disclosed herein. Other permutations of combinations of features are also contemplated. In particular, each of these numbered embodiments is contemplated as depending from or relating to every previous or subsequent numbered embodiment, independent of their order as listed.
1. A method of diagnosing an individual with a pathogen infection, the method comprising: (a) providing a biological sample from said individual; (b) contacting said biological sample from said individual with a lysis agent, to obtain a lysed biological sample; (c) performing a polymerase chain reaction (PCR) on said lysed biological sample to obtain a PCR amplified lysed biological sample, wherein said PCR reaction on said lysed biological sample is performed with a first set of PCR primers, wherein said first set of PCR primers amplifies a pathogen nucleic acid sequence; (d) sequencing said PCR amplified lysed biological sample using next generation sequencing; and (e) providing a positive diagnosis for said pathogen infection if a pathogen sequence is detected by said PCR or by said sequencing or providing a negative diagnosis for said individual if a pathogen sequence is not detected by said PCR or by said sequencing. 2. The method of embodiment 1, wherein said individual is a human individual. 3. The method of embodiment 1 or 2, wherein said pathogen infection comprises a bacterial infection, a viral infection, or a fungal infection, and combinations thereof 4. The method of any one of embodiments 1 to 3, wherein said lysis agent comprises water or PCR reaction buffer heated to at least 50 degrees Celsius. 5. The method of any one of embodiments 1 to 3, wherein said lysis agent comprises water or PCR reaction buffer heated to at least 90 degrees Celsius. 6. The method of any one of embodiments 1 to 5, wherein said bacterial infection is an infection by the genera Streptococcus, Pseudomonas, Shigella, Campylobacter, Salmonella, Clostridium, or Escherichia, and combinations thereof 7. The method of any one of embodiments 1 to 5, wherein said fungal infection is an infection by Candida, Blastomyces, Cryptococcus, Coccidoides, Histoplasma, Paracoccidioides, Sporothrix, or Pneumocystis, and combinations thereof 8. The method of any one of embodiments 1 to 5, wherein the viral infection is an infection by a DNA virus. 9. The method of embodiment 8, wherein said DNA virus comprises hepatitis B, Hepatitis C, papillomavirus, Epstein-Barr virus, varicella, or variola, and combinations thereof. 10. The method of any one of embodiments 1 to 5, wherein said viral infection is an infection by an RNA virus. 11. The method of embodiment 10, wherein said RNA virus comprises an influenza virus, a coronavirus, a polio virus, a measles virus, an Ebola virus, a retrovirus, or an Orthomyxovirus. 12. The method of embodiment 11, wherein said viral infection is a coronavirus infection. 13. The method of embodiment 12, wherein said coronavirus infection is SARS-COV-2 infection. 14. The method of any one of embodiments 1 to 13, wherein said biological sample from said individual is from a blood sample, a plasma sample, a serum sample, a cheek swab, a urine sample, a semen sample, a vaginal swab, a stool sample, a nasopharyngeal swab, mid-turbinate swab, or any combination thereof 15. The method of embodiment 14, wherein said biological sample from said individual is from a nasopharyngeal swab, a mid-turbinate swab, or any combination thereof 16. The method of any one of embodiments 1 to 15, comprising adding a synthetic nucleic acid to said lysis agent or said lysed biological sample. 17. The method of any one of embodiments 1 to 15, comprising adding a plurality of synthetic nucleic acids to said lysis agent or said lysed biological sample, the plurality comprising synthetic nucleic acids that have distinct sequences. 18. The method of embodiment 17, wherein the plurality comprises four. 19. The method of any one of embodiments 1 to 18, comprising adding a synthetic nucleic acid to said method. 20. The method of any one of embodiments 1 to 18, comprising adding a plurality of synthetic nucleic acids to said method, the plurality comprising synthetic nucleic acids that have distinct sequences. 21. The method of embodiment 20, wherein the plurality comprises four. 22. The method of embodiment 19, wherein said synthetic nucleic acid is an RNA. 23. The method of embodiment 19, wherein said synthetic nucleic acid is a DNA. 24. The method of any one of embodiments 19 or 23, wherein said synthetic nucleic acid comprises a set of sequences configured to be bound by said first set of PCR primers. 25. The method of any one of embodiments 19 to 24, wherein said first set of primers amplifies both said pathogen nucleic acid sequence and said synthetic nucleic acid. 26. The method of any one of embodiments 19 to 24, wherein said synthetic nucleic acid sequence comprises a nucleotide sequence that is not identical to said pathogen nucleic acid sequence. 27. The method of any one of embodiments 1 to 26, comprising performing a reverse transcription reaction on said lysed biological sample. 28. The method of embodiment 27, wherein said reverse transcription reaction is performed before said performing said polymerase chain reaction. 29. The method of any one of embodiments 1 to 26, wherein said reverse transcription reaction is performed without further purification of said lysed biological sample. 30. The method of any one of embodiments 1 to 26, wherein said reverse transcription reaction on said lysed biological sample produces viral cDNA. 31. The method of embodiment 30, wherein said viral cDNA is coronavirus cDNA. 32. The method of embodiment 31, wherein said coronavirus cDNA is SARS-COV-2 cDNA. 33. The method of any one of embodiments 1 to 31, wherein said reverse transcription reaction and said PCR is a single-step reaction. 34. The method of any one of embodiments 1 to 33, wherein said PCR is an end-point analysis. 35. The method of any one of embodiments 1 to 34, wherein said PCR is not a real-time PCR reaction. 36. The method of any one of embodiments 1 to 35, wherein said first set of PCR primers amplifies a coronavirus nucleic acid sequence. 37. The method of embodiment 36, wherein said coronavirus nucleic acid sequence is a SARS-COV-2 nucleic acid sequence. 38. The method of embodiment 37, wherein said SARS-COV-2 nucleic acid sequence comprises the N1 or S2 gene. 39. The method of any one of embodiments 1 to 38, comprising a second set of PCR primers wherein said second set of primers amplifies a nucleic acid sequence of said individual. 40. The method of embodiment 39, wherein said second set of PCR primers amplifies a human nucleic acid sequence. 41. The method of embodiment 40, wherein said second set of PCR primers amplifies a human nucleic acid sequence selected from GAPDH, ACTB, RPP30, and combinations thereof. 42. The method of embodiment 41, wherein said second set of PCR primers amplifies human RPP30. 43. The method of any one of embodiments 40 to 42, wherein the second set of PCR primer comprises a mixture of primers with sequencing adaptor sequences and primers without sequencing adaptor sequences. 44. The method of embodiment 43, wherein a ratio of primers with sequencing adaptor sequences to primers without sequencing adaptor sequences is about 1:1, about 1:2, about 1:3 or about 1:4. 45. The method of any one of embodiments 1 to 44, wherein said PCR comprises from 30 to 45 amplification cycles. 46. The method of embodiment 45, wherein said PCR comprises from 35 to 45 amplification cycles. 47. The method of embodiment 45, wherein said PCR comprises from 39 to 42 amplification cycles. 48. The method of any one of embodiments 1 to 47, wherein said first set of PCR primers, said second set of PCR primers, or both said first set of PCR primers and said second set of PCR primers comprises a nucleic acid sequence comprising a variable nucleotide sequence. 49. The method of embodiment 48, wherein said variable nucleotide sequence is a sample ID unique for said individual. 50. The method of any one of embodiments 1 to 49, wherein said first set of PCR primers, said second set of PCR primers, or both said first set of PCR primers and said second set of PCR primers comprises an adapter sequence for a next-generation sequencing reaction. 51. The method of any one of embodiments 39 to 50, comprising adding a second synthetic nucleic acid to said method. 52. The method of embodiment 51, wherein said second synthetic nucleic acid is an RNA. 53. The method of embodiment 51, wherein said second synthetic nucleic acid is a DNA. 54. The method of any one of embodiments 51 to 53, wherein said second synthetic nucleic acid comprises a set of sequences configured to be bound by said second set of PCR primers. 55. The method of any one of embodiments 51 to 54, wherein said second set of primers amplifies both said human nucleic acid sequence and said synthetic nucleic acid. 56. The method of any one of embodiments 51 to 54, wherein said synthetic nucleic acid sequence comprises a nucleotide sequence that is not identical to said human nucleic acid sequence. 57. The method of any one of embodiments 1 to 56, wherein said method can detect less than 10 copies of pathogen genome. 58. The method of any one of embodiments 1 to 56, wherein said method can detect less than 5 copies of pathogen genome. 59. The method of embodiment 57 or 58, wherein said pathogen genome is a coronavirus genome. 60. The method of embodiment 57 or 58, wherein said coronavirus genome is a SARS-COV-2 genome. 61. The method of any one of embodiments 1 to 60, wherein said positive diagnosis for coronavirus if a coronavirus sequence is detected by said PCR is a SARS-COV-2 diagnosis. 62. The method of any one of embodiments 1 to 61, wherein the method determines a strain of coronavirus. 63. The method of any one of embodiments 1 to 61, wherein the method determines a strain of COVD-19.
64. A method of diagnosing an individual with a pathogen infection, the method comprising amplifying nucleic acids from a biological sample from said individual using a first set of PCR primers thereby obtaining amplified nucleic acids, wherein said first set of PCR primers amplifies a pathogen nucleic acid sequence and a synthetic nucleic acid sequence from said biological sample, wherein said synthetic nucleic acid sequence differs from said pathogen nucleic acid sequence by at least one nucleotide. 65. The method of embodiment 64, wherein said individual is a human individual. 66. The method of embodiment 64 or 65, wherein said pathogen infection comprises a bacterial infection, a viral infection, or a fungal infection. 67. The method of any one of embodiments 64 to 66, wherein said bacterial infection is an infection by the genera Streptococcus, Pseudomonas, Shigella, Campylobacter, Salmonella, Clostridium, or Escherichia, and combinations thereof. 68. The method of any one of embodiments 64 to 66, wherein said fungal infection is an infection by Candida, Blastomyces, Cryptococcus, Coccidoides, Histoplasma, Paracoccidioides, Sporothrix, or Pneumocystis, and combinations thereof 69. The method of any one of embodiments 64 to 66, wherein the viral infection is an infection by a DNA virus. 70. The method of embodiment 69, wherein said DNA virus comprises hepatitis B, Hepatitis C, papillomavirus, Epstein-Barr virus, varicella, variola, or any combination thereof. 71. The method of any one of embodiments 64 to 66, wherein said viral infection is an infection by an RNA virus. 72. The method of embodiment 71, wherein said RNA virus comprises an influenza virus, a coronavirus, a polio virus, a measles virus, an Ebola virus, a retrovirus, or an Orthomyxovirus. 73. The method of embodiment 71, wherein said viral infection is a coronavirus infection. 74. The method of embodiment 73, wherein said coronavirus infection is SARS-COV-2 infection. 75. The method of any one of embodiments 64 to 74, wherein said biological sample from said individual is from a blood sample, a plasma sample, a serum sample, a cheek swab, a urine sample, a semen sample, a vaginal swab, a stool sample, a nasopharyngeal swab, mid-turbinate swab, or any combination thereof 76. The method of embodiment 75, wherein said biological sample from said individual is from a nasopharyngeal swab, a mid-turbinate swab, or any combination thereof 77. The method of any one of embodiments 64 to 76, wherein said synthetic nucleic acid is an RNA. 78. The method of any one of embodiments 64 to 76, wherein said synthetic nucleic acid is a DNA. 79. The method of any one of embodiments 64 to 78, wherein said synthetic nucleic acid comprises a set of sequences configured to be bound by said first set of PCR primers. 80. The method of any one of embodiments 64 to 79, wherein said synthetic nucleic acid sequence differs from said pathogen nucleic acid sequence by at least 5 nucleotides. 81. The method of any one of embodiments 64 to 79, wherein said pathogen infection is diagnosed based upon the ratio of synthetic nucleic acid sequence to pathogen nucleic acid sequence. 82. The method of any one of embodiments 64 to 81, comprising performing a reverse transcription reaction on said nucleic acids from said biological sample. 83. The method of any one of embodiments 64 to 81, wherein said reverse transcription reaction on said nucleic acids from said biological sample produces coronavirus cDNA. 84. The method of embodiment 83, wherein said coronavirus cDNA is SARS-COV-2 cDNA. 85. The method of any one of embodiments 64 to 84, wherein said amplifying nucleic acids comprises a PCR reaction. 86. The method of embodiment 85, wherein said PCR reaction is an end-point analysis. 87. The method of any one of embodiments 64 to 86, wherein said PCR reaction is not a real-time PCR reaction. 88. The method of any one of embodiments 64 to 87, wherein said first set of PCR primers amplifies a coronavirus nucleic acid sequence. 89. The method of embodiment 88, wherein said coronavirus nucleic acid sequence is a SARS-COV-2 nucleic acid sequence. 90. The method of embodiment 89, wherein said SARS-COV-2 nucleic acid sequence comprises the N1 or S2 gene. 91. The method of any one of embodiments 64 to 90, wherein said first set of PCR primers comprises a nucleic acid sequence comprising a variable nucleotide sequence. 92. The method of embodiment 91, wherein said variable nucleotide sequence is a sample ID unique for said individual. 93. The method of any one of embodiments 64 to 92, wherein said first set of PCR primers comprises an adapter sequence for a next-generation sequencing reaction. 94. The method of any one of embodiments 64 to 92, comprising amplifying nucleic acids from said biological sample using a second set of PCR primers, wherein said second set of PCR primers amplifies a human nucleic acid sequence. 95. The method of embodiment 94, wherein said second set of PCR primers amplifies a nucleic acid sequence selected from GAPDH, ACTB, RPP30, and combinations thereof. 96. The method of embodiment 95, wherein said second set of PCR primers amplifies human RPP30. 97. The method of any one of embodiments 94 to 96, wherein the second set of PCR primer comprises a mixture of primers with sequencing adaptor sequences and primers without sequencing adaptor sequences. 98. The method of embodiment 97, wherein a ratio of primers with sequencing adaptor sequences to primers without sequencing adaptor sequences is about 1:1, about 1:2, about 1:3 or about 1:4. 99. The method of any one of embodiments 64 to 98, wherein said PCR comprises from 30 to 45 amplification cycles. 100. The method of embodiment 99, wherein said PCR comprises from 35 to 45 amplification cycles. 101. The method of embodiment 99, wherein said PCR comprises from 39 to 42 amplification cycles. 102. The method of any one of embodiments 94 to 101, wherein said second set of PCR primers comprises a nucleic acid sequence comprising a variable nucleotide sequence. 103. The method of embodiment 102, wherein said variable nucleotide sequence is a sample ID unique for said individual. 104. The method of any one of embodiments 94 to 103, wherein said second set of PCR primers comprises an adapter sequence a next-generation sequencing reaction. 105. The method of any one of embodiments 64 to 104, comprising sequencing said amplified nucleic acids from said biological sample using a next-generation sequencing technology. 106. The method of any one of embodiments 64 to 105, wherein said method can detect less than 10 copies of pathogen genome. 107. The method of any one of embodiments 64 to 104, wherein said method can detect less than 5 copies of pathogen genome. 108. The method of embodiment 106 or 107, wherein said pathogen genome is coronavirus genome. 109. The method of embodiment 106 or 107, wherein said pathogen genome is SARS-COV-2 genome. 110. The method of any one of embodiments 64 to 109, wherein the method determines a strain of coronavirus. 111. The method of embodiment 110, wherein the method determines a strain of SARS-COV-2.
112. A synthetic nucleic acid comprising a 5′ proximal region, a 3′ proximal region and an intervening nucleic acid sequence. 113. The synthetic nucleic acid of embodiment 112, wherein said synthetic nucleic acid comprises RNA. 114. The synthetic nucleic acid of embodiment 112, wherein said synthetic nucleic acid comprises DNA. 115. The synthetic nucleic acid of any one of embodiments 112 to 114, wherein said 5′ proximal region comprises a viral nucleic acid sequence. 116. The method of embodiment 115, wherein said viral nucleic acid sequence comprises a coronavirus sequence. 117. The method of embodiment 116, wherein said viral nucleic acid sequence comprises a SARS-COV-2 sequence. 118. The synthetic nucleic acid of any one of embodiments 112 to 114, wherein said 3′ proximal region comprises a viral nucleic acid sequence. 119. The method of embodiment 118, wherein said viral nucleic acid sequence comprises a coronavirus sequence. 120. The method of embodiment 119, wherein said viral nucleic acid sequence comprises a SARS-COV-2 sequence. 121. The synthetic nucleic acid of any one of embodiments 112 to 120, wherein said 5′ proximal region, said 3′ proximal region, or both said 5′ proximal region and said 3′ proximal region are less than about 30 nucleotides in length. 122. The synthetic nucleic acid of any one of embodiments 112 to 120, wherein said 5′ proximal region, said 3′ proximal region, or both said 5′ proximal region and said 3′ proximal region are less than about 25 nucleotides in length. 123. The synthetic nucleic acid of any one of embodiments 112 to 120, wherein said 5′ proximal region, said 3′ proximal region, or both said 5′ proximal region and said 3′ proximal region are less than about 20 nucleotides in length. 124. The synthetic nucleic acid of any one of embodiments 112 to 123, wherein said 5′ proximal region is at the 5′ terminus of said synthetic nucleic acid. 125. The synthetic nucleic acid of any one of embodiments 112 to 123, wherein said 3′ proximal region is at the 3′ terminus of said synthetic nucleic acid. 126. The synthetic nucleic acid of any one of embodiments 112 to 125, wherein said intervening nucleic acid sequence is less than about 99%, 98%, 97%, 95%, 90%, 85%, 80%, or 75%, identical to a viral nucleic acid sequence. 127. The synthetic nucleic acid of embodiment 126, wherein said synthetic nucleic acid sequence is a coronavirus sequence. 128. The synthetic nucleic acid of embodiment 126, wherein said synthetic nucleic acid sequence is a SARS-COV-2 sequence. 129. Use of the synthetic nucleic acid of any one of embodiments 112 to 128 in a method to detect pathogen infection in said individual. 130. The use of embodiment 129, wherein said pathogen infection is a coronavirus infection. 131. The use of embodiment 130, wherein said viral infection is a SARS-COV-2 infection.
132. A method of nucleic acid processing for the detection of a viral infection, said method comprising: (a) providing a sample comprising a viral nucleic acid molecule and a host nucleic acid molecule; (b) generating a barcoded viral nucleic acid molecule by performing a nucleic acid extension reaction on said viral nucleic acid molecule using a first primer comprising a first barcode sequence; (c) generating a barcoded host nucleic acid molecule by performing a nucleic acid extension reaction on said host nucleic acid molecule using a second primer comprising a second barcode sequence; (d) sequencing said barcoded viral nucleic acid molecule and said barcoded host nucleic acid molecule to identify (i) said barcode sequence and (ii) a sequence corresponding to said viral nucleic acid molecule, or a derivative thereof, and said host nucleic acid molecule; and (e) providing a positive diagnosis for a viral infection if said sequence corresponding to said viral nucleic acid molecule is identified in (d). 133. The method of embodiment 132, wherein (b) and (c) are performed simultaneously. 134. The method of any one of embodiments 132 to 133, wherein said first primer further comprises one or more additional functional sequences selected from the group consisting of primer sequences, adapter sequences, primer annealing sequences, a unique molecular identifier sequence, and capture sequences. 135. The method of any one of embodiments 132 to 134, wherein said second primer further comprises one or more additional functional sequences selected from the group consisting of primer sequences, adapter sequences, primer annealing sequences, a unique molecular identifier sequence, and capture sequences. 136. The method of any one of embodiments 132 to 135, wherein (a) further comprises providing a synthetic nucleic acid molecule. 137. The method of embodiments 136, wherein the synthetic nucleic acid molecule comprises a synthetic sequence that is different from said viral nucleic acid molecule and said human nucleic acid molecule. 138. The method of embodiment 137, wherein (b) generating a barcoded synthetic nucleic acid molecule by performing said nucleic acid extension using said first primer. 139. The method of embodiment 137, wherein (c) generating a barcoded synthetic nucleic acid molecule by performing said nucleic acid extension using said second primer. 140. The method of any one of embodiments 132 to 139, wherein said nucleic acid extension reaction is a reverse transcription reaction. 141. The method of any one of embodiments 132 to 139, wherein said nucleic acid extension reaction is a polymerase chain reaction. 142. The method of any one of embodiments 132 to 139, wherein said nucleic acid extension reaction comprises a reverse transcriptase reaction, a polymerase chain reaction, or a combination thereof. 143. The method of embodiment 142, wherein (b), said nucleic acid extension reaction comprises: (i) hybridizing said first primer to said viral nucleic acid molecule; and (ii) using a reverse transcriptase enzyme to extend said primer. 144. The method of embodiment 142, wherein (b), said nucleic acid extension reaction comprises: (i) hybridizing said first primer to said viral nucleic acid molecule; and (ii) using a reverse transcriptase enzyme to extend said primer. 145. The method of embodiment 142, wherein (b), said nucleic acid extension reaction comprises: (i) hybridizing said first primer to said viral nucleic acid molecule; and (ii) using a polymerase enzyme to extend said primer. 146. The method of embodiment 142, wherein the polymerase chain reaction is an end-point polymer chain reaction. 147. The method of any one of embodiments 132 to 146, wherein the method further comprises, prior to (d), amplifying said barcoded viral nucleic acid molecule and said barcoded host nucleic acid molecule. 148. The method of any one of embodiments 132 to 147, wherein the method further comprises, prior to (d), subjecting said barcoded viral nucleic acid molecule and said barcoded host nucleic acid molecule to N cycles of a polymerase chain reaction. 149. The method of embodiment 148, wherein N is greater than 35 cycles. 150. The method of embodiment 148, wherein N is greater than 40 cycles. 151. The method of embodiment 148, wherein N is greater than 45 cycles. 152. The method of embodiment 148, wherein said polymerase chains reaction incorporates one or more additional sequences into one or both of said barcoded viral nucleic acid molecule and barcoded host nucleic acid molecule, selected from the group consisting of a sample index sequence, an adapter sequence, primer sequence, a primer binding sequence, a sequence configured to couple to the flow cell of a sequencer, and an additional barcode sequence. 153. The method of any one of embodiments 132 to 152, wherein subsequent to (b) and (c), said sample is combined with one or more samples after performing at least one nucleic acid extension reaction in (b) and (c). 154. The method of embodiment 153, wherein said sample is combined with one or more samples after performing a single round of said nucleic acid extension reaction in (b) and (c). 155. The method of any one of embodiments 132 to 154, wherein said sample comprises one or more cells. 156. The method of embodiment 155, further comprising prior to (b), releasing said viral nucleic acid molecule and said host nucleic acid molecule from said one or more cells. 157. The method of embodiment 155, further comprising prior to (b), subjecting said sample to conditions sufficient to release said viral nucleic acid molecule and said host nucleic acid molecule from said one or more cells. 158. The method of embodiment 157, wherein (b) and (c) are performed under said conditions sufficient to release said viral nucleic acid molecule and said host nucleic acid molecule from said one or more cells. 159. The method of embodiment 157, wherein said viral nucleic acid molecule and said host nucleic acid molecule is released is not purified from said sample prior to (b) and (c). 160. The method of any one of embodiments 132 to 159, further comprising in (d) using said barcode sequence to associate said viral nucleic acid molecule, or a derivative thereof, and said host nucleic acid molecule, or a derivative thereof, as being associated with said sample. 161. The method of any one of embodiments 132 to 160, further comprising in (e) using said barcode sequence of said barcoded viral nucleic acid molecule and said barcoded host nucleic acid molecule to identify said sample, wherein said sample corresponds to a subject being tested for said viral infection. 162. The method of any one of embodiments 132 to 161, wherein said sample is obtained from a subject. 163. The method of embodiment 162, wherein said subject is a human. 164. The method of embodiment 162, wherein said subject is an animal. 165. The method of embodiment 162, wherein said host nucleic acid molecule is part of a genome of said subject. 166. The method of embodiment 162, wherein said host nucleic acid molecule is part of a transcriptome of said subject. 167. The method of any one of embodiments 132 to 166, wherein said host nucleic acid molecule encodes an ubiquitously expressed protein. 168. The method of any one of embodiments 132 to 166, wherein said host nucleic acid molecule is a genomic DNA molecule. 169. The method of any one of embodiments 132 to 166, wherein said host nucleic acid molecule is an ubiquitously transcribed RNA molecule. 170. The method of any one of embodiments 132 to 169, wherein said host nucleic acid is GAPDH, ACTB, RPP30, and combinations thereof. 171. The method of any one of embodiments 132 to 170, wherein said viral nucleic acid molecule is part of a genome of a virus. 172. The method of embodiment 171, wherein said virus is a coronavirus. 173. The method of embodiment 172, wherein said coronavirus is selected from the group consisting of severe acute respiratory syndrome coronavirus 2 (SARS-COV-2), severe acute respiratory syndrome coronavirus (SARS-CoV), and Middle East respiratory syndrome coronavirus (MERS-CoV). 174. The method of embodiment 173, wherein said coronavirus is SARS-COV-2. 175. The method of embodiment 174, wherein said virus is an RNA virus. 176. The method of embodiment 175, wherein said RNA virus comprises a double-stranded RNA genome. 177. The method of embodiment 175, wherein said RNA virus comprises a single-stranded RNA genome. 178. The method of embodiment 175, wherein said RNA virus is selected from the group consisting of coronavirus, influenza, human immunodeficiency virus, and Ebola virus. 179. The method of embodiment 170, wherein said virus is a DNA virus. 180. The method of any one of embodiments 34 to 169, further comprising, prior to (a), partitioning said sample. 181. The method of embodiment 180, wherein said partition is a well. 182. The method of embodiment 180, wherein said partition is a well among a plurality of wells. 183. The method of embodiment 182, wherein said barcode sequence is unique to said well among said plurality of wells. 184. The method of any one of embodiments 132 to 183, wherein (b) and (c) are performed concurrently. 185. The method of any one of embodiments 132 to 184, wherein the viral infection is SARS-COV-2. 186. The method of any one of embodiments 132 to 183, wherein the sample is obtained from a subject via a nasopharyngeal or mid-turbinate swab.
187. A composition, comprising a synthetic nucleic acid molecule comprising a first nucleic acid sequence and a second nucleic sequence, wherein (1) the first nucleic acid sequence is identical to a sequence from a pathogen nucleic acid molecule, and (2) the second nucleic acid sequence is not identical to a sequence the pathogen nucleic acid molecule. 188. The composition of embodiment 187, wherein the first nucleic acid sequence is located to the 3′ the second nucleic acid sequence. 189. The composition of any one of embodiments 187 to 188, wherein the synthetic nucleic acid molecule further comprises a third nucleic acid sequence, wherein the third nucleic acid sequence is identical to a second sequence from the pathogen nucleic acid molecule. 190. The composition of embodiment 176, wherein the third nucleic acid sequence is 5′ to the second nucleic acid sequence. 191. The composition of any one of embodiments 187 to 190, wherein the first nucleic acid sequence or the third nucleic acid sequence is less than 5, 10, 15, 20, 25, or 30 nucleotides. 192. The composition of any one of embodiments 187 to 191, wherein the second nucleic acid sequence comprises a total number of nucleotides less than 25, 50, 100, 150, 200, or 500 nucleotides. 193. The composition of any one of embodiments 187 to 191, wherein the second nucleic acid sequence comprises a total number of nucleotides greater than 25, 50, 100, 150, 200, or 500 nucleotides. 194. The composition of any one of embodiments 187 to 193, wherein the synthetic nucleic acid molecule is a ribonucleic acid (RNA) molecule, a deoxyribonucleic acid (DNA) molecule, or an RNA-DNA hybrid molecule. 195. The composition of any one of embodiments 187 to 194, wherein the first nucleic acid sequence, the third nucleic acid sequence, or both the first nucleic acid sequence and the third nucleic acid sequence comprise a primer binding site. 196. The composition of any one of embodiments 187 to 194, wherein the composition further comprises the pathogen nucleic acid molecule. 197. The composition of embodiment 196, wherein the pathogen nucleic molecule is from a pathogen, wherein the pathogen comprises a bacterium, a virus, a fungus, or combinations thereof 198. The composition of embodiment 197, wherein the bacterium is from the genera Streptococcus, Pseudomonas, Shigella, Campylobacter, Salmonella, Clostridium, or Escherichia, and combinations thereof 199. The composition of embodiment 197, wherein the fungus is Candida, Blastomyces, Cryptococcus, Coccidoides, Histoplasma, Paracoccidioides, Sporothrix, or Pneumocystis, and combinations thereof 200. The composition of embodiment 197, wherein the virus is a DNA virus. 201. The composition of embodiment 200, wherein the DNA virus comprises hepatitis B, Hepatitis C, papillomavirus, Epstein-Barr virus, varicella, or variola, and combinations thereof 202. The composition of embodiment 197, wherein the virus is an RNA virus. 203. The composition of embodiment 202, wherein the RNA virus comprises an influenza virus, a coronavirus, a polio virus, a measles virus, an Ebola virus, a retrovirus, or an Orthomyxovirus. 204. The composition of embodiment 189, wherein the virus is a coronavirus. 205. The composition of embodiment 190, wherein the coronavirus is a SARS-COV-2 virus. 206. The composition of any one of embodiments 196 to 205, wherein the composition further comprises a plurality of primers, wherein a primer of the plurality of primers is configured to hybridize to a sequence of the synthetic nucleic acid molecule or a sequence of the pathogen nucleic acid molecule. 207. The composition of embodiment 206, wherein a sequence of the synthetic nucleic acid molecule and the sequence the pathogen nucleic acid molecule are identical. 208. The composition of any one of embodiments 187 to 207, wherein the synthetic nucleic acid molecule is amplified with the same efficiency as the pathogen nucleic acid molecule. 209. The composition of any one of embodiments 187 to 207, wherein the synthetic nucleic acid molecule is configured to create an amplification produce the same size or within 10 base pairs in size as an amplification product of the pathogen nucleic acid molecule.
210. A method of diagnosing an individual with a pathogen infection, the method comprising: (a) providing a biological sample from said individual; (c) performing a polymerase chain reaction (PCR) on said biological sample to obtain a PCR amplified biological sample, wherein said PCR reaction on said biological sample is performed with a first set of PCR primers, wherein said first set of PCR primers amplifies a pathogen nucleic acid sequence; (d) sequencing said PCR amplified lysed biological sample using next generation sequencing; and (e) providing a positive diagnosis for said pathogen infection if a pathogen sequence is detected by said PCR or by said sequencing or providing a negative diagnosis for said individual if a pathogen sequence is not detected by said PCR or by said sequencing. 211. The method of embodiment 210, wherein said individual is a human individual. 212. The method of embodiment 210 or 211, wherein said pathogen infection comprises a bacterial infection, a viral infection, or a fungal infection, and combinations thereof. 213. The method of any one of embodiments 210 to 212, wherein said bacterial infection is an infection by the genera Streptococcus, Pseudomonas, Shigella, Campylobacter, Salmonella, Clostridium, or Escherichia, and combinations thereof. 214. The method of any one of embodiments 210 to 212, wherein said fungal infection is an infection by Candida, Blastomyces, Cryptococcus, Coccidoides, Histoplasma, Paracoccidioides, Sporothrix, or Pneumocystis, and combinations thereof. 215. The method of any one of embodiments 210 to 212, wherein the viral infection is an infection by a DNA virus. 216. The method of embodiment 215, wherein said DNA virus comprises hepatitis B, Hepatitis C, papillomavirus, Epstein-Barr virus, varicella, or variola, and combinations thereof 217. The method of any one of embodiments 210 to 216, wherein said viral infection is an infection by an RNA virus. 218. The method of embodiment 217, wherein said RNA virus comprises an influenza virus, a coronavirus, a polio virus, a measles virus, an Ebola virus, a retrovirus, or an Orthomyxovirus. 219. The method of embodiment 218, wherein said viral infection is a coronavirus infection. 220. The method of embodiment 219, wherein said coronavirus infection is SARS-COV-2 infection. 221. The method of any one of embodiments 210 to 220, wherein said biological sample from said individual is from a blood sample, a plasma sample, a serum sample, a cheek swab, a urine sample, a semen sample, a vaginal swab, a stool sample, a nasopharyngeal swab, mid-turbinate swab, or any combination thereof. 222. The method of embodiment 221, wherein said biological sample from said individual is from a nasopharyngeal swab, a mid-turbinate swab, or any combination thereof. 223. The method of any one of embodiments 210 to 222, comprising adding a synthetic nucleic acid to said lysis agent or said lysed biological sample. 224. The method of any one of embodiments 210 to 222, comprising adding a synthetic nucleic acid to said method. 225. The method of embodiment 224, wherein said synthetic nucleic acid is an RNA. 226. The method of embodiment 224, wherein said synthetic nucleic acid is a DNA. 227. The method of any one of embodiments 224 to 226, wherein said synthetic nucleic acid comprises a set of sequences configured to be bound by said first set of PCR primers. 228. The method of any one of embodiments 224 to 227, wherein said first set of primers amplifies both said pathogen nucleic acid sequence and said synthetic nucleic acid. 229. The method of any one of embodiments 224 to 228, wherein said synthetic nucleic acid sequence comprises a nucleotide sequence that is not identical to said pathogen nucleic acid sequence. 230. The method of any one of embodiments 210 to 229, comprising performing a reverse transcription reaction on said lysed biological sample. 231. The method of embodiment 230, wherein said reverse transcription reaction is performed before said performing said polymerase chain reaction. 232. The method of any one of embodiments 210 to 231, wherein said reverse transcription reaction is performed without further purification of said lysed biological sample. 233. The method of any one of embodiments 210 to 231, wherein said reverse transcription reaction on said lysed biological sample produces viral cDNA. 234. The method of embodiment 233, wherein said viral cDNA is coronavirus cDNA. 235. The method of embodiment 233, wherein said coronavirus cDNA is SARS-COV-2 cDNA. 236. The method of any one of embodiments 210 to 235, wherein said reverse transcription reaction and said PCR is a single-step reaction. 237. The method of any one of embodiments 210 to 236, wherein said PCR is an end-point analysis. 238. The method of any one of embodiments 210 to 237, wherein said PCR is not a real-time PCR reaction. 239. The method of any one of embodiments 210 to 238, wherein said first set of PCR primers amplifies a coronavirus nucleic acid sequence. 240. The method of embodiment 239, wherein said coronavirus nucleic acid sequence is a SARS-COV-2 nucleic acid sequence. 241. The method of embodiment 240, wherein said SARS-COV-2 nucleic acid sequence comprises the N1 or S2 gene. 242. The method of any one of embodiments 210 to 241, comprising a second set of PCR primers wherein said second set of primers amplifies a nucleic acid sequence of said individual. 243. The method of embodiment 242, wherein said second set of PCR primers amplifies a human nucleic acid sequence. 244. The method of embodiment 243, wherein said second set of PCR primers amplifies a human nucleic acid sequence selected from GAPDH, ACTB, RPP30, and combinations thereof. 245. The method of embodiment 244, wherein said second set of PCR primers amplifies human RPP30. 246. The method of any one of embodiments 242 to 245, wherein the second set of PCR primer comprises a mixture of primers with sequencing adaptor sequences and primers without sequencing adaptor sequences. 247. The method of embodiment 246, wherein a ratio of primers with sequencing adaptor sequences to primers without sequencing adaptor sequences is about 1:1, about 1:2, about 1:3 or about 1:4. 248. The method of any one of embodiments 210 to 247, wherein said PCR comprises from 30 to 45 amplification cycles. 249. The method of embodiment 248, wherein said PCR comprises from 35 to 45 amplification cycles. 250. The method of embodiment 248, wherein said PCR comprises from 39 to 42 amplification cycles. 251. The method of any one of embodiments 210 to 250, wherein said first set of PCR primers, said second set of PCR primers, or both said first set of PCR primers and said second set of PCR primers comprises a nucleic acid sequence comprising a variable nucleotide sequence. 252. The method of embodiment 251, wherein said variable nucleotide sequence is a sample ID unique for said individual. 253. The method of any one of embodiments 210 to 252, wherein said first set of PCR primers, said second set of PCR primers, or both said first set of PCR primers and said second set of PCR primers comprises an adapter sequence for a next-generation sequencing reaction. 254. The method of any one of embodiments 210 to 253, wherein said method can detect less than 10 copies of pathogen genome. 255. The method of any one of embodiments 210 to 253, wherein said method can detect less than 5 copies of pathogen genome. 256. The method of embodiment 254 or 255, wherein said pathogen genome is a coronavirus genome. 257. The method of embodiment 254 or 255, wherein said coronavirus genome is a SARS-COV-2 genome. 258. The method of any one of embodiments 210 to 257, wherein said positive diagnosis for coronavirus if a coronavirus sequence is detected by said PCR is a SARS-COV-2 diagnosis. 259. The method of any one of embodiments 210 to 258, wherein the method determines a strain of coronavirus. 260. The method of any one of embodiments 210 to 258, wherein the method determines a strain of COVD-19.
261. A composition comprising a plurality of synthetic nucleic acids with a distinct nucleic acid sequence, said plurality of synthetic nucleic acids with a distinct nucleic acid sequence composing a common 5′ sequence identical to a pathogen nucleic acid sequence, a common 3′ sequence identical to a pathogen nucleic acid sequence, and an intervening sequence that differs among the plurality of sequences. 262. The composition of embodiment 261, wherein said plurality of synthetic nucleic acids with a distinct nucleic acid sequence are single-stranded. 263. The composition of embodiment 261, wherein said plurality of synthetic nucleic acids with a distinct nucleic acid sequence are double-stranded. 264. The composition of any one of embodiments 261 to 263, wherein said plurality of synthetic nucleic acids with a distinct nucleic acid sequences consist of or comprise RNA. 265. The composition of any one of embodiments 261 to 263, wherein said plurality of synthetic nucleic acids with a distinct nucleic acid sequences consist of or comprise DNA. 266. The composition of any one of embodiments 261 to 265, wherein said common 5′ sequence identical to a pathogen nucleic acid sequence is 30 nucleotides or less. 267. The composition of any one of embodiments 261 to 266, wherein said common 3′ sequence identical to a pathogen nucleic acid sequence is 30 nucleotides or less. 268. The composition of any one of embodiments 261 to 267, wherein said intervening sequence is 50 nucleotides or less. 269. The composition of embodiment 268, wherein said intervening sequence is 30 nucleotides or less. 270. The composition of any one of embodiments 261 to 269, wherein the pathogen nucleic acid sequence is from a bacterial pathogen, a fungal pathogen, or a viral pathogen. 271. The composition of embodiment 270, wherein said bacterial pathogen is of the genera Streptococcus, Pseudomonas, Shigella, Campylobacter, Salmonella, Clostridium, or Escherichia, and combinations thereof. 272. The composition of embodiment 270, wherein said fungal pathogen is Candida, Blastomyces, Cryptococcus, Coccidoides, Histoplasma, Paracoccidioides, Sporothrix, or Pneumocystis, and combinations thereof 273. The composition of embodiment 270, wherein said viral pathogen is a DNA virus. 274. The composition of embodiment 273, wherein said DNA virus comprises hepatitis B, Hepatitis C, papillomavirus, Epstein-Barr virus, varicella, or variola, and combinations thereof 275. The composition of any one of embodiments 1 to 3, wherein said viral pathogen is an RNA virus. 276. The composition of embodiment 270, wherein said RNA virus comprises an influenza virus, a coronavirus, a polio virus, a measles virus, an Ebola virus, a retrovirus, an Orthomyxovirus, or combinations thereof. 277. The composition of embodiment 276, wherein said viral pathogen is a coronavirus. 278. The composition of embodiment 277, wherein said coronavirus is SARS-COV-2. 279. The composition of embodiments 277 or 278, wherein said pathogen nucleic acid sequence is a nucleic acid sequence that encodes a coronavirus spike protein. 280. The composition of any one of embodiments 277 to 279, wherein said plurality of synthetic nucleic acids with a distinct nucleic acid sequence comprise or consist of a sequence selected from any one or more of S2_001, S2_002, S2_003, and S2_004. 281. The composition of any one of embodiments 261 to 280, for use in a method of diagnosing or detecting infection with a pathogen. 282. The composition of any one of embodiments 261 to 280, for use in a method of normalizing pathogen next-generation sequence reads.

### EXAMPLES

The following illustrative examples are representative of embodiments of compositions and methods described herein and are not meant to be limiting in any way.

### Example 1—SwabSeq Diagnosis of Viral Infection

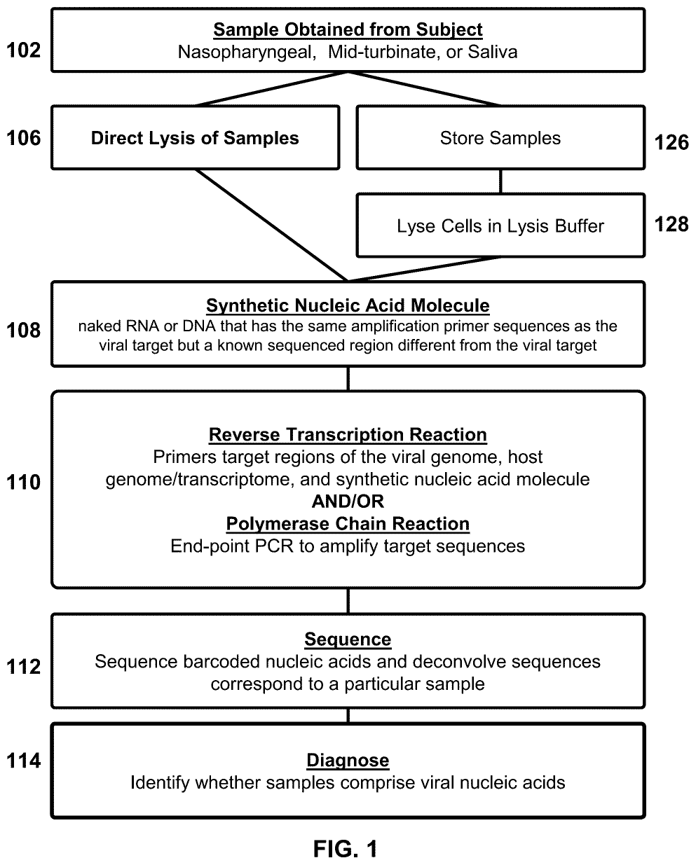

FIG. 1 shows an example workflow for collecting and processing samples. Samples can be obtained from human subjects using swaps (e.g. saliva, nasopharyngeal or mid-turbinate) as exemplified in 102. Swabs can be directly placed into the conditions sufficient to lyse the cells within the sample as exemplified in 106. No RNA isolation is needed thus (1) reducing the time it takes to perform the assay, (2) increasing sensitivity, and (3) reducing the cost of materials for the assay. Samples can optionally be stored in a buffer (e.g. TE buffer) and lysed at a later time within days of obtaining the sample as in steps 126, 128. To facilitate accurate and sensitive identification of a viral nucleic acid, the lysed sample can be spiked with a synthetic nucleic acid that can benchmark subsequent nucleic acid processing and the resulting sequence information (exemplified in 108). The synthetic nucleic acid comprises sequences able to be amplified by virus specific primers, but has a known sequence that can be readily identified in the sequence output as different from the viral target. The samples are partitioned and processed in microtiter plates that enable scales between 1 and 32 384-well plates, enabling testing of at least 384-12,288 samples.

Nucleic acid processing and library prep is then performed by reverse transcription and PCR, as exemplified in 110, within the partition/well. The primers used for detection comprises sequences complementary to (a) the target of interest (a small region from coronavirus or influenza for instance); (b) a human target (controls to make sure there is some human sequence there; e.g., RPP30 (RNaseP), which is used by the CDC in their standard qPCR based tests; and (c) an internal synthetic RNA molecule (as noted above) to ensure that the target assay amplifies, and to quantify the target, allowing for example normalization of reads from the pathogen sequences. Amplification by PCR is performed to endpoint and followed by sequencing. Endpoint PCR and the synthetic template add a level of normalization to avoid expensive normalization steps. The design of primers used for target amplification allows for the addition of indices that allow sample deconvolution and the identification of which sample comprised a positive sequencing read for a viral sequence.

The samples are subsequently sequenced to identify barcoded sequences and the presence of viral target reads. Reads are bioinformatically processed to gauge sequencing data positive or negative for a viral vector and viral load. It is possible to further develop the assay to sequence polymorphic regions and determine pathogen strain. This can be achieved by comparing the ratio of target pathogen amplicons, to human target amplicon, to spike in amplicon.

### Example 2—Detection of SARS-CoV RNA in a Sample

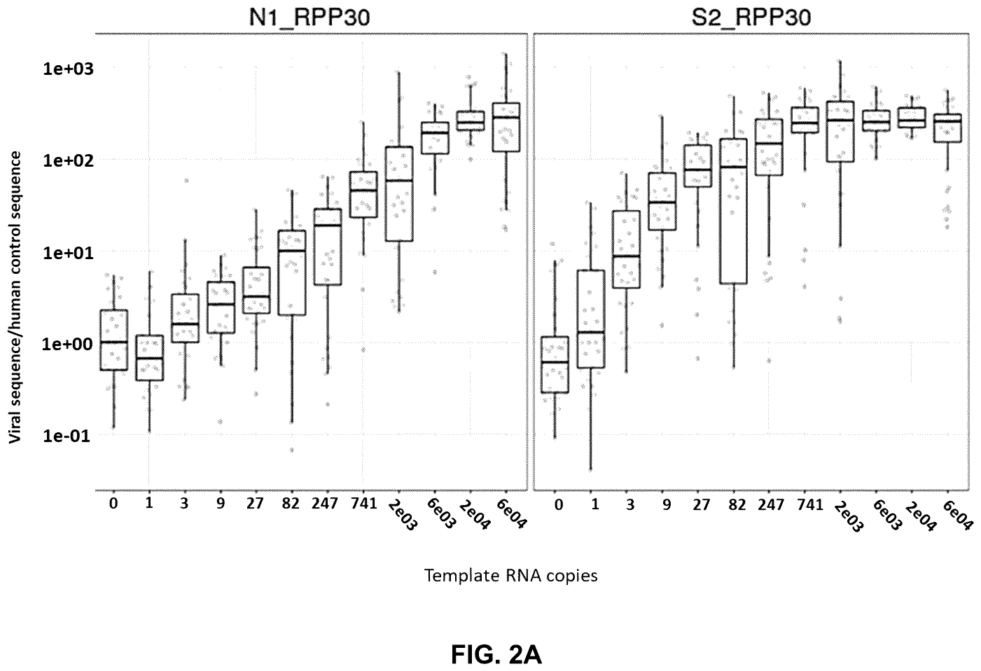

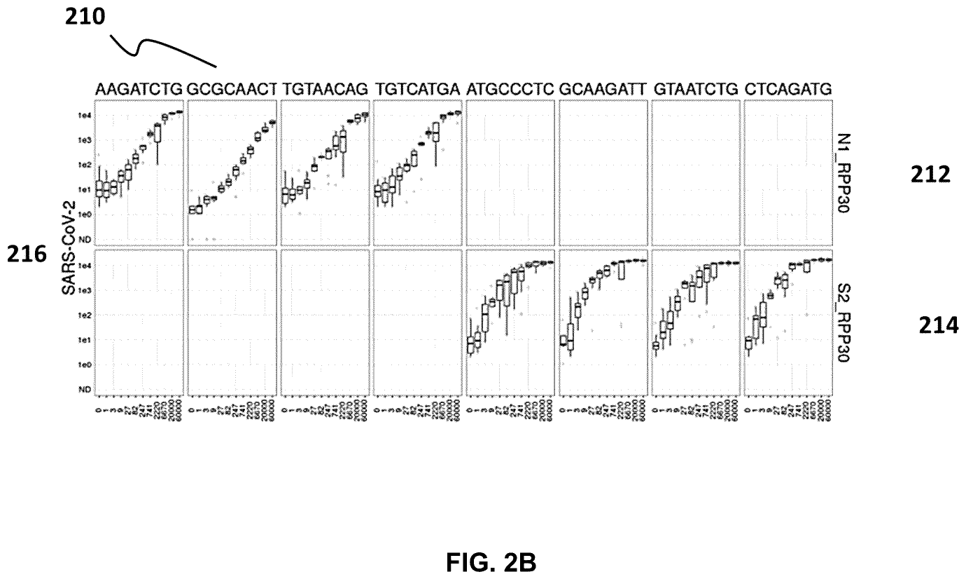

Samples comprising SARS-CoV-2 RNA were analyzed using the methods disclosed herein. Samples comprised a defined copy number of a SARS-CoV-2 RNA molecule. Primers targeting the N1 region (nucleocapsid) and subunit S2 region were used for detecting the SARS-CoV-2 RNA molecule. Resulting sequencing products were compared against human RRP30 RNA. SARS-CoV:RRP30 as a function of copy number was analyzed for reach target region 206, 216 (FIG. 2A-B). Surprisingly, targeting subunit S2 resulted in distinguishable detection within samples comprising 3 copies of the SARS-CoV-2 RNA molecule. Targeting N1 demonstrated distinguishable detection at copy numbers 2-5 times higher than that of S2, but with acceptable sensitivity. FIG. 2B demonstrates the analysis of different primer sequences 210 complementary to the N1 locus and S2 locus. Detection was dependent on primer sequences for both N1 and S2 targets. Across all primers tested, targeting S2 demonstrated distinguishable detection at copy numbers 2-5 times fewer than that of N1, conferring that targeting S2 provides higher resolution analysis for the detection of SARS-CoV RNA in a sample.

### Example 3—Viral Diagnosis by Amplification and Sequencing

Described herein is one example of an amplification and sequencing protocol for use with the methods described herein. Samples in lysis buffer may be heated before addition of QRT-PCR reaction (e.g., 75° C. for 10 minutes).

QRT-PCR Reaction Setup with Individual Reactions run in 20 μL total volume; 7 μL of sample lysate; 10 μL Luna® Universal One-Step Reaction Mix; 1 μL Enzyme Mix; 400 nM viral nucleic acid target primers; housekeeping primers (e.g., RPP3 100 nM no adaptor, 50 nM with adaptor); 100 copies of synthetic nucleic acid (same priming regions as the viral target). By reducing the concentration of RPP3 primers with sequencing adaptors while keeping overall RPP3 primer concentration high, low amounts of RPP3 are still detected while limiting the number of sequencing reads dedicated to host RNA. Additionally, this also attenuated primer dimer formation.

Cycling protocol 55° C. for 30 min (reverse transcription reaction); 95 C° for 1 min, followed by 40 cycles of {95° C. for 10 seconds and 60° for 30 seconds).

Pool 5 μL of each well for sequencing and purify using the [AxyPrep PCR Clean-up Kit](www.fishersci.com/shop/products/axygen-axyprep-mag-pcr-clean-up-kits/14223152). Quantify libraries with the [deNovix dsDNA High Sensitivity Fluorescent Assay Kit](www.denovix.com/denovix-dsdna-assays/). Can add up to 50% PhiX control library DNA. Sequence the libraries using Illumina dual-indexed single-read sequencing on a NextSeq and MiSeq.

Below is a table Table 1 describing primer pairs that can be used in the methods of detecting viral infection described herein. Primer pairs are disclosed as pairs, for example, o325_octN1_F and o326_octN1_R describe a primer pair designed to produce amplification ofviral nucleic acid. Sequences described below may also comprise sequencing adaptor and or index sequences.

TABLE 1

| SEQUENCE ID | | |
|---|---|---|
| NO. | name | seq |
| SEQ ID NO: 100 | o258_o258 SARS-CoV-2 N1 | tggggtcTTACACGGCGATCTTGCC |
| | luc index 1 primer | |
| SEQ ID NO: 101 | o259_o259 SARS-CoV-2 N1 | gggtcAACGTGTCGGCATGGATTCT |
| | oct index 1 primer | |
| SEQ ID NO: 102 | o260_o260 SARS-CoV-2 N1 | AGTTACATTCACGCCAGTTGTGtctggt |
| | luc read 1 | |
| SEQ ID NO: 103 | o261_o261 SARS-CoV-2 N2 | AGTTACATTCACGCCAGTTGTGgcg |
| | luc read 1 | |
| SEQ ID NO: 104 | o262_o262 SARS-CoV-2 N2 | gtttgtaaTTACACGGCGATCTTGCC |
| | luc index 1 primer | |
| SEQ ID NO: 105 | o263_o263 RP luc index 1 | GTCCAAATCTTTACACGGCGATCTTGCC |
| | primer | |
| SEQ ID NO: 106 | o264_o264 RP luc read 1 | AGTTACATTCACGCCAGTTGTGGAGC |
| SEQ ID NO: 107 | o265_o265 T7prom-SARS- | TAATACGACTCACTATAGggtctgataatggacc |
| | CoV-2 N | ccaaaatca |
| SEQ ID NO: 108 | o266_o266_SARS-CoV-2 N R | ttaggcctgagttgagtcagc |
| SEQ ID NO: 109 | o267_index A01 N1_F | CAAGCAGAAGACGGCATACGAGAT GTTCTATC |
| | | GACCCCAAAATCAGCGAAAT |
| SEQ ID NO: 110 | o268_index plate 1 N1_R | AATGATACGGCGACCACCGAGATCTACAC |
| | | AAGATCTG TCTGGTTACTGCCAGTTGAATCTG |
| SEQ ID NO: 111 | o269_index A01 N2_F | CAAGCAGAAGACGGCATACGAGAT GTTCTATC |
| | | TTACAAACATTGGCCGCAAA |
| SEQ ID NO: 112 | o270_index plate 1 N2_R | AATGATACGGCGACCACCGAGATCTACAC |
| | | AAGATCTG GCGCGACATTCCGAAGAA |
| SEQ ID NO: 113 | o271_index A01 RP_F | CAAGCAGAAGACGGCATACGAGAT GTTCTATC |
| | | AGATTTGGACCTGCGAGCG |
| SEQ ID NO: 114 | o272_index plate 1 RP_R | AATGATACGGCGACCACCGAGATCTACAC |
| | | AAGATCTG GAGCGGCTGTCTCCACAAGT |
| SEQ ID NO: 115 | o273_index A01 HKU_N_F | CAAGCAGAAGACGGCATACGAGAT GTTCTATC |
| | | TAATCAGACAAGGAACTGATTA |
| SEQ ID NO: 116 | o274_index plate 1 | AATGATACGGCGACCACCGAGATCTACAC |
| | HKU_N_R | AAGATCTG CGAAGGTGTGACTTCCATG |
| SEQ ID NO: 117 | o275_index A01 octN1_F | AATGATACGGCGACCACCGAGATCTACAC |
| | | AAGATCTGGATCAAAACAACGTCGGCCC |
| SEQ ID NO: 118 | o276_index plate 1 | CAAGCAGAAGACGGCATACGAGAT |
| | octN1_R | GTTCTATCCCATGTTGAGTGAGAGCGGT |
| SEQ ID NO: 119 | o277_index A01 octN2_F | AATGATACGGCGACCACCGAGATCTACAC |
| | | AAGATCTGTGGACCCCAAAATCAGCGAA |
| SEQ ID NO: 120 | o278_index plate 1 | CAAGCAGAAGACGGCATACGAGAT |
| | octN2_R | GTTCTATCACTGCGTTCTCCATTCTGGTT |
| SEQ ID NO: 121 | o279_index A01 octN3_F | AATGATACGGCGACCACCGAGATCTACAC |
| | | AAGATCTGCAGCGTTCTTCGGAATGTCG |
| SEQ ID NO: 122 | o280_index plate 1 | CAAGCAGAAGACGGCATACGAGAT |
| | octN3_R | GTTCTATCGCACCTGTGTAGGTCAACCA |
| SEQ ID NO: 123 | o281_index A01 octN4_F | AATGATACGGCGACCACCGAGATCTACAC |
| | | AAGATCTGGAAATGCACCCCGCATTACG |
| SEQ ID NO: 124 | o282_index plate 1 | CAAGCAGAAGACGGCATACGAGAT |
| | octN4_R | GTTCTATCCCCACTGCGTTCTCCATTCT |
| SEQ ID NO: 125 | o283_index A01 octN5_F | AATGATACGGCGACCACCGAGATCTACAC |
| | | AAGATCTGGTCTTGGTTCACCGCTCTCA |
| SEQ ID NO: 126 | o284_index plate 1 | CAAGCAGAAGACGGCATACGAGAT |
| | octN5_R | GTTCTATCTTGGAACGCCTTGTCCTCG |
| SEQ ID NO: 127 | o285_index A01 octN6_F | AATGATACGGCGACCACCGAGATCTACAC |
| | | AAGATCTGGCAGTCAAGCCTCTTCTCGT |
| SEQ ID NO: 128 | o286_index plate 1 | CAAGCAGAAGACGGCATACGAGAT |
| | octN6_R | GTTCTATCGAAGTTCCCCTACTGCTGCC |
| SEQ ID NO: 129 | o287_index A01 octN7_F | AATGATACGGCGACCACCGAGATCTACAC |
| | | AAGATCTGCGTTTGGTGGACCCTCAGAT |
| SEQ ID NO: 130 | o288_index plate 1 | CAAGCAGAAGACGGCATACGAGAT |
| | octN7_R | GTTCTATCGACGTTGTTTTGATCGCGCC |
| SEQ ID NO: 131 | o289_index A01 octN8_F | AATGATACGGCGACCACCGAGATCTACAC |
| | | AAGATCTGAAGGCCAACAACAACAAGGC |
| SEQ ID NO: 132 | o290_index plate 1 | CAAGCAGAAGACGGCATACGAGAT |
| | octN8_R | GTTCTATCGGCAGTACGTTTTTGCCGAG |
| SEQ ID NO: 133 | o291_index A01 octN9_F | AATGATACGGCGACCACCGAGATCTACAC |
| | | AAGATCTGACCAGAATGGAGAACGCAGT |
| SEQ ID NO: 134 | o292_index plate 1 | CAAGCAGAAGACGGCATACGAGAT |
| | octN9_R | GTTCTATCCGGTGAACCAAGACGCAGTA |
| SEQ ID NO: 135 | o293_index A01 octN10_F | AATGATACGGCGACCACCGAGATCTACAC |
| | | AAGATCTGCCGCATTACGTTTGGTGGAC |
| SEQ ID NO: 136 | o294_index plate 1 | CAAGCAGAAGACGGCATACGAGAT |
| | octN10_R | GTTCTATCGGCCGACGTTGTTTTGATCG |
| SEQ ID NO: 137 | o295_index A01 octN11_F | AATGATACGGCGACCACCGAGATCTACAC |
| | | AAGATCTGGCCTCGGCAAAAACGTACTG |
| SEQ ID NO: 138 | o296_index plate 1 | CAAGCAGAAGACGGCATACGAGAT |
| | octN11_R | GTTCTATCTTGTTCTGGACCACGTCTGC |
| SEQ ID NO: 139 | o297_index A01 octN12_F | AATGATACGGCGACCACCGAGATCTACAC |
| | | AAGATCTGAATTCCCTCGAGGACAAGGC |
| SEQ ID NO: 140 | o298_index plate 1 | CAAGCAGAAGACGGCATACGAGAT |
| | octN12_R | GTTCTATCTCGTCTGGTAGCTCTTCGGT |
| SEQ ID NO: 141 | o299_index A01 octN13_F | AATGATACGGCGACCACCGAGATCTACAC |
| | | AAGATCTGGCTTCAGCGTTCTTCGGAAT |
| SEQ ID NO: 142 | o300_index plate 1 | CAAGCAGAAGACGGCATACGAGAT |
| | octN13_R | GTTCTATCTGGCACCTGTGTAGGTCAAC |
| SEQ ID NO: 143 | o301_SARS-CoV- | AATGATACGGCGACCACCGAGATCTACAC |
| | 2_IBS_RdRP2_F | AAGATCTGAGAATAGAGCTCGCACCGTA |
| SEQ ID NO: 144 | o302_SARS-CoV- | CAAGCAGAAGACGGCATACGAGAT GTTCTATC |
| | 2_IBS_RdRP2_R | CTCCTCTAGTGGCGGCTATT |
| SEQ ID NO: 145 | o303_SARS-CoV-2_IBS_s2_F | AATGATACGGCGACCACCGAGATCTACAC |
| | | AAGATCTG GCTGGTGCTGCAGCTTATTA |
| SEQ ID NO: 146 | o304_SARS-CoV-2_IBS_s2_R | CAAGCAGAAGACGGCATACGAGAT GTTCTATC |
| | | AGGGTCAAGTGCACAGTCTA |
| SEQ ID NO: 147 | o305_SARS-CoV-2_IBS_E2_F | AATGATACGGCGACCACCGAGATCTACAC |
| | | AAGATCTG TTCGGAAGAGACAGGTACGTTA |
| SEQ ID NO: 148 | o306_SARS-CoV-2_IBS_E2_R | CAAGCAGAAGACGGCATACGAGAT GTTCTATC |
| | | AGCAGTACGCACACAATCG |
| SEQ ID NO: 149 | o307_SARS-CoV-2_IBS_N1_F | AATGATACGGCGACCACCGAGATCTACAC |
| | | AAGATCTG CAATGCTGCAATCGTGCTAC |
| SEQ ID NO: 150 | o308_SARS-CoV-2_IBS_N1_R | CAAGCAGAAGACGGCATACGAGAT GTTCTATC |
| | | GTTGCGACTACGTGATGAGG |
| SEQ ID NO: 151 | o309_RNAse P Forward | AGATTTGGACCTGCGAGCG |
| | Primer | |
| SEQ ID NO: 152 | o310_RNAse P Reverse | GAGCGGCTGTCTCCACAAGT |
| | Primer | |
| SEQ ID NO: 153 | o311_SARS-CoV- | AGAATAGAGCTCGCACCGTA |
| | 2_IBS_RdRP2_F | |
| SEQ ID NO: 154 | o312_SARS-CoV- | CTCCTCTAGTGGCGGCTATT |
| | 2_IBS_RdRP2_R | |
| SEQ ID NO: 155 | o313_SARS-CoV-2_IBS_s2_F | GCTGGTGCTGCAGCTTATTA |
| SEQ ID NO: 156 | o314_SARS-CoV-2_IBS_s2_R | AGGGTCAAGTGCACAGTCTA |
| SEQ ID NO: 157 | o315_SARS-CoV-2_IBS_E2_F | TTCGGAAGAGACAGGTACGTTA |
| SEQ ID NO: 158 | o316_SARS-CoV-2_IBS_E2_R | AGCAGTACGCACACAATCG |
| SEQ ID NO: 159 | o317_SARS-CoV-2_IBS_N1_F | CAATGCTGCAATCGTGCTAC |
| SEQ ID NO: 160 | o318_SARS-CoV-2_IBS_N1_R | GTTGCGACTACGTGATGAGG |
| SEQ ID NO: 161 | o319_2019-nCoV_N1 | GACCCCAAAATCAGCGAAAT |
| | Forward Primer | |
| SEQ ID NO: 162 | o320_2019-nCoV_N1 | TCTGGTTACTGCCAGTTGAATCTG |
| | Reverse Primer | |
| SEQ ID NO: 163 | o321_2019-nCoV_N2 | TTACAAACATTGGCCGCAAA |
| | Forward Primer | |
| SEQ ID NO: 164 | o322_2019-nCoV_N2 | GCGCGACATTCCGAAGAA |
| | Reverse Primer | |
| SEQ ID NO: 165 | o323_HKU-NF | TAATCAGACAAGGAACTGATTA |
| SEQ ID NO: 166 | o324_HKU-NR | CGAAGGTGTGACTTCCATG |
| SEQ ID NO: 167 | o325_octN1_F | GATCAAAACAACGTCGGCCC |
| SEQ ID NO: 168 | o326_octN1_R | CCATGTTGAGTGAGAGCGGT |
| SEQ ID NO: 169 | o327_octN2_F | TGGACCCCAAAATCAGCGAA |
| SEQ ID NO: 170 | o328_octN2_R | ACTGCGTTCTCCATTCTGGTT |
| SEQ ID NO: 171 | o329_octN3_F | CAGCGTTCTTCGGAATGTCG |
| SEQ ID NO: 172 | o330_octN3_R | GCACCTGTGTAGGTCAACCA |
| SEQ ID NO: 173 | o331_octN4_F | GAAATGCACCCCGCATTACG |
| SEQ ID NO: 174 | o332_octN4_R | CCCACTGCGTTCTCCATTCT |
| SEQ ID NO: 175 | o333_octN5_F | GTCTTGGTTCACCGCTCTCA |
| SEQ ID NO: 176 | o334_octN5_R | TTGGAACGCCTTGTCCTCG |
| SEQ ID NO: 177 | o335_octN6_F | GCAGTCAAGCCTCTTCTCGT |
| SEQ ID NO: 178 | o336_octN6_R | GAAGTTCCCCTACTGCTGCC |
| SEQ ID NO: 179 | o337_octN7_F | CGTTTGGTGGACCCTCAGAT |
| SEQ ID NO: 180 | o338_octN7_R | GACGTTGTTTTGATCGCGCC |
| SEQ ID NO: 181 | o339_octN8_F | AAGGCCAACAACAACAAGGC |
| SEQ ID NO: 182 | o340_octN8_R | GGCAGTACGTTTTTGCCGAG |
| SEQ ID NO: 183 | o341_octN9_F | ACCAGAATGGAGAACGCAGT |
| SEQ ID NO: 184 | o342_octN9_R | CGGTGAACCAAGACGCAGTA |
| SEQ ID NO: 185 | o343_octN10_F | CCGCATTACGTTTGGTGGAC |
| SEQ ID NO: 186 | o344_octN10_R | GGCCGACGTTGTTTTGATCG |
| SEQ ID NO: 187 | o345_octN11_F | GCCTCGGCAAAAACGTACTG |
| SEQ ID NO: 188 | o346_octN11_R | TTGTTCTGGACCACGTCTGC |
| SEQ ID NO: 189 | o347_octN12_F | AATTCCCTCGAGGACAAGGC |
| SEQ ID NO: 190 | o348_octN12_R | TCGTCTGGTAGCTCTTCGGT |
| SEQ ID NO: 191 | o349_octN13_F | GCTTCAGCGTTCTTCGGAAT |
| SEQ ID NO: 192 | o350_octN13_R | TGGCACCTGTGTAGGTCAAC |
| SEQ ID NO: 193 | o351_skpp15-1-F_RPP30 | GGGTCACGCGTAGGA GTTCTATC |
| | | AGATTTGGACCTGCGAGCG |
| SEQ ID NO: 194 | o352_skpp15-1-R_RPP30 | GTTCCGCAGCCACAC AAGATCTG |
| | | GAGCGGCTGTCTCCACAAGT |
| SEQ ID NO: 195 | o353_skpp15-2-F_RPP30 | CGCGTCGAGTAGGGT GTTCTATC |
| | | AGATTTGGACCTGCGAGCG |
| SEQ ID NO: 196 | o354_skpp15-2-R_RPP30 | GCCGTGTGAAGCTGG AAGATCTG |
| | | GAGCGGCTGTCTCCACAAGT |
| SEQ ID NO: 197 | o355_skpp15-3-F_RPP30 | CGATCGCCCTTGGTG GTTCTATC |
| | | AGATTTGGACCTGCGAGCG |
| SEQ ID NO: 198 | o356_skpp15-3-R_RPP30 | GGTTTAGCCGGCGTG AAGATCTG |
| | | GAGCGGCTGTCTCCACAAGT |
| SEQ ID NO: 199 | o357_skpp15-4-F_RPP30 | GGTCGAGCCGGAACT GTTCTATC |
| | | AGATTTGGACCTGCGAGCG |
| SEQ ID NO: 200 | o358_skpp15-4-R_RPP30 | GGATGCGCACCCAGA AAGATCTG |
| | | GAGCGGCTGTCTCCACAAGT |
| SEQ ID NO: 201 | o359_skpp15-5-F_RPP30 | TCCCGGCGTTGTCCT GTTCTATC |
| | | AGATTTGGACCTGCGAGCG |
| SEQ ID NO: 202 | o360_skpp15-5-R_RPP30 | GCTCCGTCACTGCCCAAGATCTG |
| | | GAGCGGCTGTCTCCACAAGT |
| SEQ ID NO: 203 | o361_skpp15-6-F_RPP30 | CGCAGGGTCCAGAGTGTTCTATC |
| | | AGATTTGGACCTGCGAGCG |
| SEQ ID NO: 204 | o362_skpp15-6-R_RPP30 | GTTCGCGCGAAGGAA AAGATCTG |
| | | GAGCGGCTGTCTCCACAAGT |
| SEQ ID NO: 205 | o363_skpp-1-F s_RPP30 | ATATAGATGCCGTCCTAGCG GTTCTATC |
| | | AGATTTGGACCTGCGAGCG |
| SEQ ID NO: 206 | o364_skpp-1-R s_RPP30 | AAGTATCTTTCCTGTGCCCA AAGATCTG |
| | | GAGCGGCTGTCTCCACAAGT |
| SEQ ID NO: 207 | o365_skpp-2-F s_RPP30 | CCCTTTAATCAGATGCGTCG GTTCTATC |
| | | AGATTTGGACCTGCGAGCG |
| SEQ ID NO: 208 | o366_skpp-2-R s_RPP30 | TGGTAGTAATAAGGGCGACC AAGATCTG |
| | | GAGCGGCTGTCTCCACAAGT |
| SEQ ID NO: 209 | o367_skpp-3-F s_RPP30 | TTGGTCATGTGCTTTTCGTT GTTCTATC |
| | | AGATTTGGACCTGCGAGCG |
| SEQ ID NO: 210 | o368_skpp-3-R s_RPP30 | AGGGGTATCGGATACTCAGA AAGATCTG |
| | | GAGCGGCTGTCTCCACAAGT |
| SEQ ID NO: 211 | o369_skpp-4-F s_RPP30 | GGGTGGGTAAATGGTAATGC GTTCTATC |
| | | AGATTTGGACCTGCGAGCG |
| SEQ ID NO: 212 | o370_skpp-4-R s_RPP30 | ATCGATTCCCCGGATATAGC AAGATCTG |
| | | GAGCGGCTGTCTCCACAAGT |
| SEQ ID NO: 213 | o371_skpp-5-F s_RPP30 | TCCGACGGGGAGTATATACT GTTCTATC |
| | | AGATTTGGACCTGCGAGCG |
| SEQ ID NO: 214 | o372_skpp-5-R s_RPP30 | TACTAACTGCTTCAGGCCAA AAGATCTG |
| | | GAGCGGCTGTCTCCACAAGT |
| SEQ ID NO: 215 | o373_skpp-6-F s_RPP30 | CATGTTTAGGAACGCTACCG GTTCTATC |
| | | AGATTTGGACCTGCGAGCG |
| SEQ ID NO: 216 | o374_skpp-6-R s_RPP30 | AATAATCTCCGTTCCCTCCC AAGATCTG |
| | | GAGCGGCTGTCTCCACAAGT |
| SEQ ID NO: 217 | o375_T7prom o267_o268 | TAATACGACTCACTATAGggaccccaaaatcagc |
| | spike_F | gaaatgcaccccgcattacgAAACCAggaccctc |
| | | agattcaactg |
| SEQ ID NO: 218 | o376_T7prom o267_o268 | cgcagtattattgggtaaacct |
| | spike_R | |
| SEQ ID NO: 219 | o377_T7prom o303_o304 | TAATACGACTCACTATAGggctggtgctgcagct |
| | spike_F | tattatgtgggtATAGAAcaacctaggacttttc |
| | | tattaa |
| SEQ ID NO: 220 | o378_T7prom o303_o304 | aacgtacactttgtttctgagagagg |
| | spike_R | |
| SEQ ID NO: 221 | o379_A1_N1_F | CAAGCAGAAGACGGCATACGAGATGAGTCTTCGA |
| | | CCCCAAAATCAGCGAAAT |
| SEQ ID NO: 222 | o380_A2_N1_F | CAAGCAGAAGACGGCATACGAGATGTTCTATCGA |
| | | CCCCAAAATCAGCGAAAT |
| SEQ ID NO: 223 | o381_A3_N1_F | CAAGCAGAAGACGGCATACGAGATTGGGCCAAGA |
| | | CCCCAAAATCAGCGAAAT |
| SEQ ID NO: 224 | o382_A4_N1_F | CAAGCAGAAGACGGCATACGAGATATTGTTGGGA |
| | | CCCCAAAATCAGCGAAAT |
| SEQ ID NO: 225 | o383_A5_N1_F | CAAGCAGAAGACGGCATACGAGATTCCCGTTGGA |
| | | CCCCAAAATCAGCGAAAT |
| SEQ ID NO: 226 | o384_A6_N1_F | CAAGCAGAAGACGGCATACGAGATACACACTTGA |
| | | CCCCAAAATCAGCGAAAT |
| SEQ ID NO: 227 | o385_A7_N1_F | CAAGCAGAAGACGGCATACGAGATCCATTCCAGA |
| | | CCCCAAAATCAGCGAAAT |
| SEQ ID NO: 228 | o386_A8_N1_F | CAAGCAGAAGACGGCATACGAGATCTAACGGGGA |
| | | CCCCAAAATCAGCGAAAT |
| SEQ ID NO: 229 | o387_A9_N1_F | CAAGCAGAAGACGGCATACGAGATCCATAGGAGA |
| | | CCCCAAAATCAGCGAAAT |
| SEQ ID NO: 230 | o388_A10_N1_F | CAAGCAGAAGACGGCATACGAGATCAGTGTAGGA |
| | | CCCCAAAATCAGCGAAAT |
| SEQ ID NO: 231 | o389_A11_N1_F | CAAGCAGAAGACGGCATACGAGATGATTCTCAGA |
| | | CCCCAAAATCAGCGAAAT |
| SEQ ID NO: 232 | o390_A1_N1_F | CAAGCAGAAGACGGCATACGAGATCCTTCTTAGA |
| | | CCCCAAAATCAGCGAAAT |
| SEQ ID NO: 233 | o391_B1_N1_F | CAAGCAGAAGACGGCATACGAGATTCTAAGACGA |
| | | CCCCAAAATCAGCGAAAT |
| SEQ ID NO: 234 | o392_B2_N1_F | CAAGCAGAAGACGGCATACGAGATGCGGCATAGA |
| | | CCCCAAAATCAGCGAAAT |
| SEQ ID NO: 235 | o393_B3_N1_F | CAAGCAGAAGACGGCATACGAGATATTGACGAGA |
| | | CCCCAAAATCAGCGAAAT |
| SEQ ID NO: 236 | o394_B4_N1_F | CAAGCAGAAGACGGCATACGAGATGCTCCTGAGA |
| | | CAAGCAGAAGACGGCATACGAGATGCAATCCTGA |
| SEQ ID NO: 237 | o395_B5_N1_F | CCCCAAAATCAGCGAAATS |
| | | CCCCAAAATCAGCGAAAT |
| SEQ ID NO: 238 | o396_B6_N1_F | CAAGCAGAAGACGGCATACGAGATATGTCGTTGA |
| | | CCCCAAAATCAGCGAAAT |
| SEQ ID NO: 239 | o397_B7_N1_F | CAAGCAGAAGACGGCATACGAGATTTCTCGGCGA |
| | | CCCCAAAATCAGCGAAAT |
| SEQ ID NO: 240 | o398_B8_N1_F | CAAGCAGAAGACGGCATACGAGATCAGGGCTAGA |
| | | CCCCAAAATCAGCGAAAT |
| SEQ ID NO: 241 | o399_B9_N1_F | CAAGCAGAAGACGGCATACGAGATAGCCAAGCGA |
| | | CCCCAAAATCAGCGAAAT |
| SEQ ID NO: 242 | o400_B10_N1_F | CAAGCAGAAGACGGCATACGAGATAAGCCTGAGA |
| | | CCCCAAAATCAGCGAAAT |
| SEQ ID NO: 243 | o401_B11_N1_F | CAAGCAGAAGACGGCATACGAGATCTACAGAGGA |
| | | CCCCAAAATCAGCGAAAT |
| SEQ ID NO: 244 | o402_B12_N1_F | CAAGCAGAAGACGGCATACGAGATCGTAGTCGGA |
| | | CCCCAAAATCAGCGAAAT |
| SEQ ID NO: 245 | o403_C1_N1_F | CAAGCAGAAGACGGCATACGAGATTTCTGCTCGA |
| | | CCCCAAAATCAGCGAAAT |
| SEQ ID NO: 246 | o404_C2_N1_F | CAAGCAGAAGACGGCATACGAGATGTGCACACGA |
| | | CCCCAAAATCAGCGAAAT |
| SEQ ID NO: 247 | o405_C3_N1_F | CAAGCAGAAGACGGCATACGAGATAAAGCTCAGA |
| | | CCCCAAAATCAGCGAAAT |
| SEQ ID NO: 248 | o406_C4_N1_F | CAAGCAGAAGACGGCATACGAGATGACCTCAGGA |
| | | CCCCAAAATCAGCGAAAT |
| SEQ ID NO: 249 | o407_C5_N1_F | CAAGCAGAAGACGGCATACGAGATCTTTCCAAGA |
| | | CCCCAAAATCAGCGAAAT |
| SEQ ID NO: 250 | o408_C6_N1_F | CAAGCAGAAGACGGCATACGAGATTCTTGGCTGA |
| | | CCCCAAAATCAGCGAAAT |
| SEQ ID NO: 251 | o409_C7_N1_F | CAAGCAGAAGACGGCATACGAGATCGCGTCTAGA |
| | | CCCCAAAATCAGCGAAAT |
| SEQ ID NO: 252 | o410_C8_N1_F | CAAGCAGAAGACGGCATACGAGATTCGCGCTAGA |
| | | CCCCAAAATCAGCGAAAT |
| SEQ ID NO: 253 | o411_C9_N1_F | CAAGCAGAAGACGGCATACGAGATATCCATTCGA |
| | | CCCCAAAATCAGCGAAAT |
| SEQ ID NO: 254 | o412_C10_N1_F | CAAGCAGAAGACGGCATACGAGATGCCCAGTAGA |
| | | CCCCAAAATCAGCGAAAT |
| SEQ ID NO: 255 | o413_C11_N1_F | CAAGCAGAAGACGGCATACGAGATTACCGACGGA |
| | | CCCCAAAATCAGCGAAAT |
| SEQ ID NO: 256 | o414_C12_N1_F | CAAGCAGAAGACGGCATACGAGATTCCATACGGA |
| | | CCCCAAAATCAGCGAAAT |
| SEQ ID NO: 257 | o415_D1_N1_F | CAAGCAGAAGACGGCATACGAGATAACATGTCGA |
| | | CCCCAAAATCAGCGAAAT |
| SEQ ID NO: 258 | o416_D2_N1_F | CAAGCAGAAGACGGCATACGAGATCGACTATAGA |
| | | CCCCAAAATCAGCGAAAT |
| SEQ ID NO: 259 | o417_D3_N1_F | CAAGCAGAAGACGGCATACGAGATACCCAAAGGA |
| | | CCCCAAAATCAGCGAAAT |
| SEQ ID NO: 260 | o418_D4_N1_F | CAAGCAGAAGACGGCATACGAGATATCGATCGGA |
| | | CCCCAAAATCAGCGAAAT |
| SEQ ID NO: 261 | o419_D5_N1_F | CAAGCAGAAGACGGCATACGAGATGTTGGATGGA |
| | | CCCCAAAATCAGCGAAAT |
| SEQ ID NO: 262 | o420_D6_N1_F | CAAGCAGAAGACGGCATACGAGATCTATGTGAGA |
| | | CCCCAAAATCAGCGAAAT |
| SEQ ID NO: 263 | o421_D7_N1_F | CAAGCAGAAGACGGCATACGAGATTATTTCGCGA |
| | | CCCCAAAATCAGCGAAAT |
| SEQ ID NO: 264 | o422_D8_N1_F | CAAGCAGAAGACGGCATACGAGATCCATGTATGA |
| | | CCCCAAAATCAGCGAAAT |
| SEQ ID NO: 265 | o423_D9_N1_F | CAAGCAGAAGACGGCATACGAGATGCCACGTTGA |
| | | CCCCAAAATCAGCGAAAT |
| SEQ ID NO: 266 | o424_D10_N1_F | CAAGCAGAAGACGGCATACGAGATGTCGTGTAGA |
| | | CCCCAAAATCAGCGAAAT |
| SEQ ID NO: 267 | o425_D11_N1_F | CAAGCAGAAGACGGCATACGAGATTAAAGTCGGA |
| | | CCCCAAAATCAGCGAAAT |
| SEQ ID NO: 268 | o426_D12_N1_F | CAAGCAGAAGACGGCATACGAGATCTTCGGACGA |
| | | CCCCAAAATCAGCGAAAT |
| SEQ ID NO: 269 | o427_E1_N1_F | CAAGCAGAAGACGGCATACGAGATGCACTCTCGA |
| | | CCCCAAAATCAGCGAAAT |
| SEQ ID NO: 270 | o428_E2_N1_F | CAAGCAGAAGACGGCATACGAGATTCAGATACGA |
| | | CCCCAAAATCAGCGAAAT |
| SEQ ID NO: 271 | o429_E3_N1_F | CAAGCAGAAGACGGCATACGAGATCAGTCCCTGA |
| | | CCCCAAAATCAGCGAAAT |
| SEQ ID NO: 272 | o430_E4_N1_F | CAAGCAGAAGACGGCATACGAGATGCCCTAACGA |
| | | CCCCAAAATCAGCGAAAT |
| SEQ ID NO: 273 | o431_E5_N1_F | CAAGCAGAAGACGGCATACGAGATCTGCATCAGA |
| | | CCCCAAAATCAGCGAAAT |
| SEQ ID NO: 274 | o432_E6_N1_F | CAAGCAGAAGACGGCATACGAGATCGGTATCGGA |
| | | CCCCAAAATCAGCGAAAT |
| SEQ ID NO: 275 | o433_E7_N1_F | CAAGCAGAAGACGGCATACGAGATAAGTATGGGA |
| | | CCCCAAAATCAGCGAAAT |
| SEQ ID NO: 276 | o434_E8_N1_F | CAAGCAGAAGACGGCATACGAGATATTCGCGCGA |
| | | CCCCAAAATCAGCGAAAT |
| SEQ ID NO: 277 | o435_E9_N1_F | CAAGCAGAAGACGGCATACGAGATATCAAGGTGA |
| | | CCCCAAAATCAGCGAAAT |
| SEQ ID NO: 278 | o436_E10_N1_F | CAAGCAGAAGACGGCATACGAGATTTGTGCATGA |
| | | CCCCAAAATCAGCGAAAT |
| SEQ ID NO: 279 | o437_E11_N1_F | CAAGCAGAAGACGGCATACGAGATCTGTGCTGGA |
| | | CCCCAAAATCAGCGAAAT |
| SEQ ID NO: 280 | o438_E12_N1_F | CAAGCAGAAGACGGCATACGAGATGTCCGTAGGA |
| | | CCCCAAAATCAGCGAAAT |
| SEQ ID NO: 281 | o439_F1_N1_F | CAAGCAGAAGACGGCATACGAGATGTTCAAGAGA |
| | | CCCCAAAATCAGCGAAAT |
| SEQ ID NO: 282 | o440_F2_N1_F | CAAGCAGAAGACGGCATACGAGATCACCGTTCGA |
| | | CCCCAAAATCAGCGAAAT |
| SEQ ID NO: 283 | o441_F3_N1_F | CAAGCAGAAGACGGCATACGAGATCGAGTTGAGA |
| | | CCCCAAAATCAGCGAAAT |
| SEQ ID NO: 284 | o442_F4_N1_F | CAAGCAGAAGACGGCATACGAGATGAGCACGAGA |
| | | CCCCAAAATCAGCGAAAT |
| SEQ ID NO: 285 | o443_F5_N1_F | CAAGCAGAAGACGGCATACGAGATAGTTCGTGGA |
| | | CCCCAAAATCAGCGAAAT |
| SEQ ID NO: 286 | o444_F6_N1_F | CAAGCAGAAGACGGCATACGAGATCATCAACTGA |
| | | CCCCAAAATCAGCGAAAT |
| SEQ ID NO: 287 | o445_F7_N1_F | CAAGCAGAAGACGGCATACGAGATCGAGATCTGA |
| | | CCCCAAAATCAGCGAAAT |
| SEQ ID NO: 288 | o446_F8_N1_F | CAAGCAGAAGACGGCATACGAGATTGGCCAGAGA |
| | | CCCCAAAATCAGCGAAAT |
| SEQ ID NO: 289 | o447_F9_N1_F | CAAGCAGAAGACGGCATACGAGATTTCACCATGA |
| | | CCCCAAAATCAGCGAAAT |
| SEQ ID NO: 290 | o448_F10_N1_F | CAAGCAGAAGACGGCATACGAGATGAATGCATGA |
| | | CCCCAAAATCAGCGAAAT |
| SEQ ID NO: 291 | o449_F11_N1_F | CAAGCAGAAGACGGCATACGAGATTGGACCCTGA |
| | | CCCCAAAATCAGCGAAAT |
| SEQ ID NO: 292 | o450_F12_N1_F | CAAGCAGAAGACGGCATACGAGATGATAGCACGA |
| | | CCCCAAAATCAGCGAAAT |
| SEQ ID NO: 293 | o451_G1_N1_F | CAAGCAGAAGACGGCATACGAGATACGACGACGA |
| | | CCCCAAAATCAGCGAAAT |
| SEQ ID NO: 294 | o452_G2_N1_F | CAAGCAGAAGACGGCATACGAGATCTCAGTATGA |
| | | CCCCAAAATCAGCGAAAT |
| SEQ ID NO: 295 | o453_G3_N1_F | CAAGCAGAAGACGGCATACGAGATCTTAGCTAGA |
| | | CCCCAAAATCAGCGAAAT |
| SEQ ID NO: 296 | o454_G4_N1_F | CAAGCAGAAGACGGCATACGAGATCTGTTTACGA |
| | | CCCCAAAATCAGCGAAAT |
| SEQ ID NO: 297 | o455_G5_N1_F | CAAGCAGAAGACGGCATACGAGATTGTCCCACGA |
| | | CCCCAAAATCAGCGAAAT |
| SEQ ID NO: 298 | o456_G6_N1_F | CAAGCAGAAGACGGCATACGAGATTCCTGAGGGA |
| | | CCCCAAAATCAGCGAAAT |
| SEQ ID NO: 299 | o457_G7_N1_F | CAAGCAGAAGACGGCATACGAGATTAGTCCAGA |
| | | CCCCAAAATCAGCGAAAT |
| SEQ ID NO: 300 | o458_G8_N1_F | CAAGCAGAAGACGGCATACGAGATCATGACTCGA |
| | | CCCCAAAATCAGCGAAAT |
| SEQ ID NO: 301 | o459_G9_N1_F | CAAGCAGAAGACGGCATACGAGATGTAAGCGCGA |
| | | CCCCAAAATCAGCGAAAT |
| SEQ ID NO: 302 | o460_G10_N1_F | CAAGCAGAAGACGGCATACGAGATAACCCAGTGA |
| | | CCCCAAAATCAGCGAAAT |
| SEQ ID NO: 303 | o461_G11_N1_F | CAAGCAGAAGACGGCATACGAGATTTTGAGGGGA |
| | | CCCCAAAATCAGCGAAAT |
| SEQ ID NO: 304 | o462_G12_N1_F | CAAGCAGAAGACGGCATACGAGATAGCCGACAGA |
| | | CCCCAAAATCAGCGAAAT |
| SEQ ID NO: 305 | o463_H1_N1_F | CAAGCAGAAGACGGCATACGAGATAAACCCGCGA |
| | | CCCCAAAATCAGCGAAAT |
| SEQ ID NO: 306 | o464_H2_N1_F | CAAGCAGAAGACGGCATACGAGATGTAGGGCTGA |
| | | CCCCAAAATCAGCGAAAT |
| SEQ ID NO: 307 | o465_H3_N1_F | CAAGCAGAAGACGGCATACGAGATAGACGATTGA |
| | | CCCCAAAATCAGCGAAAT |
| SEQ ID NO: 308 | o466_H4_N1_F | CAAGCAGAAGACGGCATACGAGATAGGATGATGA |
| | | CCCCAAAATCAGCGAAAT |
| SEQ ID NO: 309 | o467_H5_N1_F | CAAGCAGAAGACGGCATACGAGATATAATGGCGA |
| | | CCCCAAAATCAGCGAAAT |
| SEQ ID NO: 310 | o468_H6_N1_F | CAAGCAGAAGACGGCATACGAGATCTTGGCGTGA |
| | | CCCCAAAATCAGCGAAAT |
| SEQ ID NO: 311 | o469_H7_N1_F | CAAGCAGAAGACGGCATACGAGATAGCTGTGCGA |
| | | CCCCAAAATCAGCGAAAT |
| SEQ ID NO: 312 | o470_H8_N1_F | CAAGCAGAAGACGGCATACGAGATGAGTCCAAGA |
| | | CCCCAAAATCAGCGAAAT |
| SEQ ID NO: 313 | o471_H9_N1_F | CAAGCAGAAGACGGCATACGAGATGAATACCAGA |
| | | CCCCAAAATCAGCGAAAT |
| SEQ ID NO: 314 | o472_H10_N1_F | CAAGCAGAAGACGGCATACGAGATAGGAGCTTGA |
| | | CCCCAAAATCAGCGAAAT |
| SEQ ID NO: 315 | o473_H11_N1_F | CAAGCAGAAGACGGCATACGAGATGTGACTTAGA |
| | | CCCCAAAATCAGCGAAAT |
| SEQ ID NO: 316 | o474_H12_N1_F | CAAGCAGAAGACGGCATACGAGATTTTGGAACGA |
| | | CCCCAAAATCAGCGAAAT |
| SEQ ID NO: 317 | o475_A1_S2_R | CAAGCAGAAGACGGCATACGAGATGAGTCTTCAG |
| | | GGTCAAGTGCACAGTCTA |
| SEQ ID NO: 318 | o476_A2_S2_R | CAAGCAGAAGACGGCATACGAGATGTTCTATCAG |
| | | GGTCAAGTGCACAGTCTA |
| SEQ ID NO: 319 | o477_A3_S2_R | CAAGCAGAAGACGGCATACGAGATTGGGCCAAAG |
| | | GGTCAAGTGCACAGTCTA |
| SEQ ID NO: 320 | o478_A4_S2_R | CAAGCAGAAGACGGCATACGAGATATTGTTGGAG |
| | | GGTCAAGTGCACAGTCTA |
| SEQ ID NO: 321 | o479_A5_S2_R | CAAGCAGAAGACGGCATACGAGATTCCCGTTGAG |
| | | GGTCAAGTGCACAGTCTA |
| SEQ ID NO: 322 | o480_A6_S2_R | CAAGCAGAAGACGGCATACGAGATACACACTTAG |
| | | GGTCAAGTGCACAGTCTA |
| SEQ ID NO: 323 | o481_A7_S2_R | CAAGCAGAAGACGGCATACGAGATCCATTCCAAG |
| | | GGTCAAGTGCACAGTCTA |
| SEQ ID NO: 324 | o482_A8_S2_R | CAAGCAGAAGACGGCATACGAGATCTAACGGGAG |
| | | GGTCAAGTGCACAGTCTA |
| SEQ ID NO: 325 | o483_A9_S2_R | CAAGCAGAAGACGGCATACGAGATCCATAGGAAG |
| | | GGTCAAGTGCACAGTCTA |
| SEQ ID NO: 326 | o484_A10_S2_R | CAAGCAGAAGACGGCATACGAGATCAGTGTAGAG |
| | | GGTCAAGTGCACAGTCTA |
| SEQ ID NO: 327 | o485_A11_S2_R | CAAGCAGAAGACGGCATACGAGATGATTCTCAAG |
| | | GGTCAAGTGCACAGTCTA |
| SEQ ID NO: 328 | o486_A12_S2_R | CAAGCAGAAGACGGCATACGAGATCCTTCTTAAG |
| | | GGTCAAGTGCACAGTCTA |
| SEQ ID NO: 329 | o487_B1_S2_R | CAAGCAGAAGACGGCATACGAGATTCTAAGACAG |
| | | GGTCAAGTGCACAGTCTA |
| SEQ ID NO: 330 | o488_B2_S2_R | CAAGCAGAAGACGGCATACGAGATGCGGCATAAG |
| | | GGTCAAGTGCACAGTCTA |
| SEQ ID NO: 331 | o489_B3_S2_R | CAAGCAGAAGACGGCATACGAGATATTGACGAAG |
| | | GGTCAAGTGCACAGTCTA |
| SEQ ID NO: 332 | o490_B4_S2_R | CAAGCAGAAGACGGCATACGAGATGCTCCTGAAG |
| | | GGTCAAGTGCACAGTCTA |
| SEQ ID NO: 333 | o491_B5_S2_R | CAAGCAGAAGACGGCATACGAGATGCAATCCTAG |
| | | GGTCAAGTGCACAGTCTA |
| SEQ ID NO: 334 | o492_B6_S2_R | CAAGCAGAAGACGGCATACGAGATATGTCGTTAG |
| | | GGTCAAGTGCACAGTCTA |
| SEQ ID NO: 335 | o493_B7_S2_R | CAAGCAGAAGACGGCATACGAGATTTCTCGGCAG |
| | | GGTCAAGTGCACAGTCTA |
| SEQ ID NO: 336 | o494_B8_S2_R | CAAGCAGAAGACGGCATACGAGATCAGGGCTAAG |
| | | GGTCAAGTGCACAGTCTA |
| SEQ ID NO: 337 | o495_B9_S2_R | CAAGCAGAAGACGGCATACGAGATAGCCAAGCAG |
| | | GGTCAAGTGCACAGTCTA |
| SEQ ID NO: 338 | o496_B10_S2_R | CAAGCAGAAGACGGCATACGAGATAAGCCTGAAG |
| | | GGTCAAGTGCACAGTCTA |
| SEQ ID NO: 339 | o497_B11_S2_R | CAAGCAGAAGACGGCATACGAGATCTACAGAGAG |
| | | GGTCAAGTGCACAGTCTA |
| SEQ ID NO: 340 | o498_B12_S2_R | CAAGCAGAAGACGGCATACGAGATCGTAGTCGAG |
| | | GGTCAAGTGCACAGTCTA |
| SEQ ID NO: 341 | o499_C1_S2_R | CAAGCAGAAGACGGCATACGAGATTTCTGCTCAG |
| | | GGTCAAGTGCACAGTCTA |
| SEQ ID NO: 342 | o500_C2_S2_R | CAAGCAGAAGACGGCATACGAGATGTGCACACAG |
| | | GGTCAAGTGCACAGTCTA |
| SEQ ID NO: 343 | o501_C3_S2_R | CAAGCAGAAGACGGCATACGAGATAAAGCTCAAG |
| | | GGTCAAGTGCACAGTCTA |
| SEQ ID NO: 344 | o502_C4_S2_R | CAAGCAGAAGACGGCATACGAGATGACCTCAGAG |
| | | GGTCAAGTGCACAGTCTA |
| SEQ ID NO: 345 | o503_C5_S2_R | CAAGCAGAAGACGGCATACGAGATCTTTCCAAAG |
| | | GGTCAAGTGCACAGTCTA |
| SEQ ID NO: 346 | o504_C6_S2_R | CAAGCAGAAGACGGCATACGAGATTCTTGGCTAG |
| | | GGTCAAGTGCACAGTCTA |
| SEQ ID NO: 347 | o505_C7_S2_R | CAAGCAGAAGACGGCATACGAGATCGCGTCTAAG |
| | | GGTCAAGTGCACAGTCTA |
| SEQ ID NO: 348 | o506_C8_S2_R | CAAGCAGAAGACGGCATACGAGATTCGCGCTAAG |
| | | GGTCAAGTGCACAGTCTA |
| SEQ ID NO: 349 | o507_C9_S2_R | CAAGCAGAAGACGGCATACGAGATATCCATTCAG |
| | | GGTCAAGTGCACAGTCTA |
| SEQ ID NO: 350 | o508_C10_S2_R | CAAGCAGAAGACGGCATACGAGATGCCCAGTAAG |
| | | GGTCAAGTGCACAGTCTA |
| SEQ ID NO: 351 | o509_C11_S2_R | CAAGCAGAAGACGGCATACGAGATTACCGACGAG |
| | | GGTCAAGTGCACAGTCTA |
| SEQ ID NO: 352 | o510_C12_S2_R | CAAGCAGAAGACGGCATACGAGATTCCATACGAG |
| | | GGTCAAGTGCACAGTCTA |
| SEQ ID NO: 353 | o511_D1_S2_R | CAAGCAGAAGACGGCATACGAGATAACATGTCAG |
| | | GGTCAAGTGCACAGTCTA |
| SEQ ID NO: 354 | o512_D2_S2_R | CAAGCAGAAGACGGCATACGAGATCGACTATAAG |
| | | GGTCAAGTGCACAGTCTA |
| SEQ ID NO: 355 | o513_D3_S2_R | CAAGCAGAAGACGGCATACGAGATACCCAAAGAG |
| | | GGTCAAGTGCACAGTCTA |
| SEQ ID NO: 356 | o514_D4_S2_R | CAAGCAGAAGACGGCATACGAGATATCGATCGAG |
| | | GGTCAAGTGCACAGTCTA |
| SEQ ID NO: 357 | o515_D5_S2_R | CAAGCAGAAGACGGCATACGAGATGTTGGATGAG |
| | | GGTCAAGTGCACAGTCTA |
| SEQ ID NO: 358 | o516_D6_S2_R | CAAGCAGAAGACGGCATACGAGATCTATGTGAAG |
| | | GGTCAAGTGCACAGTCTA |
| SEQ ID NO: 359 | o517_D7_S2_R | CAAGCAGAAGACGGCATACGAGATTATTTCGCAG |
| | | GGTCAAGTGCACAGTCTA |
| SEQ ID NO: 360 | o518_D8_S2_R | CAAGCAGAAGACGGCATACGAGATCCATGTATAG |
| | | GGTCAAGTGCACAGTCTA |
| SEQ ID NO: 361 | o519_D9_S2_R | CAAGCAGAAGACGGCATACGAGATGCCACGTTAG |
| | | GGTCAAGTGCACAGTCTA |
| SEQ ID NO: 362 | o520_D10_S2_R | CAAGCAGAAGACGGCATACGAGATGTCGTGTAAG |
| | | GGTCAAGTGCACAGTCTA |
| SEQ ID NO: 363 | o521_D11_S2_R | CAAGCAGAAGACGGCATACGAGATTAAAGTCGAG |
| | | GGTCAAGTGCACAGTCTA |
| SEQ ID NO: 364 | o522_D12_S2_R | CAAGCAGAAGACGGCATACGAGATCTTCGGACAG |
| | | GGTCAAGTGCACAGTCTA |
| SEQ ID NO: 365 | o523_E1_s2_R | CAAGCAGAAGACGGCATACGAGATGCACTCTCAG |
| | | GGTCAAGTGCACAGTCTA |
| SEQ ID NO: 366 | o524_E2_S2_R | CAAGCAGAAGACGGCATACGAGATTCAGATACAG |
| | | GGTCAAGTGCACAGTCTA |
| SEQ ID NO: 367 | o525_E3_S2_R | CAAGCAGAAGACGGCATACGAGATCAGTCCCTAG |
| | | GGTCAAGTGCACAGTCTA |
| SEQ ID NO: 368 | o526_E4_S2_R | CAAGCAGAAGACGGCATACGAGATGCCCTAACAG |
| | | GGTCAAGTGCACAGTCTA |
| SEQ ID NO: 369 | o527_E5_S2_R | CAAGCAGAAGACGGCATACGAGATCTGCATCAAG |
| | | GGTCAAGTGCACAGTCTA |
| SEQ ID NO: 370 | o528_E6_S2_R | CAAGCAGAAGACGGCATACGAGATCGGTATCGAG |
| | | GGTCAAGTGCACAGTCTA |
| SEQ ID NO: 371 | o529_E7_s2_R | CAAGCAGAAGACGGCATACGAGATAAGTATGGAG |
| | | GGTCAAGTGCACAGTCTA |
| SEQ ID NO: 372 | o530_E8_S2_R | CAAGCAGAAGACGGCATACGAGATATTCGCGCAG |
| | | GGTCAAGTGCACAGTCTA |
| SEQ ID NO: 373 | o531_E9_S2_R | CAAGCAGAAGACGGCATACGAGATATCAAGGTAG |
| | | GGTCAAGTGCACAGTCTA |
| SEQ ID NO: 374 | o532_E10_S2_R | CAAGCAGAAGACGGCATACGAGATTTGTGCATAG |
| | | GGTCAAGTGCACAGTCTA |
| SEQ ID NO: 375 | o533_E11_s2_R | CAAGCAGAAGACGGCATACGAGATCTGTGCTGAG |
| | | GGTCAAGTGCACAGTCTA |
| SEQ ID NO: 376 | o534_E12_S2_R | CAAGCAGAAGACGGCATACGAGATGTCCGTAGAG |
| | | GGTCAAGTGCACAGTCTA |
| SEQ ID NO: 377 | o535_F1_s2_R | CAAGCAGAAGACGGCATACGAGATGTTCAAGAAG |
| | | GGTCAAGTGCACAGTCTA |
| SEQ ID NO: 378 | o536_F2_S2_R | CAAGCAGAAGACGGCATACGAGATCACCGTTCAG |
| | | GGTCAAGTGCACAGTCTA |
| SEQ ID NO: 379 | o537_F3_S2_R | CAAGCAGAAGACGGCATACGAGATCGAGTTGAAG |
| | | GGTCAAGTGCACAGTCTA |
| SEQ ID NO: 380 | o538_F4_S2_R | CAAGCAGAAGACGGCATACGAGATGAGCACGAAG |
| | | GGTCAAGTGCACAGTCTA |
| SEQ ID NO: 381 | o539_F5_s2_R | CAAGCAGAAGACGGCATACGAGATAGTTCGTGAG |
| | | GGTCAAGTGCACAGTCTA |
| SEQ ID NO: 382 | o540_F6_S2_R | CAAGCAGAAGACGGCATACGAGATCATCAACTAG |
| | | GGTCAAGTGCACAGTCTA |
| SEQ ID NO: 383 | o541_F7_S2_R | CAAGCAGAAGACGGCATACGAGATCGAGATCTAG |
| | | GGTCAAGTGCACAGTCTA |
| SEQ ID NO: 384 | o542_F8_S2_R | CAAGCAGAAGACGGCATACGAGATTGGCCAGAAG |
| | | GGTCAAGTGCACAGTCTA |
| SEQ ID NO: 385 | o543_F9_S2_R | CAAGCAGAAGACGGCATACGAGATTTCACCATAG |
| | | GGTCAAGTGCACAGTCTA |
| SEQ ID NO: 386 | o544_F10_S2_R | CAAGCAGAAGACGGCATACGAGATGAATGCATAG |
| | | GGTCAAGTGCACAGTCTA |
| SEQ ID NO: 387 | o545_F11_S2_R | CAAGCAGAAGACGGCATACGAGATTGGACCCTAG |
| | | GGTCAAGTGCACAGTCTA |
| SEQ ID NO: 388 | o546_F12_S2_R | CAAGCAGAAGACGGCATACGAGATGATAGCACAG |
| | | GGTCAAGTGCACAGTCTA |
| SEQ ID NO: 389 | o547_G1_S2_R | CAAGCAGAAGACGGCATACGAGATACGACGACAG |
| | | GGTCAAGTGCACAGTCTA |
| SEQ ID NO: 390 | o548_G2_S2_R | CAAGCAGAAGACGGCATACGAGATCTCAGTATAG |
| | | GGTCAAGTGCACAGTCTA |
| SEQ ID NO: 391 | o549_G3_S2_R | CAAGCAGAAGACGGCATACGAGATCTTAGCTAAG |
| | | GGTCAAGTGCACAGTCTA |
| SEQ ID NO: 392 | o550_G4_S2_R | CAAGCAGAAGACGGCATACGAGATCTGTTTACAG |
| | | GGTCAAGTGCACAGTCTA |
| SEQ ID NO: 393 | o551_G5_S2_R | CAAGCAGAAGACGGCATACGAGATTGTCCCACAG |
| | | GGTCAAGTGCACAGTCTA |
| SEQ ID NO: 394 | o552_G6_S2_R | CAAGCAGAAGACGGCATACGAGATTCCTGAGGAG |
| | | GGTCAAGTGCACAGTCTA |
| SEQ ID NO: 395 | o553_G7_S2_R | CAAGCAGAAGACGGCATACGAGATTAGTTCCAAG |
| | | GGTCAAGTGCACAGTCTA |
| SEQ ID NO: 396 | o554_G8_S2_R | CAAGCAGAAGACGGCATACGAGATCATGACTCAG |
| | | GGTCAAGTGCACAGTCTA |
| SEQ ID NO: 397 | o555_G9_s2_R | CAAGCAGAAGACGGCATACGAGATGTAAGCGCAG |
| | | GGTCAAGTGCACAGTCTA |
| SEQ ID NO: 398 | o556_G10_S2_R | CAAGCAGAAGACGGCATACGAGATAACCCAGTAG |
| | | GGTCAAGTGCACAGTCTA |
| SEQ ID NO: 399 | o557_G11_s2_R | CAAGCAGAAGACGGCATACGAGATTTTGAGGGAG |
| | | GGTCAAGTGCACAGTCTA |
| SEQ ID NO: 400 | o558_G12_S2_R | CAAGCAGAAGACGGCATACGAGATAGCCGACAAG |
| | | GGTCAAGTGCACAGTCTA |
| SEQ ID NO: 401 | o559_H1_S2_R | CAAGCAGAAGACGGCATACGAGATAAACCCGCAG |
| | | GGTCAAGTGCACAGTCTA |
| SEQ ID NO: 402 | o560_H2_S2_R | CAAGCAGAAGACGGCATACGAGATGTAGGGCTAG |
| | | GGTCAAGTGCACAGTCTA |
| SEQ ID NO: 403 | o561_H3_S2_R | CAAGCAGAAGACGGCATACGAGATAGACGATTAG |
| | | GGTCAAGTGCACAGTCTA |
| SEQ ID NO: 404 | o562_H4_S2_R | CAAGCAGAAGACGGCATACGAGATAGGATGATAG |
| | | GGTCAAGTGCACAGTCTA |
| SEQ ID NO: 405 | o563_H5_s2_R | CAAGCAGAAGACGGCATACGAGATATAATGGCAG |
| | | GGTCAAGTGCACAGTCTA |
| SEQ ID NO: 406 | o564_H6_S2_R | CAAGCAGAAGACGGCATACGAGATCTTGGCGTAG |
| | | GGTCAAGTGCACAGTCTA |
| SEQ ID NO: 407 | o565_H7_s2_R | CAAGCAGAAGACGGCATACGAGATAGCTGTGCAG |
| | | GGTCAAGTGCACAGTCTA |
| SEQ ID NO: 408 | o566_H8_S2_R | CAAGCAGAAGACGGCATACGAGATGAGTCCAAAG |
| | | GGTCAAGTGCACAGTCTA |
| SEQ ID NO: 409 | o567_H9_S2_R | CAAGCAGAAGACGGCATACGAGATGAATACCAAG |
| | | GGTCAAGTGCACAGTCTA |
| SEQ ID NO: 410 | o568_H10_S2_R | CAAGCAGAAGACGGCATACGAGATAGGAGCTTAG |
| | | GGTCAAGTGCACAGTCTA |
| SEQ ID NO: 411 | o569_H11_S2_R | CAAGCAGAAGACGGCATACGAGATGTGACTTAAG |
| | | GGTCAAGTGCACAGTCTA |
| SEQ ID NO: 412 | o570_H12_S2_R | CAAGCAGAAGACGGCATACGAGATTTTGGAACAG |
| | | GGTCAAGTGCACAGTCTA |
| SEQ ID NO: 413 | o571_plate1_N1_R | AATGATACGGCGACCACCGAGATCTACACAAGAT |
| | | CTGTCTGGTTACTGCCAGTTGAATCTG |
| SEQ ID NO: 414 | o572_plate2_N1_R | AATGATACGGCGACCACCGAGATCTACACTGTCA |
| | | TGATCTGGTTACTGCCAGTTGAATCTG |
| SEQ ID NO: 415 | o573_plate3_N1_R | AATGATACGGCGACCACCGAGATCTACACTGTAA |
| | | CAGTCTGGTTACTGCCAGTTGAATCTG |
| SEQ ID NO: 416 | o574_plate4_N1_R | AATGATACGGCGACCACCGAGATCTACACGCGCA |
| | | ACTTCTGGTTACTGCCAGTTGAATCTG |
| SEQ ID NO: 417 | o575_plate1_N1_R_shortP5 | AATGATACGGCGACCACCGATCGATGGCTCTGGT |
| | | TACTGCCAGTTGAATCTG |
| SEQ ID NO: 418 | o576_plate2_N1_R_shortP5 | AATGATACGGCGACCACCGACATGGTTTTCTGGT |
| | | TACTGCCAGTTGAATCTG |
| SEQ ID NO: 419 | o577_plate3_N1_R_shortP5 | AATGATACGGCGACCACCGAACGAAAGCTCTGGT |
| | | TACTGCCAGTTGAATCTG |
| SEQ ID NO: 420 | o578_plate4_N1_R_shortP5 | AATGATACGGCGACCACCGAGCTCTGATTCTGGT |
| | | TACTGCCAGTTGAATCTG |
| SEQ ID NO: 421 | o579_plate1_S2_F | AATGATACGGCGACCACCGAGATCTACACATGCC |
| | | CTCGCTGGTGCTGCAGCTTATTA |
| SEQ ID NO: 422 | o580_plate2_S2_F | AATGATACGGCGACCACCGAGATCTACACCTCAG |
| | | ATGGCTGGTGCTGCAGCTTATTA |
| SEQ ID NO: 423 | o581_plate3_S2_F | AATGATACGGCGACCACCGAGATCTACACGTAAT |
| | | CTGGCTGGTGCTGCAGCTTATTA |
| SEQ ID NO: 424 | o582_plate4_S2_F | AATGATACGGCGACCACCGAGATCTACACGCAAG |
| | | ATTGCTGGTGCTGCAGCTTATTA |
| SEQ ID NO: 425 | o583_plate1_S2_F_shortP5 | AATGATACGGCGACCACCGAATACGCCAGCTGGT |
| | | GCTGCAGCTTATTA |
| SEQ ID NO: 426 | o584_plate2_S2_F_shortP5 | AATGATACGGCGACCACCGAACCAGTCGGCTGGT |
| | | GCTGCAGCTTATTA |
| SEQ ID NO: 427 | o585_plate3_S2_F_shortP5 | AATGATACGGCGACCACCGAAACACGGAGCTGGT |
| | | GCTGCAGCTTATTA |
| SEQ ID NO: 428 | o586_plate4_S2_F_shortP5 | AATGATACGGCGACCACCGACTGCCTAGGCTGGT |
| | | GCTGCAGCTTATTA |
| SEQ ID NO: 429 | o587_N1_read1 | tctggttactgccagttgaatctgagggtcc |
| SEQ ID NO: 430 | o588_N1_i7 | ggtgcatttcgctgattttggggtc |
| SEQ ID NO: 431 | o589_N1_i5 | ggaccctcagattcaactggcagtaaccaga |
| SEQ ID NO: 432 | o590_S2_read1 | gctggtgctgcagcttattatgtgggt |
| SEQ ID NO: 433 | o591_S2_i7 | agatgctgtagactgtgcacttgaccct |
| SEQ ID NO: 434 | o592_S2_i5 | acccacataataagctgcagcaccagc |
| SEQ ID NO: 435 | o593_RP_read1 | gagcggctgtctccacaagtccg |
| SEQ ID NO: 436 | o594_RP_i7 | acccgctcgcaggtccaaatctg |
| SEQ ID NO: 437 | o595_RP_i5 | cggacttgtggagacagccgctc |
| SEQ ID NO: 438 | o596_A1_RP_F | CAAGCAGAAGACGGCATACGAGATGAGTCTTCAG |
| | | ATTTGGACCTGCGAGCG |
| SEQ ID NO: 439 | o597_A2_RP_F | CAAGCAGAAGACGGCATACGAGATGTTCTATCAG |
| | | ATTTGGACCTGCGAGCG |
| SEQ ID NO: 440 | o598_A3_RP_F | CAAGCAGAAGACGGCATACGAGATTGGGCCAAAG |
| | | ATTTGGACCTGCGAGCG |
| SEQ ID NO: 441 | o599_A4_RP_F | CAAGCAGAAGACGGCATACGAGATATTGTTGGAG |
| | | ATTTGGACCTGCGAGCG |
| SEQ ID NO: 442 | o600_A5_RP_F | CAAGCAGAAGACGGCATACGAGATTCCCGTTGAG |
| | | ATTTGGACCTGCGAGCG |
| SEQ ID NO: 443 | o601_A6_RP_F | CAAGCAGAAGACGGCATACGAGATACACACTTAG |
| | | ATTTGGACCTGCGAGCG |
| SEQ ID NO: 444 | o602_A7_RP_F | CAAGCAGAAGACGGCATACGAGATCCATTCCAAG |
| | | ATTTGGACCTGCGAGCG |
| SEQ ID NO: 445 | o603_A8_RP_F | CAAGCAGAAGACGGCATACGAGATCTAACGGGAG |
| | | ATTTGGACCTGCGAGCG |
| SEQ ID NO: 446 | o604_A9_RP_F | CAAGCAGAAGACGGCATACGAGATCCATAGGAAG |
| | | ATTTGGACCTGCGAGCG |
| SEQ ID NO: 447 | o605_A10_RP_F | CAAGCAGAAGACGGCATACGAGATCAGTGTAGAG |
| | | ATTTGGACCTGCGAGCG |
| SEQ ID NO: 448 | o606_A11_RP_F | CAAGCAGAAGACGGCATACGAGATGATTCTCAAG |
| | | ATTTGGACCTGCGAGCG |
| SEQ ID NO: 449 | o607_A12_RP_F | CAAGCAGAAGACGGCATACGAGATCCTTCTTAAG |
| | | ATTTGGACCTGCGAGCG |
| SEQ ID NO: 450 | o608_B1_RP_F | CAAGCAGAAGACGGCATACGAGATTCTAAGACAG |
| | | ATTTGGACCTGCGAGCG |
| SEQ ID NO: 451 | o609_B2_RP_F | CAAGCAGAAGACGGCATACGAGATGCGGCATAAG |
| | | ATTTGGACCTGCGAGCG |
| SEQ ID NO: 452 | o610_B3_RP_F | CAAGCAGAAGACGGCATACGAGATATTGACGAAG |
| | | ATTTGGACCTGCGAGCG |
| SEQ ID NO: 453 | o611_B4_RP_F | CAAGCAGAAGACGGCATACGAGATGCTCCTGAAG |
| | | ATTTGGACCTGCGAGCG |
| SEQ ID NO: 454 | o612_B5_RP_F | CAAGCAGAAGACGGCATACGAGATGCAATCCTAG |
| | | ATTTGGACCTGCGAGCG |
| SEQ ID NO: 455 | o613_B6_RP_F | CAAGCAGAAGACGGCATACGAGATATGTCGTTAG |
| | | ATTTGGACCTGCGAGCG |
| SEQ ID NO: 456 | o614_B7_RP_F | CAAGCAGAAGACGGCATACGAGATTTCTCGGCAG |
| | | ATTTGGACCTGCGAGCG |
| SEQ ID NO: 457 | o615_B8_RP_F | CAAGCAGAAGACGGCATACGAGATCAGGGCTAAG |
| | | ATTTGGACCTGCGAGCG |
| SEQ ID NO: 458 | o616_B9_RP_F | CAAGCAGAAGACGGCATACGAGATAGCCAAGCAG |
| | | ATTTGGACCTGCGAGCG |
| SEQ ID NO: 459 | o617_B10_RP_F | CAAGCAGAAGACGGCATACGAGATAAGCCTGAAG |
| | | ATTTGGACCTGCGAGCG |
| SEQ ID NO: 460 | o618_B11_RP_F | CAAGCAGAAGACGGCATACGAGATCTACAGAGAG |
| | | ATTTGGACCTGCGAGCG |
| SEQ ID NO: 461 | o619_B12_RP_F | CAAGCAGAAGACGGCATACGAGATCGTAGTCGAG |
| | | ATTTGGACCTGCGAGCG |
| SEQ ID NO: 462 | o620_C1_RP_F | CAAGCAGAAGACGGCATACGAGATTTCTGCTCAG |
| | | ATTTGGACCTGCGAGCG |
| SEQ ID NO: 463 | o621_C2_RP_F | CAAGCAGAAGACGGCATACGAGATGTGCACACAG |
| | | ATTTGGACCTGCGAGCG |
| SEQ ID NO: 464 | o622_C3_RP_F | CAAGCAGAAGACGGCATACGAGATAAAGCTCAAG |
| | | ATTTGGACCTGCGAGCG |
| SEQ ID NO: 465 | o623_C4_RP_F | CAAGCAGAAGACGGCATACGAGATGACCTCAGAG |
| | | ATTTGGACCTGCGAGCG |
| SEQ ID NO: 466 | o624_C5_RP_F | CAAGCAGAAGACGGCATACGAGATCTTTCCAAAG |
| | | ATTTGGACCTGCGAGCG |
| SEQ ID NO: 467 | o625_C6_RP_F | CAAGCAGAAGACGGCATACGAGATTCTTGGCTAG |
| | | ATTTGGACCTGCGAGCG |
| SEQ ID NO: 468 | o626_C7_RP_F | CAAGCAGAAGACGGCATACGAGATCGCGTCTAAG |
| | | ATTTGGACCTGCGAGCG |
| SEQ ID NO: 469 | o627_C8_RP_F | CAAGCAGAAGACGGCATACGAGATTCGCGCTAAG |
| | | ATTTGGACCTGCGAGCG |
| SEQ ID NO: 470 | o628_C9_RP_F | CAAGCAGAAGACGGCATACGAGATATCCATTCAG |
| | | ATTTGGACCTGCGAGCG |
| SEQ ID NO: 471 | o629_C10_RP_F | CAAGCAGAAGACGGCATACGAGATGCCCAGTAAG |
| | | ATTTGGACCTGCGAGCG |
| SEQ ID NO: 472 | o630_C11_RP_F | CAAGCAGAAGACGGCATACGAGATTACCGACGAG |
| | | ATTTGGACCTGCGAGCG |
| SEQ ID NO: 473 | o631_C12_RP_F | CAAGCAGAAGACGGCATACGAGATTCCATACGAG |
| | | ATTTGGACCTGCGAGCG |
| SEQ ID NO: 474 | o632_D1_RP_F | CAAGCAGAAGACGGCATACGAGATAACATGTCAG |
| | | ATTTGGACCTGCGAGCG |
| SEQ ID NO: 475 | o633_D2_RP_F | CAAGCAGAAGACGGCATACGAGATCGACTATAAG |
| | | ATTTGGACCTGCGAGCG |
| SEQ ID NO: 476 | o634_D3_RP_F | CAAGCAGAAGACGGCATACGAGATACCCAAAGAG |
| | | ATTTGGACCTGCGAGCG |
| SEQ ID NO: 477 | o635_D4_RP_F | CAAGCAGAAGACGGCATACGAGATATCGATCGAG |
| | | ATTTGGACCTGCGAGCG |
| SEQ ID NO: 478 | o636_D5_RP_F | CAAGCAGAAGACGGCATACGAGATGTTGGATGAG |
| | | ATTTGGACCTGCGAGCG |
| SEQ ID NO: 479 | o637_D6_RP_F | CAAGCAGAAGACGGCATACGAGATCTATGTGAAG |
| | | ATTTGGACCTGCGAGCG |
| SEQ ID NO: 480 | o638_D7_RP_F | CAAGCAGAAGACGGCATACGAGATTATTTCGCAG |
| | | ATTTGGACCTGCGAGCG |
| SEQ ID NO: 481 | o639_D8_RP_F | CAAGCAGAAGACGGCATACGAGATCCATGTATAG |
| | | ATTTGGACCTGCGAGCG |
| SEQ ID NO: 482 | o640_D9_RP_F | CAAGCAGAAGACGGCATACGAGATGCCACGTTAG |
| | | ATTTGGACCTGCGAGCG |
| SEQ ID NO: 483 | o641_D10_RP_F | CAAGCAGAAGACGGCATACGAGATGTCGTGTAAG |
| | | ATTTGGACCTGCGAGCG |
| SEQ ID NO: 484 | o642D11_RP_F | CAAGCAGAAGACGGCATACGAGATTAAAGTCGAG |
| | | ATTTGGACCTGCGAGCG |
| SEQ ID NO: 485 | o643_D12_RP_F | CAAGCAGAAGACGGCATACGAGATCTTCGGACAG |
| | | ATTTGGACCTGCGAGCG |
| SEQ ID NO: 486 | o644_E1_RP_F | CAAGCAGAAGACGGCATACGAGATGCACTCTCAG |
| | | ATTTGGACCTGCGAGCG |
| SEQ ID NO: 487 | o645_E2_RP_F | CAAGCAGAAGACGGCATACGAGATTCAGATACAG |
| | | ATTTGGACCTGCGAGCG |
| SEQ ID NO: 488 | o646_E3_RP_F | CAAGCAGAAGACGGCATACGAGATCAGTCCCTAG |
| | | ATTTGGACCTGCGAGCG |
| SEQ ID NO: 489 | o647_E4_RP_F | CAAGCAGAAGACGGCATACGAGATGCCCTAACAG |
| | | ATTTGGACCTGCGAGCG |
| SEQ ID NO: 490 | o648_E5_RP_F | CAAGCAGAAGACGGCATACGAGATCTGCATCAAG |
| | | ATTTGGACCTGCGAGCG |
| SEQ ID NO: 491 | o649_E6_RP_F | CAAGCAGAAGACGGCATACGAGATCGGTATCGAG |
| | | ATTTGGACCTGCGAGCG |
| SEQ ID NO: 492 | o650_E7_RP_F | CAAGCAGAAGACGGCATACGAGATAAGTATGGAG |
| | | ATTTGGACCTGCGAGCG |
| SEQ ID NO: 493 | o651_E8_RP_F | CAAGCAGAAGACGGCATACGAGATATTCGCGCAG |
| | | ATTTGGACCTGCGAGCG |
| SEQ ID NO: 494 | o652_E9_RP_F | CAAGCAGAAGACGGCATACGAGATATCAAGGTAG |
| | | ATTTGGACCTGCGAGCG |
| SEQ ID NO: 495 | o653_E10_RP_F | CAAGCAGAAGACGGCATACGAGATTTGTGCATAG |
| | | ATTTGGACCTGCGAGCG |
| SEQ ID NO: 496 | o654_E11_RP_F | CAAGCAGAAGACGGCATACGAGATCTGTGCTGAG |
| | | ATTTGGACCTGCGAGCG |
| SEQ ID NO: 497 | o655_E12_RP_F | CAAGCAGAAGACGGCATACGAGATGTCCGTAGAG |
| | | ATTTGGACCTGCGAGCG |
| SEQ ID NO: 498 | o656_F1_RP_F | CAAGCAGAAGACGGCATACGAGATGTTCAAGAAG |
| | | ATTTGGACCTGCGAGCG |
| SEQ ID NO: 499 | o657_F2_RP_F | CAAGCAGAAGACGGCATACGAGATCACCGTTCAG |
| | | ATTTGGACCTGCGAGCG |
| SEQ ID NO: 500 | o658_F3_RP_F | CAAGCAGAAGACGGCATACGAGATCGAGTTGAAG |
| | | ATTTGGACCTGCGAGCG |
| SEQ ID NO: 501 | o659_F4_RP_F | CAAGCAGAAGACGGCATACGAGATGAGCACGAAG |
| | | ATTTGGACCTGCGAGCG |
| SEQ ID NO: 502 | o660_F5_RP_F | CAAGCAGAAGACGGCATACGAGATAGTTCGTGAG |
| | | ATTTGGACCTGCGAGCG |
| SEQ ID NO: 503 | o661_F6_RP_F | CAAGCAGAAGACGGCATACGAGATCATCAACTAG |
| | | ATTTGGACCTGCGAGCG |
| SEQ ID NO: 504 | o662_F7_RP_F | CAAGCAGAAGACGGCATACGAGATCGAGATCTAG |
| | | ATTTGGACCTGCGAGCG |
| SEQ ID NO: 505 | o663_F8_RP_F | CAAGCAGAAGACGGCATACGAGATTGGCCAGAAG |
| | | ATTTGGACCTGCGAGCG |
| SEQ ID NO: 506 | o664_F9_RP_F | CAAGCAGAAGACGGCATACGAGATTTCACCATAG |
| | | ATTTGGACCTGCGAGCG |
| SEQ ID NO: 507 | o665_F10_RP_F | CAAGCAGAAGACGGCATACGAGATGAATGCATAG |
| | | ATTTGGACCTGCGAGCG |
| SEQ ID NO: 508 | o666_F11_RP_F | CAAGCAGAAGACGGCATACGAGATTGGACCCTAG |
| | | ATTTGGACCTGCGAGCG |
| SEQ ID NO: 509 | o667_F12_RP_F | CAAGCAGAAGACGGCATACGAGATGATAGCACAG |
| | | ATTTGGACCTGCGAGCG |
| SEQ ID NO: 510 | o668_G1_RP_F | CAAGCAGAAGACGGCATACGAGATACGACGACAG |
| | | ATTTGGACCTGCGAGCG |
| SEQ ID NO: 511 | o669_G2_RP_F | CAAGCAGAAGACGGCATACGAGATCTCAGTATAG |
| | | ATTTGGACCTGCGAGCG |
| SEQ ID NO: 512 | o670_G3_RP_F | CAAGCAGAAGACGGCATACGAGATCTTAGCTAAG |
| | | ATTTGGACCTGCGAGCG |
| SEQ ID NO: 513 | o671_G4_RP_F | CAAGCAGAAGACGGCATACGAGATCTGTTTACAG |
| | | ATTTGGACCTGCGAGCG |
| SEQ ID NO: 514 | o672_G5_RP_F | CAAGCAGAAGACGGCATACGAGATTGTCCCACAG |
| | | ATTTGGACCTGCGAGCG |
| SEQ ID NO: 515 | o673_G6_RP_F | CAAGCAGAAGACGGCATACGAGATTCCTGAGGAG |
| | | ATTTGGACCTGCGAGCG |
| SEQ ID NO: 516 | o674_G7_RP_F | CAAGCAGAAGACGGCATACGAGATTAGTTCCAAG |
| | | ATTTGGACCTGCGAGCG |
| SEQ ID NO: 517 | o675_G8_RP_F | CAAGCAGAAGACGGCATACGAGATCATGACTCAG |
| | | ATTTGGACCTGCGAGCG |
| SEQ ID NO: 518 | o676_G9_RP_F | CAAGCAGAAGACGGCATACGAGATGTAAGCGCAG |
| | | ATTTGGACCTGCGAGCG |
| SEQ ID NO: 519 | o677_G10_RP_F | CAAGCAGAAGACGGCATACGAGATAACCCAGTAG |
| | | ATTTGGACCTGCGAGCG |
| SEQ ID NO: 520 | o678_G11_RP_F | CAAGCAGAAGACGGCATACGAGATTTTGAGGGAG |
| | | ATTTGGACCTGCGAGCG |
| SEQ ID NO: 521 | o679_G12_RP_F | CAAGCAGAAGACGGCATACGAGATAGCCGACAAG |
| | | ATTTGGACCTGCGAGCG |
| SEQ ID NO: 522 | o680_H1_RP_F | CAAGCAGAAGACGGCATACGAGATAAACCCGCAG |
| | | ATTTGGACCTGCGAGCG |
| SEQ ID NO: 523 | o681_H2_RP_F | CAAGCAGAAGACGGCATACGAGATGTAGGGCTAG |
| | | ATTTGGACCTGCGAGCG |
| SEQ ID NO: 524 | o682_H3_RP_F | CAAGCAGAAGACGGCATACGAGATAGACGATTAG |
| | | ATTTGGACCTGCGAGCG |
| SEQ ID NO: 525 | o683_H4_RP_F | CAAGCAGAAGACGGCATACGAGATAGGATGATAG |
| | | ATTTGGACCTGCGAGCG |
| SEQ ID NO: 526 | o684_H5_RP_F | CAAGCAGAAGACGGCATACGAGATATAATGGCAG |
| | | ATTTGGACCTGCGAGCG |
| SEQ ID NO: 527 | o685_H6_RP_F | CAAGCAGAAGACGGCATACGAGATCTTGGCGTAG |
| | | ATTTGGACCTGCGAGCG |
| SEQ ID NO: 528 | o686_H7_RP_F | CAAGCAGAAGACGGCATACGAGATAGCTGTGCAG |
| | | ATTTGGACCTGCGAGCG |
| SEQ ID NO: 529 | o687_H8_RP_F | CAAGCAGAAGACGGCATACGAGATGAGTCCAAAG |
| | | ATTTGGACCTGCGAGCG |
| SEQ ID NO: 530 | o688_H_RP_F | CAAGCAGAAGACGGCATACGAGATGAATACCAAG |
| | | ATTTGGACCTGCGAGCG |
| SEQ ID NO: 531 | o689_H10_RP_F | CAAGCAGAAGACGGCATACGAGATAGGAGCTTAG |
| | | ATTTGGACCTGCGAGCG |
| SEQ ID NO: 532 | o690_H11_RP_F | CAAGCAGAAGACGGCATACGAGATGTGACTTAAG |
| | | ATTTGGACCTGCGAGCG |
| SEQ ID NO: 533 | o691_H12_RP_F | CAAGCAGAAGACGGCATACGAGATTTTGGAACAG |
| | | ATTTGGACCTGCGAGCG |
| SEQ ID NO: 534 | o692_plate1_RP_R | AATGATACGGCGACCACCGAGATCTACACCTCTC |
| | | TATGAGCGGCTGTCTCCACAAGT |
| SEQ ID NO: 535 | o693_plate2_RP_R | AATGATACGGCGACCACCGAGATCTACACTATCC |
| | | TCTGAGCGGCTGTCTCCACAAGT |
| SEQ ID NO: 536 | o694_plate3_RP_R | AATGATACGGCGACCACCGAGATCTACACGTAAG |
| | | GAGGAGCGGCTGTCTCCACAAGT |
| SEQ ID NO: 537 | o695_plate4_RP_R | AATGATACGGCGACCACCGAGATCTACACACTGC |
| | | ATAGAGCGGCTGTCTCCACAAGT |
| SEQ ID NO: 538 | o696_plate1_RP_R_shortP5 | AATGATACGGCGACCACCGAAAGGAGTAGAGCGG |
| | | CTGTCTCCACAAGT |
| SEQ ID NO: 539 | o697_plate2_RP_R_shortP5 | AATGATACGGCGACCACCGACTAAGCCTGAGCGG |
| | | CTGTCTCCACAAGT |
| SEQ ID NO: 540 | o698_plate3_RP_R_shortP5 | AATGATACGGCGACCACCGACGTCTAATGAGCGG |
| | | CTGTCTCCACAAGT |
| SEQ ID NO: 541 | o699_plate4_RP_R_shortP5 | AATGATACGGCGACCACCGATCTCTCCGGAGCGG |
| | | CTGTCTCCACAAGT |
| SEQ ID NO: 542 | o700_plate1_RP_R | AATGATACGGCGACCACCGAGATCTACACAAGAT |
| | | CTGGAGCGGCTGTCTCCACAAGT |
| SEQ ID NO: 543 | o701_plate2_RP_R | AATGATACGGCGACCACCGAGATCTACACTGTCA |
| | | TGAGAGCGGCTGTCTCCACAAGT |
| SEQ ID NO: 544 | o702_plate3_RP_R | AATGATACGGCGACCACCGAGATCTACACTGTAA |
| | | CAGGAGCGGCTGTCTCCACAAGT |
| SEQ ID NO: 545 | o703_plate4_RP_R | AATGATACGGCGACCACCGAGATCTACACGCGCA |
| | | ACTGAGCGGCTGTCTCCACAAGT |
| SEQ ID NO: 546 | o704_plate1_RP_R_shortP5 | AATGATACGGCGACCACCGATCGATGGCGAGCGG |
| | | CTGTCTCCACAAGT |
| SEQ ID NO: 547 | o705_plate2_RP_R_shortP5 | AATGATACGGCGACCACCGACATGGTTTGAGCGG |
| | | CTGTCTCCACAAGT |
| SEQ ID NO: 548 | o706_plate3_RP_R_shortP5 | AATGATACGGCGACCACCGAACGAAAGCGAGCGG |
| | | CTGTCTCCACAAGT |
| SEQ ID NO: 549 | o707_plate4_RP_R_shortP5 | AATGATACGGCGACCACCGAGCTCTGATGAGCGG |
| | | CTGTCTCCACAAGT |
| SEQ ID NO: 550 | o708_plate1_RP_R | AATGATACGGCGACCACCGAGATCTACACATGCC |
| | | CTCGAGCGGCTGTCTCCACAAGT |
| SEQ ID NO: 551 | o709_plate2_RP_R | AATGATACGGCGACCACCGAGATCTACACCTCAG |
| | | ATGGAGCGGCTGTCTCCACAAGT |
| SEQ ID NO: 552 | o710_plate3_RP_R | AATGATACGGCGACCACCGAGATCTACACGTAAT |
| | | CTGGAGCGGCTGTCTCCACAAGT |
| SEQ ID NO: 553 | o711_plate4_RP_R | AATGATACGGCGACCACCGAGATCTACACGCAAG |
| | | ATTGAGCGGCTGTCTCCACAAGT |
| SEQ ID NO: 554 | o712_plate1_RP_R_shortP5 | AATGATACGGCGACCACCGAATACGCCAGAGCGG |
| | | CTGTCTCCACAAGT |
| SEQ ID NO: 555 | o713_plate2_RP_R_shortP5 | AATGATACGGCGACCACCGAACCAGTCGGAGCGG |
| | | CTGTCTCCACAAGT |
| SEQ ID NO: 556 | o714_plate3_RP_R_shortP5 | AATGATACGGCGACCACCGAAACACGGAGAGCGG |
| | | CTGTCTCCACAAGT |
| SEQ ID NO: 557 | o715_plate4_RP_R_shortP5 | AATGATACGGCGACCACCGACTGCCTAGGAGCGG |
| | | CTGTCTCCACAAGT |
| SEQ ID NO: 558 | o716_skpp15-1-F_N1 | GGGTCACGCGTAGGA |
| | | GTTCTATCGACCCCAAAATCAGCGAAAT |
| SEQ ID NO: 559 | o717_skpp15-1-R_N1 | GTTCCGCAGCCACAC |
| | | AAGATCTGTCTGGTTACTGCCAGTTGAATCTG |
| SEQ ID NO: 560 | o718_skpp15-2-F_N1 | CGCGTCGAGTAGGGT |
| | | GTTCTATCGACCCCAAAATCAGCGAAAT |
| SEQ ID NO: 561 | o719_skpp15-2-R_N1 | GCCGTGTGAAGCTGG |
| | | AAGATCTGTCTGGTTACTGCCAGTTGAATCTG |
| SEQ ID NO: 562 | o720_skpp15-3-F_N1 | CGATCGCCCTTGGTG |
| | | GTTCTATCGACCCCAAAATCAGCGAAAT |
| SEQ ID NO: 563 | o721_skpp15-3-R_N1 | GGTTTAGCCGGCGTG |
| | | AAGATCTGTCTGGTTACTGCCAGTTGAATCTG |
| SEQ ID NO: 564 | o722_skpp15-4-F_N1 | GGTCGAGCCGGAACT |
| | | GTTCTATCGACCCCAAAATCAGCGAAAT |
| SEQ ID NO: 565 | o723_skpp15-4-R_N1 | GGATGCGCACCCAGA |
| | | AAGATCTGTCTGGTTACTGCCAGTTGAATCTG |
| SEQ ID NO: 566 | o724_skpp15-5-F_N1 | TCCCGGCGTTGTCCT |
| | | GTTCTATCGACCCCAAAATCAGCGAAAT |
| SEQ ID NO: 567 | o725_skpp15-5-R_N1 | GCTCCGTCACTGCCC |
| | | AAGATCTGTCTGGTTACTGCCAGTTGAATCTG |
| SEQ ID NO: 568 | o726_skpp15-6-F_N1 | CGCAGGGTCCAGAGT |
| | | GTTCTATCGACCCCAAAATCAGCGAAAT |
| SEQ ID NO: 569 | o727_skpp15-6-R_N1 | GTTCGCGCGAAGGAA |
| | | AAGATCTGTCTGGTTACTGCCAGTTGAATCTG |
| SEQ ID NO: 570 | o728_skpp-1-F_N1 | ATATAGATGCCGTCCTAGCG |
| | | GTTCTATCGACCCCAAAATCAGCGAAAT |
| SEQ ID NO: 571 | o729_skpp-1-R_N1 | AAGTATCTTTCCTGTGCCCA |
| | | AAGATCTGTCTGGTTACTGCCAGTTGAATCTG |
| SEQ ID NO: 572 | o730_skpp-2-F_N1 | CCCTTTAATCAGATGCGTCG |
| | | GTTCTATCGACCCCAAAATCAGCGAAAT |
| SEQ ID NO: 573 | o731_skpp-2-R_N1 | TGGTAGTAATAAGGGCGACC |
| | | AAGATCTGTCTGGTTACTGCCAGTTGAATCTG |
| SEQ ID NO: 574 | o732_skpp-3-F_N1 | TTGGTCATGTGCTTTTCGTT |
| | | GTTCTATCGACCCCAAAATCAGCGAAAT |
| SEQ ID NO: 575 | o733_skpp-3-R_N1 | AGGGGTATCGGATACTCAGA |
| | | AAGATCTGTCTGGTTACTGCCAGTTGAATCTG |
| SEQ ID NO: 576 | o734_skpp-4-F_N1 | GGGTGGGTAAATGGTAATGC |
| | | GTTCTATCGACCCCAAAATCAGCGAAAT |
| SEQ ID NO: 577 | o735_skpp-4-R_N1 | ATCGATTCCCCGGATATAGC |
| | | AAGATCTGTCTGGTTACTGCCAGTTGAATCTG |
| SEQ ID NO: 578 | o736_skpp-5-F_N1 | TCCGACGGGGAGTATATACT |
| | | GTTCTATCGACCCCAAAATCAGCGAAAT |
| SEQ ID NO: 579 | o737_skpp-5-R_N1 | TACTAACTGCTTCAGGCCAA |
| | | AAGATCTGTCTGGTTACTGCCAGTTGAATCTG |
| SEQ ID NO: 580 | o738_skpp-6-F_N1 | CATGTTTAGGAACGCTACCG |
| | | GTTCTATCGACCCCAAAATCAGCGAAAT |
| SEQ ID NO: 581 | o739_skpp-6-R_N1 | AATAATCTCCGTTCCCTCCC |
| | | AAGATCTGTCTGGTTACTGCCAGTTGAATCTG |
| SEQ ID NO: 582 | o740_skpp15-1-F_S2 | GGGTCACGCGTAGGA |
| | | GTTCTATCGCTGGTGCTGCAGCTTATTA |
| SEQ ID NO: 583 | o741_skpp15-1-R_S2 | GTTCCGCAGCCACAC |
| | | AAGATCTGAGGGTCAAGTGCACAGTCTA |
| SEQ ID NO: 584 | o742_skpp15-2-F_S2 | CGCGTCGAGTAGGGT |
| | | GTTCTATCGCTGGTGCTGCAGCTTATTA |
| SEQ ID NO: 585 | o743_skpp15-2-R_S2 | GCCGTGTGAAGCTGG |
| | | AAGATCTGAGGGTCAAGTGCACAGTCTA |
| SEQ ID NO: 586 | o744_skpp15-3-F_S2 | CGATCGCCCTTGGTG |
| | | GTTCTATCGCTGGTGCTGCAGCTTATTA |
| SEQ ID NO: 587 | o745_skpp15-3-R_S2 | GGTTTAGCCGGCGTG |
| | | AAGATCTGAGGGTCAAGTGCACAGTCTA |
| SEQ ID NO: 588 | o746_skpp15-4-F_S2 | GGTCGAGCCGGAACT |
| | | GTTCTATCGCTGGTGCTGCAGCTTATTA |
| SEQ ID NO: 589 | o747_skpp15-4-R_S2 | GGATGCGCACCCAGA |
| | | AAGATCTGAGGGTCAAGTGCACAGTCTA |
| SEQ ID NO: 590 | o748_skpp15-5-F_S2 | TCCCGGCGTTGTCCT |
| | | GTTCTATCGCTGGTGCTGCAGCTTATTA |
| SEQ ID NO: 591 | o749_skpp15-5-R_S2 | GCTCCGTCACTGCCC |
| | | AAGATCTGAGGGTCAAGTGCACAGTCTA |
| SEQ ID NO: 592 | o750_skpp15-6-F_S2 | CGCAGGGTCCAGAGT |
| | | GTTCTATCGCTGGTGCTGCAGCTTATTA |
| SEQ ID NO: 593 | o751_skpp15-6-R_S2 | GTTCGCGCGAAGGAA |
| | | AAGATCTGAGGGTCAAGTGCACAGTCTA |
| SEQ ID NO: 594 | o752_skpp-1-F s_S2 | ATATAGATGCCGTCCTAGCG |
| | | GTTCTATCGCTGGTGCTGCAGCTTATTA |
| SEQ ID NO: 595 | o753_skpp-1-R s_S2 | AAGTATCTTTCCTGTGCCCA |
| | | AAGATCTGAGGGTCAAGTGCACAGTCTA |
| SEQ ID NO: 596 | o754_skpp-2-F s_S2 | CCCTTTAATCAGATGCGTCG |
| | | GTTCTATCGCTGGTGCTGCAGCTTATTA |
| SEQ ID NO: 597 | o755_skpp-2-R s_S2 | TGGTAGTAATAAGGGCGACC |
| | | AAGATCTGAGGGTCAAGTGCACAGTCTA |
| SEQ ID NO: 598 | o756_skpp-3-F s_S2 | TTGGTCATGTGCTTTTCGTT |
| | | GTTCTATCGCTGGTGCTGCAGCTTATTA |
| SEQ ID NO: 599 | o757_skpp-3-R s_S2 | AGGGGTATCGGATACTCAGA |
| | | AAGATCTGAGGGTCAAGTGCACAGTCTA |
| SEQ ID NO: 600 | o758_skpp-4-F s_S2 | GGGTGGGTAAATGGTAATGC |
| | | GTTCTATCGCTGGTGCTGCAGCTTATTA |
| SEQ ID NO: 601 | o759_skpp-4-R s_S2 | ATCGATTCCCCGGATATAGC |
| | | AAGATCTGAGGGTCAAGTGCACAGTCTA |
| SEQ ID NO: 602 | o760_skpp-5-F s_S2 | TCCGACGGGGAGTATATACT |
| | | GTTCTATCGCTGGTGCTGCAGCTTATTA |
| SEQ ID NO: 603 | o761_skpp-5-R s_S2 | TACTAACTGCTTCAGGCCAA |
| | | AAGATCTGAGGGTCAAGTGCACAGTCTA |
| SEQ ID NO: 604 | o762_skpp-6-F s_S2 | CATGTTTAGGAACGCTACCG |
| | | GTTCTATCGCTGGTGCTGCAGCTTATTA |
| SEQ ID NO: 605 | o763_skpp-6-R s_S2 | AATAATCTCCGTTCCCTCCC |
| | | AAGATCTGAGGGTCAAGTGCACAGTCTA |

### Example 4—SwabSeq: A High-Throughput Platformfor Massively Scaled Up SARS-CoV-2 Testing

In the absence of an effective vaccine, public health strategies remain the only tools for controlling the spread of SARS-CoV-2, the cause of COVID-19. In contrast to SARS-CoV-1, for which infectivity is associated with symptoms, infectivity of SARS-CoV-2 is high during the asymptomatic/presymptomatic phase. As a consequence, containing transmission based solely on symptoms is impossible, which makes screening for SARS-CoV-2 essential for pandemic control.

As regional lockdowns have been lifted and people have returned to work and resumed other activities, rates of infection have started to rise again. In many parts of the United States, the rise in cases has overwhelmed the capacity of quantitative RT-PCR tests that make up the majority of FDA-approved tests for COVID-19. Even in those parts of the country that have expanded testing capacity, the cost of each test, at approximately $100, prohibits the deployment of clinical testing for population screening. Furthermore, the long delays in returning results are due to the insufficient testing capacity and not due to the few hours of assay time, which renders testing useless in preventing viral transmission and suppressing local outbreaks. Frequent, inexpensive mass testing, combined with contact tracing and isolation of infected individuals, offers the best chance of stopping the spread of virus. Described herein is SwabSeq, a SARS-CoV-2 testing platform that leverages next-generation sequencing to massively scale up testing capacity.

SwabSeq improves on one-step reverse transcription and polymerase chain reaction (RT-PCR) approaches in several key areas. SwabSeq uses molecular barcodes that are embedded in the RT and PCR primers to uniquely label each sample. After the RT-PCR step, the barcoded samples are combined into a single sequencing library, which enables multiplexing of up to hundreds of thousands of samples on an Illumina sequencer. The readout of the test is not fluorescence (thus obviating the need for expensive qPCR machines) but the count of viral sequencing reads. A short read length (26 base pairs) is sufficient to identify the target sequence and keeps total sequencing time down to approximately 5 hours.

The key to SwabSeqs robust capability to identify SARS-CoV-2 is the inclusion of an internal in vitro RNA standard that allows normalization of viral read counts within each well. This internal standard turns a count-based assay into a ratiometric one. This is important, as it allows for the ability to accurately call positive and negative samples despite heterogeneity in RT- and PCR-inhibition in clinical samples. Reliance on sequencing as the readout, along with the in vitro RNA standard, allows SwabSeq to use amplification performed for up to 50 cycles until primers are consumed. This endpoint PCR overcomes enzyme inhibition present in extraction-free samples, enabling further streamlining of the workflow with minimal loss of analytical sensitivity. These features enable testing at much higher throughput than the current RT-quantitativePCR (RT-qPCR) diagnostic tests, which require RNA purification and RT-qPCR-costly, labor- and reagent-intensive steps that are constrained by the availability of the necessary instrumentation. In contrast, SwabSeq relies only on readily available and underutilized thermocyclers and DNA sequencers. SwabSeq can be performed at the scale of tens of thousands of samples per day without the extensive automation that is typically required for scale beyond hundreds of samples per day.

As demonstrated herein, SwabSeq has extremely high sensitivity and specificity for the detection of viral RNA in purified samples. The disclosure and examples herein further demonstrate a low limit of detection in extraction-free lysates from mid-nasal swabs and oral fluids. These results demonstrate the potential of SwabSeq to be used for SARS-CoV-2 testing on an unprecedented scale.

### Results

SwabSeq is a simple and scalable protocol, consisting of 5 steps (FIG. 4, panel A): (1) sample collection, (2) reverse transcription and PCR using primers that contain unique molecular indices at the i7 and i5 positions (FIG. 4, panel B, FIG. 6), (3) pooling of the uniquely barcoded samples for library preparation, (4) sequencing of the pooled library, and (5) computational assignment of barcoded sequencing reads to each sample for counting and viral detection.

The assay consists of two primers sets that amplify two genes: the S2 gene of SARS-CoV-2 and the human Ribonuclease PIMRP Subunit P30 (RPP30). The assay includes a synthetic in vitro transcribed control RNA that is identical to the viral sequence targeted for amplification, except for a short-altered stretch (FIG. 4, panel C) that allows distinguishing of sequencing reads corresponding to the synthetic control from those corresponding to the native sequence. The primers amplify both the native and the synthetic sequences with equal efficiency (FIG. 7).

The S2 internal control serves two purposes. First, inhibitors of PCR and reverse transcription, as well as other sources of well-to-well variation, are likely to affect the amplification of both the control and the sample in the same way. The ratio of the number of native reads to the number of control reads provides a more accurate and quantitative measure of viral load than native read counts alone (FIG. 4, panels E and D). Second, the internal control allows the retention of linearity over a large range of viral input despite the use of endpoint PCR (FIG. 8). With this approach, the final amount of DNA in each well is largely defined by the total primer concentration rather than by the viral load—a negative sample is expected to have a similar total number of S2 reads as a positive one. The difference is that in a negative sample all S2 amplicon reads map to the control synthetic sequence. In addition to viral S2, the assay/method comprises reverse-transcribing and amplifying a human housekeeping gene to control for specimen quality and collection efficiency (FIG. 4 panel F).

After RT-PCR samples are combined at equal volumes, purified, and used to generate one sequencing library. Both the Illumina MiSeq and/or the Illumina NextSeq can be used to sequence these libraries (FIG. 9). Instrument sequencing time is minnized by only sequencing 26 base pairs of the amplicons (Methods). Each read is classified as deriving from native or control S2 or RPP30 based on its sequence, and assigned to a sample based on the associated index sequences (barcodes). To maximize specificity and avoid false-positive signals arising from incorrect classification or assignment, conservative edit distance thresholds are used for this matching operation (Methods and Supplemental Results). A sequencing read is discarded if it does not match one of the expected sequences. Counts for native and control S2 and RPP30 reads are obtained for each sample and used for downstream analyses (Methods, below).

It was observed that a few thousand reads are sufficient to detect and quantify the presence of viral RNA in a sample (10,000 conservatively). This translates to 1,500 samples per run on a MiSeq v3 flow cell, 20,000 samples per run on a NextSeq, and up to 150,000 samples per run on a NovaSeq S2 flow cell. Computational analysis takes only minutes per run. SwabSeq is a streamlined and scalable protocol for COVID-19 testing. The SwabSeq protocol was further optimized by identifying and eliminating multiple sources of noise (FIG. 21, panels A, B, C, and D).

### Validation of SwabSeq as a Diagnostic Platform

SwabSeq was first validated on purified RNA samples that were previously tested by the UCLA Clinical Laboratory with a standard RT-qPCR assay. To determine the analytical limit of detection, inactivated virus was diluted with pooled, remnant clinical nasopharyngeal swab specimens. The remnant samples were all confirmed to be negative for SARS-CoV-2. In these remnant samples, a serial, 2-fold dilution of heat-inactivated SARS-CoV-2 (ATCC® VR-1986HK) was performed, in the range from 8000 to 125 genome copy equivalents (GCE) per mL. SARS-CoV-2 in all samples down to 250 GCE per mL were detected, and in most samples to 125 GCE per mL (FIG. 2A). These results established that SwabSeq is highly sensitive, with an analytical limit of detection (LOD) of 250 GCE per mL. This limit of detection is lower than many currently approved and highly sensitive RT-qPCR assays. This comparison demonstrates that SwabSeq can be highly effective as a clinical diagnostic test.

SwabSeq detects virus with high sensitivity and specificity. Confirmed positive (n=31) and negative (n=33) samples from the UCLA Clinical Microbiology Laboratory were retested. 100% agreement with RT-qPCR results for all samples (FIG. 5) was observed. The libraries were sequenced on both a MiSeq and a NextSeq550 (FIG. 9), with 100% concordance between the different sequencing instruments.

One of the major bottlenecks in scaling up RT-qPCR diagnostic tests is the RNA purification step. RNA extraction is challenging to automate, and supply chains have not been able to keep up with the demand for necessary reagents during the course of the pandemic. The ability of SwabSeq to detect SARS-CoV-2 directly from a variety of extraction-free sample types was explored as a way to circumvent the bottleneck of RNA purification.

There are several types of media that are recommended by the CDC for nasal swab collection: viral transport medium (VTM), Amies transport medium, and normal saline. A main technical challenge arises from RT or PCR inhibition by ingredients in the collection buffers. Dilution of specimens with water overcame the RT and PCR inhibition and allowed for the ability to detect viral RNA in contrived and positive clinical patient samples (FIG. 11). Nasal swabs that had been collected directly into the Tris-EDTA (TE) buffer, diluted 1:1 with water were tested. This approach yielded a limit of detection of 558 GCE/mL (FIG. 5, panel C). A comparison between the extraction free-protocol for nasopharyngeal samples collected into normal saline and RT-qPCR conducted by the UCLA Clinical Microbiology Lab showed 100% agreement for all samples (FIG. 2D).

Extraction-free saliva protocols in which saliva is collected directly into a matrix tube using a funnel-like collection device were also tested (FIG. 12). The main technical challenge has been preventing the various components in saliva from degrading viral RNA and ensuring accurate pipetting of this heterogeneous and viscous sample type. Heating the saliva samples to 95° C. for 30 minutes reduced PCR inhibition and improved detection of the S2 amplicon as compared to no preheating (FIG. 13). After the heat step, samples were diluted at a 1:1 volume of 2×TBE with 0.1% Tween-20. Using this method, a LoD of 2000 GCE/mL was obtained (FIG. 5, panel E).

### DISCUSSION

Swabseq has the potential to alleviate existing bottlenecks in diagnostic clinical testing. Further, SwabSeq has even greater potential to enable testing on a scale necessary for pandemic suppression via population surveillance. The technology represents a novel use of massively parallel next-generation sequencing for infectious disease surveillance and diagnostics. SwabSeq can detect SARS-CoV-2 RNA in clinical specimens from both purified RNA and extraction-free lysates, and maintain high clinical and analytical sensitivity and specificity comparable to RT-qPCR performed in a clinical diagnostic laboratory. SwabSeq was also further optimized to prioritize scale and low cost, as these are the key factors that are missing from current COVID-19 diagnostic tests.

SwabSeq should be evaluated as a tool for surveillance testing, rather than for clinical testing. Clinical testing informs clinical decision making, and thus requires high sensitivity and specificity, while for surveillance testing the most important factors are the breadth and frequency of testing and the turn-around-time. Sufficiently broad and frequent testing with rapid return of results can effectively suppress viral outbreaks by selective quarantine of infectious individuals rather than blanket stay-at-home orders. Epidemiological modeling of surveillance testing on university campuses has shown that even diagnostic tests with 70% sensitivity, but performed frequently with a short turn-around time, can suppress transmission. Major challenges for practical implementation of frequent testing include the cost of testing and the logistics of collecting and processing hundreds and thousands of samples per day.

The use of Illumina sequencing in diagnostic testing has garnered concern with regards to turn-around-time and cost. SwabSeq uses short sequencing runs that read out the molecular indexes and 26 base pairs of the target sequence in 5 hours, followed by computational analysis that can be performed on a desktop computer in 5 minutes. The cost of sequencing reagents when 1,000 samples are analyzed in one MiSeq run is less than $1 per sample. Running 10,000 samples on a NextSeq550, which generates 13 times more reads per flow cell, can reduce this cost approximately 10-fold. Further optimization to decrease reaction volumes and to use less expensive RT-PCR reagents can further decrease the total cost per test.

Finally, scaling up testing for SARS-CoV-2 requires high throughput sample collection and processing workflows. Manual processes, common in most academic clinical laboratories, are not easily compatible with simple automation. The current protocols with nasopharyngeal swabs into VTM, Amies or NS are collection methods that date back to the pre-molecular-genetics era, when live viral culture was used to identify cytopathic effects on cell lines, and when testing was performed manually with low throughput. A fresh perspective on collection methods that scale would be enormously beneficial to the testing community.

Several groups, have piloted “lightweight” sample collection approaches, which push sample registration and patient information collection directly onto the individual tested via a smartphone app. Much of the labor of sample requisition is due to a lack of interoperability between electronic health systems, with laboratory professionals manually entering information for every sample by hand. By developing a HIPAA-compliant registration process, labor-intensive sample requisition can be streamlined. To promote scalability, sample collection protocols that use smaller-volume tubes that are compatible with simple automation, such as automated capper-decapper and 96-head liquid handlers can beused. These approaches decrease the amount of hands-on work required in the laboratory to process and perform testing.

The SwabSeq diagnostic platform complements traditional clinical diagnostics tests, as well as the growing arsenal of point-of-care rapid diagnostic platforms emerging for COVID-19, by increasing test capacity to meet the needs of both diagnostic and widespread surveillance testing. Looking forward, SwabSeq is easily extensible to accommodate additional pathogens and viral targets. This could further increase the usefulness of the test, particularly during the winter cold and flu season, when multiple respiratory pathogens circulate in the populations and cannot be easily differentiated based on symptoms alone. Surveillance testing is likely to become a part of the new normal as society aims to safely reopen the educational, business and recreational sectors of society.

### Methods

Sample Collection: All patient samples used in the study were deidentified. All samples were obtained with UCLA IRB Approval. Nasopharyngeal samples were collected by health care providers in patients with suspicion of COVID19.

Creation of Contrived Specimens: For the clinical limit of detection experiments, confirmed COVID-19 negative remnant nasopharyngeal swab specimens collected by the UCLA Clinical Microbiology Laboratory were pooled. Pooled clinical samples were then spiked with ATCC Inactivated Virus (ATCC 1986-HK) at specified concentrations and extracted as described below. For the clinical purified RNA samples, they were collected as nasopharyngeal swabs and purified using the KingFisherFlex (Thermofisher Scientific) instrument using the MagMax bead extraction. All extractions were performed according to manufacturer's protocols. For Extraction free samples, samples were first contrived at specified concentrations into pooled, confirmed negative clinical samples and performed diluted samples in TE buffer or water prior to adding to the RT-PCR master mix.

Processing of Extraction-Free Saliva Specimens: Direct Saliva is collected into a matrix tube using a small funnel (part number from Amazon). The saliva samples were collected into a matrix tube and heated to 95 C for 30 minutes. Samples were then either frozen or processed by dilution with 2×TBE with 1% Tween-20, for final concentration of 1×TBE and 0.5% Tween-2. 1× Tween with Qiagen Protease and RNA Secure (ThermoFisher) was tested, which also works but resulted in more sample-to-sample variability and required additional incubation steps.

Processing of Extraction-Free Nasal Swab Lysates: All extraction free lysates were inactivated using a heat inactivation at 56 C for 30 minutes. Samples were then diluted with water at a ratio of 1:4 and then directly added to mastermix. Dilution amounts varied depending on the liquid media that was used. Of the CDC recommended media, normal saline performed the most robustly. Viral Transport Media and Amies Buffer showed significant PCR inhibition that was difficult to overcome, even with dilution in water. In an embodiment, the swab is placed directly into the diluted TE buffer, which has no effects on PCR inhibition.

Barcode Primer Design: Barcode primers were chosen from a set of 1,536 unique 10 bp i5 barcodes and a set of 1,536 unique 10 bp i7 barcodes. These 10 bp barcodes satisfy the criteria that there is a minimum levenshtein distance of 3 between any two indices (within the i5 and i7 sets) and that the barcodes contain no homopolymer repeats greater than 2 nucleotides. Additionally, barcodes were chosen to minimize homo- and hetero-dimerization using helper functions in the python API to Primer3.

Construction of S2 and RPP30 Synthetic Spike: RT-PCR was performed using primers in Table 2 on gRNA of SARS-CoV-2 (Twist BioSciences, #1) for construction of a S2 spike-in DNA template. RT PCR (FP_1, R) and a second round of PCR (FP_2, R) was performed on HEK293T lysate for construction of a RPP30 spike-in DNA template. Products were run on a gel to identify specific products at −150 bp. DNA was purified using Ampure beads (Axygen) using a ratio of 1.8 ratio of beads:sample volume. Mixture was vortexed and incubated for 5 minutes at room temperature. A magnet was used to bind beads for 1 minute, washed twice with 70% EtOH, beads were air-dried for 5 minutes, and then removed from the magnet and eluted in 100 μL of IDTE Buffer. Bead solution was placed back on the magnet and the eluate was removed after 1 minute. DNA was quantified by nanodrop (Denovix).

This prepared DNA template was used for standard HiScribe T7 in vitro transcription (NEB). IVT reactions prepared according to manufacturer's instructions using 300 ng of template DNA per 20 uL reaction with a 16 hour incubation at 37 degrees. IVT reactions were treated with DNAseI according to the manufacturer's instructions. RNA was purified with an RNA Clean & Concentrator-25 kit (Zymo Research) according to the manufacturer's instructions and eluted into water. RNA spike-in was quantified both by nanodrop and with a RNA screen tape kit for the TapeStation according to the manufacturer's instructions (Agilent) to verify the RNA was the correct size (˜133 nt).

TABLE 2

| SEQ ID NO: | S2_FP | TAATACGACTCACTATAGGGCTGGTGCTGCAGCTTATTATGTGG |
|---|---|---|
| 13 | | GTATAGAACAACCTAGGACTTTTCTATTAA |
| SEQ ID NO: | S2_RP | AACGTACACTTTGTTTCTGAGAGAGG |
| 14 | | |
| SEQ ID NO: | RPP30_FP_1 | CTGACCTGAAGGCTGACGCCGGACTTGTGGAGACAGC |
| 15 | | |
| SEQ ID NO: | RPP30_FP2 | TAATACGACTCACTATAGGGAGATTTGGACCTGCGAGCGGGTTC |
| 16 | | TGACCTGAAGGCTGA |
| SEQ ID NO: | RPP30_R | |
| 17 | | GGTTTTTCAATTTCCTGTTTCTTTTCCTTAAAGTCAACG |

One-Step RT-PCR: RT-PCR were performed using either the Luna® Universal One-Step RT-qPCR Kit (New England BioSciences E3005) or the TaqPath™ 1-Step RT-qPCR Master Mix (Thermofisher Scientific, A15300) with a reaction volume of 20 μL. Both kits were used according to the manufacturer's protocol. The final concentration of primers in the mastermix was 50 nM for RPP30 F and R primers and 400 nM for S2 F and R primers. Synthetic S2 RNA was added directly to mastermix at a copy number of 500 copies per reaction. Sample was loaded into a 20 μL reaction. All reactions were run on a 96- or 384-well format and thermocycler conditions were run according to the manufacturer's protocol. For purified RNA samples 40 cycles of PCR was performed. For unpurified samples, endpoint PCR for 50 cycles was performed.

Multiplex Library Preparation: After the RT-PCR reaction, samples were pooled using multichannel pipet or Integra Viaflow Benchtop liquid handler. 6 μL from each well was combined into a sterile reservoir and the entire volume was transferred into a 15 mL conical tube and vortexed. 100 uL of the total volume was transferred to a 1.7 mL eppendorf tube for a double-sided SPRI cleanup. Briefly, 50 μL of AmpureXP beads (A63880) were added to 100 μL of the pooled PCR volume and vortexed. After 5 minutes, a magnet was used to collect beads for 1 minute and supernatant transferred to a new eppendorf tube. An additional 130 μL of Ampure XP beads were added to the 150 uL of supernatant and vortexed. After an additional 5 minutes, the magnet was used to collect beads for 1 minute and the beads were washed twice with fresh 70% EtOH. DNA was eluted off the beads in 40 uL of giagen EB buffer. The magnet was used to collect beads for 1 minute and 33 uL of supernatant was transferred to a new tube.

Sequencing Protocol: Libraries were sequenced on either an Illumina MiSeq (2012) or Nextseq550. Prior to each MiSeq run, a bleach wash was performed using a sodium hypochlorite solution (Sigma Aldrich, 239305) according to Illumina protocols. The pooled and quantitated library was diluted to a concentration of 6 nM (based on Qubit 4 Fluorometer and Illumina's formula for conversion between ng/ul and nM) and was loaded on the sequencer at either 25 pM (MiSeq) or 1.5 pM (NextSeq). PhiX Control v3 (Illumina, FC-110-3001) was spiked into the library at an estimated 30-40% of the library. PhiX provides additional sequence diversity to Read 1, which assists with template registration and improves run and base quality.

For this application, the MiSeq requires 2 custom sequencing primer mixes, the Read1 primer mix and the i7 primer mix. Both mixes have a final concentration of 20 uM of primers (10 uM of each amplicon's sequencing primer). The NextSeq requires an additional sequencing primer mix, the i5 primer mix, which also has a final concentration of 20 uM. The MiSeq Reagent Kit v3 (150-cycle; MS-102-3001) is loaded with 30 uL of Read1 sequencing primer mix into reservoir 12 and 30 μL of the i7 sequencing primer primer mix into reservoir 13. The NextSeq 500/550 Mid Output Kit is loaded with 52 ul of Read1 sequencing primer mix into reservoir 20, 85 ul of i7 sequencing primer mix into reservoir 22, and 85 ul of i5 sequencing primer mix into reservoir 22. Index 1 and 2 are each 10 bp, and Read 1 is 26 bp.

Analysis: The bioinformatic analysis consists of standard conversion of BCL files into FASTQ sequencing files using Illumina's bcl2fastq software (v2.20.0.422). Demultiplexing and read counts per sample are performed using the custom software. Here read1 is matched to one of the three expected amplicons allowing for the possibility of a single nucleotide error in the amplicon sequence. The hamming distance is the number of positions at which the corresponding sequences are different from each other and is a commonly used measure of distance between sequences. Samples are demultiplexed using the two index reads in order to identify which sample the read originated from. Observed index reads are matched to the expected index sequences allowing for the possibility of a single nucleotide error in one or both of the index sequences. The set of three reads are discarded if both index1 and index2 have hamming distances greater than 1 from the expected index sequences. The count of reads for each amplicon and each sample is calculated. In this analysis a few custom scripts were written in R that rely on the ShortRead and stringdist packages for processing fastq files and calculating hamming distances between observed and expected amplicons and indices. This approach is very conservative and gave very low-level control of the sequencing analysis. However, continued development of the kallisto and bustools SwabSeq analysis tools can provide a more user-friendly and computationally efficient solution for other groups implementing SwabSeq.

Criteria for Classification of Purified Patient Samples: For the analytic pipeline, QC metrics for each type of specimen were developed. For purified RNA, each sample required that there are at least 10 reads are detected for RPP30 and that the sum of S2 and S2 synthetic spike-in reads exceeds 2,000 reads. If these conditions are not met, the sample is rerun one time and if there is a second fail a resample is requested. To determine if SARS-CoV-2 is present the ratio of S2 to S2 spike exceeds 0.003 is calculated. (adding 1 count to both S2 and S2 spike before calculating this ratio facilitates plotting the results on a logarithmic scale.) If the ratio is greater than 0.003 it is concluded that SARS-CoV-2 is detected for that sample and if it is less than or equal to 0.003 it is concluded that SARS-CoV-2 is not detected (FIG. 10, panels A, B, and C).

The same pair of primers will amplify both the S and S spike amplicons. Because the run is an endpoint assay, the primers will be the limiting reagent to continued amplification. In developing this assay, observed was that as S2 counts increase for a sample, the S2 spike counts will decrease (FIG. 8). At the very high viral loads, S2 spike read counts decreased to less than 1000 reads. Therefore, considering S2 and S2 spike together allowed the QC to call SARS-CoV-2 even at extremely high viral titers. Thus, accounting for the scenario where S2 spike counts are low because S2 amplicon counts are very high and the sample contains large amounts of SARS-CoV-2 RNA is also important (FIG. 8).

Therefore, since the S2 and S2 spike are derived from the same primer pair, it was required in the QC that the sum of S2 and S2 spike counts together exceeds 2000. For example, detection greater than 2000 S2 counts and 0 S2 spike counts this would certainly be a SARS-CoV-2 positive sample and would result: SARS-CoV-2 detected.

Analysis of index mis-assignment: Unique dual indices and amplicon specific indices were used to study index mis-assignment. In this scheme, each sample was assigned two unique indices for the S or Spike amplicon and two unique indices for the RPP30 amplicon for a total of four unique indices per sample. A count matrix with all possible pairwise combinations of each index pair (one i7 and one i5) was used to generate a matching matrix. The counts on the diagonal of the matching matrix correspond to real samples and counts off of the diagonal correspond to index swapping events. The extent of index mis-assignment for the i7 and i5 index was determined by taking the row and column sum, respectively, of the off-diagonal elements of the matching matrix. The observed rate of index swapping to wells with a known zero amount of viral RNA was computed by taking the mean of the viral S counts to spike ratio for those wells.

### Supplemental Results

Improving Limit of Detection Requires Minimizing Sources of Noise: One of the major challenges in running a highly sensitive molecular diagnostic assay is that even a single contaminant or source of noise can decrease the test's analytical sensitivity. In the process of developing SwabSeq, S2 reads from control samples in which no SARS-CoV-2 RNA was present was observed (FIG. 4, panel D). These reads are referred to as “no template control” (NTC) reads. A key part of SwabSeq optimization has been understanding and minimizing the sources of NTC reads in order to improve the limit of detection (LoD) of the assay. Two sources of NTC reads: molecular contamination and mis-assignment sequencing reads were identified.

To minimize molecular contamination, protocols and procedures that are commonly used in molecular genetic diagnostic laboratories were followed. To limit molecular contamination, a dedicated hood for making dilutions of the synthetic RNA controls and master mix was used. At the start of each new run, the pipettes were sterilized, dilution solutions, and PCR plates with 10% bleach, followed by UV-light treatment for 15 minutes.

To prevent post-PCR products that are high concentration contaminating the pre-PCR processes, pre- and post-PCR steps were physically separated into two separate rooms, where any amplified plates were never opened within the pre-PCR laboratory space. To further protect from post-PCR contamination, RT-PCR mastermixes with or without Uracil-N-glycosylase (UNG) were compared. The presence of UNG in the TaqPath™ 1-Step RT-qPCR Master Mix (Thermofisher Scientific) showed a significant improvement reducing post-PCR contamination of S2 reads present in the negative patient samples as compared with the Luna One Step RT-PCR Mix (New England Biosciences) (FIG. 14). The RT-PCR mastermix contains a mix of dTTP and dUTP such that post-PCR amplicons are uracil containing DNA. These post-PCR that are remnants of previously run SwabSeq experiments therefore can be selectively eliminated by UNG.

A third source of molecular contamination was carryover contamination on the sequencer template line of the Illumina MiSeq. Without a bleach maintenance wash, indices that were run on the previous sequencing run were identified in an experiment where those indices were not included. While the number of reads for some indices were present at a number of S2 reads, the presence of carryover contamination affects the sensitivity and specificity of the assay. After an extra maintenance and bleach wash, the amount of carryover reads present to less than 10 reads was substantially reduced (FIG. 15).

Another source of NTC reads is mis-assignment of amplicons. Mis-assignment of amplicons occurs when sequencing (and perhaps at a lower rate, oligo synthesis) errors result in an amplicon sequence that originates from the S2 spike but is mistakenly assigned to the S2 sequence within a given sample. Only 6 bp distinguishes S2 from S2 spike at the beginning of read 1. Sequencing errors can result in S2 spike reads being misclassified as S2 reads as error rates appear to be higher in the beginning of the read (FIG. 16, panel A). If computational error correction of the amplicon reads is too tolerant, these reads may be inadvertently counted to the wrong category. To reduce this source of S2 read misassignment, a more conservative thresholding on edit distance was used (FIG. 16, panel B). Future redesigns or extensions to additional viral amplicons should consider engineering longer regions of sequence diversity here.

An additional source of NTC reads is when S2 amplicon reads are mis-assigned to the wrong sample based on the indexing strategy. In the assay, individual samples are identified by pairs of index reads (FIG. 4, panel B). Mis-assignment of samples to the wrong index could occur due to contamination of index primer sequences, synthesis errors in the index sequence, sequencing errors in the index sequences or “index hopping”.

Multiple indexing strategies in the development of SwabSeq were leveraged, from fully combinatorial indexing (where each possible combination of i5 and i7 indices are used to tag samples in the assay) to unique-dual indexing (UDI) where each sample has distinct and unrelated i7 and i5 indices (FIG. 17). However, the ability to scale can be limited due to the substantial upfront cost of developing that many unique primers. Fully combinatorial indexing approaches significantly expand the number of unique primer combinations. A compromise strategy between fully combinatorial indexing and UDI where sets of indices are only shared between small subsets of samples can also be used. Such designs reduce the effect of sample mis-assignment and facilitates scaling to tens of thousands of patient samples (FIG. 17). With a fully combinatorial indexing (FIG. 17) NTC read depth was correlated with the total number of S2 reads summed across all samples that shared the same i7 sequence (FIG. 18, panel A). This is consistent with the effect of indexing hopping from samples with high S2 viral reads to samples that shared the same indices. When positive samples are randomized across indices it is possible to computationally correct for this effect, for example using a linear mixed model.

Finally, the challenges associated with combinatorial and semi-combinatorial indexing strategies can be mitigated by using unique dual indexing (UDI), which is a strategy to reduce the number of index-hopped reads by two orders of magnitude. Consistently lower S2 viral reads for negative control samples UDI was observed. It also enables quantification of index mis-assignment by counting reads for index combinations that should not occur in the assay (FIG. 21, panels A and B). The number of index hopping events is correlated with the total number of S2+S2 spike reads (FIG. 21, panels C and D), indicating that hopped reads are more likely to come from wells where the expected index has strong viral signal. The overall rate of hopping as 1-2% was quantified on a MiSeq and is known to be higher on patterned flow cell instruments.

There are many sources of noise in amplicon-based sequencing, from environmental contamination in the RT-PCR and sequencing steps to misassignment of reads based on computational correction and “index-hopping” on the Illumina flow cells. Preventing and correcting these sources of error drastically improves the limit of detection of the SwabSeq assay.

### Example 5—Effect of Primer Concentration on SARS-CoV-2 Testing

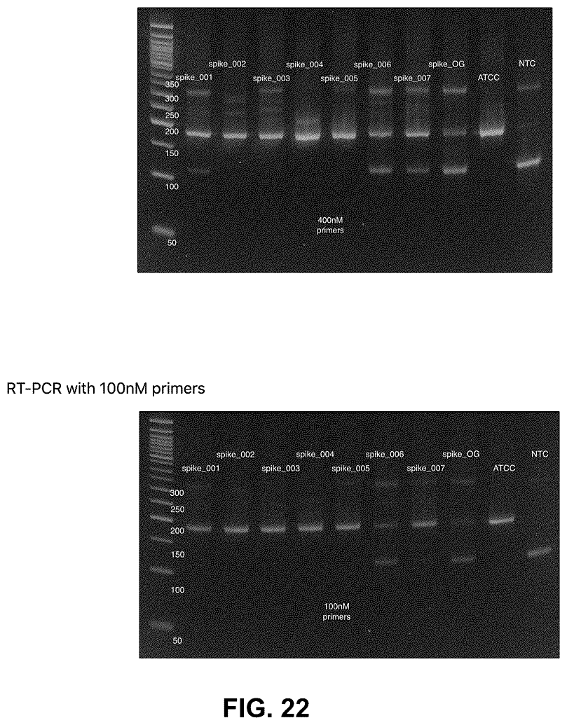

The effect of lowered primer concentrations was tested for the S2 and N1 amplicon. The concentration of primers was lowered from 400 nM to 100 nM in a 20 uL reaction using either saliva (diluted 1:1) or nasal swabs. The RPP30 amplicon primer concentration was the same at 50 nM. Lowering the primer concentration allows retention of quantitative ability. The lower primer concentrations also lead to less primer dimers and non-specific amplification products that take up sequencing reads. In the gels shown in FIG. 22 this primer dimer band was at ˜120 bp in the no template control (NTC) lanes. Each gel was loaded with the same amount of DNA. In the gel showing the RT-PCR done with 400 nM primer, it can see that this primer dimer band at 120 bp in the NTC lane is much brighter than in the gel showing the RT-PCR done with 100 nM primer. Using a 200 nM concentration for primers in the amplification reaction also showed marked improvement and reduction of primer dimers and non-specific products.

### Example 6—Diversified Synthetic Sequences

The methods described herein are improved by the presence of a synthetic nucleic acid sequence, that acts as an internal control sequence that is co-extracted, transcribed, and amplified with a sample.

### S2 Synthetic Nucleic Acid Sequences

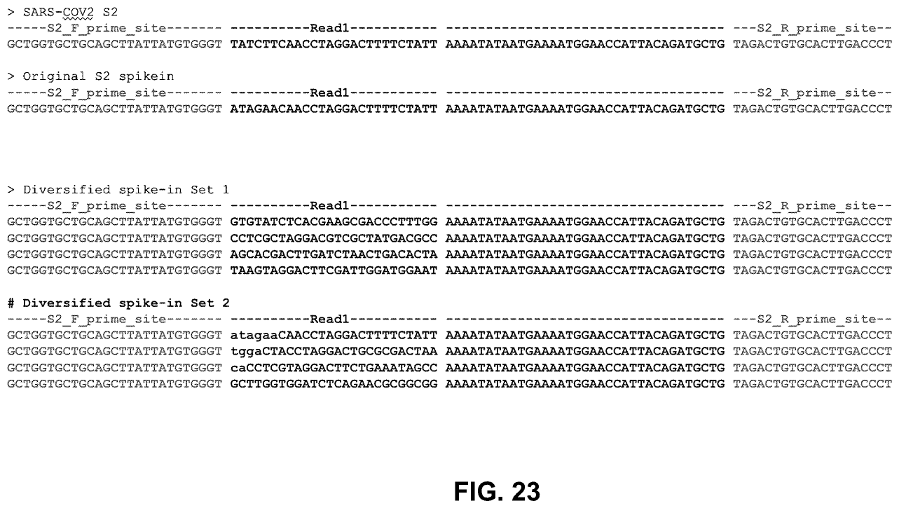

Four synthetic RNA S2 oligos were designed to increase nucleotide base diversity during sequencing which enables sufficient base diversity to ensure robust base calling during sequencing without the need for PhiX. The synthetic RNA oligos were pooled together in an equimolar fashion and spiked into the RT-PCR mastermix at a total concentration of 250 copies of RNA per reaction. Several combinations, but found that set #1 worked best. These sequences were derived from the wildtype S2 sequence with specific basepair changes to equally represent each of the four bases at each position (targeting 25% A, 25% T, 25% G, 25% C). Achieving equal distribution of nucleotides required sequence randomization, leading to final diversified S2 spike sequences that do not resemble the original template in the 26-bp read region. The sequences were optimized to reduce secondary structure and all have a similar melting temperature. The flanking sequences share homology with S2 amplicon. The LoD using this spike was as good, sequences shown in FIG. 23. Primers used for this analysis were forward GCTGGTGCTGCAGCTTATTATGTGGGT (SEQ ID NO: 18) and reverse AGGGTCAAGTGCACAGTCTA (SEQ ID NO: 19).

### N1 Synthetic Nucleic Acid Sequences

This was also apparent when using diversified synthetic sequences that could be amplified using N1 specific primers. These N1 sequences were amplified with 2019-nCoV_N1-F; GACCCCAAAATCAGCGAAAT (SEQ ID NO: 20) and 2019-nCoV_N1-R; TCTGGTTACTGCCAGTTGAATCTG (SEQ ID NO: 21).

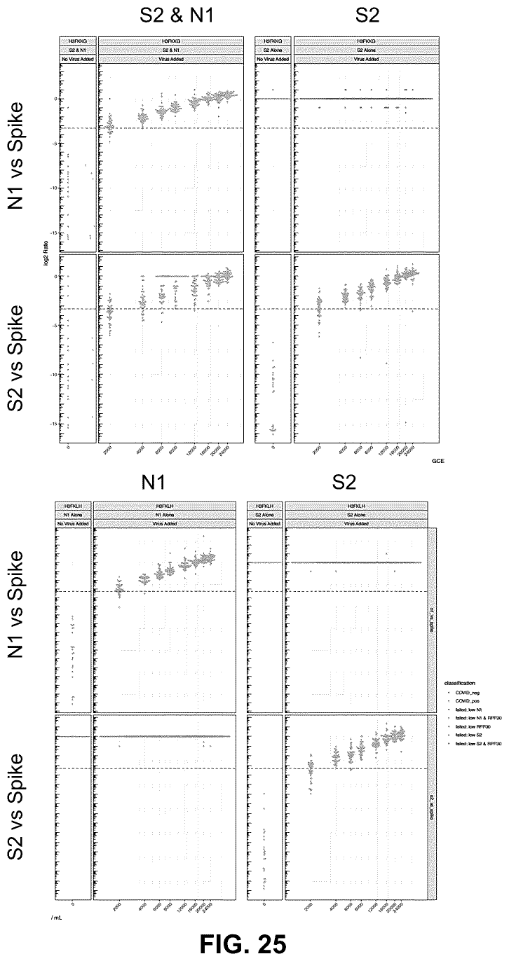

Preliminary limit of detection experiments were done with contrived samples (using ATCC heat inactivated SARS-CoV-2 standard) spiked into negative saliva and diluted serially. This was done using three primer conditions: both N1 and S2, just N1, and just S2. RPP30 was present at final reaction concentration of 50 nM, S2 at 100 nM, and N1 at 100 nM. The sensitivity of our assay with just S2 was determined to be 8000 GCE/mL. A preliminary LoD of N1 from this experiment is 6000 GCE/mL, and a preliminary LoD of N1+S2 is 4000 GCE/mL, indicating adding N1 improves the sensitivity of the SwabSEQ assay, as shown in FIG. 25.

The inclusion of the diversified synthetic sequences allowed for improvements in sensitivity and accuracy of the SwabSEQ test while reducing the reliance on PhiX during the sequencing process. Exemplary synthetic sequences are shown in table 3 below.

TABLE 3
Sequences for COVID 19 S2 and N1 synthetic spike sequences

| | |
|---|---|
| S2_001 | GCTGGTGCTGCAGCTTATTATGTGGGTGTGTATCTCACGAAGCGACCCTTTGGA |
| | AAATATAATGAAAATGGAACCATTACAGATGCTGTAGACTGTGCACTTGACCCT |
| S2_002 | GCTGGTGCTGCAGCTTATTATGTGGGTCCTCGCTAGGACGTCGCTATgacgccAAA |
| | ATATAATGAAAATGGAACCATTACAGATGCTGTAGACTGTGCACTTGACCCT |
| S2_003 | GCTGGTGCTGCAGCTTATTATGTGGGTAGCACGACTTGATCTAACTgacactaAAAA |
| | TATAATGAAAATGGAACCATTACAGATGCTGTAGACTGTGCACTTGACCCT |
| S2_004 | GCTGGTGCTGCAGCTTATTATGTGGGTTAAGTAGGACTTCGATTggaTggaatAAAA |
| | TATAATGAAAATGGAACCATTACAGATGCTGTAGACTGTGCACTTGACCCT |
| N1_001 | TCTGGTTACTGCCAGTTGAATCTGAGGGTCCGGACGGATATCGCACTAAGTGTA |
| | CCTGGTGCATTTCGCTGATTTTGGGGTC |
| N1_002 | TCTGGTTACTGCCAGTTGAATCTGAGGGTCCCATGACCATGTCACTGGCTACAC |
| | TGAGGTGCATTTCGCTGATTTTGGGGTC |
| N1_003 | TCTGGTTACTGCCAGTTGAATCTGAGGGTCCTTGACATGGCATGTGACTCCACT |
| | GTCGGTGCATTTCGCTGATTTTGGGGTC |
| N1_004 | TCTGGTTACTGCCAGTTGAATCTGAGGGTCCACCTTTGCCAGATGACTGAGTGG |
| | AAGGGTGCATTTCGCTGATTTTGGGGTC |
| N1_005 | TCTGGTTACTGCCAGTTGAATCTGAGGGTCCAGCTTGAAGCGTTCGCGACAAGT |
| | GTCGGTGCATTTCGCTGATTTTGGGGTC |
| N1_006 | TCTGGTTACTGCCAGTTGAATCTGAGGGTCCTCACGTCCTGAGATCAACTGCTA |
| | CATGGTGCATTTCGCTGATTTTGGGGTC |
| N1_007 | TCTGGTTACTGCCAGTTGAATCTGAGGGTCCGTTGACTGATCACATGCTGCTCC |
| | ACGGGTGCATTTCGCTGATTTTGGGGTC |
| N1_008 | TCTGGTTACTGCCAGTTGAATCTGAGGGTCCCAGACAGTCATCGGATTGATGAG |
| | TGAGGTGCATTTCGCTGATTTTGGGGTC |

### Example 7—SwabSeq can Detect Multiple Different SARS-CoV-2 Amplicons

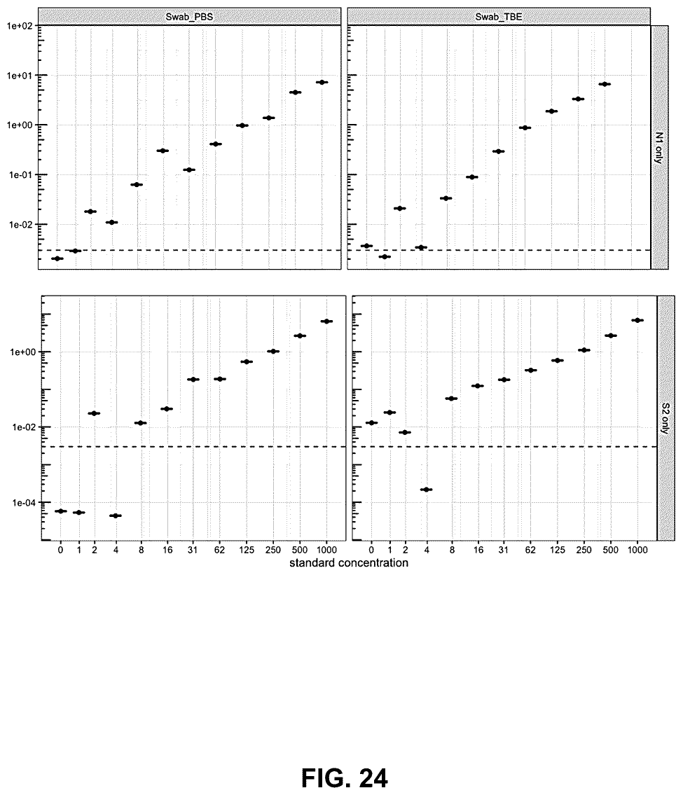

SwabSeq is able to detect two SARS-CoV-2 genes: Ni and S2 and synthetic Ni and S2 controls, increasing the ability to call positive samples. Data for each amplicon is shown in FIG. 24. N1 F: CAAGCAGAAGACGGCATACGAGAT XXXXXXXXXX ACCCCAAAATCAGCGAAAT (SEQ ID NO:22); Ni R: AATGATACGGCGACCACCGAGATCTACAC XXXXXXXXXX TCTGGTTACTGCCAGTTGAATCTG (SEQ ID NO:23); X is representative of the unique dual index (UDI) for each primer.

### Example 8—SwabSeq Performance with Different Samples

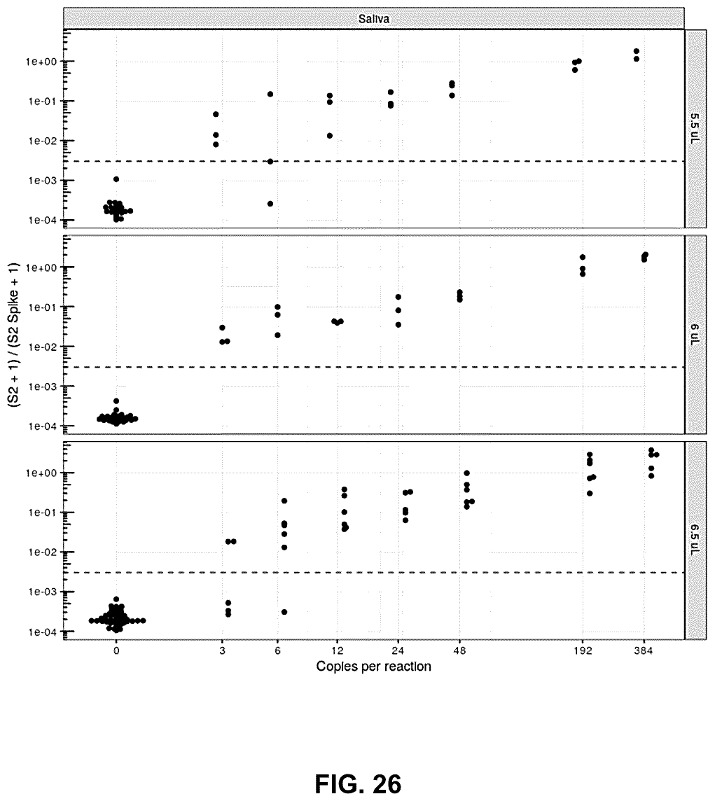

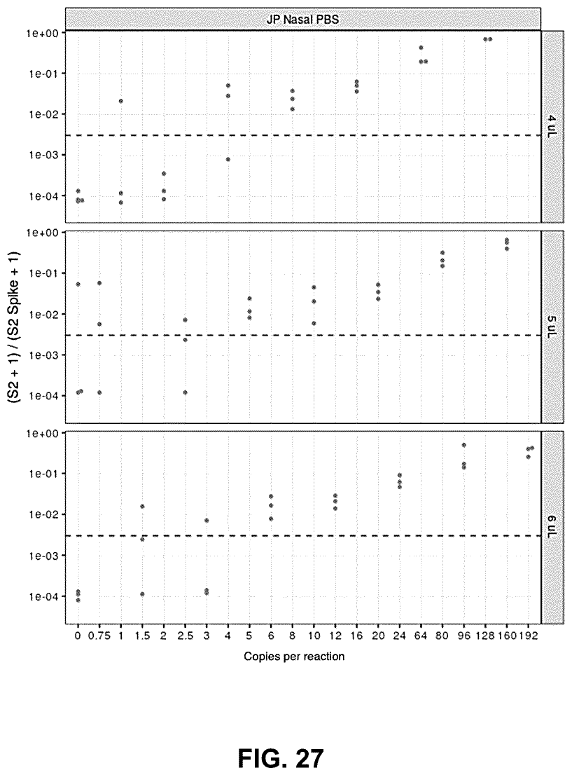

The ability to work from minimal or small samples is important for testing, especially if less invasive saliva samples can be used. FIG. 26 shows that up to 10 uL of saliva sample can be used during library preparation without inhibiting RT or PCR, and FIG. 27 shows that up to 6 uL of nasal swab sample (nasal swab inoculated into 1 mL of 1×PBS) can be used during library preparation without inhibiting RT or PCR.

### Example 9—SwabSeq can be Used to Detect Both Influenza Virus and SARS-CoV-2 Virus

Sequencing and detection systems and platforms that can detect multiple viruses simultaneously are advantageous. Experiments for codetection of SARS-CoV-2 and Influenza A and Influenza B were conducted.

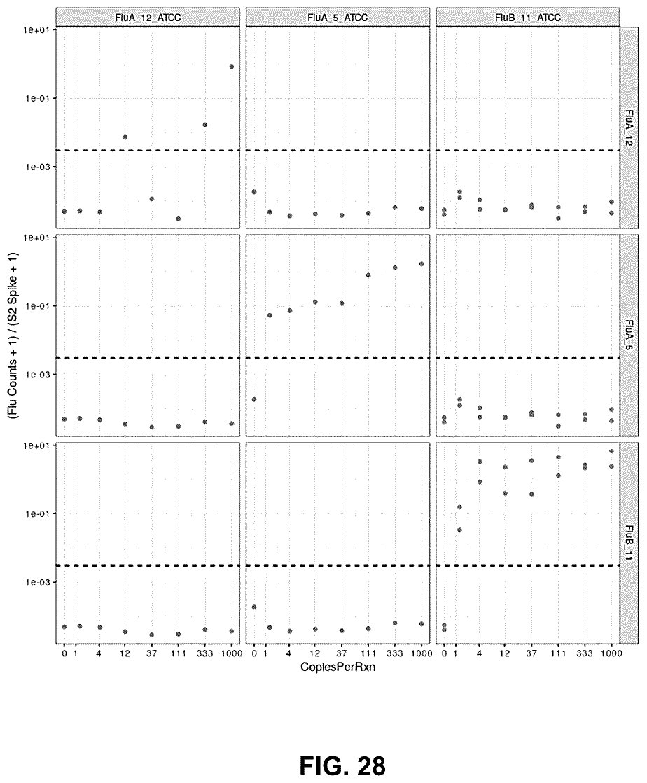

Specifically, the ability of SwabSeq to identify target amplicons for FluA MP, FluA NS and FluB NS. genome sequences were downloaded from the NCBI Influenza Database. Preliminary primers were then aligned to these sequences and the target amplification regions of each of these genomes were extracted. This information was then used to further optimize the primers to target as many of these amplicons as possible. All unique amplification regions were then exported and added to the target list. Results shown in FIG. 28 indicates that SwabSeq can detect influenza A or B in contrived samples.

To implement Swabseq for other respiratory pathogens, universal Influenza A and Influenza B Swabseq primers were designed. For Influenza A, 2 primer pairs targeting Influenza A were tested M1 (FluA_5_f1 GACCAATCYTGTCACCTCTGAC (SEQ ID NO: 24), FluA_5_f2 GACCAATYCTGTCACCTYTGAC (SEQ ID NO: 25), FluA_5_r AGGGCATTTTGGAYAAAGCGTCTA (SEQ ID NO: 26))0 and NS1 (FluA_12_f TTGGGGTCCTCATCGGAG (SEQ ID NO: 27), FluA_12_r TTCTCCAAGCGAATCTCTGTA, (SEQ ID NO: 28)) genes.

For Influenza B, a primer pair specific for the NS1 (FluB_11_f AAGATGGCCATCGGATCC, (SEQ ID NO: 29) FluB_11_r GTCTCCCTCTTCTGGTGATAATC, (SEQ ID NO: 30)) gene. Synthetic Nucleic Acid Spike sequences are shown below:

FluA_5_spike_004
(SEQ ID NO: 31)
GACCGATCCTGTCACCTCTGACTAAGGGTGGCACAACGCAGTGTGTTGAG
CTCCCTTAGTAGGGTGACTAGCACCGGCAGCGTAGACGCTTTGTCCAAAA
TGCCCT
FluA_12_spike_002
(SEQ ID NO: 32)
TTGGGGTCCTCATCGGAGGACGCTTAAATAATAGTAGGAACGTTCGAGTC
TCTAAAAATATACAGAGATTCGCTTGGAGAA
FluB_11_spike_004
(SEQ ID NO: 33)
AAGATGGCCATCGGATCCTCAACTCACTCCTGTAATGAGTCGTAGACAAG
GATAAGAGGCCCGATCGGCAGTATTTCGCCCTTTCTAAAAATAATGTGAC
CTGGGACGCACTGCACCGATTATCACCAGAAGAGGGAGAC

### Example 10—Inclusivity and Cross Reactivity of S2 Primers

Given the high rate of mutation of SARS-CoV-2 it is important that primers utilized in sequencing tests be able to amplify many different variants. In silico analysis of the S2 primer sets and S2 amplicon sequence were performed to evaluate the inclusivity of the SwabSeq test. For the primer analysis, SARS-CoV-2 sequences available on GISAID were evaluated. We first filtered out low quality genomes, which we defined as having greater than or equal to 1% unidentified nucleotides (N's). A BLASTn (NCBI) analysis was performed on the remaining 324,355 high quality genomes to quantify the level of primer homology across these sequences by querying each of the two SARS-CoV-2 primer sequences against the downloaded SARS-CoV-2 sequences. The analysis showed that 91.24% of all analyzed strains have 100% homology to both primer sequences and 28,398 strains (or 8.76%) of the 324,355 complete genomes have less than 100% homology to a primer. For the forward S2 primers, 303,840 (93.67%) GISAID genomes have 100% homology. For the reverse S2 primers, 305,019 (94.04%) GISAID genomes have 100% homology. The S2 amplicon is 26 base pairs and 303,934 (93.70%) GISAID genomes have 100% homology.

In silico analysis was performed to evaluate the cross-reactivity of the SwabSeq primers with representative common respiratory pathogens. For the primer analysis, 38 non-SARS-CoV-2 consensus genomes were downloaded from NCBI as the negative sample cohort. A BLASTn (NCBI) analysis was then performed to quantify the number of primer pairs with more than 80% homology with each of the genomes in the cohort. None of the pathogens exhibit greater than 80% homology with any of the primers.

### Example 11—Analysis of Sequencing Data

Presented herein is an example of an analysis platform the determination of infection with a pathogen, that can be combined with the extraction, amplification, and sequencing steps of SwabSeq.

First, all single nucleotide substitutions, deletions, and insertions for all targeted amplicons and barcode indices are generated. These are then used to as keys in a nested data structure that contains the original sequence, name of target, and type of match (exact match, mismatch, deletion, insertion). Since the keys in this data structure are indexed, exact string matching can be performed on sequencing data and the target information retrieved quickly within predetermined matching tolerances (single base substitutions, deletions, and insertions). All duplicated sequences are removed, and the match type of the remaining unique sequence is re-classified to “undetermined.”

The sequencing data are then extracted from the FASTQ files and matched with their corresponding dictionaries (index 1, index 2, or amplicons). Prior to matching, the instrument and kit information is read in to determine which sequences need to be reverse-complimented prior to matching.

The matching function returns either the original target sequence (used for the indices) or the name of the target (used for amplicons). This is a key step since many targets, including the diversified spikes and Influenza targets have several sequences that require aggregation together. Other data such as match type can be returned as well.

Matched sequences are then aggregated by their indices and amplicon target and a table of total reads per index pair is returned. This information is used downstream for quality control (can be used to determine carryover contamination, index hopping, failed amplification, etc.) as well as presence or absence of COVID-19 and Influenza A/B.

This information is returned as an automatically generated PDF that contains quality control plots as well as tables with information about each sample such as QC pass/fail, COVID-19 positive/negative, Influenza A/B positive/negative.

Dictionary-based exact matching of amplicons and barcodes enables fast analysis without depending on existing bioinformatics analysis libraries while accounting for all single base deletions, insertions and mismatches.

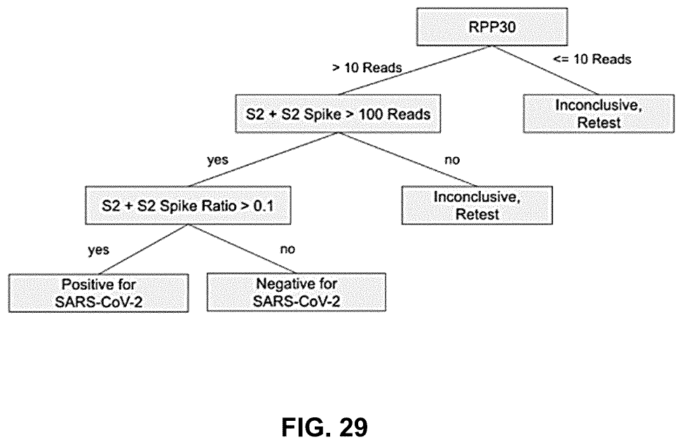

An example of an algorithm for calling positive samples when using the S2 primers, S2 synthetic nucleic acid and RPP30 primers is shown in table 4 below and FIG. 29 and requires an absolute number ofreads ofRPP30 amplicon, an absolute number of S2+S2 spike, and a ratio of S2/S2 spike greater than 0.1.

TABLE 4

| Well-controls | | Results | | | |
|---|---|---|---|---|---|
| Total | RPP30 | S2/S2 | | | |
| S2 + S2 | read | spike | | | |
| Spike | count | ratio | Result | Interpretation | Action |
| >100 | >10 | >0.1 | SARS- | Positive for SARS- | Report results to |
| | | | CoV-2 | CoV-2 for the Sample | physician, patient, and |
| | | | Detected | ID. | appropriate public health |
| | | | | | authorities. |
| >100 | >10 | <0.1 | SARS- | Negative for SARS- | Report results to |
| | | | CoV-2 | CoV-2 | physician, patient, and |
| | | | Not | for the Sample | appropriate public health |
| | | | Detected | ID. | authorities. |
| <100 | >10 | — | Inconclusive | Invalid for the | Quality control for the |
| | | | | Sample ID. | Sample ID is FAIL. |
| | | | | | Repeat sample or |
| | | | | | Recollect sample |
| <100 | <10 | — | Inconclusive | Invalid for the | Quality control for the |
| | | | | Sample ID. | Sample ID is FAIL. |
| | | | | | Repeat sample or |
| | | | | | Recollect sample |

### Example 12—Limit of Detection

A limit of detection (LoD) study was performed by spiking in heat inactivated SARS-CoV-2 virus (ATCC, VR-1986) in negative saliva specimens using a dilution series. Eleven extraction replicates were performed per concentration. The preliminary LoD was defined as the lowest concentration with 11 of 11 replicates that test positive. The LoD determination was carried out independently for the Luna® Probe One-Step RT-qPCR 4×Mix with UDG and the TaqPath™ 1-Step RT-qPCR Master Mix. For both RT-qPCR mastermixes, the preliminary LoD was determined to be 8,000 GCE/ml. See results in table 5 below.

TABLE 5
Preliminary LoD Determination Results

| Viral Concentration | Detection Rate (%) | Detection Rate (%) |
|---|---|---|
| (GCE/ml) | Luna ® | TaqPath ™ |
| 24,000 | 11/11 (100%) | 11/11 (100%) |
| 20,000 | 11/11 (100%) | 11/11 (100%) |
| 16,000 | 11/11 (100%) | 11/11 (100%) |
| 12,000 | 11/11 (100%) | 11/11 (100%) |
| 8,000 | 11/11 (100%) | 11/11 (100%) |
| 6,000 | 11/11 (100%) | 10/11 (90.9%) |
| 4,000 | 6/11 (54.5%) | 6/11 (54.5%) |
| 2,000 | 1/11 (9.1%) | 1/11 (9.1%) |

A limit of detection confirmation study for both the Luna® Probe One-Step RT-qPCR 4× Mix with UDG and the TaqPath™ 1-Step RT-qPCR Master Mix was performed by spiking in heat-inactivated SARS-CoV-2 virus (ATCC, VR-1986) in negative saliva specimens at a final concentration of 12,000 GCE/mL and 8,000 GCE/mL, corresponding to 1.5× and 1× the determined limit of detection. Ten (10) extraction replicates were performed for each concentration. The LoD confirmation at these two concentrations was carried out independently for the Luna© Probe One-Step RT-qPCR 4× Mix with UDG and the TaqPath™ 1-Step RT-qPCR Master Mix. The minimal LoD in both RT-qPCR mastermixes was confirmed to be 8,000 GCE/mL for both Luna® Probe One-Step RT-qPCR 4× Mix with UDG and the TaqPath™ 1-Step RT-qPCR Master Mix.

### Example 13—Clinical Evaluation

A study was performed to evaluate the performance of SwabSeq comparing saliva clinical samples that had previously been analyzed and confirmed as positive or negative using a laboratory-developed test (LDT).

Saliva samples were collected and run through using the MiniSeq Illumina sequencer. Results using the Luna Probe One-Step RT-qPCR 4× Mix with UDG are summarized in Table 6. Using Luna Probe One-Step RT-qPCR 4× Mix with UDG, the positive agreement (25/25) was 100% and negative agreement (93/93) was 100%. Results using Taqpath 1-Step RT-qPCR Master Mix are summarized in Table 7. The positive agreement (25/25) was 100% and negative agreement (93/93) was 100%.

TABLE 6
Evaluation with Clinical Specimens Using Luna ® Probe
One-Step RT-qPCR 4X Mix with UDG

| | | LDT Comparator Assay | | |
|---|---|---|---|---|
| | | Positive | Negative | Total |
| Ginkgo SARS-CoV-2 | Positive | 25 | 0 | 25 |
| NGS Test (Octant | Negative | 0 | 93 | 93 |
| v1) - Luna ® | Inconclusive | 0 | 0 | 0 |
| | Total | 25 | 93 | 118 |

| | |
|---|---|
| Positive Agreement | 100% (25/25); |
| | 82.4%-100%¹ |
| Negative Agreement | 100% (93/93); |
| | 96.1%-100%¹ |
| Overall Agreement | 100% (118/118); |
| | 96.9%-100%¹ |

¹Two-sided 95% score confidence intervals

TABLE 7
Evaluation with Clinical Specimens Using TaqPath ™ 1-
Step RT-qPCR Master Mix

| | | LDT Comparator Assay | | |
|---|---|---|---|---|
| | | Positive | Negative | Total |
| Ginkgo SARS-CoV-2 | Positive | 25 | 0 | 25 |
| NGS Test (Octant | Negative | 0 | 93 | 93 |
| v1) - TaqPath ™ | Inconclusive | 0 | 0 | 0 |
| | Total | 25 | 93 | 118 |

| | |
|---|---|
| Positive Agreement | 100% (25/25); |
| | 82.4%-100%¹ |
| Negative Agreement | 100% (93/93); |
| | 96.1%-100%¹ |
| Overall Agreement | 100% (118/118); |
| | 96.9%-100%¹ |

¹Two-sided 95% score confidence intervals

An intermediate precision study was performed on a second day using the same clinical samples to evaluate the reliability of SwabSeq. The person performing the test and instruments used in the test (Viaflo-96, manual pipette set, MiniSeq Sequencer, Thermal Cycler) used on the second day were all different than the first. Saliva clinical samples were then compared that had previously been analyzed and confirmed as positive or negative using a laboratory-developed test (LDT).

Results using SwabSeq are summarized in Table 8 and Table 9. Clinical samples analyzed using the Luna Probe One-Step RT-qPCR 4× Mix with UDG are summarized in Table 8. The positive agreement (25/25) was 1000% and negative agreement (93/93) was 100% o, and the concordance across the two intermediate precision runs was 10000. Clinical samples analyzed using Taqpath 1-Step RT-qPCR Master Mix are summarized in Table 9. The positive agreement (25/25) was 100% and negative agreement (93/93) was 100% o, and the concordance across the two intermediate precision runs was 100% o.

TABLE 8
Evaluation with Clinical Specimens Using Luna ® Probe
One-Step RT-qPCR 4X Mix with UDG

| | | LDT Comparator Assay | | |
|---|---|---|---|---|
| | | Positive | Negative | Total |
| Ginkgo SARS-CoV-2 | Positive | 25 | 0 | 25 |
| NGS Test (Octant | Negative | 0 | 93 | 93 |
| v1) - Luna ® | Inconclusive | 0 | 0 | 0 |
| | Total | 25 | 93 | 118 |

| | |
|---|---|
| Positive Agreement | 100% (25/25); |
| | 82.4%-100%¹ |
| Negative Agreement | 100% (93/93); |
| | 96.1%-100%¹ |
| Overall Agreement | 100% (118/118); |
| | 96.9%-100%¹ |

¹Two-sided 95% score confidence intervals

TABLE 9
Evaluation with Clinical Specimens Using TaqPath ™ 1-
Step RT-qPCR Master Mix

| | | LDT Comparator Assay | | |
|---|---|---|---|---|
| | | Positive | Negative | Total |
| Ginkgo SARS-CoV-2 | Positive | 25 | 0 | 25 |
| NGS Test (Octant | Negative | 0 | 93 | 93 |
| v1) - TaqPath ™ | Inconclusive | 0 | 0 | 0 |
| | Total | 25 | 93 | 118 |

| | |
|---|---|
| Positive Agreement | 100% (25/25); |
| | 82.4%-100%¹ |
| Negative Agreement | 100% (93/93); |
| | 96.1%-100%¹ |
| Overall Agreement | 100% (118/118); |
| | 96.9%-100%¹ |

¹Two-sided 95% score confidence intervals

The two intermediate precision runs performed using SwabSeq comparing saliva clinical samples that had previously been analyzed and confirmed as positive or negative show a 100% concordance.

### Example 14—Precision

To assess within run and between run precision, replicates at two contrived concentrations of virus/mL: 12,000 copies/mL and 24,000 copies/mL across 3 runs on the MiniSeq Sequencers were run. This was repeated so that both the Luna® Probe One-Step RT-qPCR 4× Mix with UDG (Table 10) and TaqPath™ 1-Step RT-qPCR Master Mix (Table 11) were used. These concentrations represent 1.5× and 3× the limit of detection of our assay. It was observed that the agreement of our third precision run using the Taqpath mastermix was not 100% (see Table 11). It was sometimes observed that contrived samples consisting of a viral RNA SARS-CoV-2 standard spiked into negative control saliva can degrade if not processed quickly enough. A fourth precision run for both Luna and Taqpath where run with contrived samples did not sit for so long at room temperature and found that there was 10000 agreement in the fourth run.

TABLE 10
Precision of Contrived Samples Using Luna ® Probe
One-Step RT-qPCR 4X Mix with UDG

| | Detection rate % Luna ® | | | | Between-run |
|---|---|---|---|---|---|
| copies/mL | Run1 | Run2 | Run3 | Run4 | Concordance |
| 12,000 | 6/6 | 6/6 | 6/6 | 6/6 | 24/24 |
| | (100%) | (100%) | (100%) | (100%) | (100%) |
| 24,000 | 6/6 | 6/6 | 6/6 | 6/6 | 24/24 |
| | (100%) | (100%) | (100%) | (100%) | (100%) |
| In-run | 12/12 | 12/12 | 12/12 | 12/12 | |
| Concordance | (100%) | (100%) | (100%) | (100%) | |

TABLE 11
Precision of Contrived Samples Using TaqPath ™ 1-
Step RT-qPCR Master Mix

| | Detection rate % TaqPath ™ | | | | Between Run |
|---|---|---|---|---|---|
| copies/mL | Run1 | Run2 | Run3 | Run4 | Concordance |
| 12,000 | 6/6 | 6/6 | 5/6 | 6/6 | 23/24 |
| | (100%) | (100%) | (83.3%) | (100%) | (95.8%) |
| 24,000 | 6/6 | 6/6 | 4/6 | 6/6 | 22/24 |
| | (100%) | (100%) | (66.6%) | (100%) | (91.6%) |
| In-run | 12/12 | 12/12 | 9/12 | 12/12 | |
| Concordance | (100%) | (100%) | (75%) | (100%) | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A method of detecting an influenza infection in an individual, the method comprising:
(a) providing a sample, wherein said sample comprises a biological sample from said individual and an influenza synthetic RNA, wherein a nucleic acid sequence of said influenza synthetic RNA differs from a naturally occurring influenza nucleic acid sequence;
(b) lysing said sample, thereby producing a lysed sample;
(c) performing a reverse transcription reaction on said lysed sample to obtain a lysed, reverse transcribed sample, wherein RNA from said lysed sample is not purified before performing said reverse transcription reaction;
(d) performing an amplification reaction on said lysed, reverse transcribed sample to obtain an amplified biological sample, wherein said amplification reaction on said lysed, reverse transcribed sample is performed with a set of influenza primers specific for an influenza nucleic acid sequence, wherein said set of influenza primers amplify said influenza nucleic acid sequence and said influenza synthetic RNA; and
(e) sequencing said amplified sample.

2. The method of claim 1, further comprising providing a positive diagnosis for influenza infection if sequence reads from said influenza nucleic acid sequence are detected.

3. The method of claim 1, wherein said influenza infection is an Influenza A infection.

4. The method of claim 1, wherein said influenza infection is an Influenza B infection.

5. The method of claim 2, wherein said positive diagnosis for said influenza infection is provided when a ratio or a mathematical equivalent thereof of said sequence reads from said influenza nucleic acid sequence to sequence reads of said influenza synthetic RNA exceed about 0.1.

6. The method of claim 2, wherein said positive diagnosis for influenza infection is provided when said sequence reads from said influenza nucleic acid sequence and said sequence reads of influenza synthetic RNA exceed a threshold amount of total sequence reads.

7. The method of claim 1, wherein the method further comprises detecting a coronavirus infection.

8. The method of claim 1, wherein lysing said sample comprises thermal lysis.

9. The method of claim 8, wherein said thermal lysis comprises heating said sample to a temperature of at least about 50° C.

10. The method of claim 1, wherein said sample from said individual comprises a plurality of influenza synthetic RNA sequences, wherein said plurality of influenza synthetic RNA sequences comprise at least two distinct synthetic influenza RNA sequences.

11. The method of claim 10, wherein said plurality of synthetic influenza RNA sequences comprise at least four distinct synthetic influenza RNA nucleic acid sequences.

12. The method of claim 1, wherein said amplification reaction on said lysed sample is further performed with a set of influenza primers specific for influenza nucleic acid sequence.

13. The method of claim 1, wherein said influenza synthetic RNA is present at a concentration from about 10 copies per/reaction to about 500 copies per reaction.

14. The method of claim 1, wherein sequencing said amplified sample comprises single molecule real-time sequencing, sequencing-by-synthesis, or ion semiconductor sequencing.

15. The method of claim 1, wherein sequencing of said amplified sample is not Sanger sequencing.

16. The method of claim 1, wherein sequencing said amplified sample generates at least 1 million sequence reads.

17. The method of claim 7, wherein the coronavirus infection comprises a Sars-Cov-2 infection.

18. The method of claim 1, wherein said amplification reaction on said lysed biological sample is performed with a primer pair specific for a sample control.

19. The method of claim 18, wherein said sample control is a housekeeping gene.

20. The method of claim 1, wherein said influenza synthetic RNA comprises an RNA sequence at least about 90% homologous to any one or more of the sequences set forth in any one of SEQ ID NOs: 31-33.
